(19) United States
(12) Patent Application Publication
(10) Pub. No.: US 2024/0361690 A1
(43) Pub. Date: Oct. 31, 2024

(54) Resist Material, Resist Composition, Patterning Process, And Monomer

(71) Applicant: SHIN-ETSU CHEMICAL CO., LTD., Tokyo (JP)

(72) Inventors: Jun HATAKEYAMA, Joetsu-shi (JP); Yutaro OTOMO, Joetsu-shi (JP); Tomomi WATANABE, Joetsu-shi (JP); Kazuhiro KATAYAMA, Joetsu-shi (JP)

(73) Assignee: SHIN-ETSU CHEMICAL CO., LTD., Tokyo (JP)

(21) Appl. No.: 18/635,999

(22) Filed: Apr. 15, 2024

(30) Foreign Application Priority Data
Apr. 19, 2023 (JP) 2023-68859
Oct. 27, 2023 (JP) 2023-184363

(51) Int. Cl. G03F 7/039; C07C 25/18; C07C 69/54; C07C 69/78; C07C 69/88; C07C 309/73; C07C 381/12; C07D 333/76; C08F 212/14; C08F 220/18; C08F 220/22

(52) U.S. Cl. CPC G03F 7/039; C07C 25/18; C07C 69/54; C07C 69/78; C07C 69/88; C07C 309/73; C07C 381/12; C07D 333/76; C08F 212/24; C08F 220/1806; C08F 220/1807; C08F 220/22

(57) ABSTRACT

The present invention is a resist material containing a repeating unit-a containing a sulfonium salt or iodonium salt of a monovalent aromatic carboxylic acid that is iodinated, is substituted or unsubstituted, and is bonded to a polymer main chain via an ester bond. This provides: a resist material having higher sensitivity and higher resolution than conventional positive resist materials, small edge roughness and size variation, and excellent pattern profile after exposure; a resist composition containing the resist material; a patterning process; and a monomer to be an ingredient for the resist material.

### TECHNICAL FIELD

The present invention relates to: a resist material; a resist composition; a patterning process; and a monomer.

### BACKGROUND ART

As LSIs advance toward higher integration and higher processing speed, miniaturization of pattern rule is progressing rapidly. This is because the spread of high-speed communication of 5 G and artificial intelligence (AI) has progressed, and high-performance devices for processing these are needed. As a cutting-edge technology for miniaturization, 5-nm node devices have been mass-produced by extreme ultraviolet ray (EUV) lithography at a wavelength of 13.5 nm. Furthermore, studies are also in progress on employing EUV lithography in next-generation 3-nm node and the following-generation 2-nm node devices.

As the miniaturization progresses, image blurs due to acid diffusion become a problem. To ensure resolution for fine patterns with dimensional sizes of 45 nm and smaller, there is a proposal that it is important to not only improve dissolution contrast as previously reported, but also control acid diffusion (Non Patent Document 1). Nevertheless, since chemically-amplified resist materials enhance the sensitivity and contrast through acid diffusion, an attempt to minimize acid diffusion by reducing the temperature and/or time of post-exposure bake (PEB) results in significant reductions of sensitivity and contrast.

A triangular tradeoff relationship among sensitivity, resolution, and edge roughness has been pointed out. Specifically, resolution improvement requires suppression of acid diffusion, whereas shortening acid diffusion distance results in the reduction of sensitivity.

The addition of an acid generator capable of generating a bulky acid is effective in suppressing acid diffusion. Hence, it has been proposed to incorporate in a polymer a repeating unit derived from an onium salt having a polymerizable unsaturated bond. In this case, the polymer also functions as an acid generator (polymer-bound acid generator). Patent Document 1 proposes a sulfonium and iodonium salt having a polymerizable unsaturated bond that generates a particular sulfonic acid. Patent Document 2 proposes a sulfonium salt having a sulfonic acid moiety directly bonded to the main chain.

For suppressing acid diffusion, proposed is a resist material containing a polymer-bound quencher containing, as a base polymer, a sulfonium salt of a weak acid having a pKa of −0.8 or higher and having a polymerizable group (Patent Documents 3 to 5). Patent Document 3 mentions, as weak acids, carboxylic acids, sulfonamides, phenols, hexafluoroalcohols, etc.

### CITATION LIST

### Patent Literature

Patent Document 1: JP 2006-045311 A
Patent Document 2: JP 2006-178317 A
Patent Document 3: WO 2019/167737 A1
Patent Document 4: WO 2022/264845 A1
Patent Document 5: JP 2022-115072 A

### Non Patent Literature

Non Patent Document 1: SPIE Vol. 6520 65203L-1 (2007)

### SUMMARY OF INVENTION

### Technical Problem

The present invention has been made in view of the above-described circumstances. An object of the present invention is to provide: a resist material having higher sensitivity and higher resolution than conventional positive resist materials, small edge roughness and size variation, and excellent pattern profile after exposure; a resist composition containing the resist material; a patterning process; and a monomer to be an ingredient for the resist material.

### Solution to Problem

To achieve the object, the present invention provides a resist material comprising a repeating unit-a containing a sulfonium salt or iodonium salt of a monovalent aromatic carboxylic acid that is iodinated, is substituted or unsubstituted, and is bonded to a polymer main chain via an ester bond.

Such a resist material has high sensitivity and high resolution exceeding those of conventional positive resist materials, and has small edge roughness, small size variation, and an excellent pattern profile after exposure.

In the present invention, the repeating unit-a preferably contains a repeating unit represented by the following general formula (a)-1 or (a)-2,

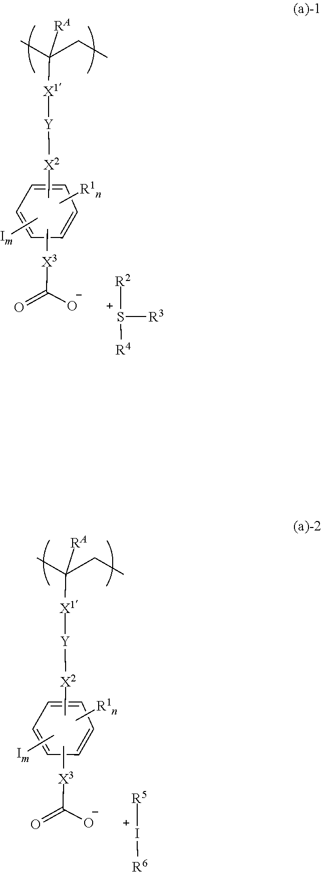

wherein R^{A} represents a hydrogen atom or a methyl group; X^{1′} represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group; X² represents a single bond or a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; Y represents a group selected from an ester group and a sulfonic acid ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

Such structures are preferable as the repeating unit-a.

In this event, in the general formulae (a)-1 and (a)-2, the X² preferably represents a linear, branched, or cyclic alkylene group having 1 to 12 carbon atoms and optionally containing one or more selected from an ester group, an ether group, an amide group, a lactone ring, a sultone ring, and a halogen atom.

Such structures are more preferable as the repeating unit-a.

In the present invention, the resist material preferably further comprises a repeating unit-b, in which a hydrogen atom of a carboxy group, a phenolic hydroxy group, or both is substituted with an acid-labile group.

Such a resist material has higher sensitivity and smaller variation in size.

In this event, the repeating unit-b is preferably at least one kind of repeating unit selected from repeating units represented by the following general formulae (b1) and (b2),

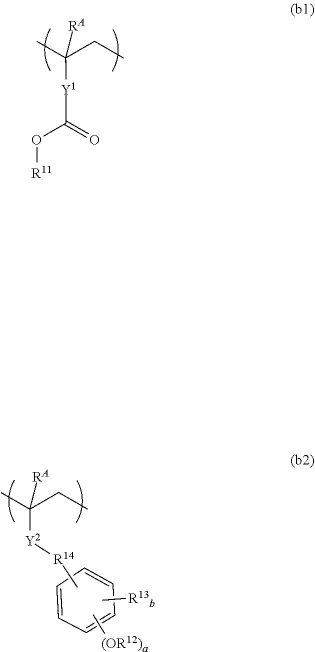

wherein each R^{A} independently represents a hydrogen atom or a methyl group; Y¹ represents a single bond, a phenylene group, a naphthylene group, or a linking group having 1 to 12 carbon atoms and having an ester bond, an ether bond, or a lactone ring; Y² represents a single bond, an ester bond, or an amide bond; R¹¹ and R¹² each represent an acid-labile group; R¹³ represents a fluorine atom, a trifluoromethyl group, a cyano group, or an alkyl group having 1 to 6 carbon atoms; R¹⁴ represents a single bond or a linear or branched alkanediyl group having 1 to 6 carbon atoms, part of carbon atoms of the alkanediyl group optionally being substituted with an ether bond or an ester bond; “a” represents 1 or 2; and “b” represents an integer of 0 to 4.

Such structures are preferable as the repeating unit-b.

In the present invention, the resist material preferably further comprises a repeating unit-c having an adhesive group selected from a hydroxy group, a carboxy group, a lactone ring, a carbonate group, a thiocarbonate group, a carbonyl group, a cyclic acetal group, an ether bond, an ester bond, a sulfonic acid ester bond, a cyano group, an amide group, —O—C(═O)—S—, and —O—C(═O)—NH—.

Such a resist material has excellent adhesiveness to a substrate.

In the present invention, the resist material preferably further comprises at least one kind of repeating unit-d selected from repeating units represented by the following general formulae (d1) to (d3),

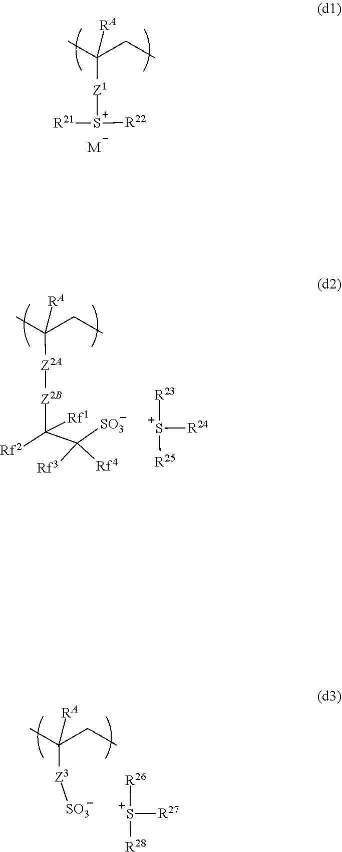

wherein each R^{A} independently represents a hydrogen atom or a methyl group; Z¹ represents a single bond, a phenylene group, —O—Z¹¹—, —C(═O)—O—Z¹¹—, or —C(═O)—NH—Z¹¹—; Z¹¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, and a hydroxy group; Z^{2A} represents a single bond or an ester bond; Z^{2B} represents a single bond or a divalent group having 1 to 12 carbon atoms and optionally containing one or more selected from an ester bond, an ether bond, a lactone ring, a bromine atom, and an iodine atom; Z³ represents a single bond, a methylene group, an ethylene group, a phenylene group, a fluorinated phenylene group, —O—Z³¹—, —C(═O)—O—Z³¹—, or —C(═O)—NH—Z³¹—; Z³¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, a halogen atom, and a hydroxy group; Rf¹ to Rf⁴ each independently represent a hydrogen atom, a fluorine atom, or a trifluoromethyl group, provided that at least one of Rf¹ to Rf⁴ is a fluorine atom; R²¹ to R²⁸ each independently represent a monovalent hydrocarbon group having 1 to 20 carbon atoms and optionally containing a heteroatom; any two of R²³, R²⁴, and R²⁵ or any two of R²⁶, R²⁷, and R²⁸ are optionally bonded to each other to form a ring together with a sulfur atom bonded thereto; and M represents a non-nucleophilic counter ion.

Such a resist material can reduce acid diffusion and prevent the degradation of resolution due to blurring caused by acid diffusion.

In this event, at least one iodine atom is preferably contained in the Z^{2B}.

Such a structure is more preferable as the repeating unit-d.

In the present invention, the resist material preferably has a molecular weight of 1,000 to 100,000.

Such a resist material has excellent heat resistance, maintains alkali solubility, and does not cause a trailing phenomenon after pattern formation.

The present invention also provides a resist composition comprising the above-described resist material.

Such a resist composition has high sensitivity and high resolution exceeding those of conventional positive resist materials, and has small edge roughness, small size variation, and an excellent pattern profile after exposure.

In this event, the resist composition preferably further comprises one or more selected from an acid generator, an organic solvent, a quencher, and a surfactant.

Such additives can be contained in the inventive resist composition.

The present invention also provides a patterning process comprising the steps of:
forming a resist film on a substrate by using the above-described resist composition;
exposing the resist film to a high-energy beam; and
developing the exposed resist film by using a developer.

According to such a patterning process, a pattern having an excellent pattern profile can be formed.

In this event, the high-energy beam is preferably an i-line, a KrF excimer laser beam, an ArF excimer laser beam, an electron beam, or an extreme ultraviolet ray having a wavelength of 3 to 15 nm.

Such high-energy beams can be used in the inventive patterning process.

In addition, the present invention provides a monomer represented by the following general formula (a)-1M or (a)-2M,

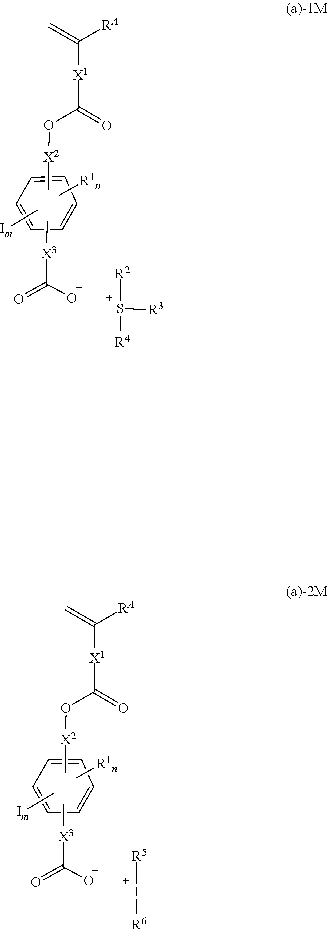

wherein R^{A} represents a hydrogen atom or a methyl group; X¹ represents a single bond, a phenylene group, a naphthylene group, or a linking group having 1 to 12 carbon atoms, including an ester bond or an ether bond, and optionally having a halogen atom; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

In addition, the present invention provides a monomer represented by the following general formula (a)-1′M or (a)-2′M,

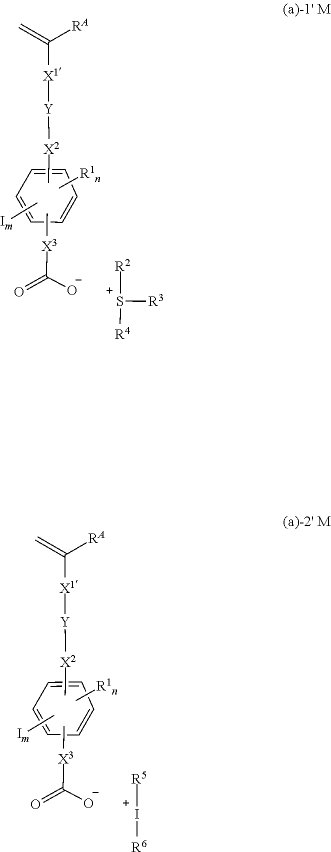

wherein R^{A} represents a hydrogen atom or a methyl group; X^{1′} represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; Y represents a group selected from an ester group and a sulfonic acid ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

Such a monomer can be used as an ingredient for a resist material that has high sensitivity and high resolution exceeding those of conventional positive resist materials, small edge roughness, small size variation, and an excellent pattern profile after exposure.

### Advantageous Effects of Invention

The inventive resist material can enhance the decomposition efficiency of an acid generator, and therefore, has a high effect of suppressing acid diffusion, has high sensitivity and high resolution, and allows excellent pattern profile and small and excellent edge roughness and size variation after exposure. The inventive resist material has such excellent properties, and therefore, has extremely high practicality, and in particular, is very useful for manufacturing very large-scale integrated circuits, and is very useful as a material for fine pattern formation of a photomask by EB writing or as a material for pattern formation for EB or EUV exposure. A positive resist material of the present invention can be applied to, for example, not only lithography in the formation of semiconductor circuits, but also for the formation of mask circuit patterns, micro-machines, and the formation of thin-film magnetic head circuits.

### BRIEF DESCRIPTION OF DRAWINGS

FIG. 1 is a figure showing a ¹H-NMR (500 MHZ, DMSO-d₆) spectrum of Compound 2 synthesized in Synthesis Example 1-1.

FIG. 2 is a figure showing a ¹H-NMR (500 MHZ, DMSO-d₆) spectrum of Compound 3 synthesized in Synthesis Example 1-1.

FIG. 3 is a figure showing a ¹H-NMR (500 MHz, DMSO-d₆) spectrum of Compound 4 synthesized in Synthesis Example 1-1.

FIG. 4 is a figure showing a ¹H-NMR (500 MHZ, DMSO-d₆) spectrum of Monomer 1 synthesized in Synthesis Example 1-1.

### DESCRIPTION OF EMBODIMENTS

As stated above, it has been desired to develop: a resist material having higher sensitivity and higher resolution than conventional positive resist materials, small edge roughness, small variation in size, and excellent pattern profile after exposure; a resist composition containing the resist material; a patterning process; and a monomer to be an ingredient for the resist material.

The present inventors have studied earnestly to achieve a positive resist material which has high resolution and small edge roughness (LWR) and size variation (CDU), desired in recent years, and found out that, to achieve this, it is necessary to minimize the acid diffusion distance and to realize a uniform acid diffusion distance at the molecular level. The present inventors have also found out that when a polymer including a repeating unit having a sulfonium salt or iodonium salt of a monovalent aromatic carboxylic acid that is iodinated, is substituted or unsubstituted, and is bonded to a polymer main chain via an ester bond is contained as a base polymer, acid diffusion is very small, and by the iodinated monovalent aromatic carboxylic acid generated by exposure having a low-swelling alkali dissolution property, the two effects of providing a uniform acid diffusion distance and providing low swelling make the polymer effective as a base polymer of a chemically-amplified resist material that allows excellent LWR and CDU.

The present inventors have further found out that, by introducing a repeating unit in which a hydrogen atom of a carboxy group or phenolic hydroxy group is substituted with an acid-labile group in order to enhance dissolution contrast, it is possible to obtain a resist material that has high sensitivity and a considerably high contrast in the alkali dissolution rate before and after exposure, a high effect of suppressing acid diffusion with high sensitivity, high resolution, small and excellent pattern profile, edge roughness, and size variation after exposure, and that is particularly suitable as a material for fine pattern formation for manufacturing very large-scale integrated circuits or for photomasks. Thus, the present invention has been completed.

That is, the present invention is a resist material comprising a repeating unit-a containing a sulfonium salt or iodonium salt of a monovalent aromatic carboxylic acid that is iodinated, is substituted or unsubstituted, and is bonded to a polymer main chain via an ester bond.

In addition, the present invention is a monomer represented by the following general formula (a)-1M or (a)-2M,

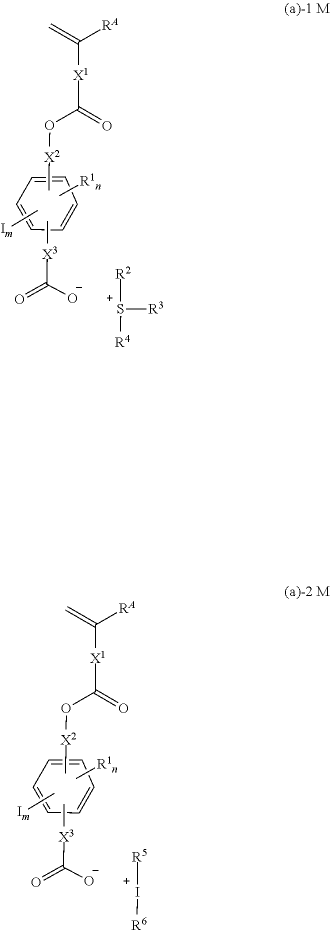

wherein R^{A} represents a hydrogen atom or a methyl group; X¹ represents a single bond, a phenylene group, a naphthylene group, or a linking group having 1 to 12 carbon atoms, including an ester bond or an ether bond, and optionally having a halogen atom; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

In addition, the present invention is a monomer represented by the following general formula (a)-1′M or (a)-2′M,

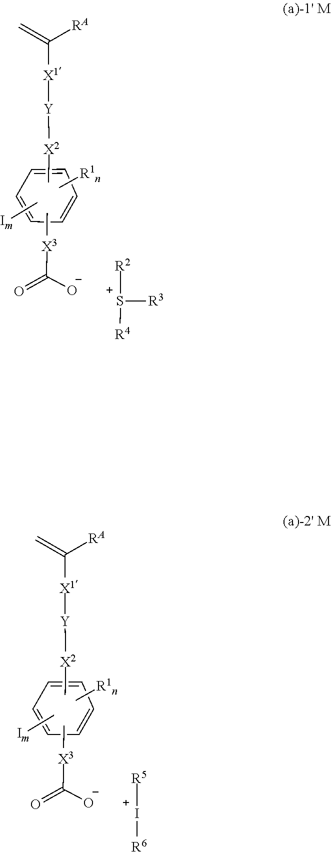

wherein R^{A} represents a hydrogen atom or a methyl group; X^{1′} represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; Y represents a group selected from an ester group and a sulfonic acid ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

Hereinafter, the present invention will be described in detail, but the present invention is not limited thereto.

### Monomer

The present invention is a monomer represented by the following general formula (a)-1M or (a)-2M.

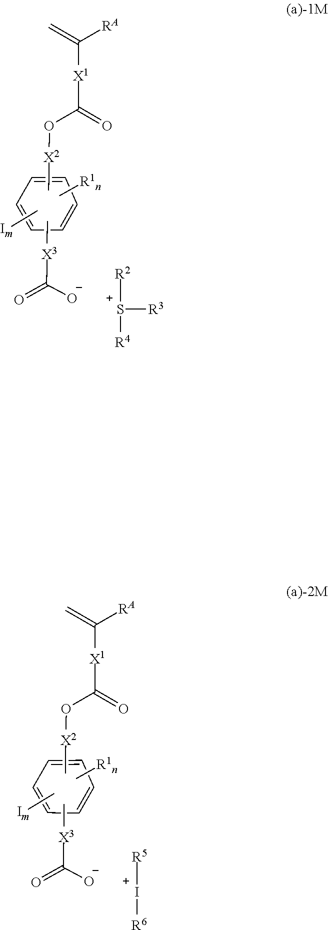

In the formulae, R^{A} represents a hydrogen atom or a methyl group; X¹ represents a single bond, a phenylene group, a naphthylene group, or a linking group having 1 to 12 carbon atoms, including an ester bond or an ether bond, and optionally having a halogen atom; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

In the formulae, R^{A} represents a hydrogen atom or a methyl group, preferably a methyl group. X¹ represents a single bond, a phenylene group, a naphthylene group, or a linking group having 1 to 12 carbon atoms, including an ester bond or an ether bond, and optionally having a halogen atom, preferably a single bond or a group including a phenylene group. X² represents a linear, branched, or cyclic alkylene group having 1 to 12 carbon atoms and optionally containing one or more selected from an ester group, an ether group, an amide group, a lactone ring, a sultone ring, and a halogen atom, preferably a group containing an ether group. X³ represents a single bond or a linear or branched alkylene group having 1 to 6 carbon atoms, containing no fluorine atom, and optionally having one or more selected from an ether group and an ester group, preferably a single bond, a methylene group, an ethylene group, or a propylene group. Each R¹ independently represents a linear or branched alkyl group, alkoxy group, or acyloxy group having 1 to 4 carbon atoms, or a halogen atom other than an iodine atom, preferably a fluorine atom. “m” represents an integer of 1 to 4, “n” represents an integer of 0 to 3, and preferably, “m” is 1 or 2 and “n” is 0 or 1. R² to R⁶ each independently represent a monovalent hydrocarbon group having 1 to 25 carbon atoms and optionally containing a heteroatom, preferably an aromatic hydrocarbon group, and more preferably a phenyl group, a naphthyl group, a biphenyl group, a phenyl sulfide group, a phenyl ether group, or a benzophenone group.

In addition, the present invention is a monomer represented by the following general formula (a)-1′M or (a)-2′M.

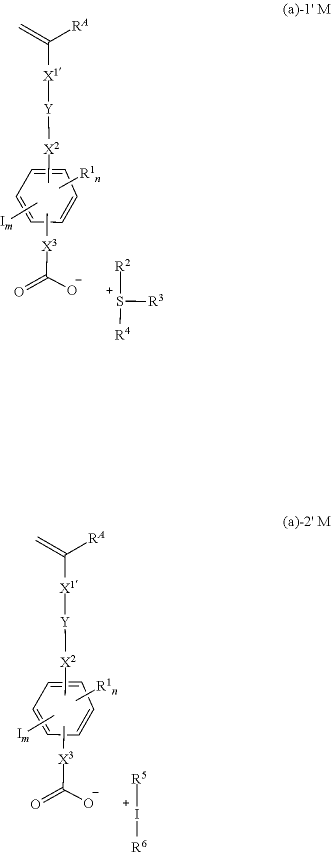

In the formulae, R^{A} represents a hydrogen atom or a methyl group; X^{1′} represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; Y represents a group selected from an ester group and a sulfonic acid ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

In the formulae, R^{A} represents a hydrogen atom or a methyl group, preferably a methyl group. X^{1′} represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group, preferably a single bond or a group containing a phenylene group. X² represents a linear, branched, or cyclic alkylene group having 1 to 12 carbon atoms and optionally containing one or more selected from an ester group, an ether group, an amide group, a lactone ring, a sultone ring, and a halogen atom, preferably a group containing an ether group. X³ represents a single bond or a linear or branched alkylene group having 1 to 6 carbon atoms, containing no fluorine atom, and optionally having one or more selected from an ether group and an ester group, preferably a single bond, a methylene group, an ethylene group, or a propylene group. Y represents a group selected from an ester group and a sulfonic acid ester group. Each R¹ independently represents a linear or branched alkyl group, alkoxy group, or acyloxy group having 1 to 4 carbon atoms, or a halogen atom other than an iodine atom, preferably a fluorine atom. “m” represents an integer of 1 to 4, “n” represents an integer of 0 to 3, and preferably, “m” is 1 or 2 and “n” is 0 or 1. R² to R⁶ each independently represent a monovalent hydrocarbon group having 1 to 25 carbon atoms and optionally containing a heteroatom, preferably an aromatic hydrocarbon group, and more preferably a phenyl group, a naphthyl group, a biphenyl group, a phenyl sulfide group, a phenyl ether group, or a benzophenone group.

### Resist Material

The inventive resist material contains a repeating unit-a containing a sulfonium salt or iodonium salt of a monovalent aromatic carboxylic acid that is iodinated, is substituted or unsubstituted, and is bonded to a polymer main chain via an ester bond. As the monovalent aromatic carboxylic acid, a benzoic acid is preferable. The repeating unit-a preferably includes a repeating unit represented by the following general formulae (a)-1 or (a)-2.

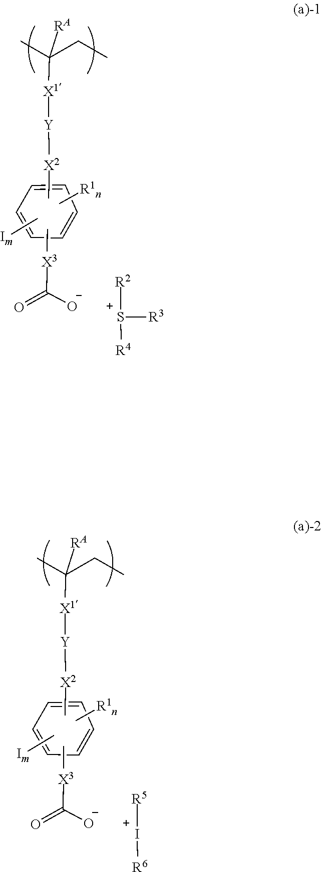

In the formulae, R^{A} represents a hydrogen atom or a methyl group; X^{1′} represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group; X² represents a single bond or a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; Y represents a group selected from an ester group and a sulfonic acid ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

In the general formulae (a)-1 and (a)-2, the X² preferably represents a linear, branched, or cyclic alkylene group having 1 to 12 carbon atoms and optionally containing one or more selected from an ester group, an ether group, an amide group, a lactone ring, a sultone ring, and a halogen atom. The presence of a structure other than a single bond in X² increases the flexibility of sulfonium salts and iodonium salts and their ability to quench acids.

The sulfonium or iodonium is dissociated by photodissociation to produce an iodinated monovalent aromatic carboxylic acid bonded to the polymer. Iodinated monovalent aromatic carboxylic acids have a characteristic that they hardly swell in an alkaline developer. The low swelling characteristic reduces degradation of critical dimension uniformity due to swelling during development of a contact hole pattern, and reduces stress applied to a pattern during spin-drying for drying a line-and-space pattern after rinsing with pure water. Thus, pattern collapse after pattern formation can be reduced.

The repeating unit-a is a quencher having the structure of a sulfonium salt or iodonium salt of a substituted or unsubstituted iodinated monovalent aromatic carboxylic acid, and is a quencher-bound polymer. Quencher-bound polymers have a high effect of suppressing acid diffusion, and as explained above, have a characteristic of having excellent resolution. This makes it possible to achieve high resolution, low LWR, and low CDU.

Examples of monomers to give a repeating unit-a1, represented by the general formula (a)-1, and a repeating unit-a2, represented by the general formula (a)-2, include the inventive monomer described above. Examples of the anion moiety of the monomer include the following, but are not limited thereto. Incidentally, in the following formulae, R^{A} is as defined above.

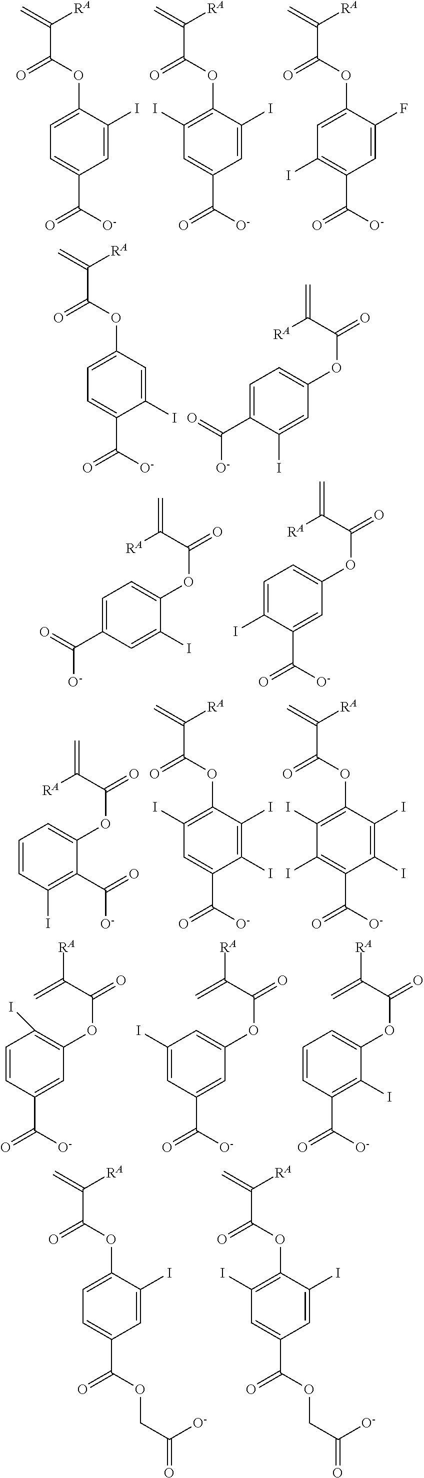

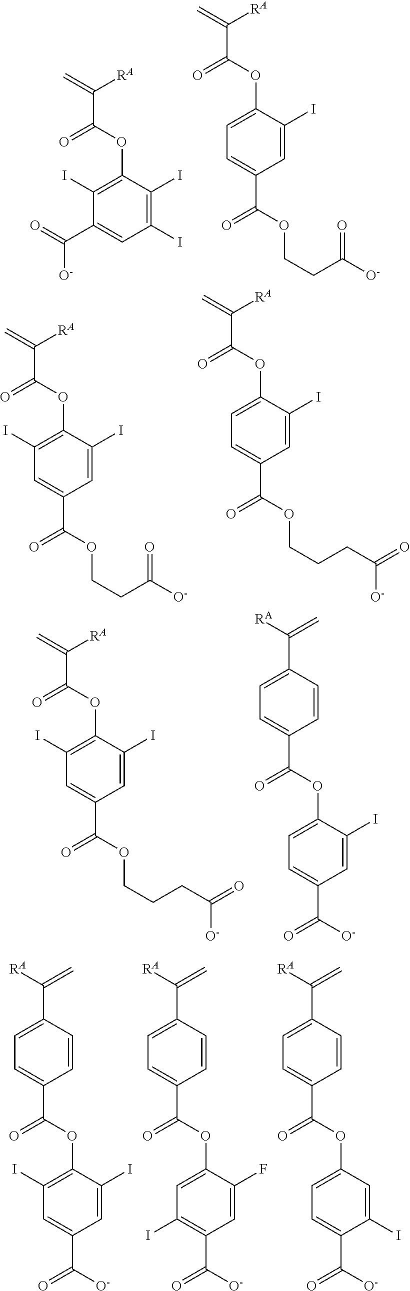

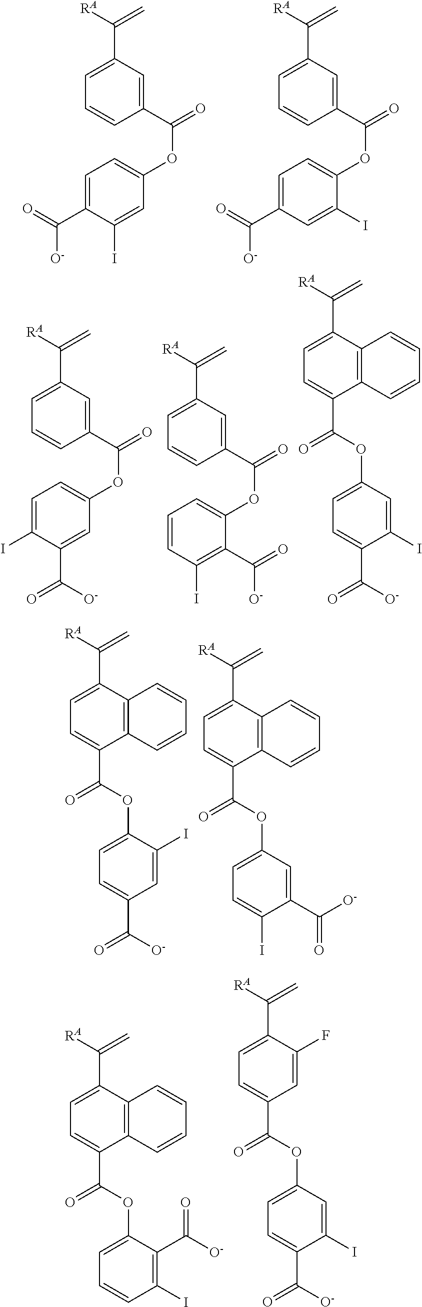

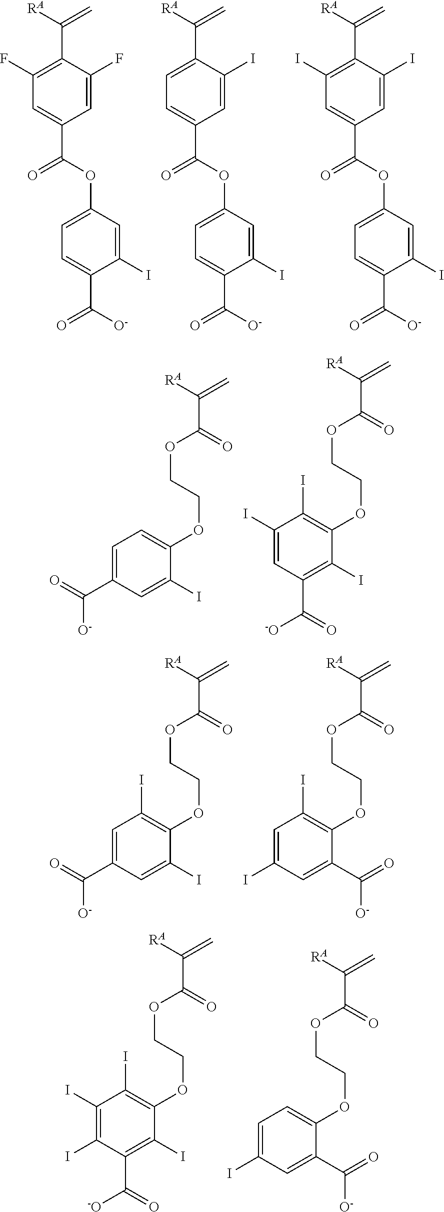

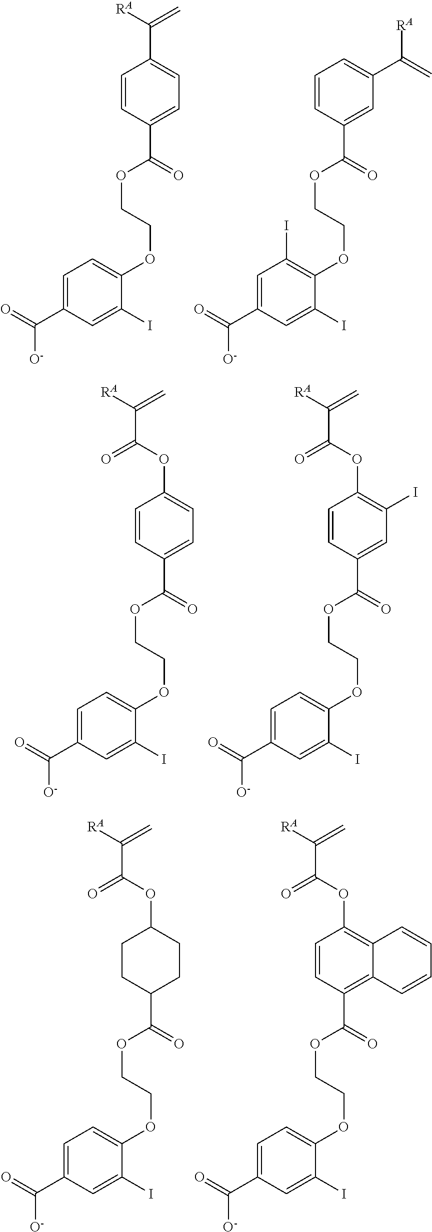

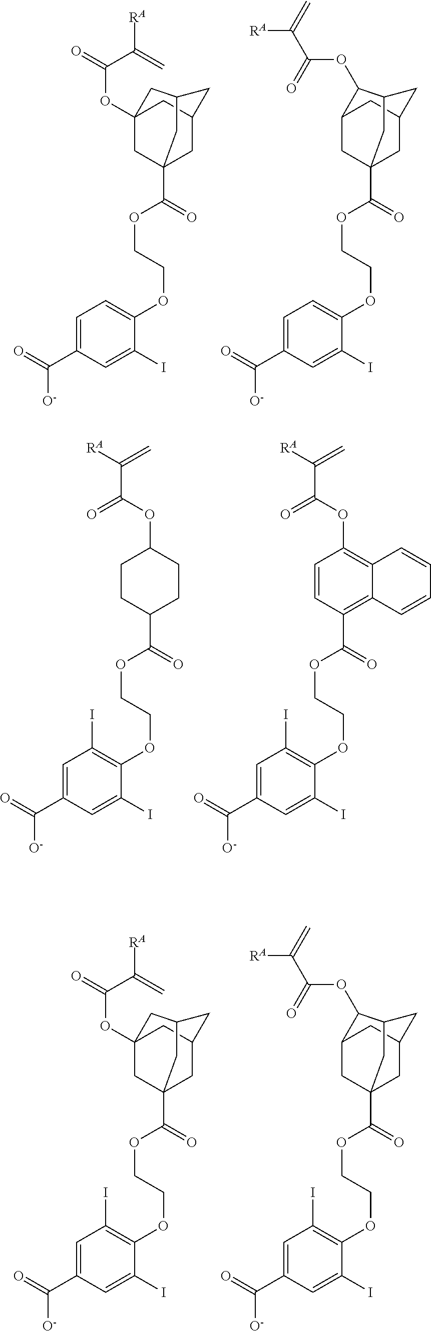

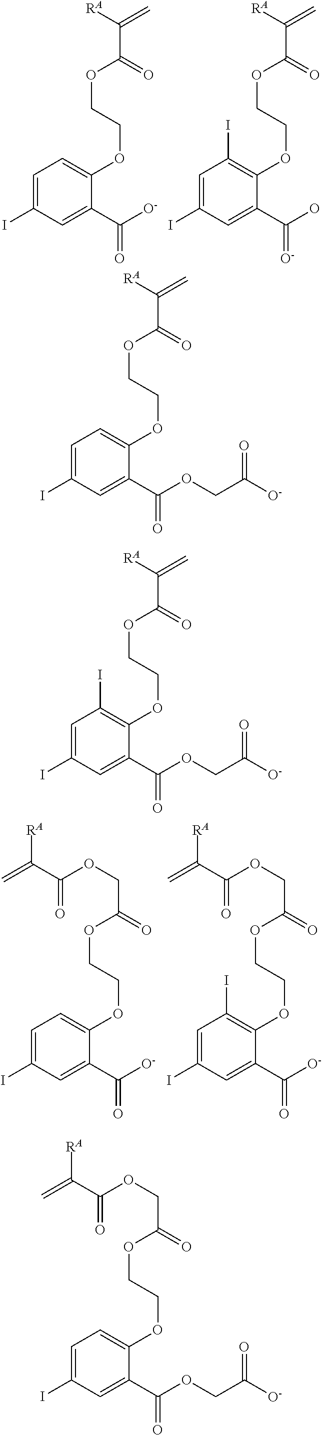

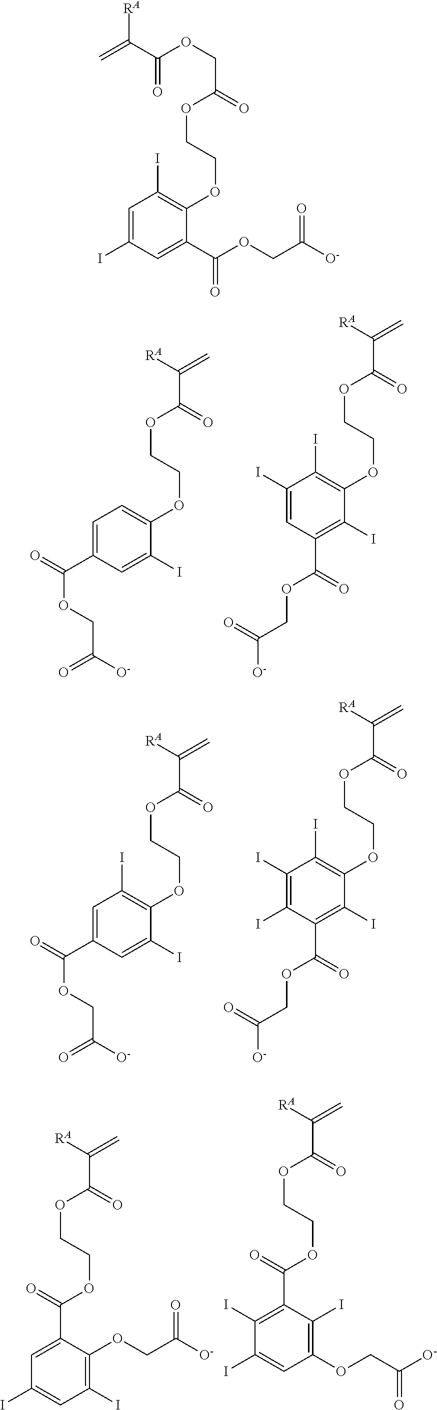

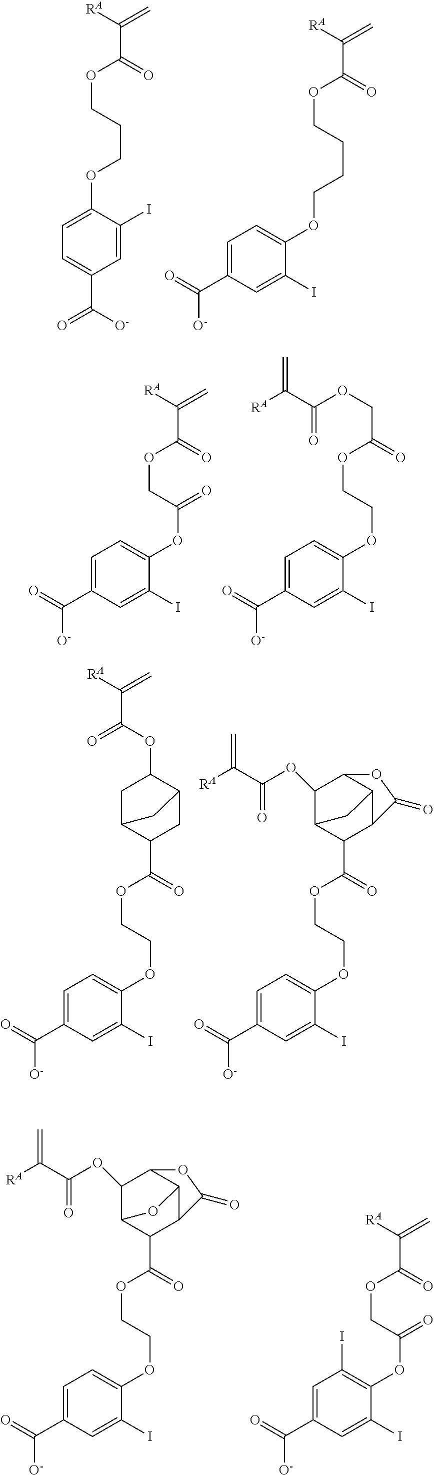

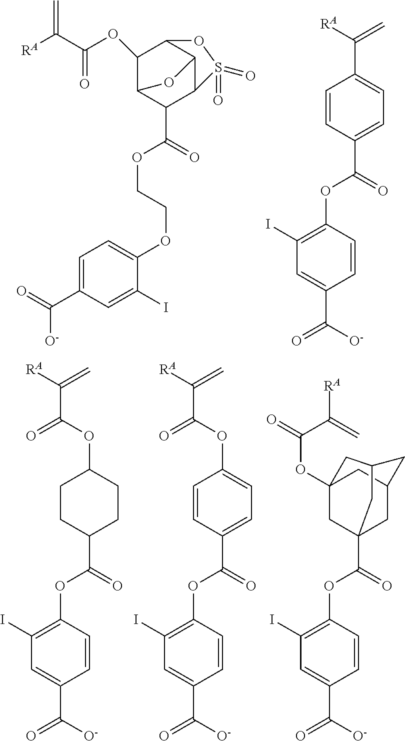

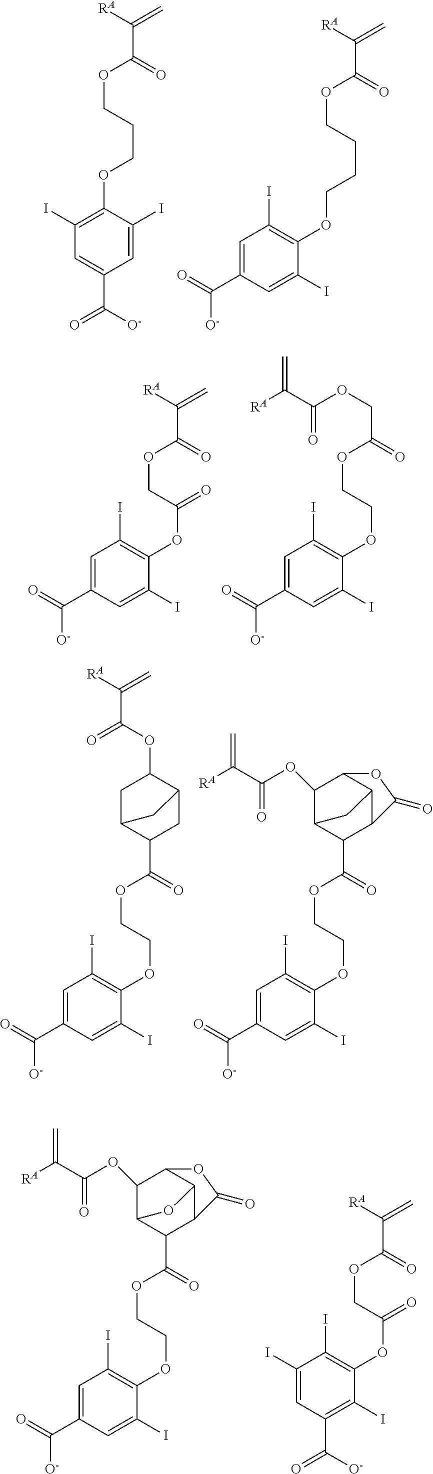

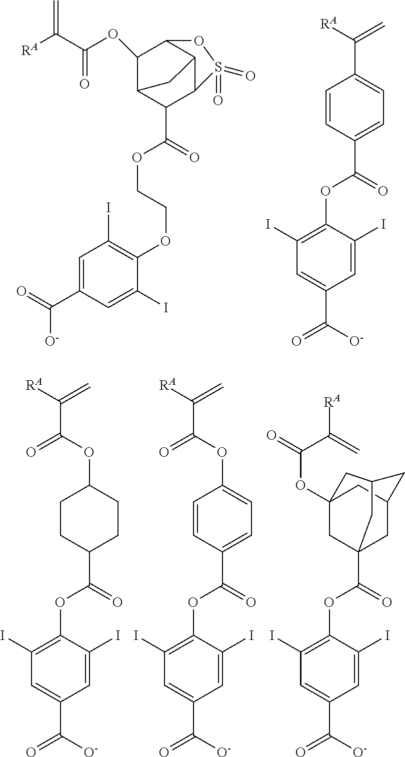

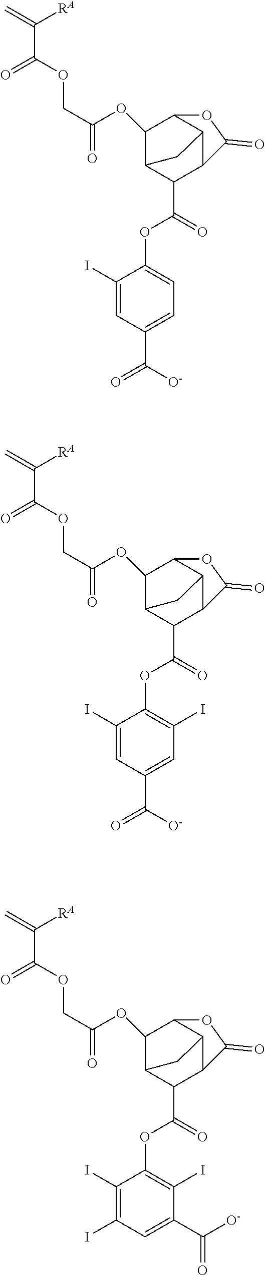

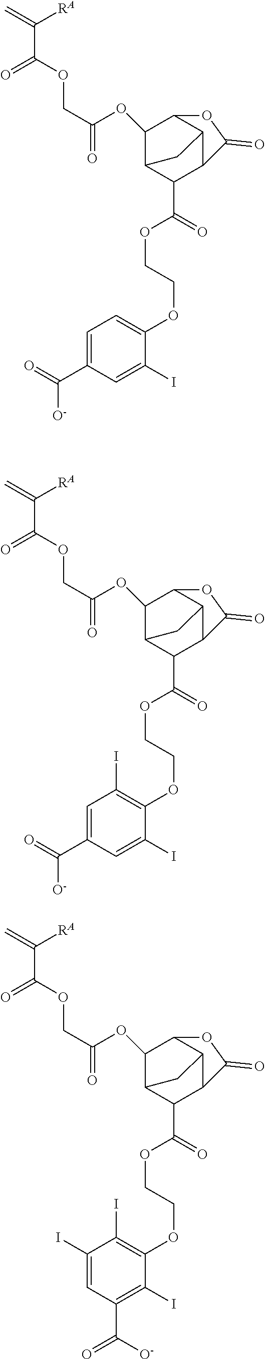

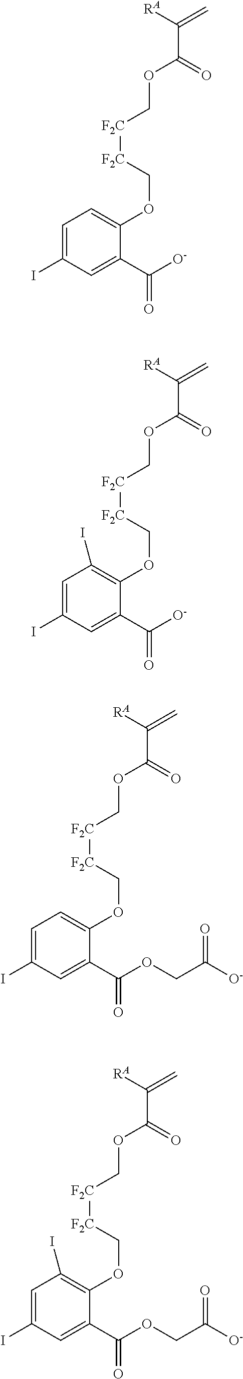

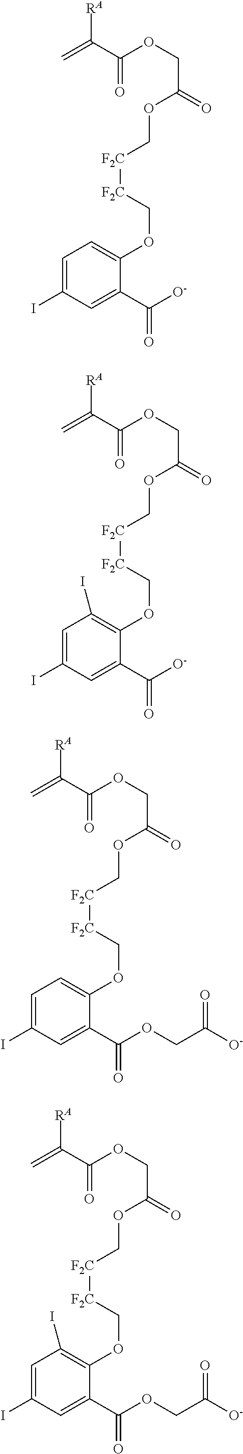

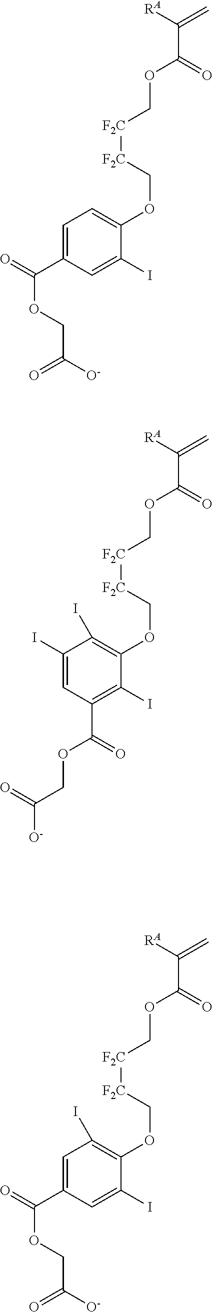

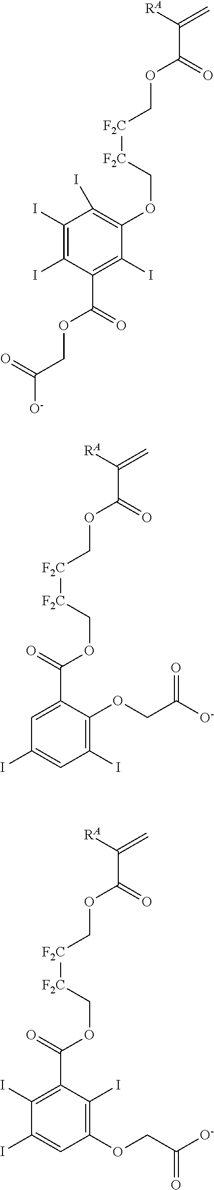

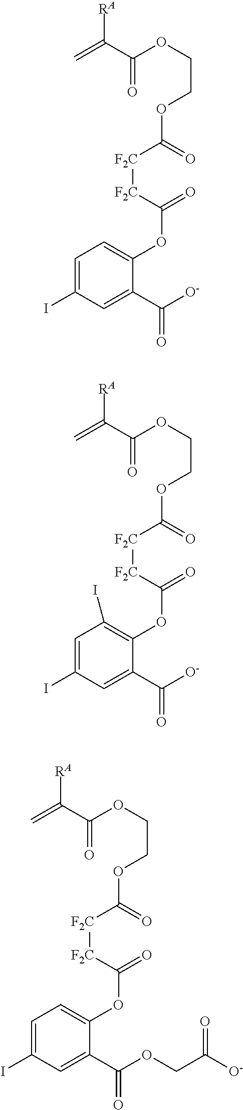

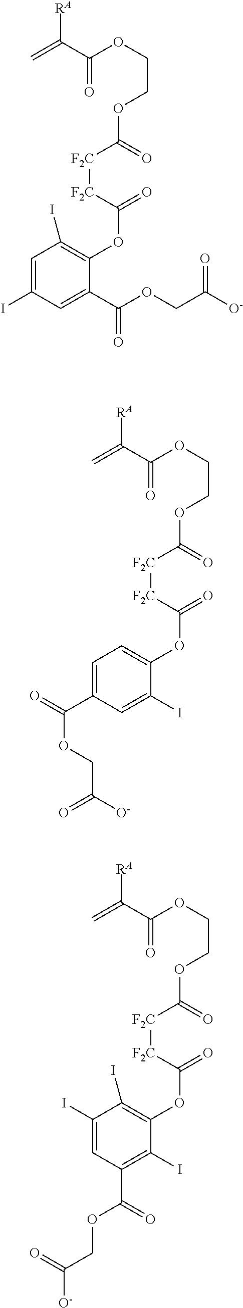

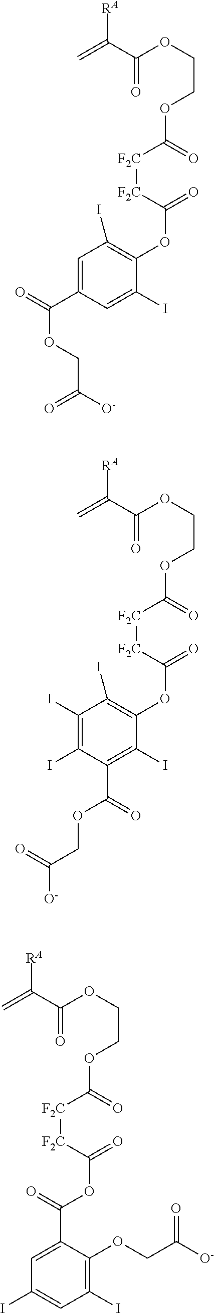

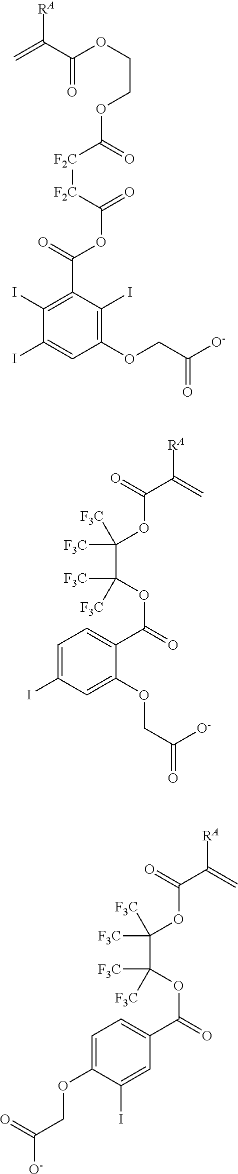

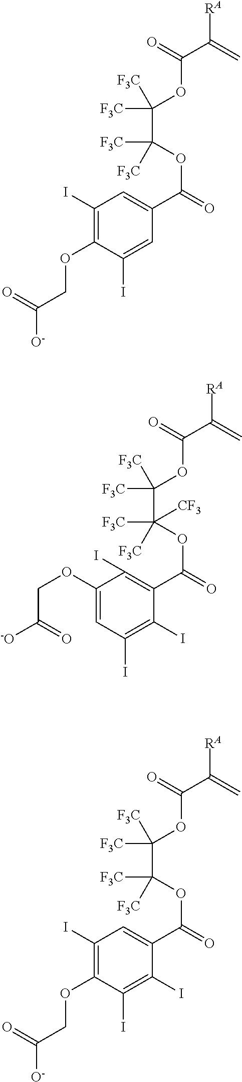

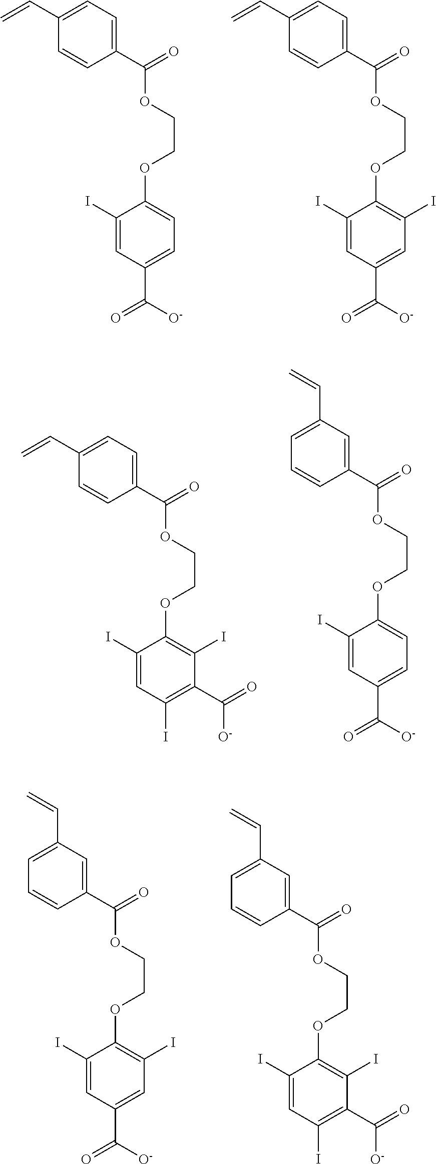

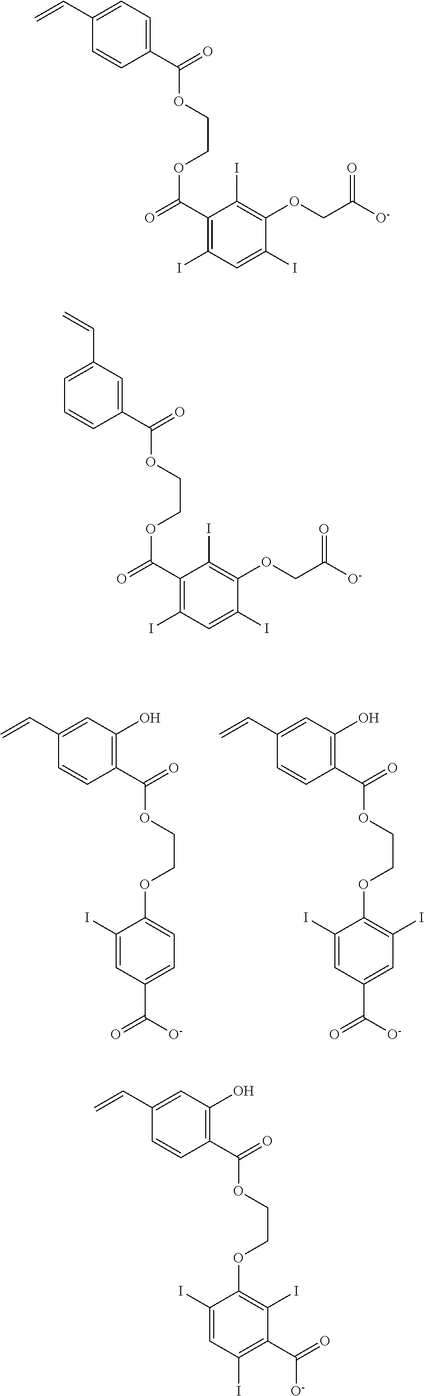

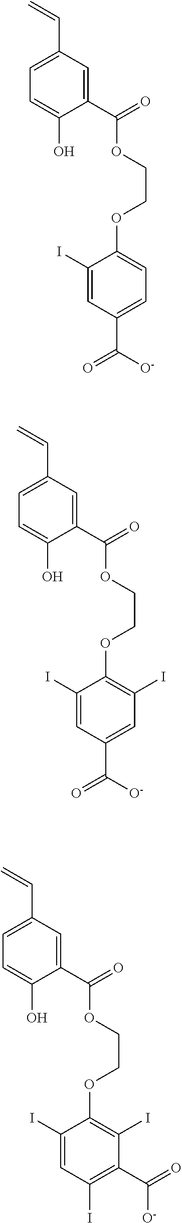

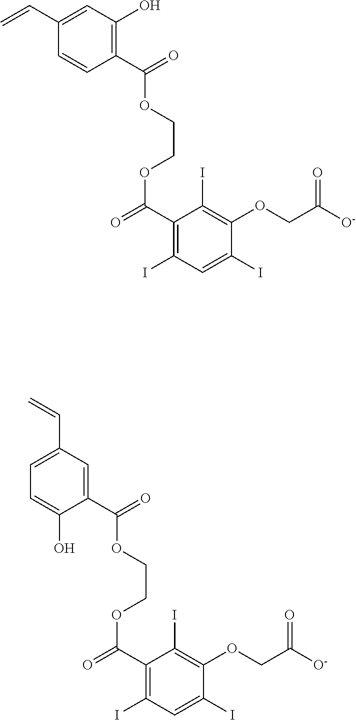

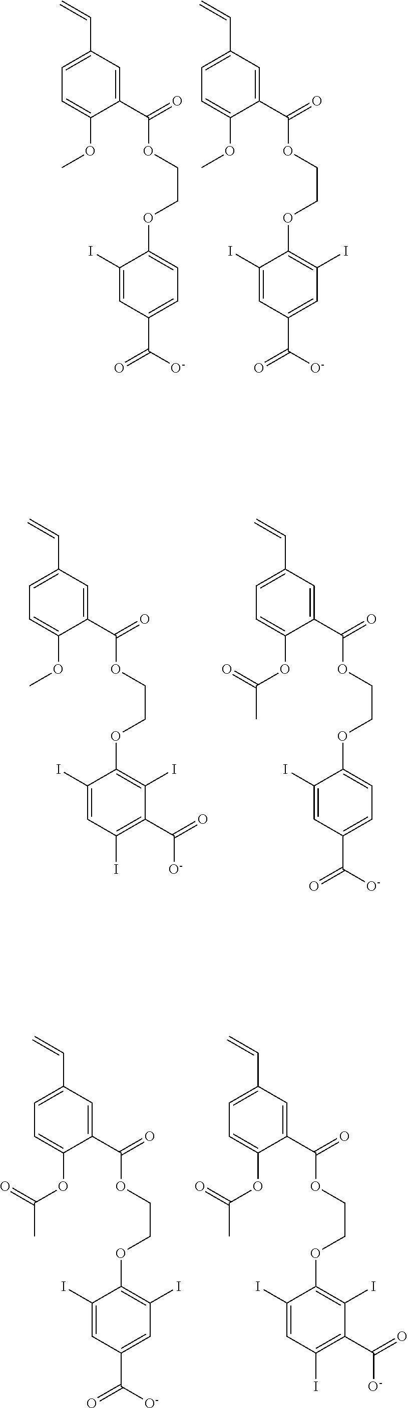

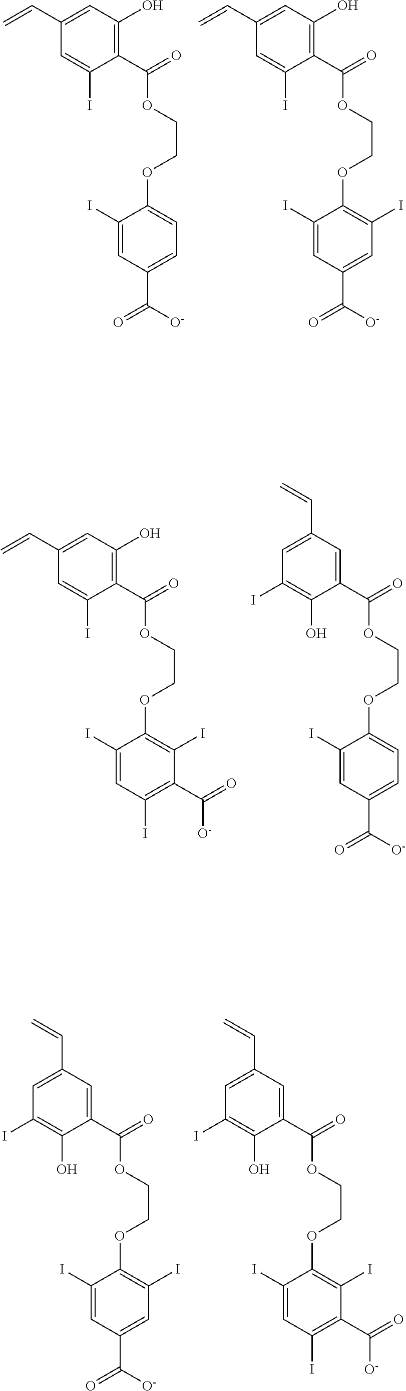

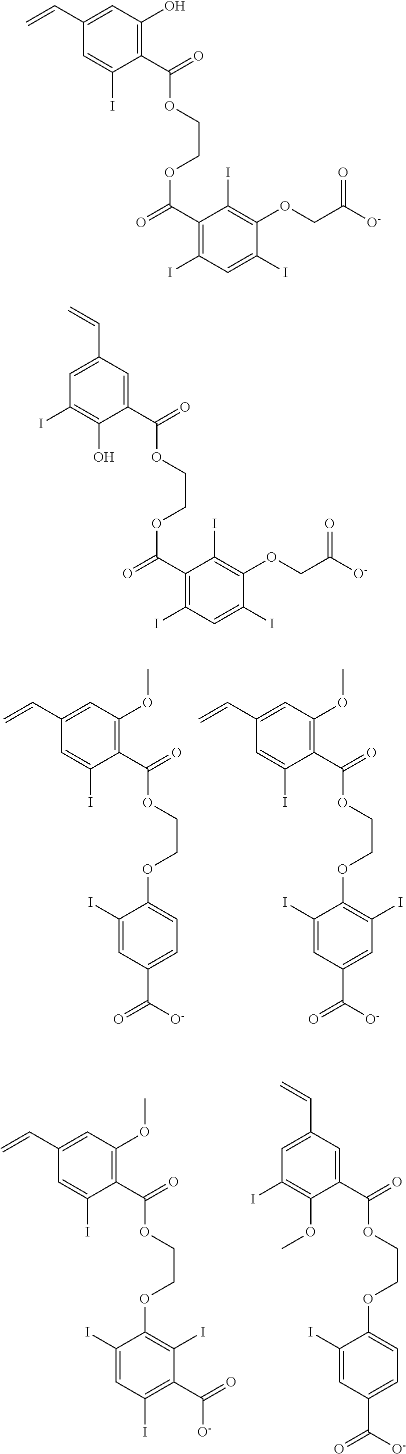

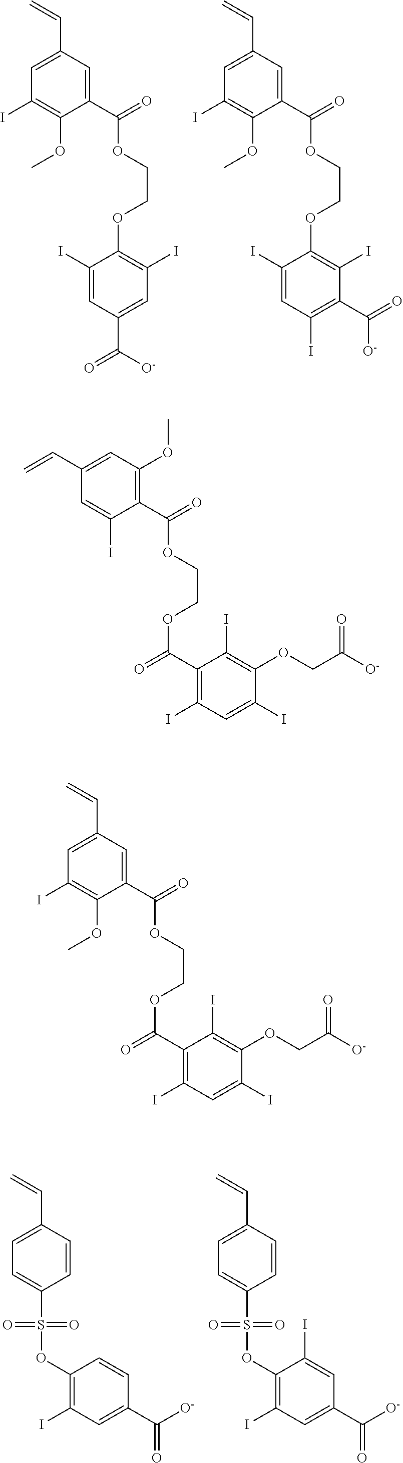

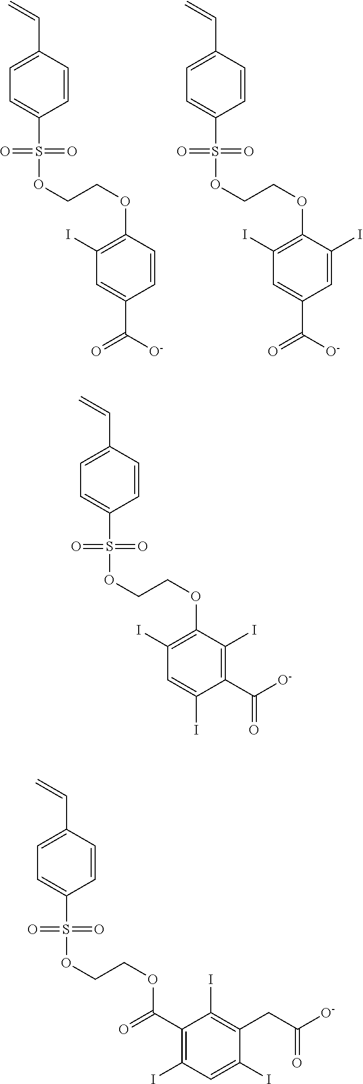

Examples of the cation of the sulfonium salt represented by the general formula (a)-1 include the following, but are not limited thereto.

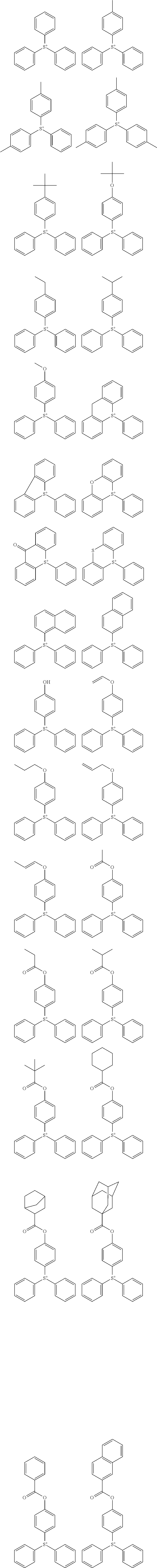

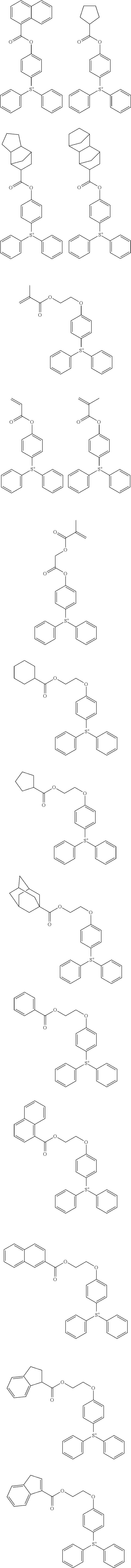

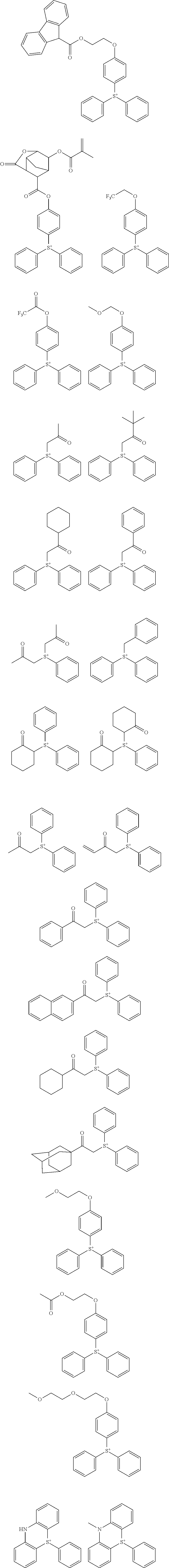

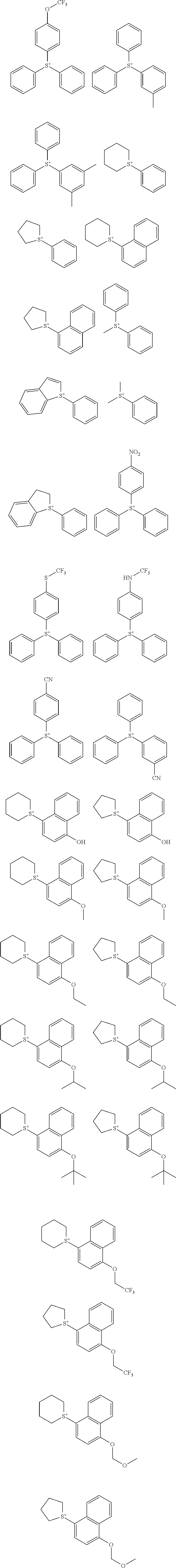

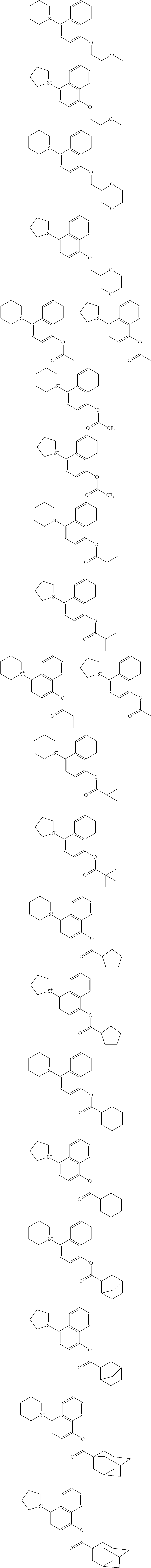

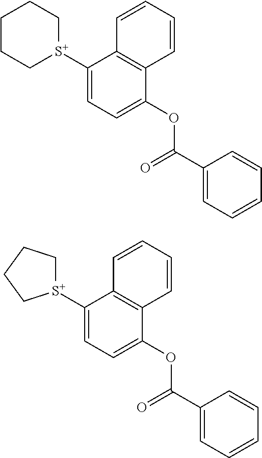

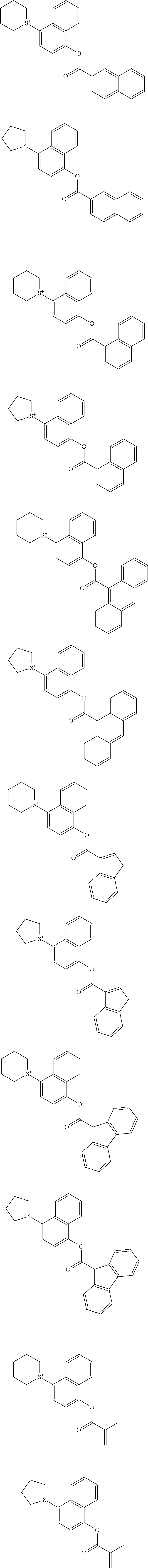

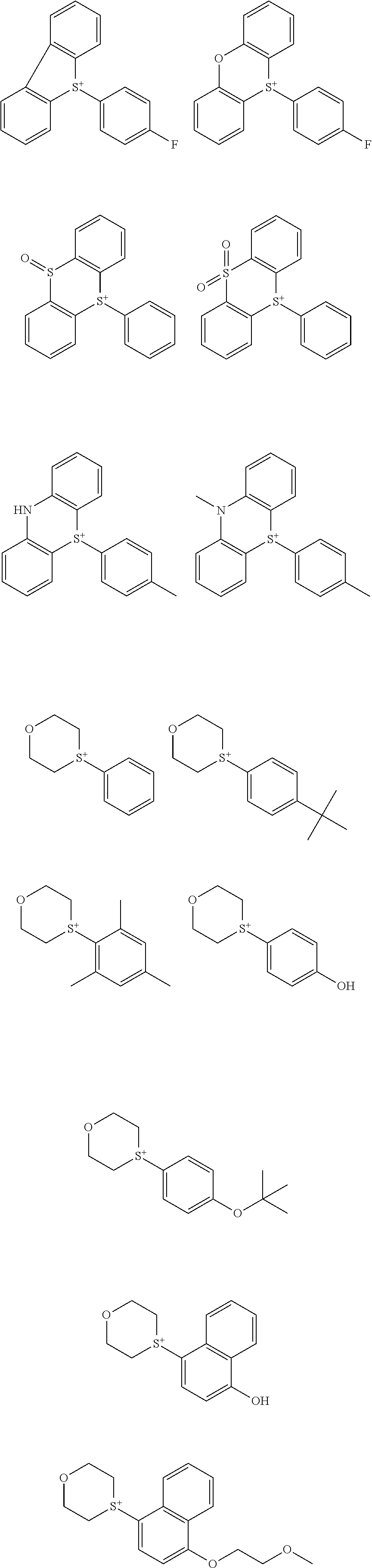

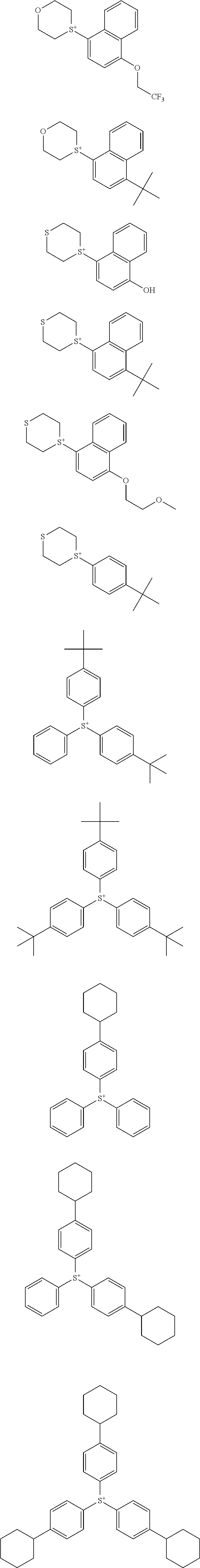

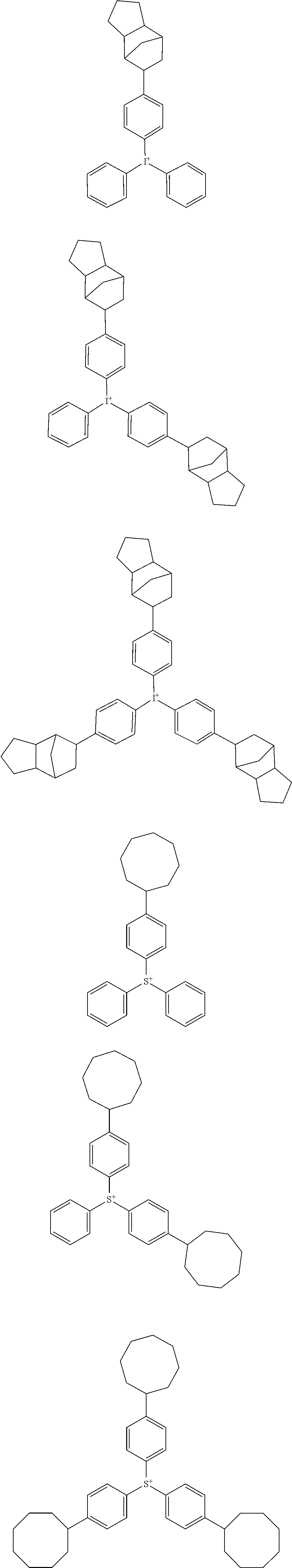

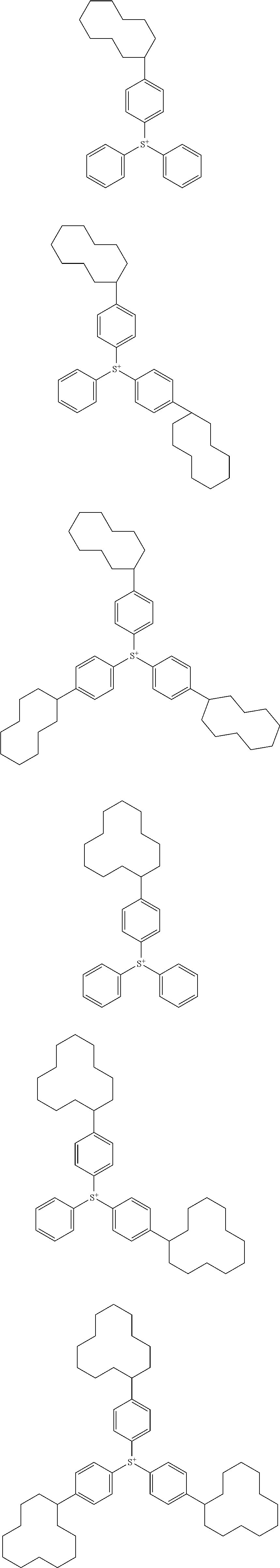

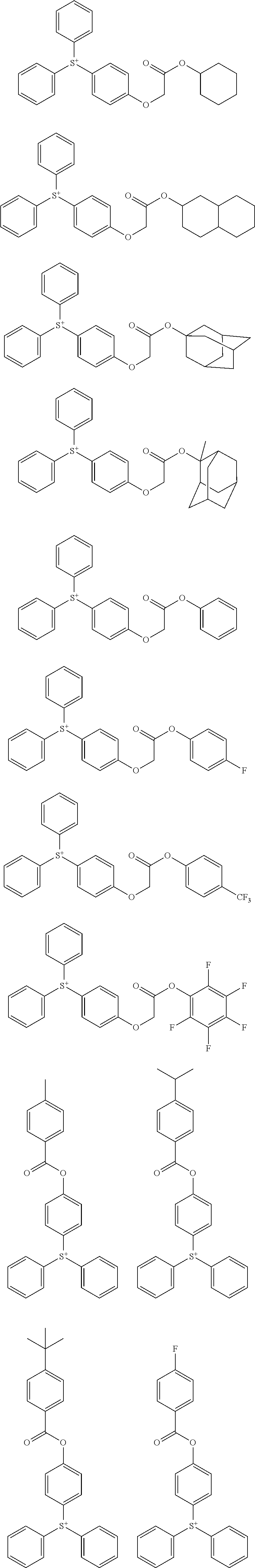

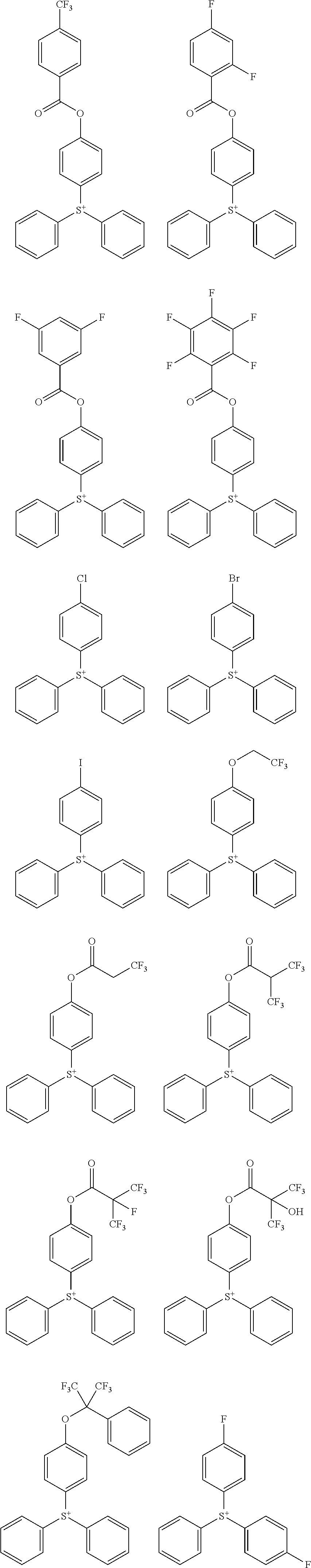

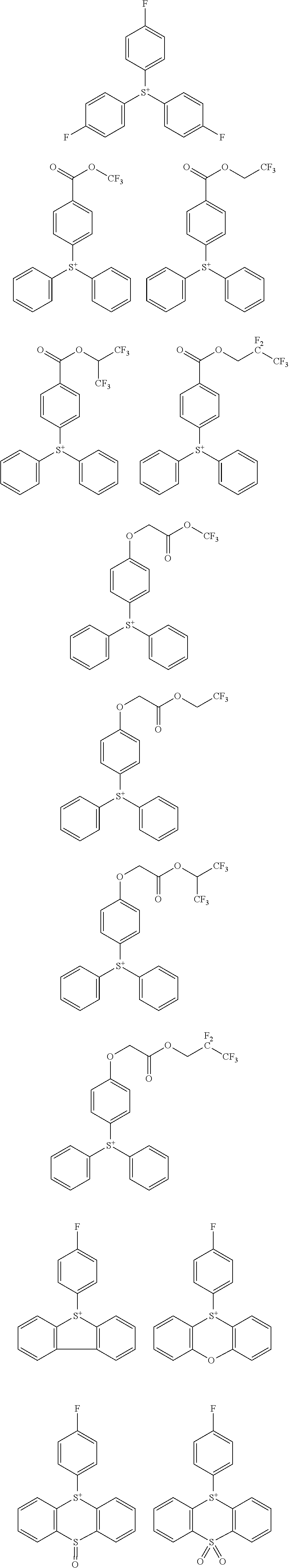

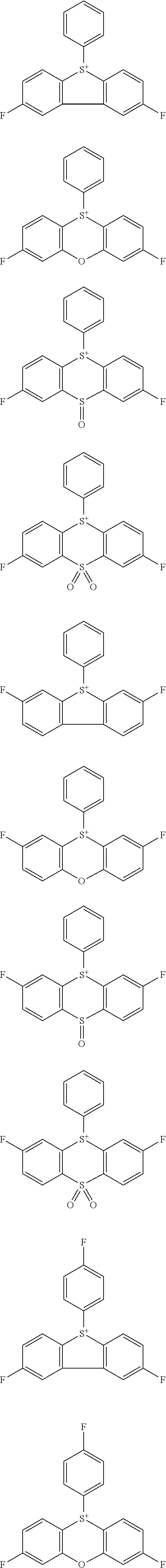

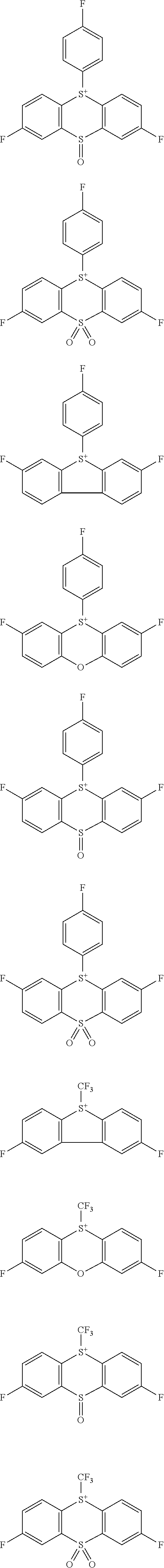

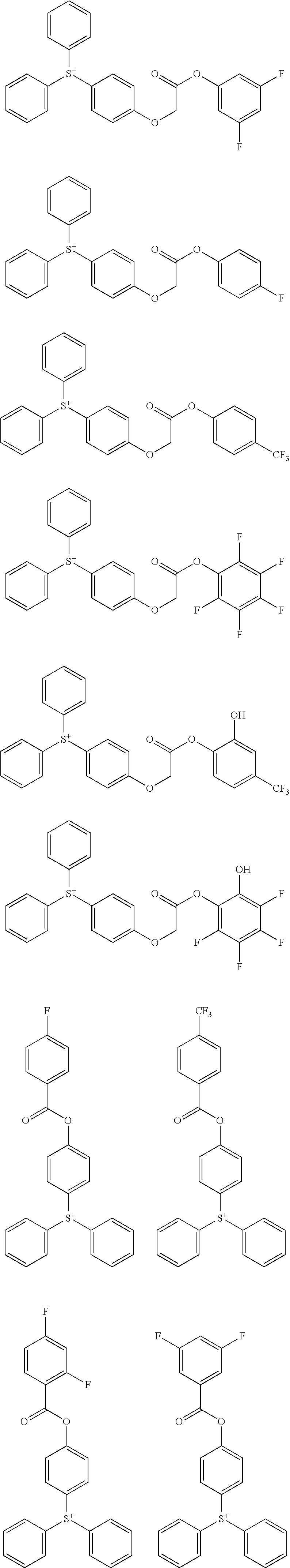

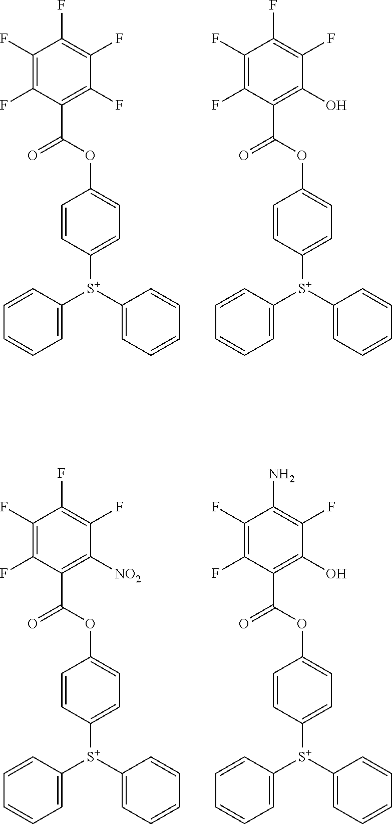

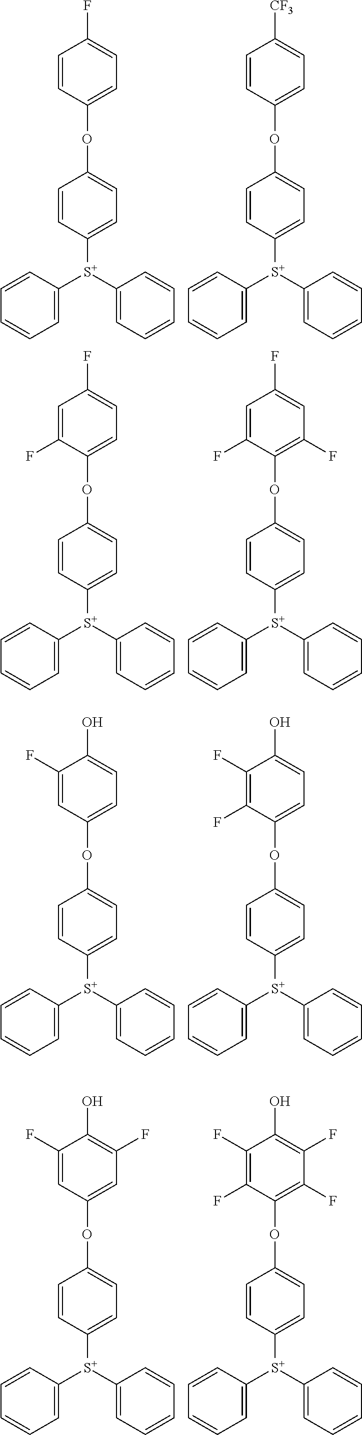

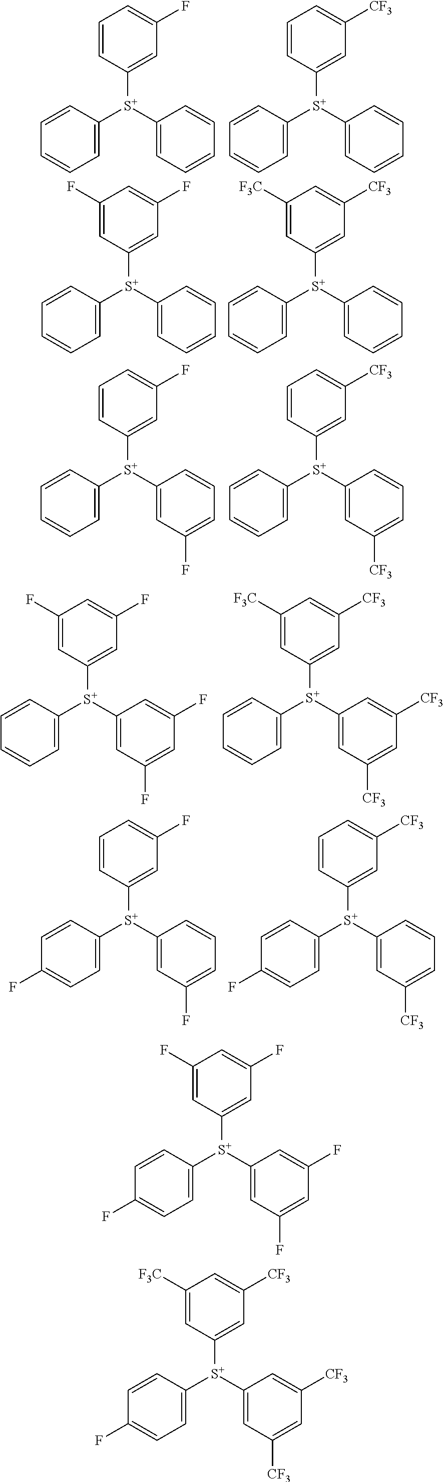

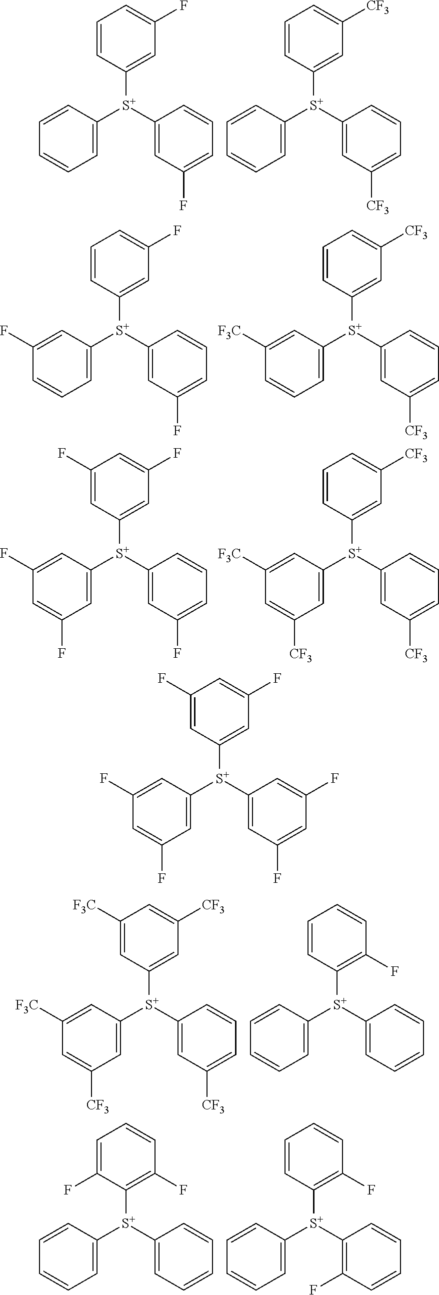

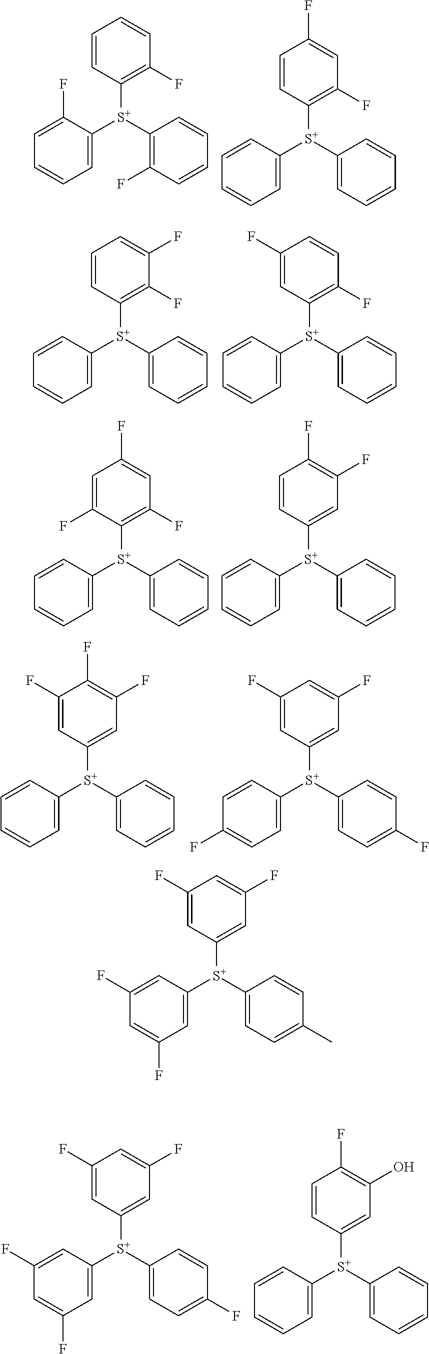

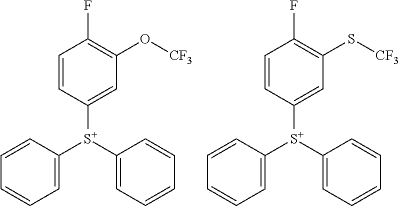

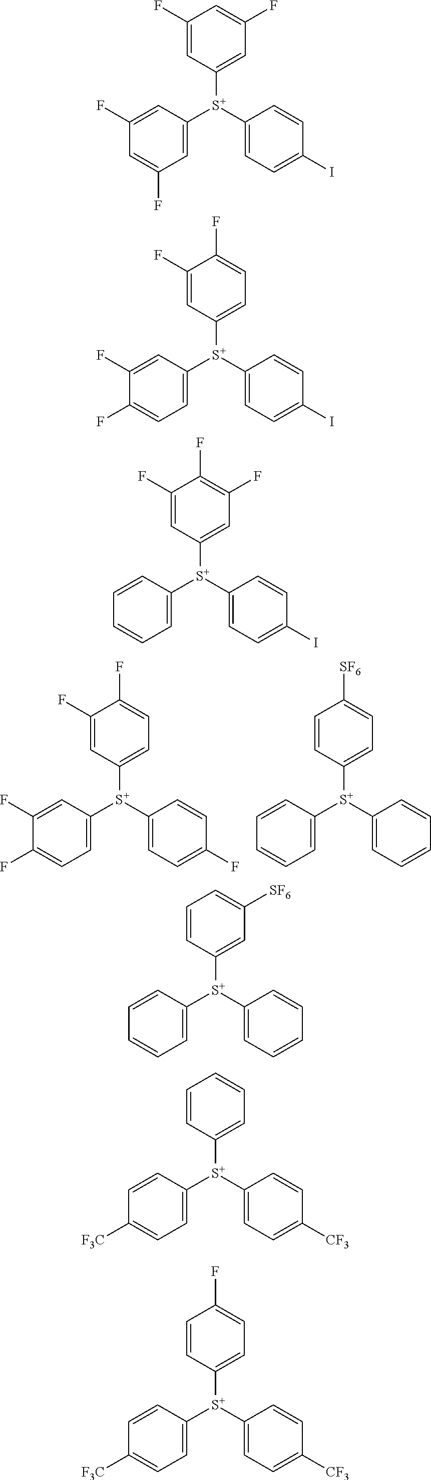

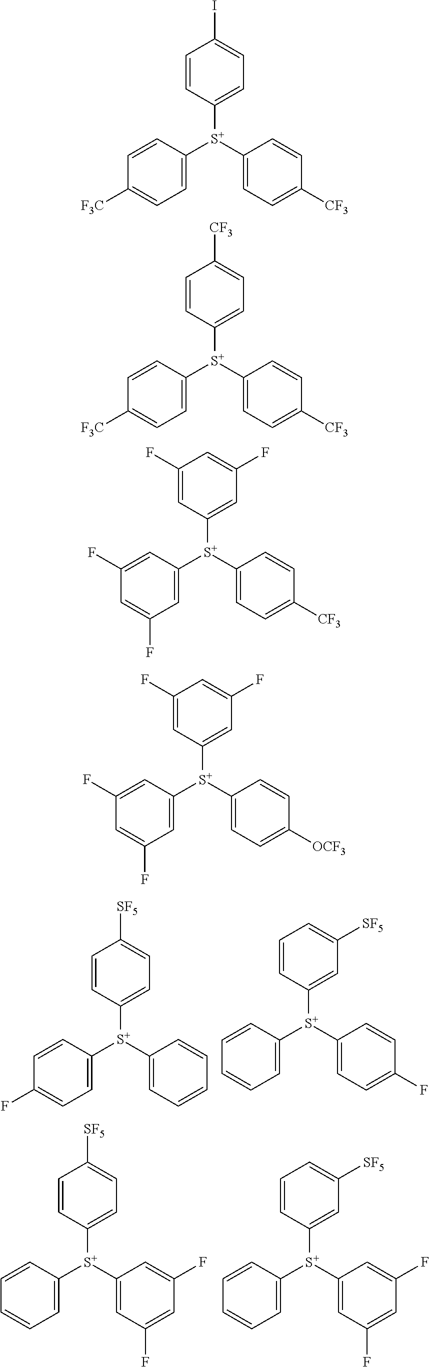

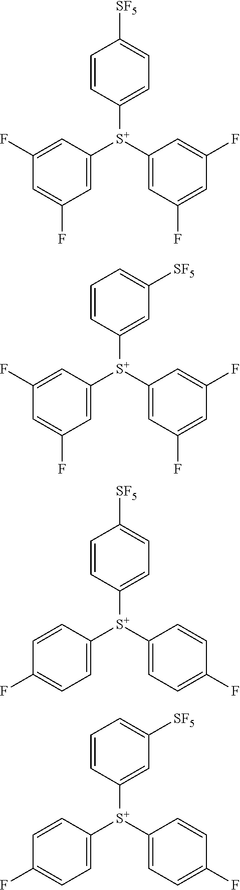

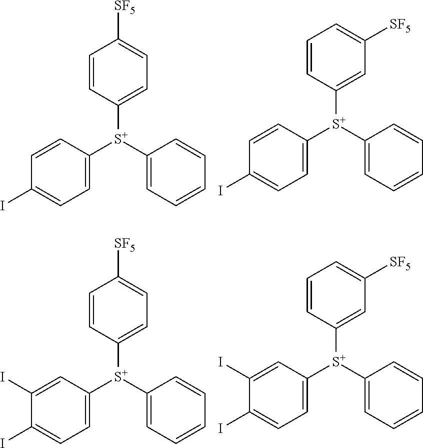

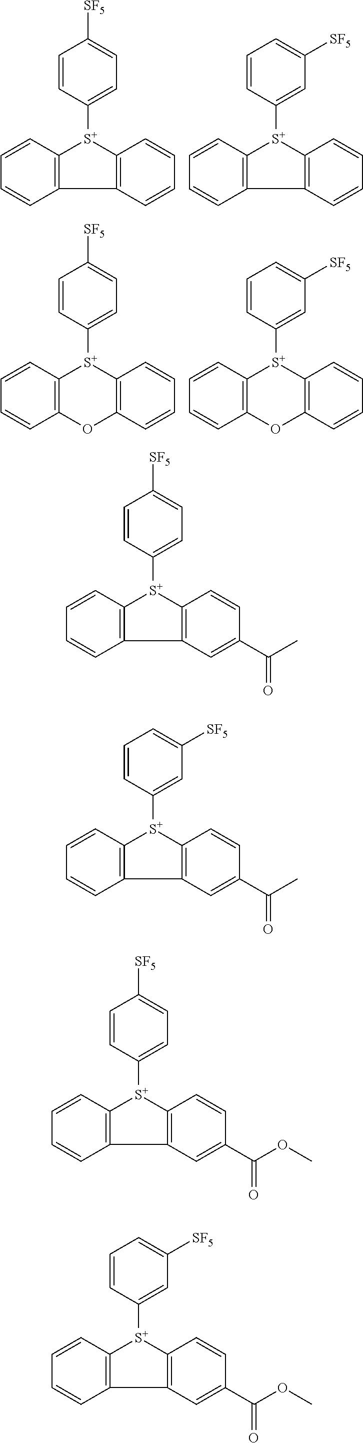

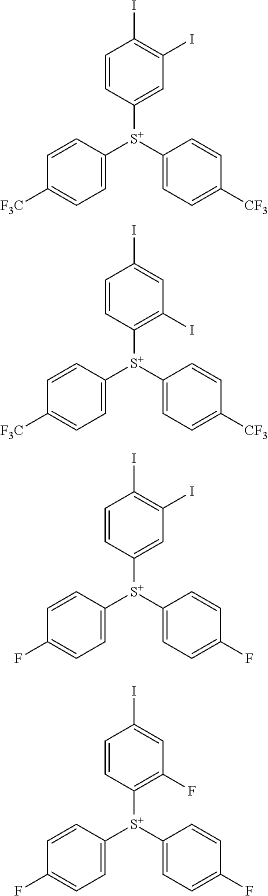

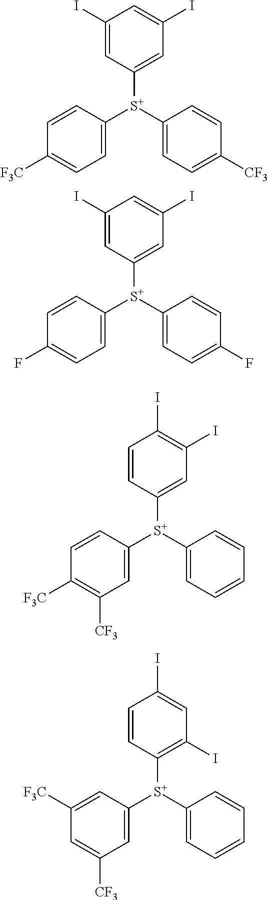

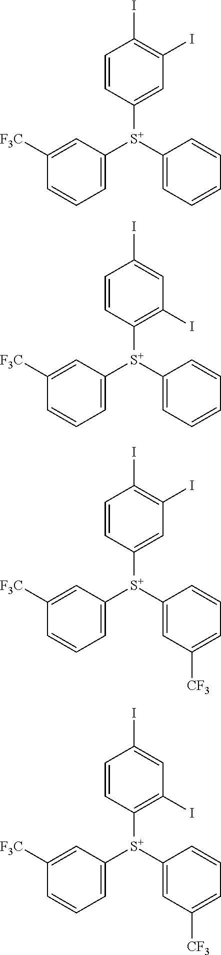

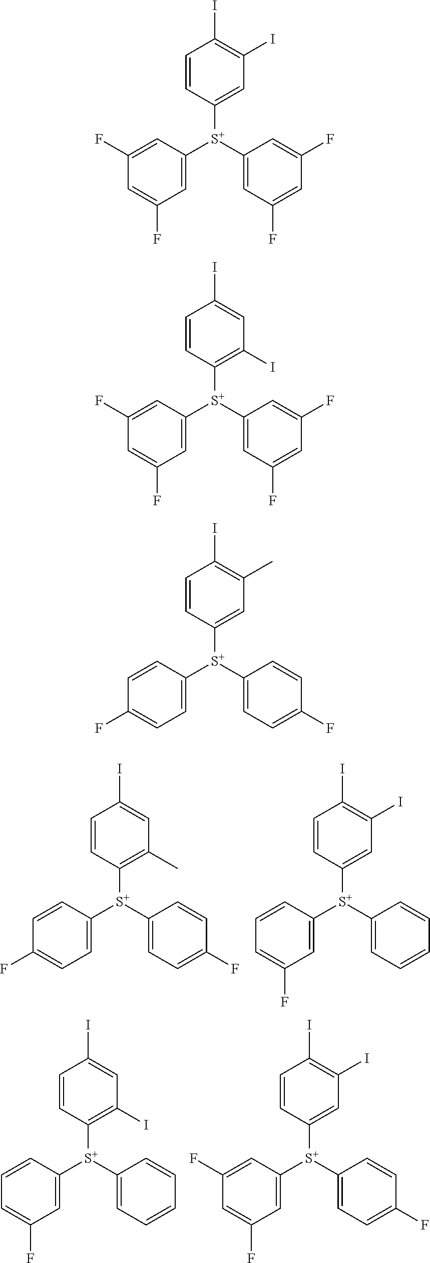

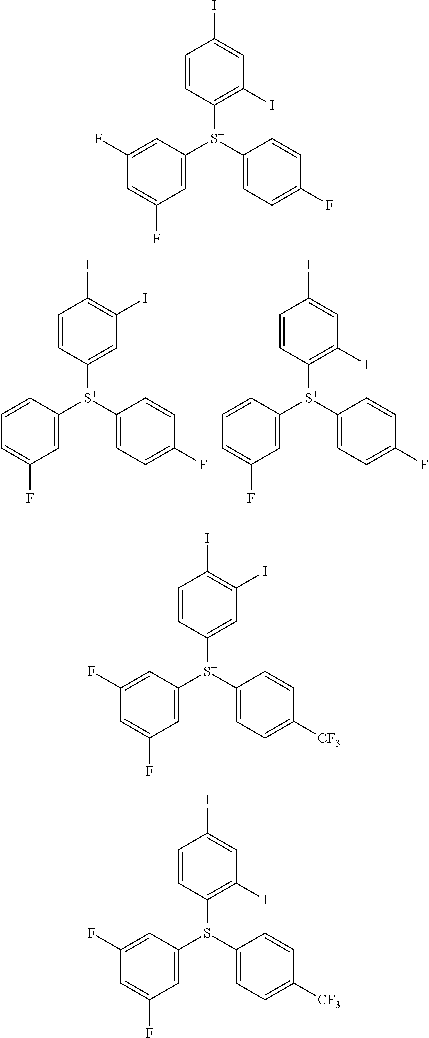

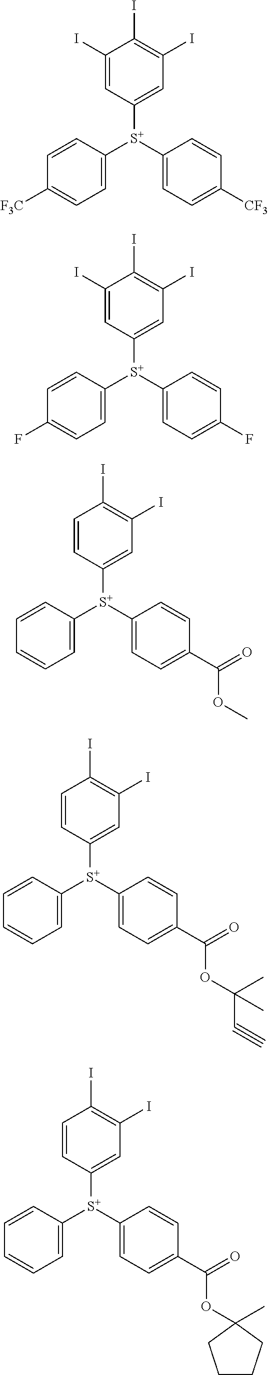

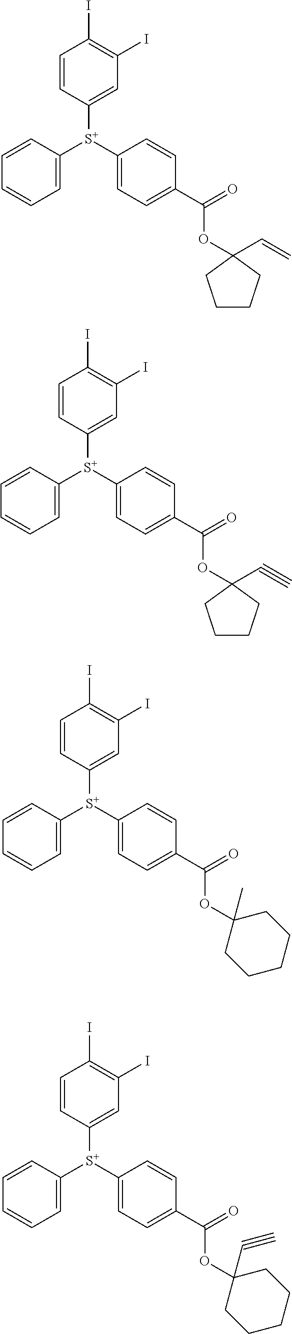

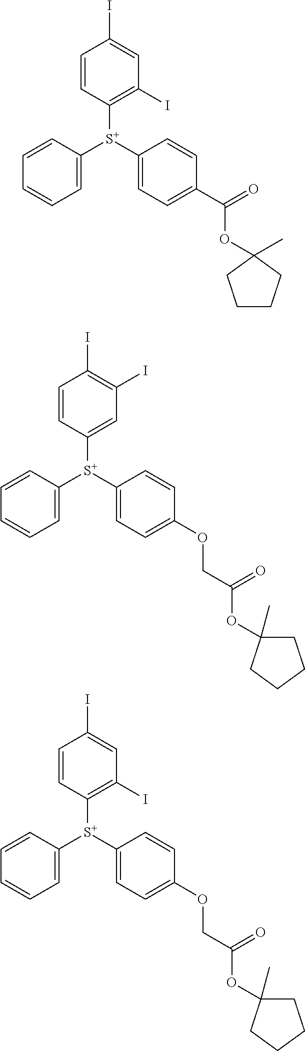

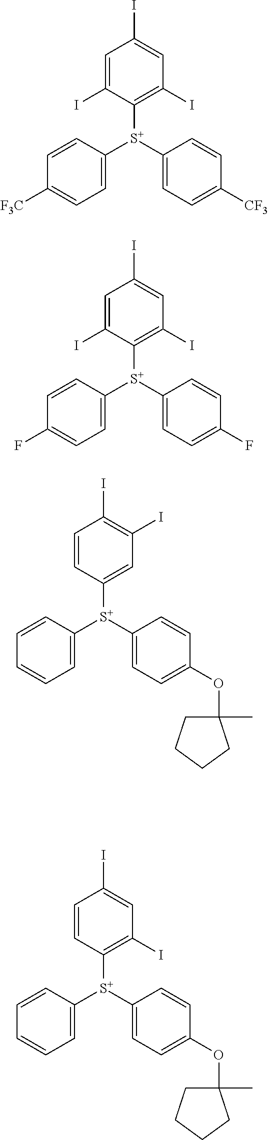

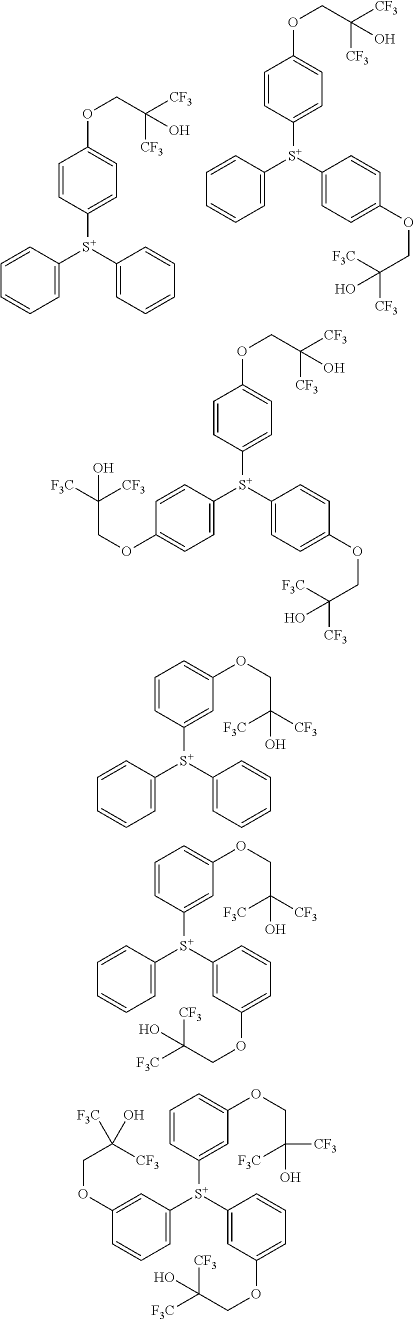

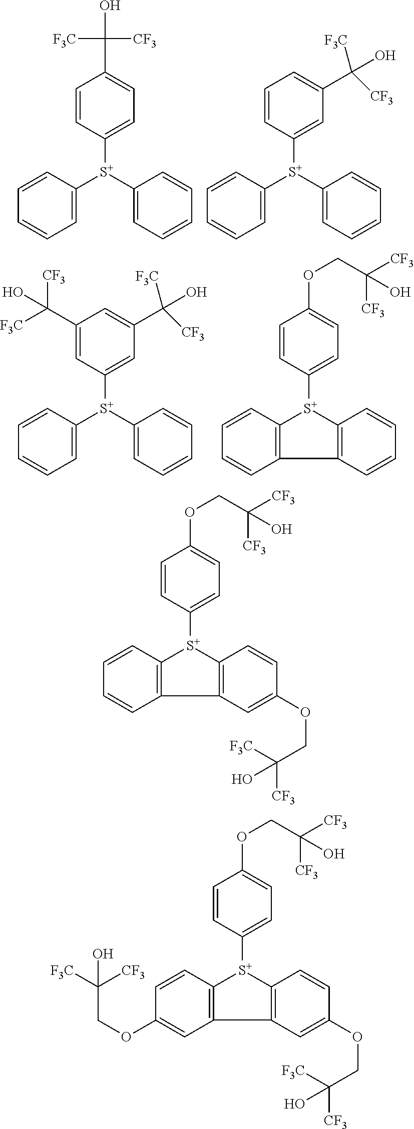

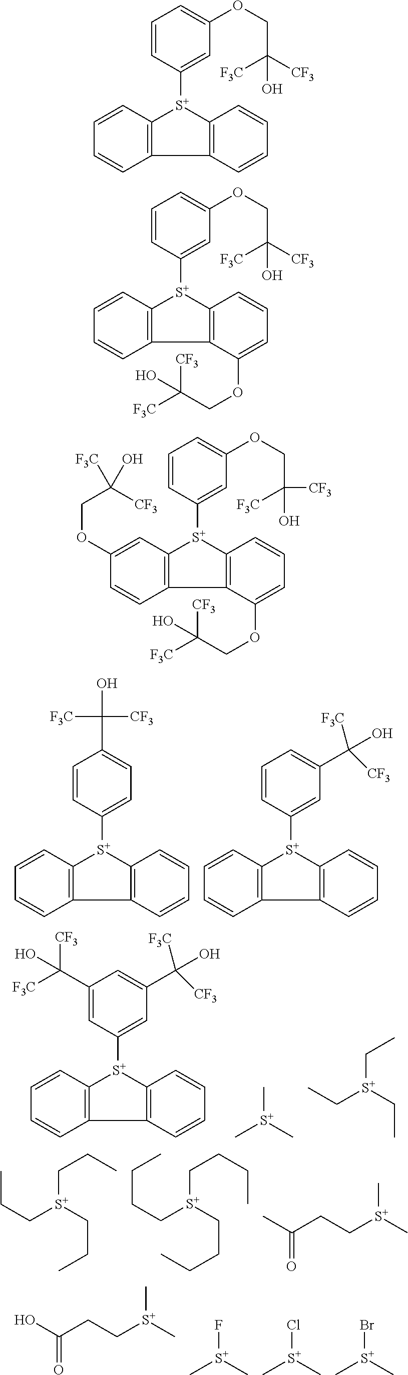

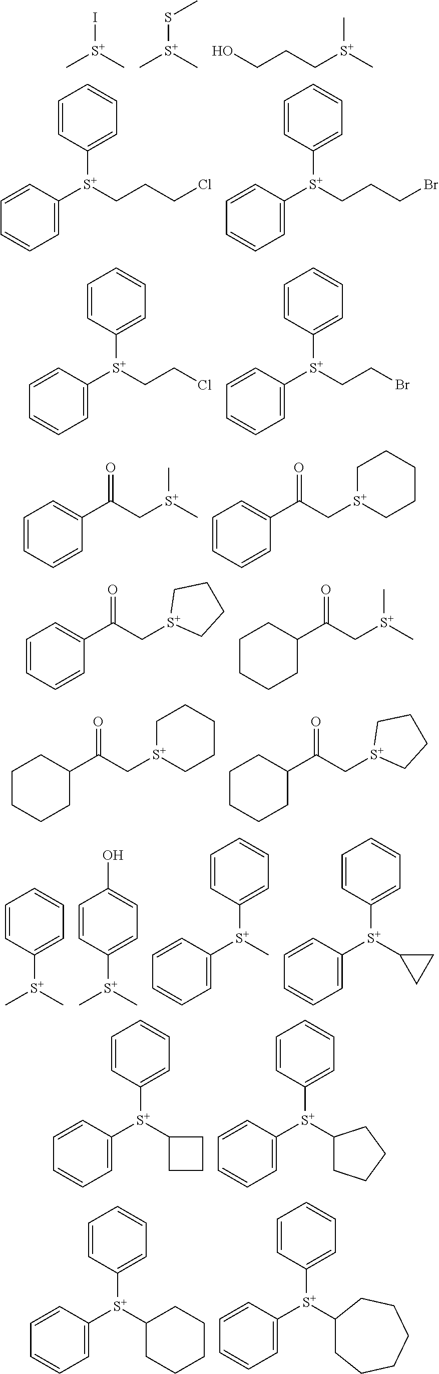

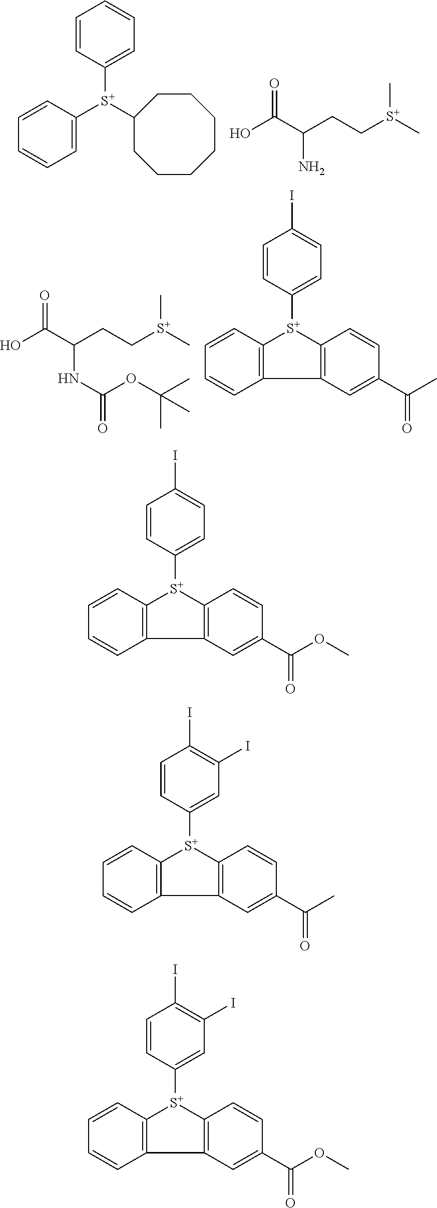

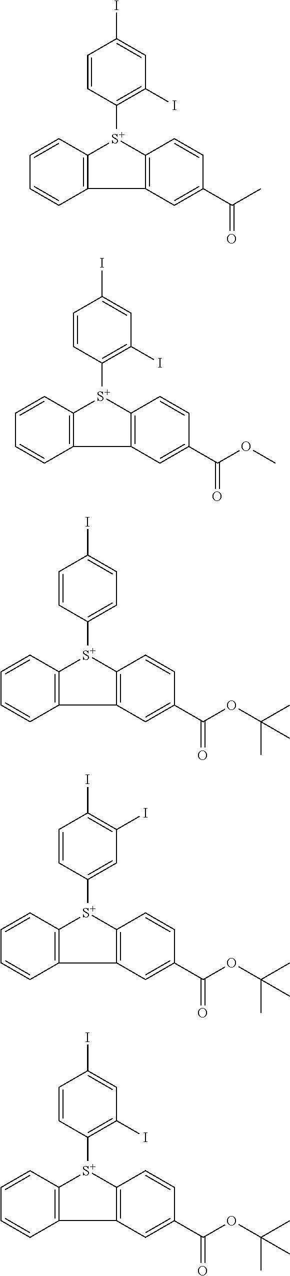

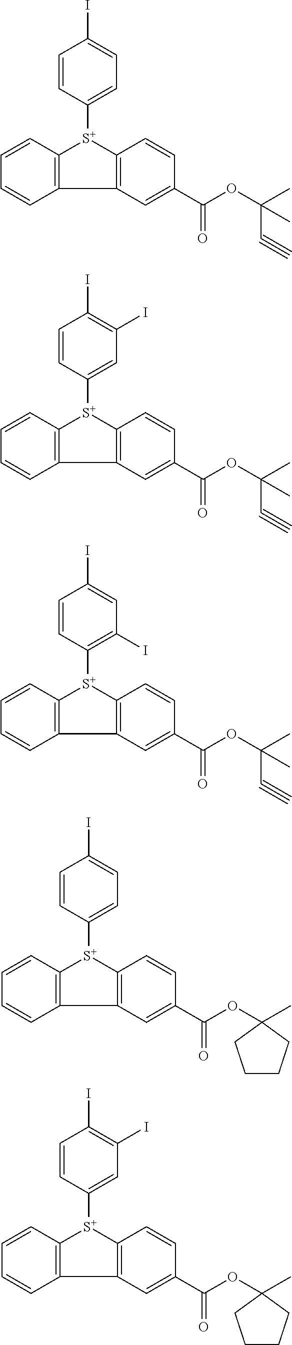

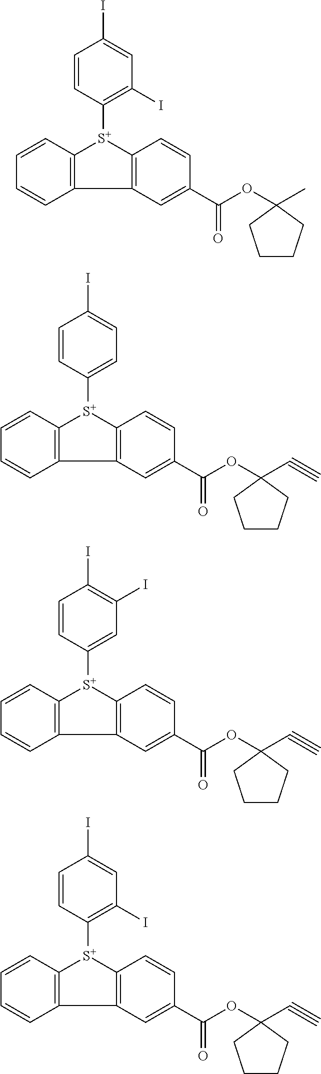

Examples of the cation of the iodonium salt represented by the general formula (a)-2 include the following, but are not limited thereto.

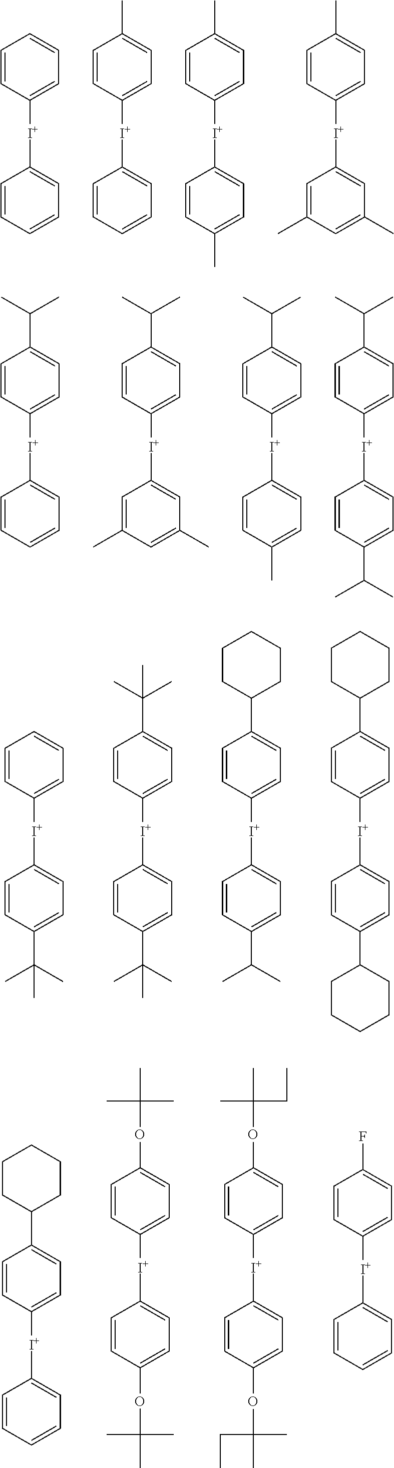

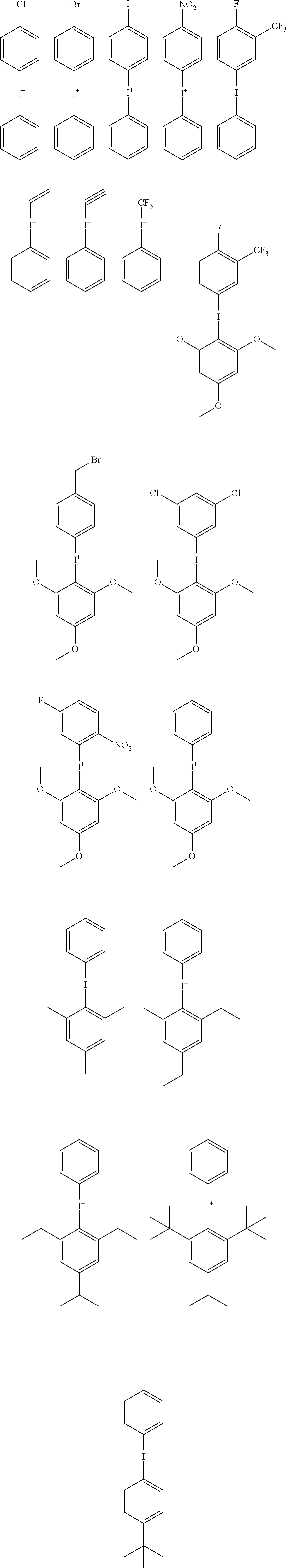

In order to enhance dissolution contrast, the resist material preferably further contains a repeating unit-b, in which a hydrogen atom of a carboxy group, a phenolic hydroxy group, or both is substituted with an acid-labile group. Furthermore, the repeating unit-b is preferably at least one kind of repeating unit selected from the repeating units represented by the following general formulae (b1) and (b2). Hereinafter, a repeating unit in which a hydrogen atom of a carboxy group is substituted with an acid-labile group is referred to as a repeating unit-b1, and a repeating unit in which a hydrogen atom of a phenolic hydroxy group is substituted with an acid-labile group is referred to as a repeating unit-b2.

Examples of the repeating units-b1 and -b2 include those represented by the following general formulae (b1) and (b2) respectively.

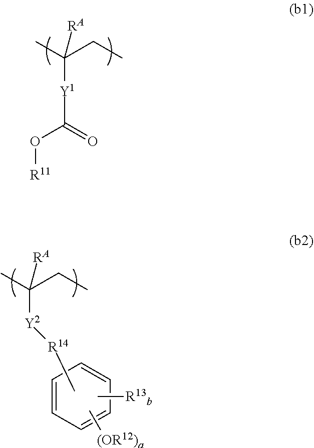

In the formulae, each R^{A} independently represents a hydrogen atom or a methyl group; Y¹ represents a single bond, a phenylene group, a naphthylene group, or a linking group having 1 to 12 carbon atoms and having an ester bond, an ether bond, or a lactone ring; Y² represents a single bond, an ester bond, or an amide bond; R¹¹ and R¹² each represent an acid-labile group; R¹³ represents a fluorine atom, a trifluoromethyl group, a cyano group, or an alkyl group having 1 to 6 carbon atoms; R¹⁴ represents a single bond or a linear or branched alkanediyl group having 1 to 6 carbon atoms, part of carbon atoms of the alkanediyl group optionally being substituted with an ether bond or an ester bond; “a” represents 1 or 2; and “b” represents an integer of 0 to 4.

In the formulae, each R^{A} independently represents a hydrogen atom or a methyl group, preferably a methyl group. Y¹ represents a single bond, a phenylene group, a naphthylene group, or a linking group having 1 to 12 carbon atoms and having an ester bond, an ether bond, or a lactone ring, preferably a single bond or a phenylene group. Y² represents a single bond, an ester bond, or an amide bond, preferably a single bond. R¹¹ and R¹² each represent an acid-labile group. R¹³ represents a fluorine atom, a trifluoromethyl group, a cyano group, or an alkyl group having 1 to 6 carbon atoms, preferably a fluorine atom. R¹⁴ represents a single bond or a linear or branched alkanediyl group having 1 to 6 carbon atoms, part of carbon atoms of the alkanediyl group optionally being substituted with an ether bond or an ester bond, preferably an ester bond. “a” represents 1 or 2, preferably 1. “b” represents an integer of 0 to 4, preferably 0 or 1.

Examples of monomers to give the repeating unit-b1 include the following, but are not limited thereto. Incidentally, in the following formulae, R^{A} and R¹¹ are as defined above.

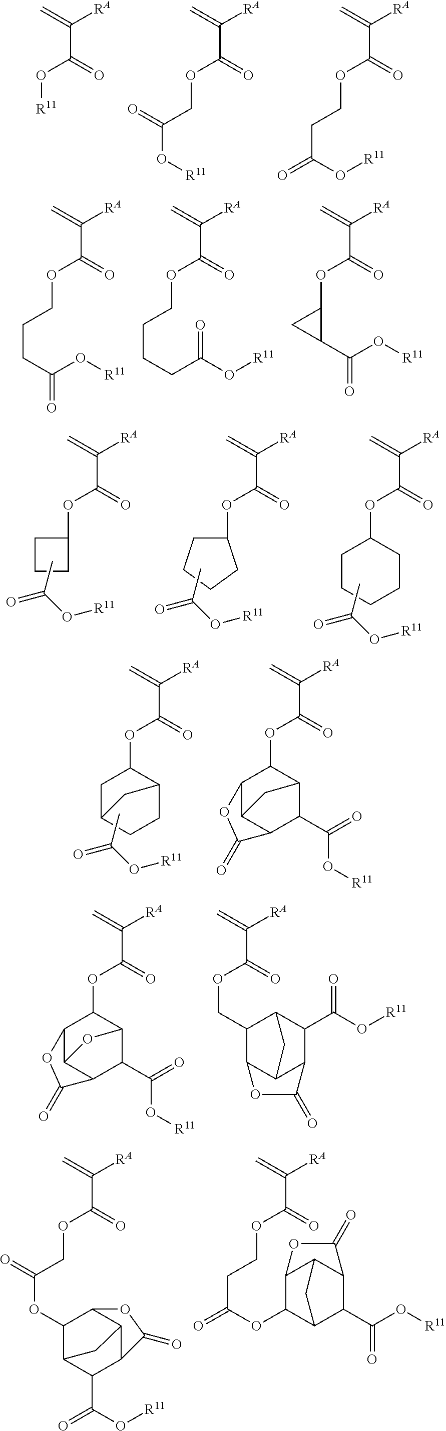

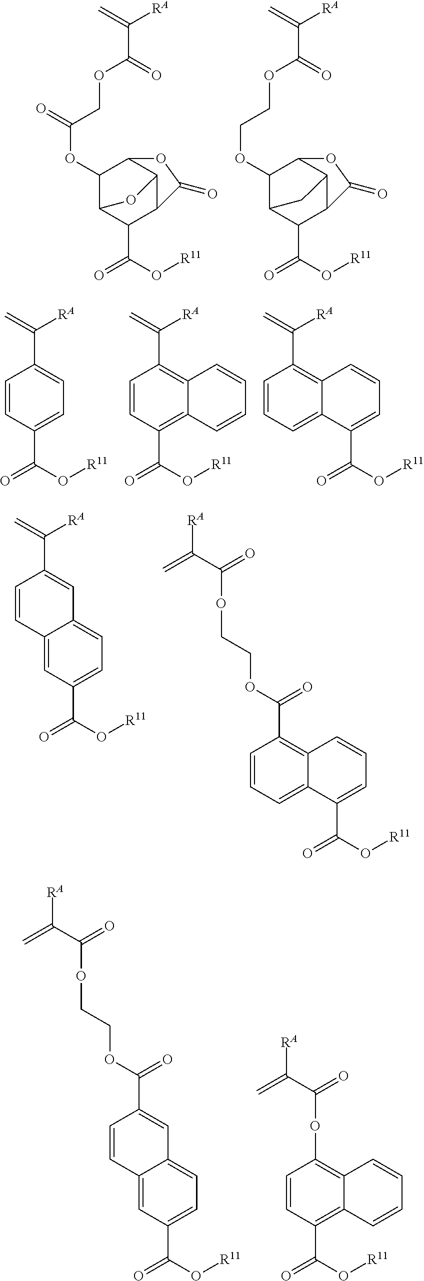

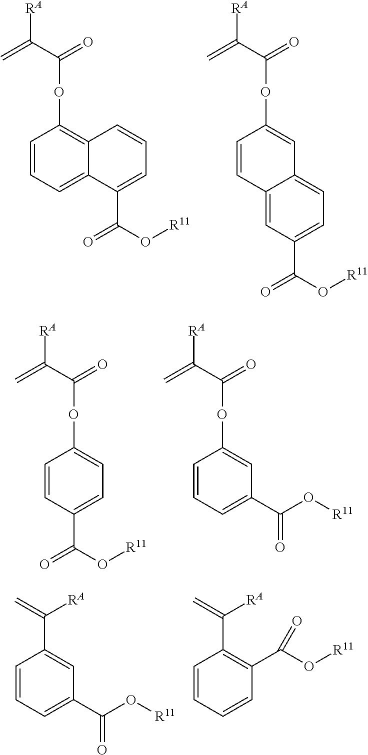

Examples of monomers to give the repeating unit-b2 include the following, but are not limited thereto. Incidentally, in the following formulae, R^{A} and R¹² are as defined above.

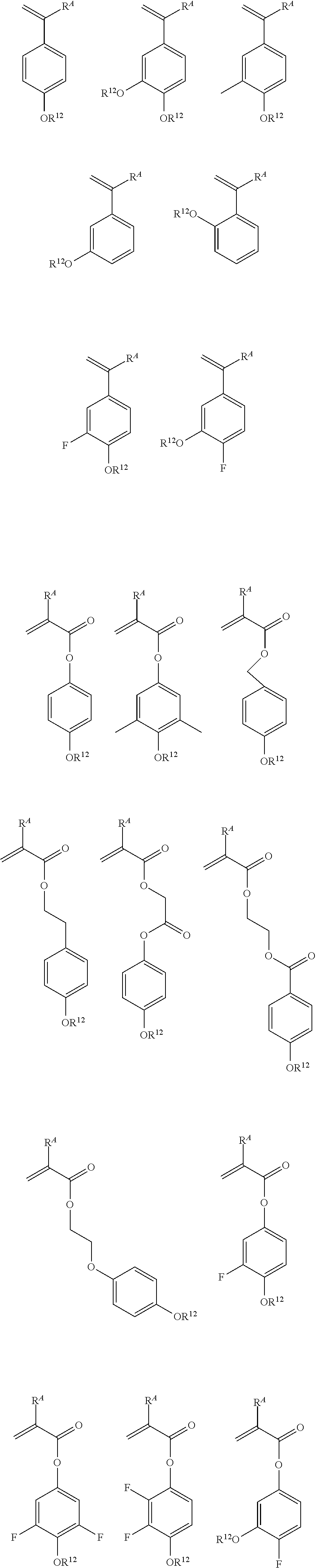

Various groups may be selected as the acid-labile group represented by R¹¹ or R¹², and examples include acid-labile groups represented by the following general formulae (AL-1) to (AL-3).

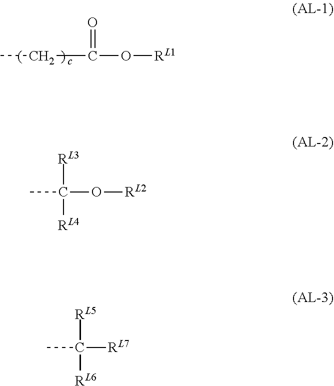

In the general formula (AL-1), “c” represents an integer of 0 to 6. R^{L1} represents a tertiary hydrocarbyl group having 4 to 61, preferably 4 to 15 carbon atoms, a trihydrocarbylsilyl group in which each hydrocarbyl group is a saturated hydrocarbyl group having 1 to 6 carbon atoms, a saturated hydrocarbyl group having 4 to 20 carbon atoms and containing a carbonyl group, an ether bond, or an ester bond, or a group represented by the general formula (AL-3).

The tertiary hydrocarbyl group represented by R^{L1} may be saturated or unsaturated, and may be branched or cyclic. Specific examples include a tert-butyl group, a tert-pentyl group, a 1,1-diethylpropyl group, a 1-ethylcyclopentyl group, a 1-butylcyclopentyl group, a 1-ethylcyclohexyl group, a 1-butylcyclohexyl group, a 1-ethyl-2-cyclopentenyl group, a 1-ethyl-2-cyclohexenyl group, a 2-methyl-2-adamantyl group, etc. Examples of the trihydrocarbylsilyl group include a trimethylsilyl group, a triethylsilyl group, a dimethyl-tert-butylsilyl group, etc. The saturated hydrocarbyl group containing a carbonyl group, an ether bond, or an ester bond may be linear, branched, or cyclic, but is preferably cyclic. Specific examples thereof include a 3-oxocyclohexyl group, a 4-methyl-2-oxooxan-4-yl group, a 5-methyl-2-oxooxolan-5-yl group, a 2-tetrahydropyranyl group, a 2-tetrahydrofuranyl group, etc.

Examples of the acid-labile group represented by the general formula (AL-1) include a tert-butoxycarbonyl group, a tert-butoxycarbonylmethyl group, a tert-pentyloxycarbonyl group, a tert-pentyloxycarbonylmethyl group, a 1,1-diethylpropyloxycarbonyl group, a 1,1-diethylpropyloxycarbonylmethyl group, a 1-ethylcyclopentyloxycarbonyl group, a 1-ethylcyclopentyloxycarbonylmethyl group, a 1-ethyl-2-cyclopentenyloxycarbonyl group, a 1-ethyl-2-cyclopentenyloxycarbonylmethyl group, a 1-ethoxyethoxycarbonylmethyl group, a 2-tetrahydropyranyloxycarbonylmethyl group, a 2-tetrahydrofuranyloxycarbonylmethyl group, etc.

Other examples of the acid-labile group represented by the general formula (AL-1) include groups represented by the following general formulae (AL-1)-1 to (AL-1)-10.

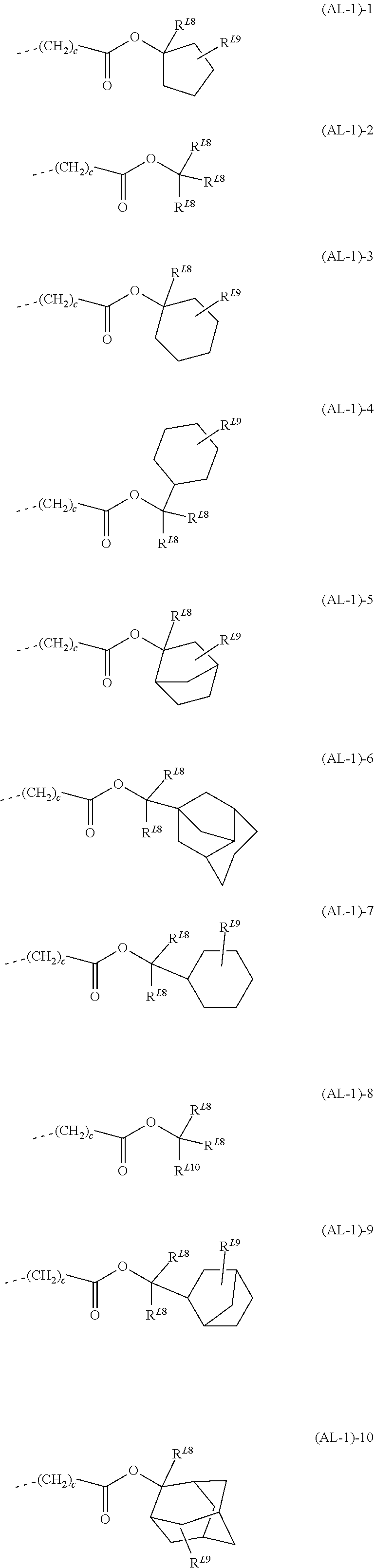

In the formulae, a broken line represents an attachment point.

In the general formulae (AL-1)-1 to (AL-1)-10, “c” is as defined above. Each R^{L8} independently represents a saturated hydrocarbyl group having 1 to 10 carbon atoms or an aryl group having 6 to 20 carbon atoms. R^{L9} represents a hydrogen atom or a saturated hydrocarbyl group having 1 to 10 carbon atoms. R^{L10} represents a saturated hydrocarbyl group having 2 to 10 carbon atoms or an aryl group having 6 to 20 carbon atoms. The saturated hydrocarbyl groups may be linear, branched, or cyclic.

In the general formula (AL-2), R^{L2} and R^{L3} each independently represent a hydrogen atom or a saturated hydrocarbyl group having 1 to 18, preferably 1 to 10 carbon atoms. The saturated hydrocarbyl group may be linear, branched, or cyclic, and specific examples thereof include a methyl group, an ethyl group, a propyl group, an isopropyl group, an n-butyl group, a sec-butyl group, a tert-butyl group, a cyclopentyl group, a cyclohexyl group, a 2-ethylhexyl group, an n-octyl group, etc.

In the general formula (AL-2), R^{L4} represents a hydrocarbyl group having 1 to 18, preferably 1 to 10 carbon atoms and optionally containing a heteroatom. The hydrocarbyl group may be saturated or unsaturated, and may be linear, branched, or cyclic. Examples of the hydrocarbyl group include saturated hydrocarbyl groups having 1 to 18 carbon atoms, etc., and part of the hydrogen atoms thereof may be substituted with a hydroxy group, an alkoxy group, an oxo group, an amino group, an alkylamino group, etc. Examples of such a saturated hydrocarbyl group having a substituent include those shown below.

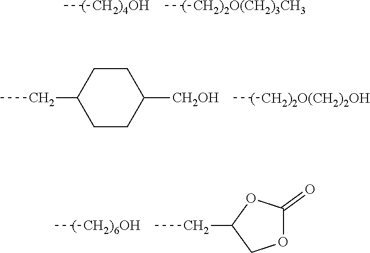

In the formulae, a broken line represents an attachment point.

R^{L2} and R^{L3}, R^{L2} and R^{L4}, or R^{L3} and R^{L4} may be bonded with each other to form a ring together with the carbon atom bonded thereto or together with the carbon atom and the oxygen atom bonded thereto. In this case, the R^{L2} and R^{L3}, R^{L2} and R^{L4}, or R^{L3} and R^{L4} that are involved in the formation of the ring each independently represent an alkanediyl group having 1 to 18, preferably 1 to 10 carbon atoms. The number of carbon atoms in the ring obtained by these groups being bonded is preferably 3 to 10, more preferably 4 to 10.

Among the acid-labile groups represented by the general formula (AL-2), examples of linear and branched groups include those represented by the following formulae (AL-2)-1 to (AL-2)-69, but are not limited thereto. Note that, in the following formulae, a broken line represents an attachment point.

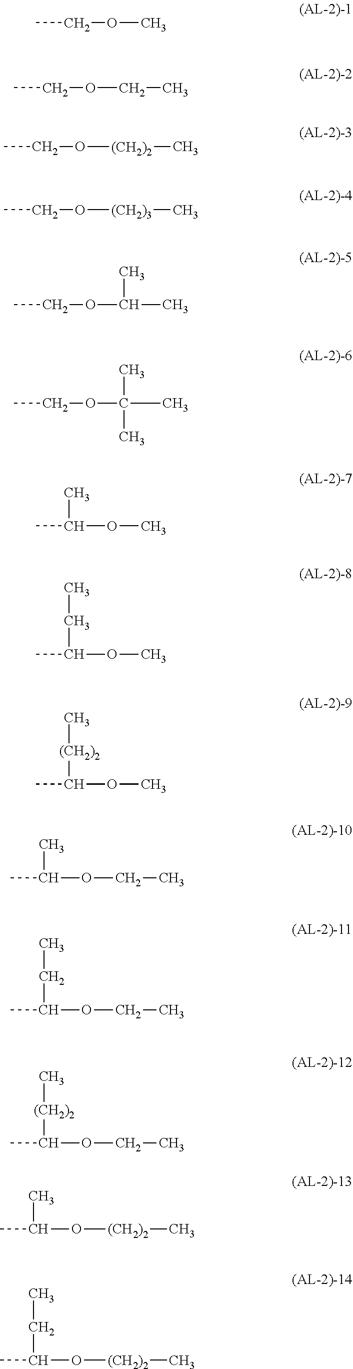

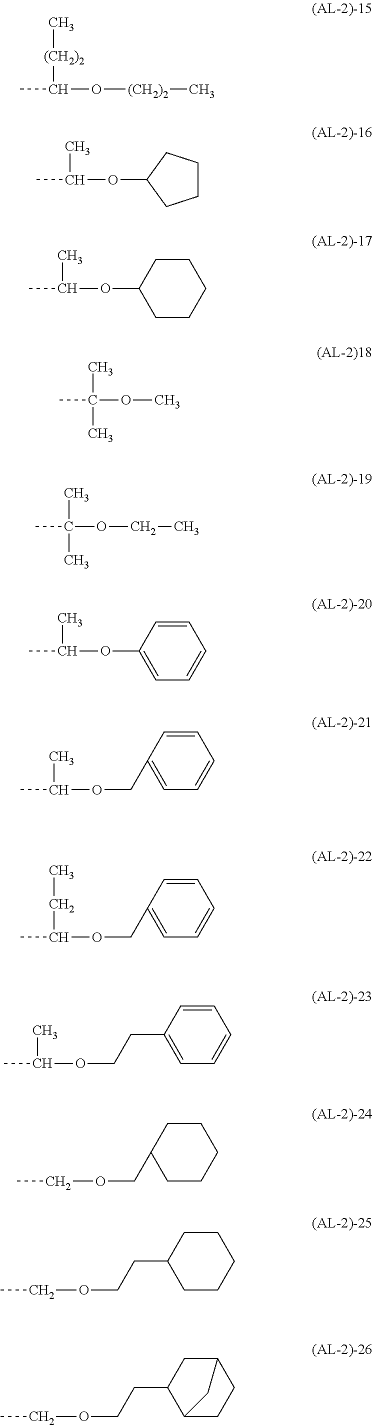

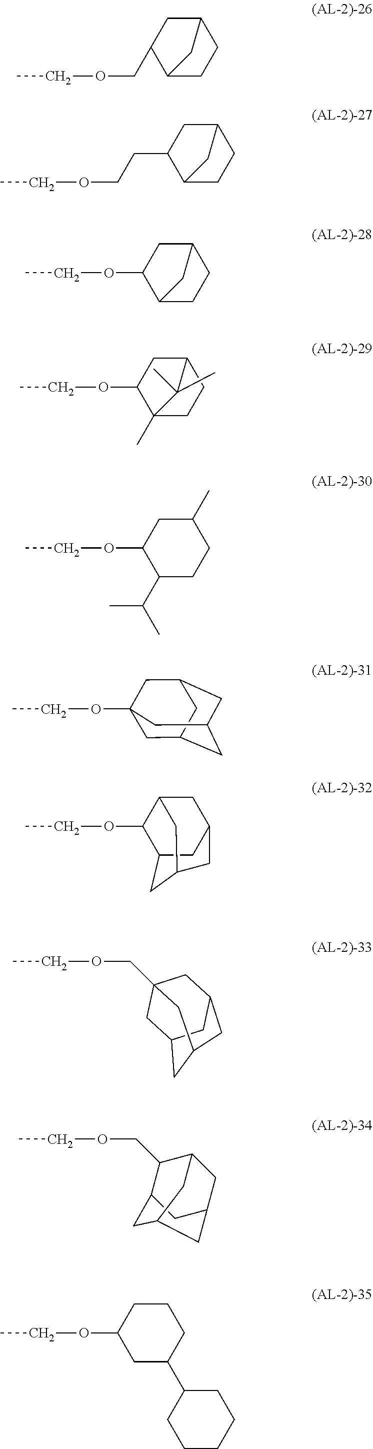

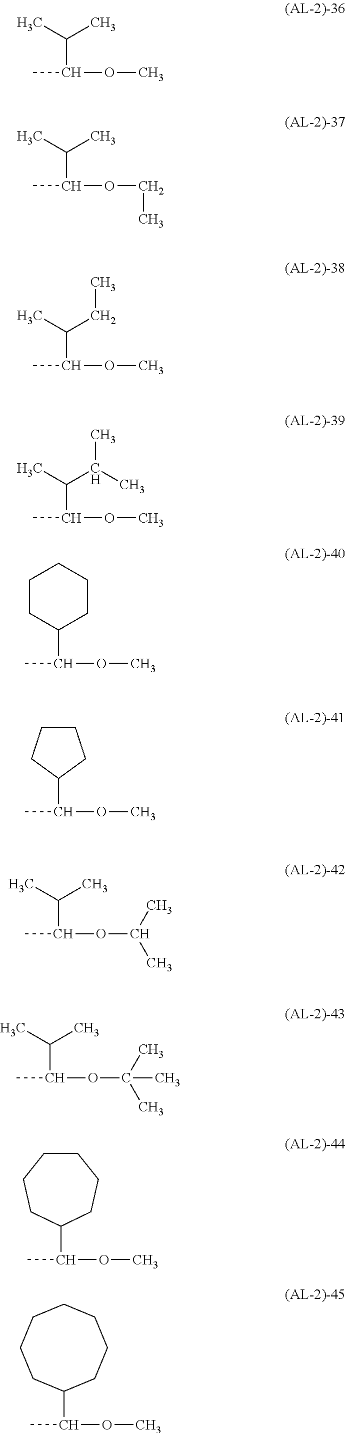

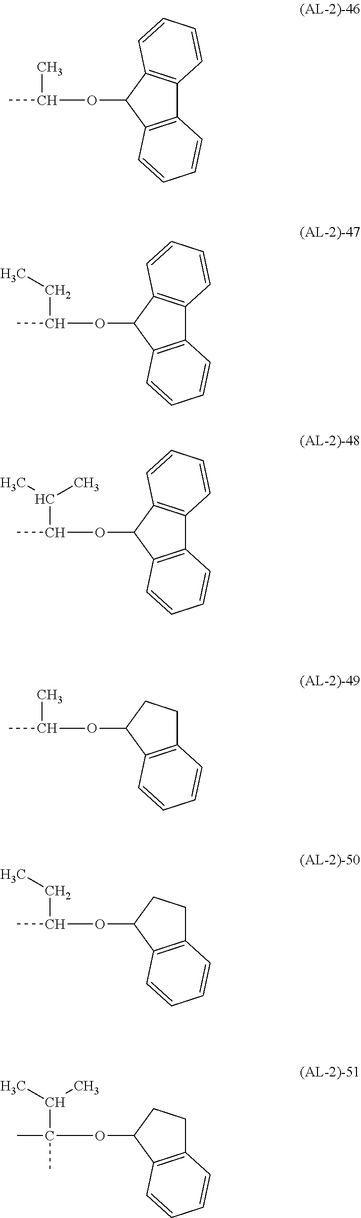

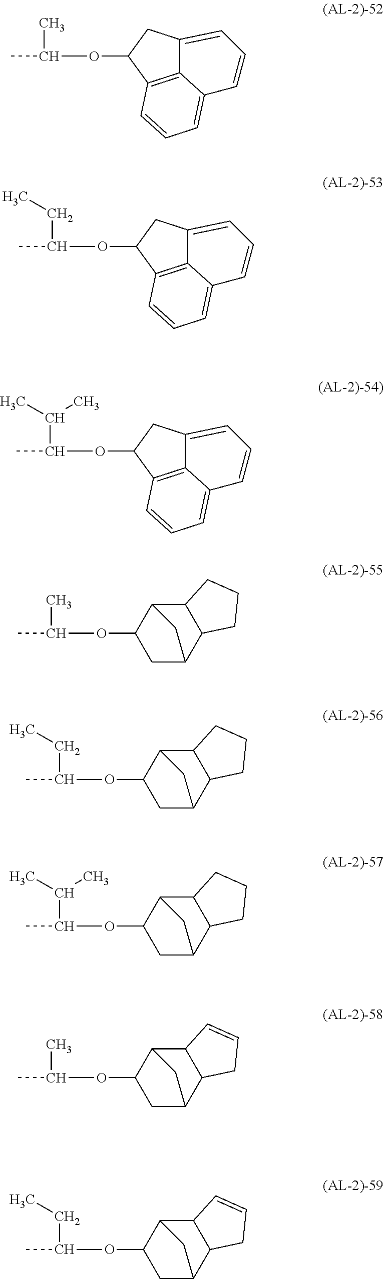

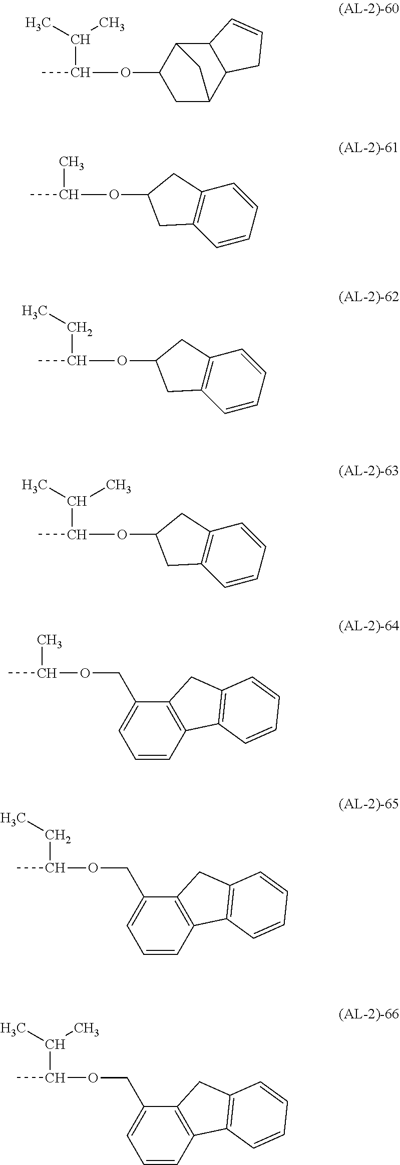

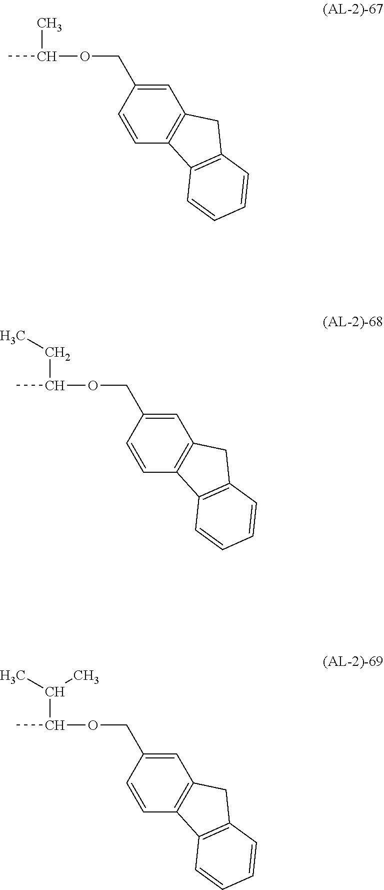

Among the acid-labile groups represented by the general formula (AL-2), examples of cyclic groups include a tetrahydrofuran-2-yl group, a 2-methyltetrahydrofuran-2-yl group, a tetrahydropyran-2-yl group, a 2-methyltetrahydropyran-2-yl group, etc.

Examples of the acid-labile group include groups represented by the following general formula (AL-2a) or (AL-2b). The resist material may be intermolecularly or intramolecularly crosslinked by the acid-labile group.

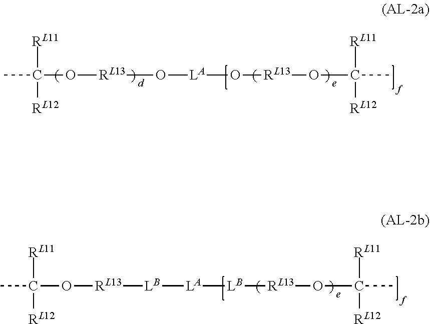

In the formulae, a broken line represents an attachment point.

In the general formula (AL-2a) or (AL-2b), R^{L11} and R^{L12} each independently represent a hydrogen atom or a saturated hydrocarbyl group having 1 to 8 carbon atoms. The saturated hydrocarbyl group may be linear, branched, or cyclic. Furthermore, R^{L11} and R^{L12} may be bonded to each other to form a ring together with the carbon atom bonded thereto, and in such a case, R^{L11} and R^{L12} each independently represent an alkanediyl group having 1 to 8 carbon atoms. Each R^{L13} independently represents a saturated hydrocarbylene group having 1 to 10 carbon atoms, and the saturated hydrocarbylene group may be linear, branched, or cyclic. “d” and “e” each independently represent an integer of 0 to 10, preferably an integer of 0 to 5, and “f” represents an integer of 1 to 7, preferably an integer of 1 to 3.

In the general formula (AL-2a) or (AL-2b), LA represents an aliphatic saturated hydrocarbon group having a valency of f+1 and having 1 to 50 carbon atoms, an alicyclic saturated hydrocarbon group having a valency of f+1 and having 3 to 50 carbon atoms, an aromatic hydrocarbon group having a valency of f+1 and having 6 to 50 carbon atoms, or a heterocyclic group having a valency of f+1 and having 3 to 50 carbon atoms. In addition, part of the carbon atoms of these groups may be substituted with a heteroatom-containing group, and part of the hydrogen atoms bonded to the carbon atoms of these groups may be substituted with a hydroxy group, a carboxy group, an acyl group, or a fluorine atom. Preferable examples of LA include saturated hydrocarbon groups, such as saturated hydrocarbylene groups having 1 to 20 carbon atoms, trivalent saturated hydrocarbon groups, and tetravalent saturated hydrocarbon groups, arylene groups having 6 to 30 carbon atoms, etc. The saturated hydrocarbon groups may be linear, branched, or cyclic. L^{B} represents —C(═O)—O—, —NH—C(═O)—O—, or —NH—C(═O)—NH—.

Examples of crosslinking acetal groups represented by the general formula (AL-2a) or (AL-2b) include groups represented by the following formulae (AL-2)-70 to (AL-2)-77, etc.

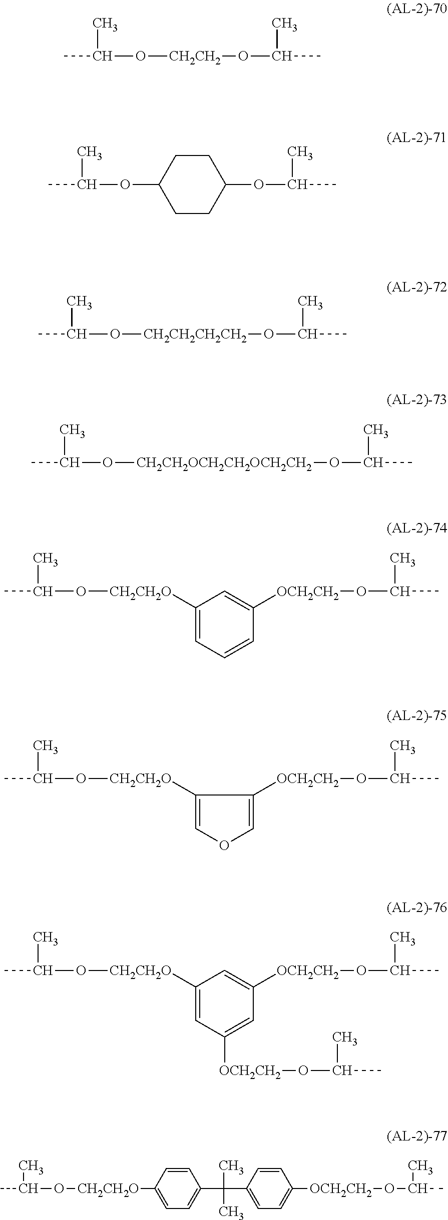

In the formulae, a broken line represents an attachment point.

In the general formula (AL-3), R^{L5}, R^{L6}, and R^{L7} each independently represent a hydrocarbyl group having 1 to 20 carbon atoms, and may contain a heteroatom such as an oxygen atom, a sulfur atom, a nitrogen atom, and a fluorine atom. The hydrocarbyl group may be saturated or unsaturated, and may be linear, branched, or cyclic. Specific examples of the hydrocarbyl group include alkyl groups having 1 to 20 carbon atoms, cyclic saturated hydrocarbyl groups having 3 to 20 carbon atoms, alkenyl groups having 2 to 20 carbon atoms, cyclic unsaturated hydrocarbyl groups having 3 to 20 carbon atoms, aryl groups having 6 to 10 carbon atoms, etc. Furthermore, R^{L5} and R^{L6}, R^{L5} and R^{L7}, or R^{L6} and R^{L7} may be bonded to each other to form an aliphatic ring having 3 to 20 carbon atoms together with the carbon atom bonded thereto.

Examples of the group represented by the general formula (AL-3) include a tert-butyl group, a 1,1-diethylpropyl group, a 1-ethylnorbornyl group, a 1-methylcyclopentyl group, a 1-isopropylcyclopentyl group, a 1-ethylcyclopentyl group, a 1-methylcyclohexyl group, a 2-(2-methyl) adamantyl group, a 2-(2-ethyl) adamantyl group, a tert-pentyl group, etc.

Examples of the group represented by the general formula (AL-3) also include groups represented by the following general formulae (AL-3)-1 to (AL-3)-19.

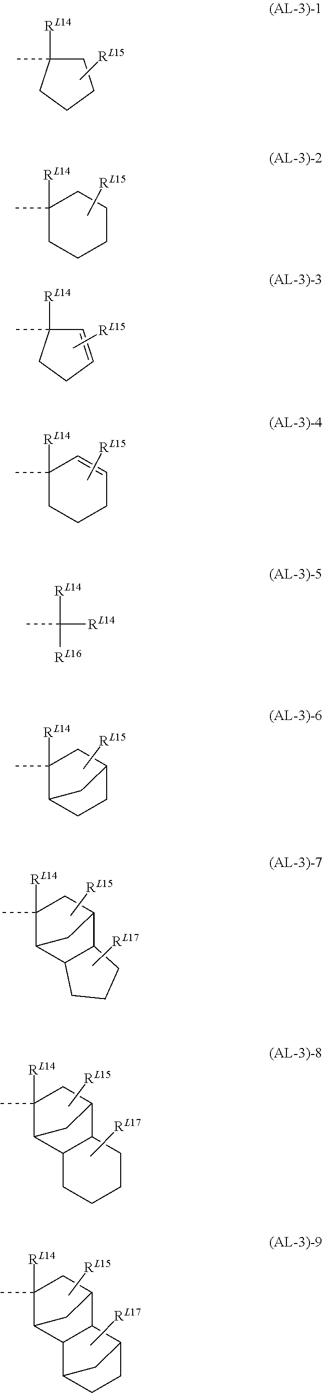

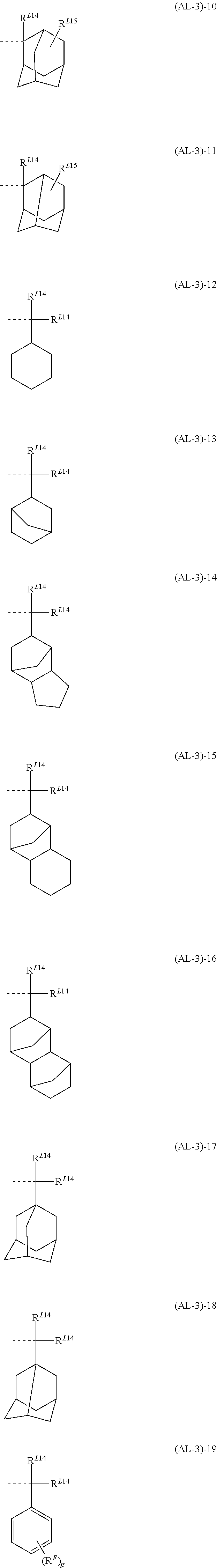

In the formulae, a broken line represents an attachment point.

In the general formulae (AL-3)-1 to (AL-3)-19, each R^{L14} independently represents a saturated hydrocarbyl group having 1 to 8 carbon atoms or an aryl group having 6 to 20 carbon atoms. R^{L15} and R^{L17} each independently represent a hydrogen atom or a saturated hydrocarbyl group having 1 to 20 carbon atoms. R^{L16} represents an aryl group having 6 to 20 carbon atoms. The saturated hydrocarbyl group may be linear, branched, or cyclic. As the aryl group, a phenyl group or the like is preferable. R^{F} represents a fluorine atom or a trifluoromethyl group. “g” represents an integer of 1 to 5.

Further examples of the acid-labile group include groups represented by the following general formula (AL-3)-20 or (AL-3)-21. The resist material may be intramolecularly or intermolecularly crosslinked by the acid-labile group.

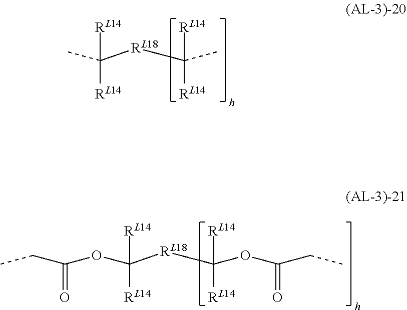

In the formulae, a broken line represents an attachment point.

In the general formulae (AL-3)-20 and (AL-3)-21, R^{L14} is as defined above. R^{L18} represents a saturated hydrocarbylene group having 1 to 20 carbon atoms and having a valency of h+1 or an arylene group having 6 to 20 carbon atoms and having a valency of h+1, and may contain a heteroatom such as an oxygen atom, a sulfur atom, or a nitrogen atom. The saturated hydrocarbylene group may be linear, branched, or cyclic. “h” represents an integer of 1 to 3.

Examples of monomers to give a repeating unit containing the acid-labile group represented by the general formula (AL-3) include (meth)acrylate including an exo-form structure represented by the following general formula (AL-3)-22.

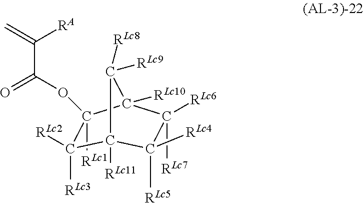

In the general formula (AL-3)-22, R^{A} is as defined above. R^{Lc1} represents a saturated hydrocarbyl group having 1 to 8 carbon atoms or an aryl group having 6 to 20 carbon atoms, the aryl group optionally being substituted. The saturated hydrocarbyl group may be linear, branched, or cyclic. R^{Lc2} to R^{Lc11} each independently represent a hydrogen atom or a hydrocarbyl group having 1 to 15 carbon atoms and optionally containing a heteroatom. Examples of the heteroatom include an oxygen atom etc. Examples of the hydrocarbyl group include alkyl groups having 1 to 15 carbon atoms, aryl groups having 6 to 15 carbon atoms, etc. R^{Lc2} and R^{Lc3}, R^{Lc4} and R^{Lc6}, R^{Lc4} and R^{Lc7}, R^{Lc5} and R^{Lc7}, R^{Lc5} and R^{Lc11}, R^{Lc6} and R^{Lc10}, R^{Lc8} and R^{Lc9}, or R^{Lc9} and R^{Lc10} may be bonded to each other to form a ring together with the carbon atom(s) bonded thereto. In such a case, the groups that are involved in the bonding are hydrocarbylene groups having 1 to 15 carbon atoms and optionally containing a heteroatom. Furthermore, R^{Lc2} and R^{Lc11}, R^{Lc8} and R^{Lc11}, or R^{Lc4} and R^{Lc6}, bonded to adjoining carbon atoms, may be bonded to each other directly and form a double bond. Note that the formula also represents an enantiomer.

Here, examples of monomers that give the repeating unit represented by the general formula (AL-3)-22 include those disclosed in JP 2000-327633 A etc. Specific examples include the following, but are not limited thereto. Note that in the following formulae, R^{A} is as defined above.

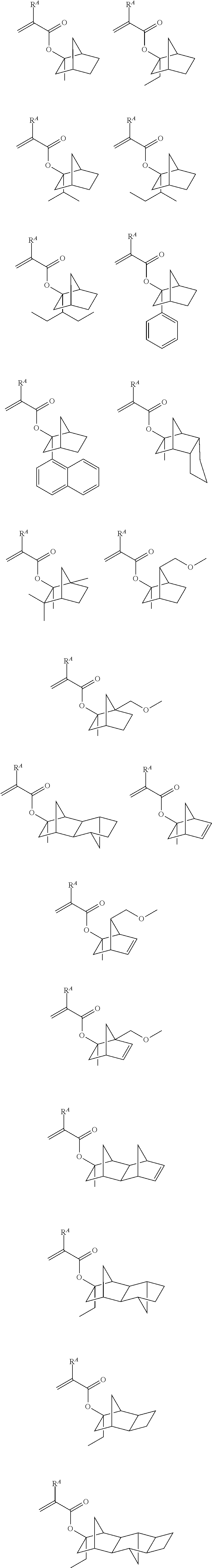

Examples of monomers to give a repeating unit containing an acid-labile group represented by the general formula (AL-3) also include (meth)acrylate represented by the following general formula (AL-3)-23, containing a furandiyl group, a tetrahydrofurandiyl group, or an oxanorbornanediyl group.

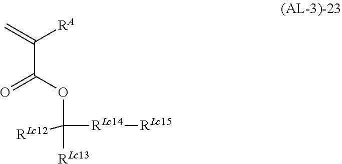

In the general formula (AL-3)-23, R^{A} is as defined above. R^{Lc12} and R^{Lc13} each independently represent a hydrocarbyl group having 1 to 10 carbon atoms. R^{Lc12} and R^{Lc13} may be bonded with each other to form an aliphatic ring together with the carbon atom bonded thereto. R^{Lc14} represents a furandiyl group, a tetrahydrofurandiyl group, or an oxanorbornanediyl group. R^{Lc15} represents a hydrogen atom or a hydrocarbyl group having 1 to 10 carbon atoms and optionally containing a heteroatom. The hydrocarbyl group may be linear, branched, or cyclic. Specific examples thereof include saturated hydrocarbyl groups having 1 to 10 carbon atoms etc.

Examples of monomers that give the repeating unit represented by the general formula (AL-3)-23 include the following, but are not limited thereto. Note that in the following formulae, R^{A} is as defined above, Ac represents an acetyl group, and Me represents a methyl group.

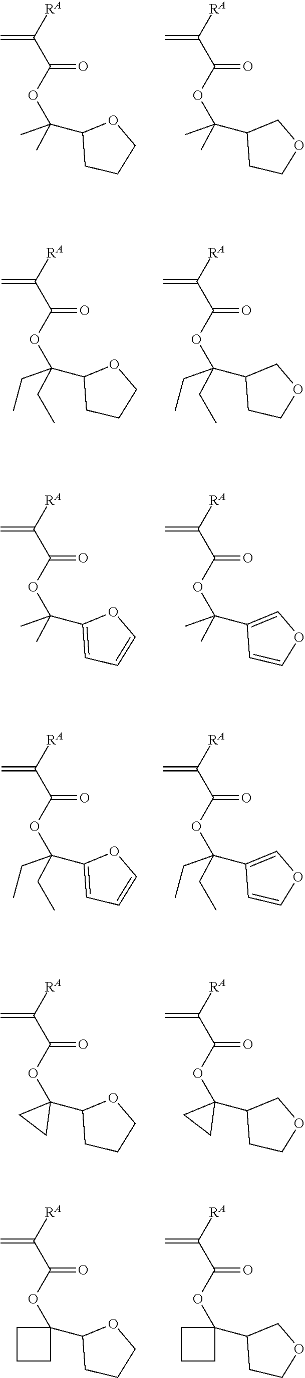

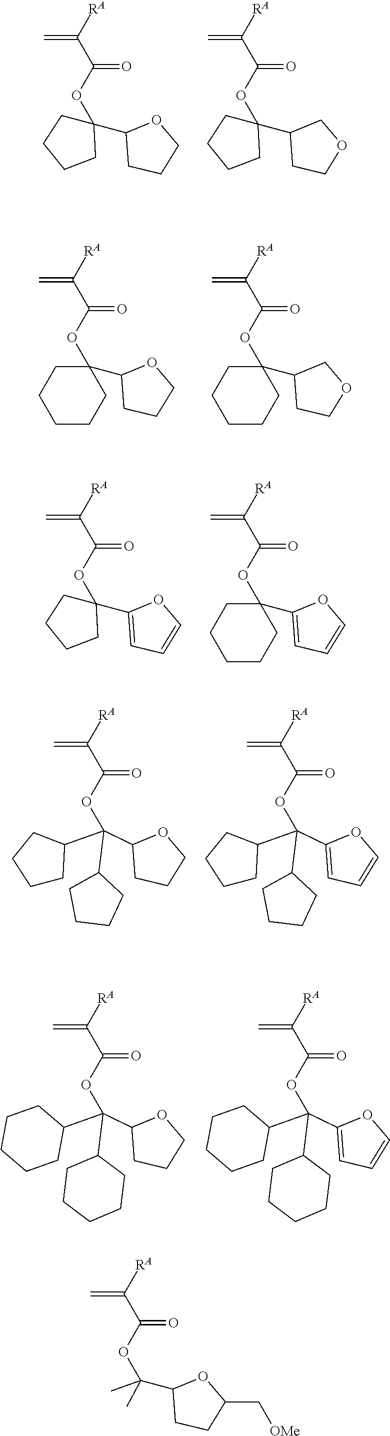

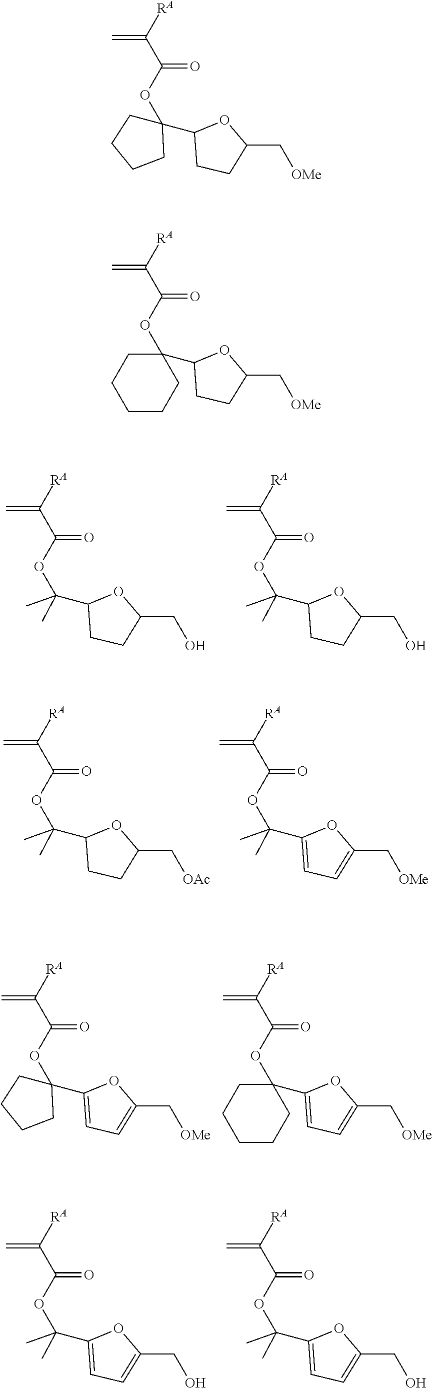

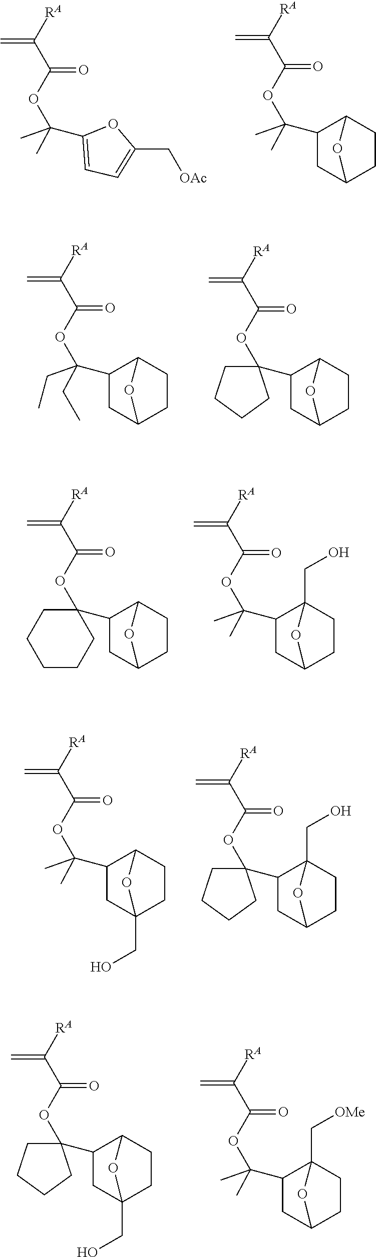

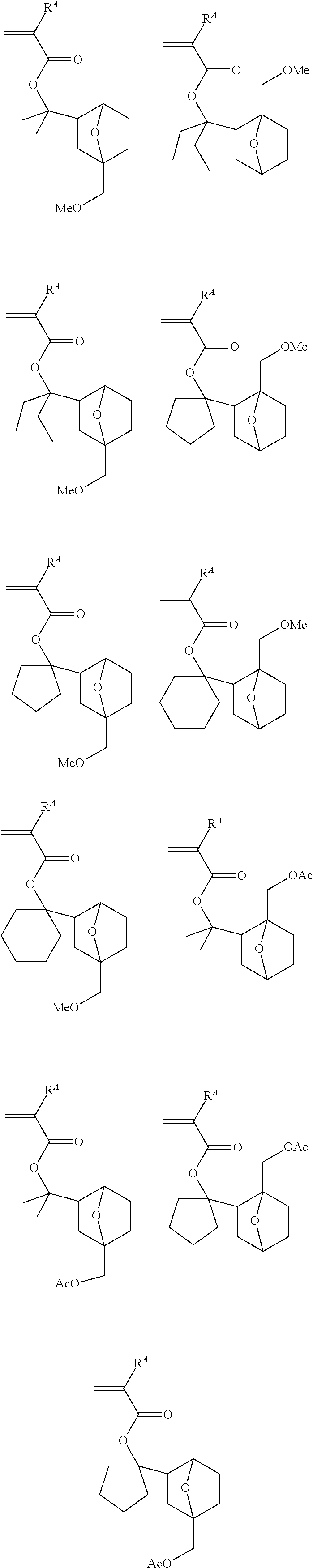

The resist material may further contain a repeating unit-c having an adhesive group selected from a hydroxy group, a carboxy group, a lactone ring, a carbonate group, a thiocarbonate group, a carbonyl group, a cyclic acetal group, an ether bond, an ester bond, a sulfonic acid ester bond, a cyano group, an amide group, —O—C(═O)—S—, and —O—C(═O)—NH—.

Examples of monomers that give the repeating unit-c include the following, but are not limited thereto. Incidentally, in the following formulae, R^{A} is as defined above.

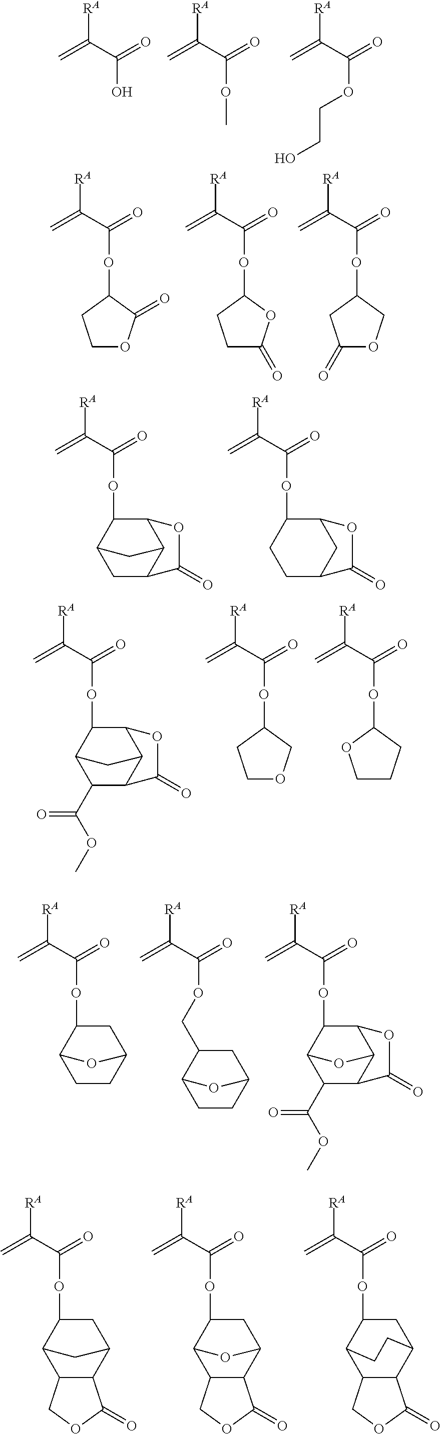

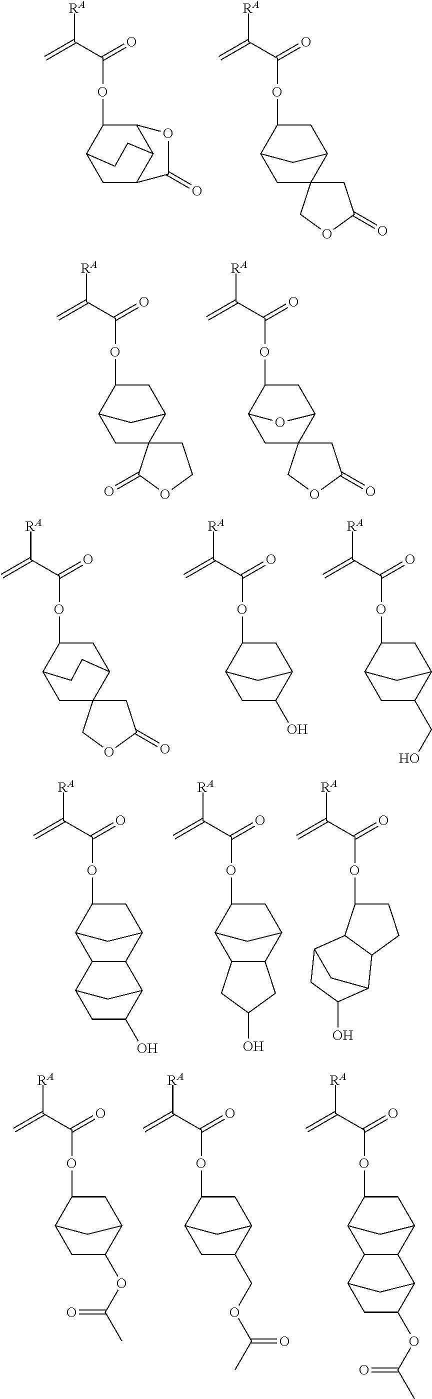

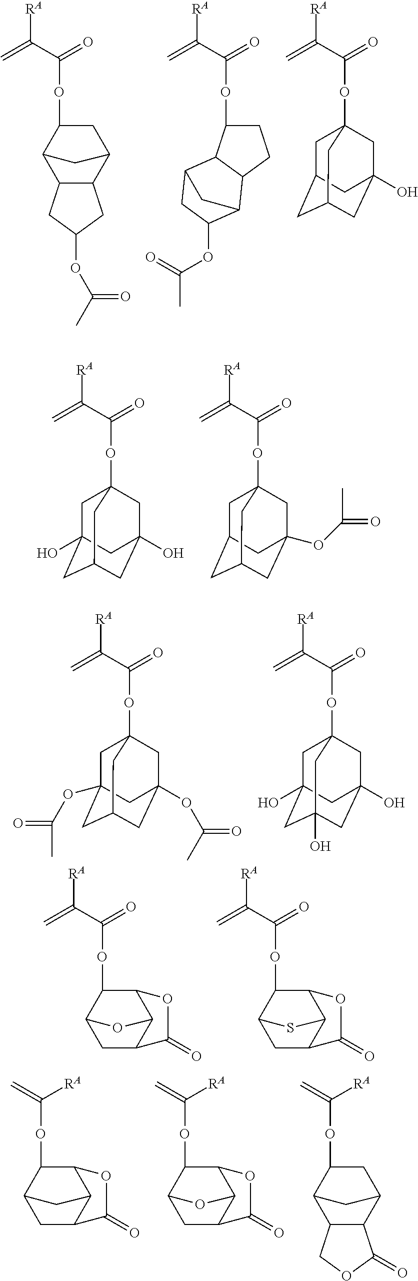

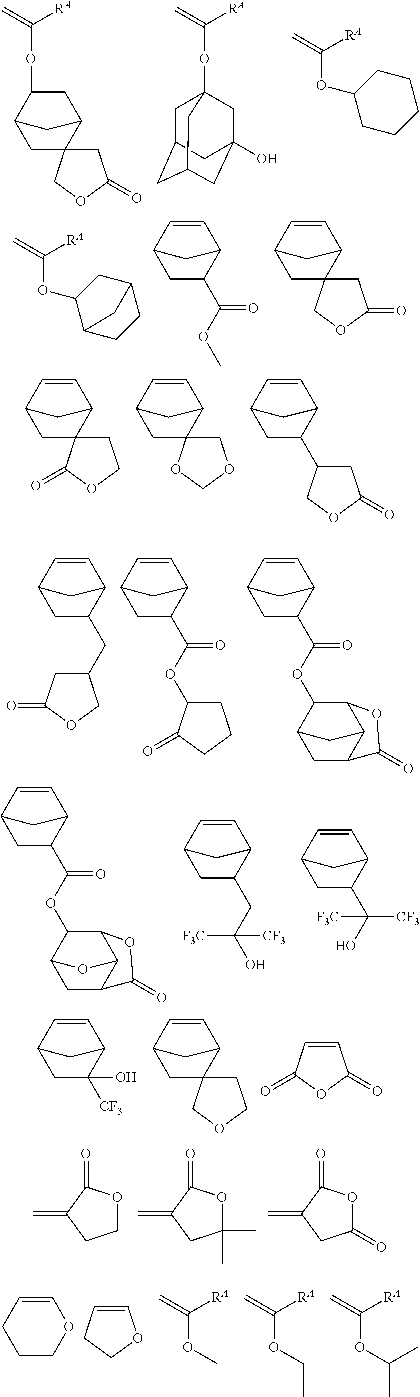

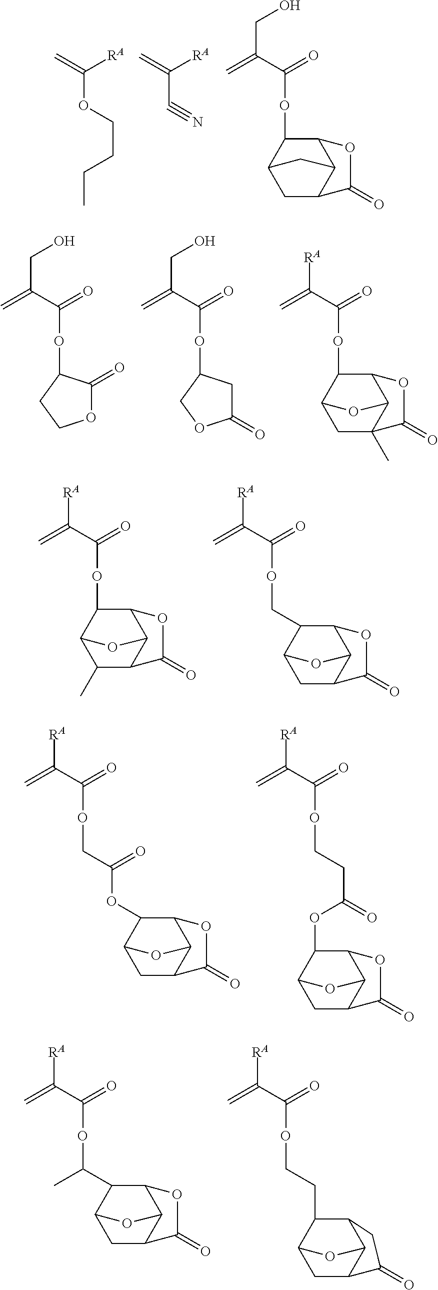

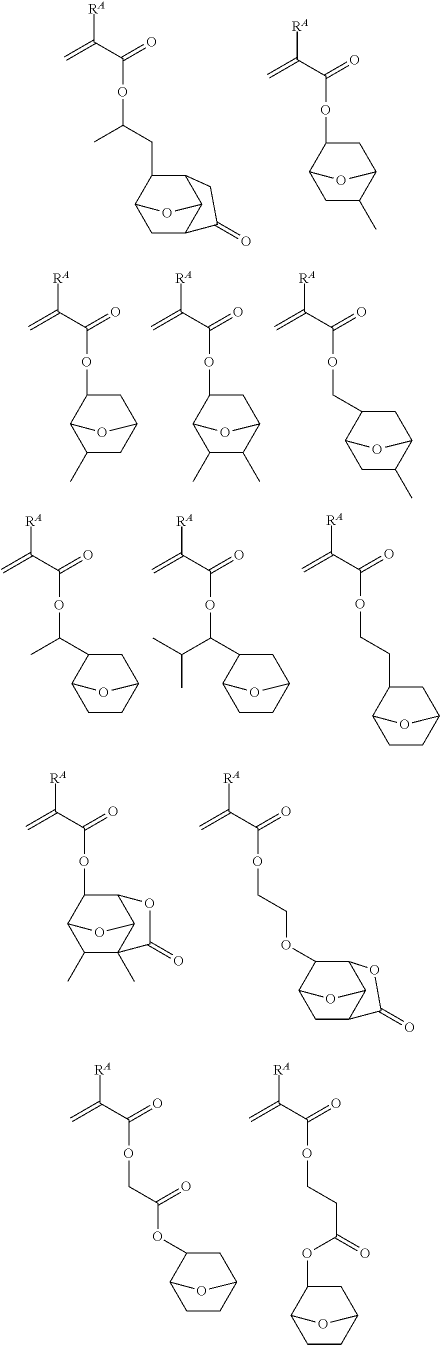

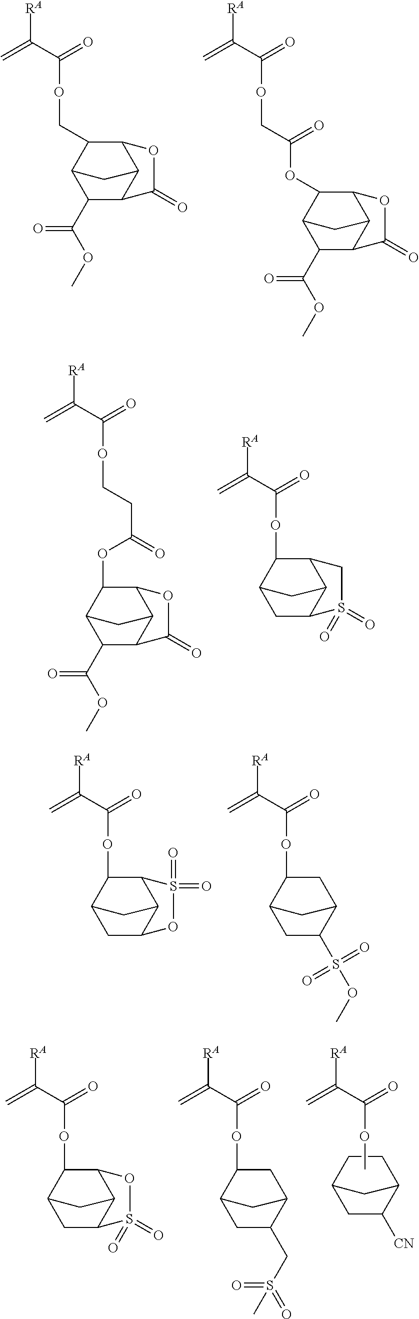

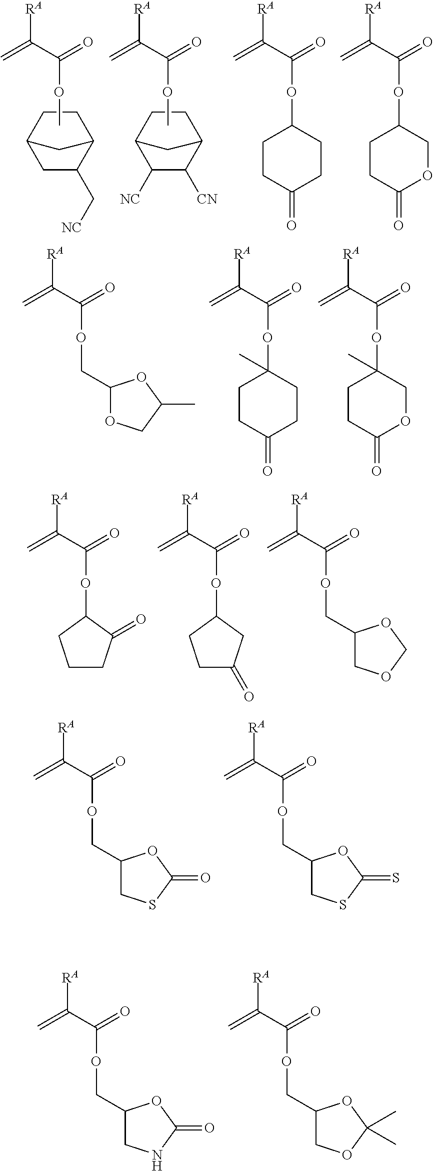

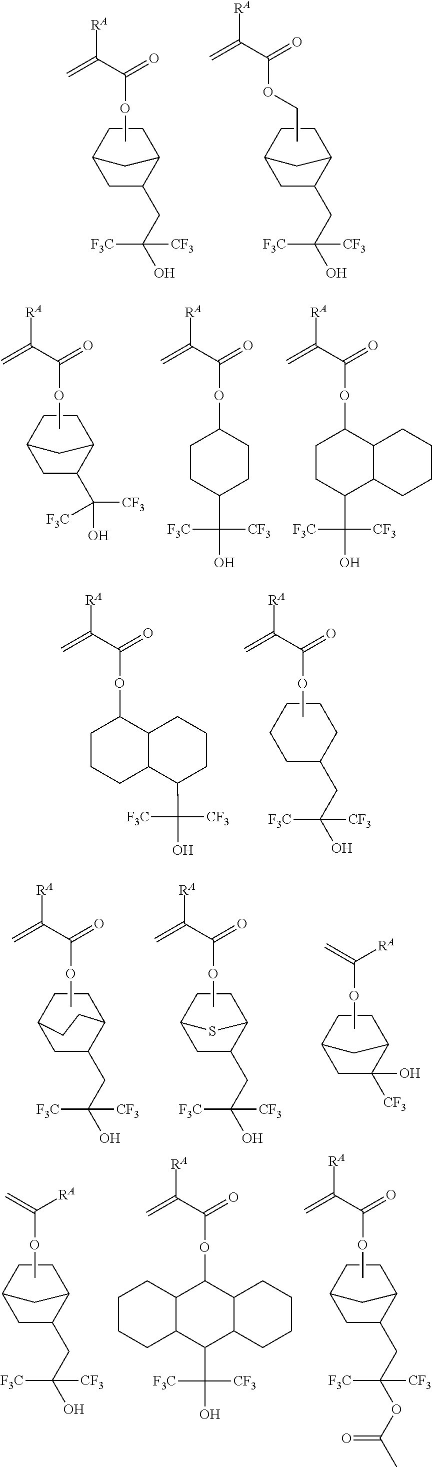

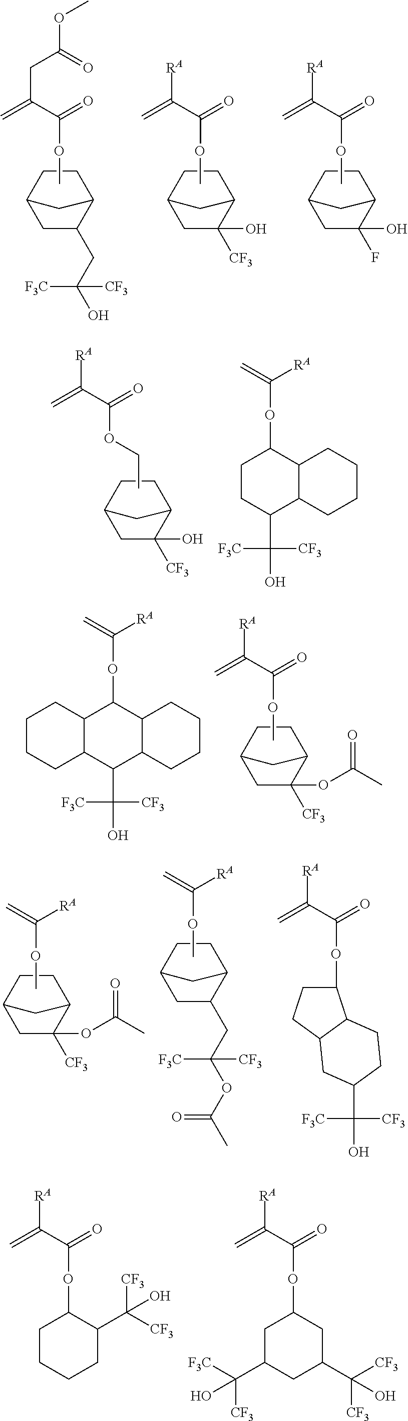

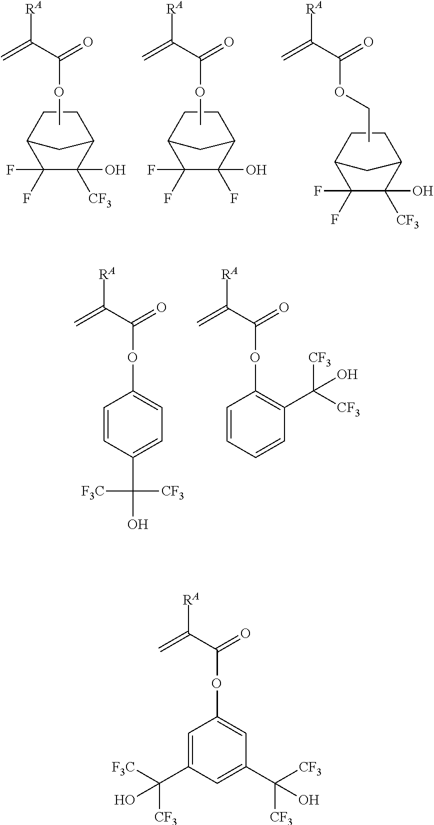

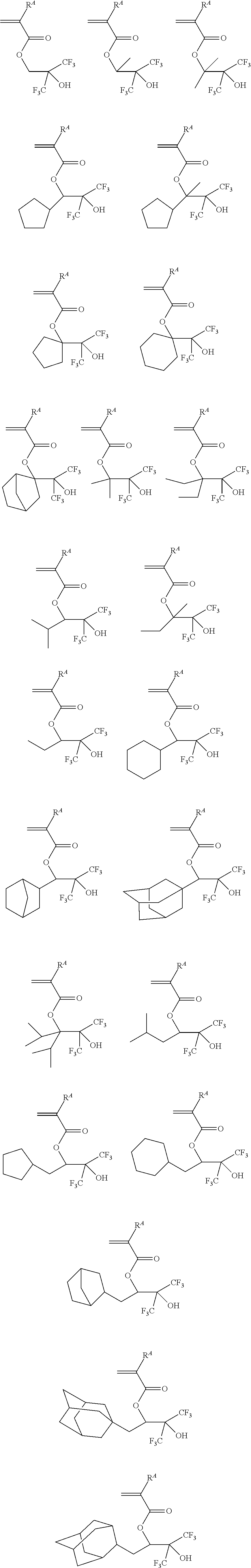

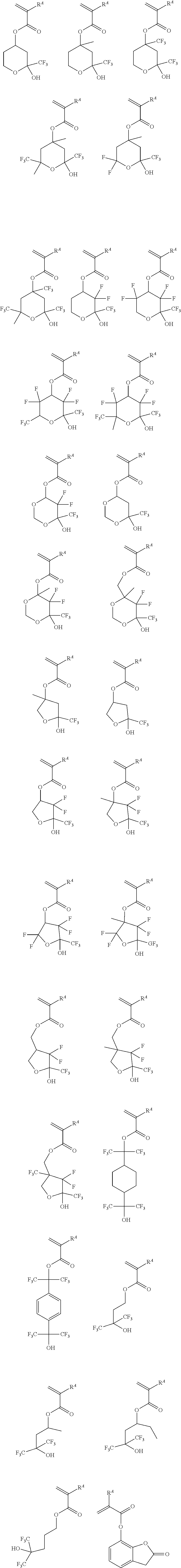

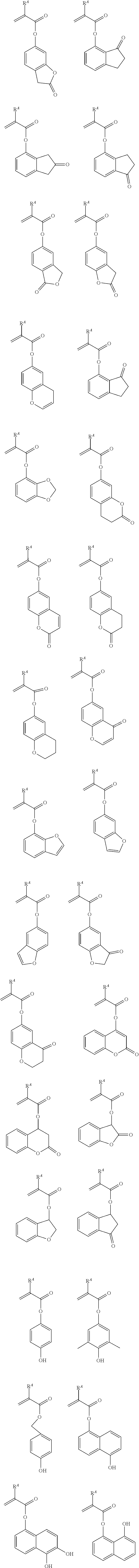

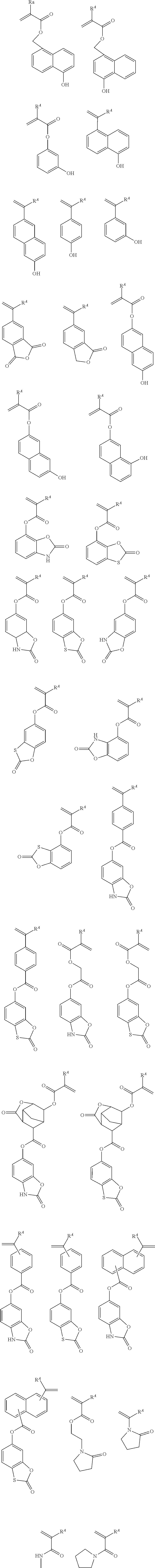

The resist material may further include a repeating unit-d derived from an onium salt containing a polymerizable unsaturated bond. Examples of preferable repeating units-d include repeating units represented by the following general formula (d1) (hereinafter, also referred to as repeating units-d1), repeating units represented by the following general formula (d2) (hereinafter, also referred to as repeating units-d2), and repeating units represented by the following general formula (d3) (hereinafter, also referred to as repeating units-d3). Incidentally, one kind of the repeating units-d1 to -d3 can be used, or two or more kinds thereof can be used in combination. That is, the resist material preferably further contains at least one kind of repeating unit-d selected from repeating units represented by the following general formulae (d1) to (d3).

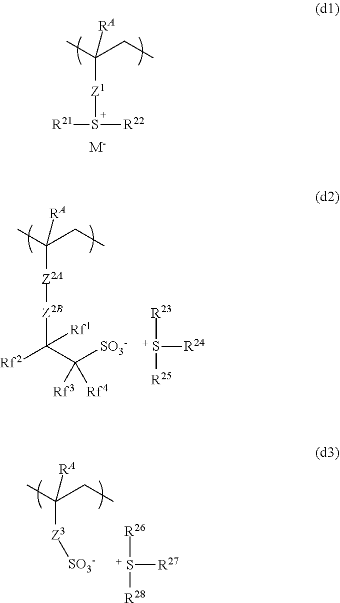

In the formulae, each R^{A} independently represents a hydrogen atom or a methyl group; Z¹ represents a single bond, a phenylene group, —O—Z¹¹—, —C(═O)—O—Z¹¹—, or —C(═O)—NH—Z¹¹—; Z¹¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, and a hydroxy group; Z^{2A} represents a single bond or an ester bond; Z^{2B} represents a single bond or a divalent group having 1 to 12 carbon atoms and optionally containing one or more selected from an ester bond, an ether bond, a lactone ring, a bromine atom, and an iodine atom; Z³ represents a single bond, a methylene group, an ethylene group, a phenylene group, a fluorinated phenylene group, —O—Z³¹—, —C(═O)—O—Z³¹—, or —C(═O)—NH—Z³¹—; Z³¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, a halogen atom, and a hydroxy group; Rf¹ to Rf⁴ each independently represent a hydrogen atom, a fluorine atom, or a trifluoromethyl group, provided that at least one of Rf¹ to Rf⁴ is a fluorine atom; R²¹ to R²⁸ each independently represent a monovalent hydrocarbon group having 1 to 20 carbon atoms and optionally containing a heteroatom; any two of R²³, R²⁴, and R²⁵ or any two of R²⁶, R²⁷, and R²⁸ are optionally bonded to each other to form a ring together with a sulfur atom bonded thereto; and M represents a non-nucleophilic counter ion.

In the formulae, each R^{A} independently represents a hydrogen atom or a methyl group, preferably a methyl group. Z¹ represents a single bond, a phenylene group, —O—Z¹¹—, —C(═O)—O—Z¹¹—, or —C(═O)—NH—Z¹¹—; and Z¹¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, and a hydroxy group, preferably ethyl ether. Z^{2A} represents a single bond or an ester bond, preferably an ester bond. Z^{2B} represents a single bond or a divalent group having 1 to 12 carbon atoms and optionally containing one or more selected from an ester bond, an ether bond, a lactone ring, a bromine atom, and an iodine atom. Z³ represents a single bond, a methylene group, an ethylene group, a phenylene group, a fluorinated phenylene group, —O—Z³¹—, —C(═O)—O—Z³¹—, or
—C(═O)—NH—Z³¹—; and Z³¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, a halogen atom, and a hydroxy group. Rf¹ to Rf⁴ each independently represent a hydrogen atom, a fluorine atom, or a trifluoromethyl group, provided that at least one of Rf¹ to Rf⁴ is a fluorine atom.

In the general formula (d2), Rf¹ to Rf⁴ each independently represent a hydrogen atom, a fluorine atom, or a trifluoromethyl group, provided that at least one of Rf¹ to Rf⁴ is a fluorine atom. In particular, at least one of Rf³ and Rf⁴ is preferably a fluorine atom, and more preferably, both Rf³ and Rf⁴ are fluorine atoms.

In the general formulae (d1) to (d3), R²¹ to R²⁸ each independently represent a monovalent hydrocarbon group having 1 to 20 carbon atoms and optionally containing a heteroatom. In addition, any two of R²³, R²⁴, and R²⁵ or any two of R²⁶, R²⁷, and R²⁸ are optionally bonded to each other to form a ring together with a sulfur atom bonded thereto. The monovalent hydrocarbon group may be linear, branched, or cyclic, and specific examples include alkyl groups having 1 to 12 carbon atoms, aryl groups having 6 to 12 carbon atoms, and aralkyl groups having 7 to 20 carbon atoms. Furthermore, part or all of the hydrogen atoms of these groups may each be substituted with an alkyl group having 1 to 10 carbon atoms, a halogen atom, a trifluoromethyl group, a cyano group, a nitro group, a hydroxy group, a mercapto group, an alkoxy group having 1 to 10 carbon atoms, an alkoxycarbonyl group having 2 to 10 carbon atoms, or an acyloxy group having 2 to 10 carbon atoms, and part of the carbon atoms of these groups may be substituted with a carbonyl group, an ether bond, or an ester bond.

Specific examples of the sulfonium cations in the general formulae (d2) and (d3) include those given as examples of the cation of the sulfonium salt represented by the general formula (1-1) described later.

In the general formula (d1), M-represents a non-nucleophilic counter ion. Examples of the non-nucleophilic counter ion include halide ions, such as chloride and bromide ions; fluoroalkylsulfonate ions, such as triflate, 1,1,1-trifluoroethanesulfonate, and nonafluorobutanesulfonate ions; arylsulfonate ions, such as tosylate, benzenesulfonate, 4-fluorobenzenesulfonate, and 1,2,3,4,5-pentafluorobenzenesulfonate ions; alkylsulfonate ions, such as mesylate and butanesulfonate ions; imidate ions, such as bis(trifluoromethylsulfonyl) imide, bis(perfluoroethylsulfonyl) imide, and bis(perfluorobutylsulfonyl) imide ions; and methide ions such as tris(trifluoromethylsulfonyl) methide and tris(perfluoroethylsulfonyl) methide ions.

Examples of the non-nucleophilic counter ion further include: sulfonate ions represented by the following general formula (d1-1), having a substituting fluorine atom at a position; sulfonate ions represented by the following general formula (d1-2), having a substituting fluorine atom at a position and having a substituting trifluoromethyl group at β position; etc.

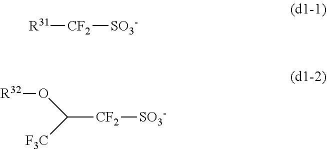

In the general formula (d1-1), R³¹ represents a hydrogen atom, an alkyl group having 1 to 20 carbon atoms, an alkenyl group having 2 to 20 carbon atoms, or an aryl group having 6 to 20 carbon atoms, and optionally contains an ether bond, an ester bond, a carbonyl group, a lactone ring, or a fluorine atom. The alkyl group and the alkenyl group may be linear, branched, or cyclic.

In the general formula (d1-2), R³² represents a hydrogen atom, an alkyl group having 1 to 30 carbon atoms, an acyl group having 2 to 30 carbon atoms, an alkenyl group having 2 to 20 carbon atoms, an aryl group having 6 to 20 carbon atoms, or an aryloxy group having 6 to 20 carbon atoms, and optionally contains an ether bond, an ester bond, a carbonyl group, or a lactone ring. The alkyl group, the acyl group, and the alkenyl group may be linear, branched, or cyclic.

In addition, at least one iodine atom is preferably contained in the Z^{2B}.

Examples of monomers to give the repeating unit-d1 include the following, but are not limited thereto. Incidentally, in the following formulae, R^{A} and M⁻ are as defined above.

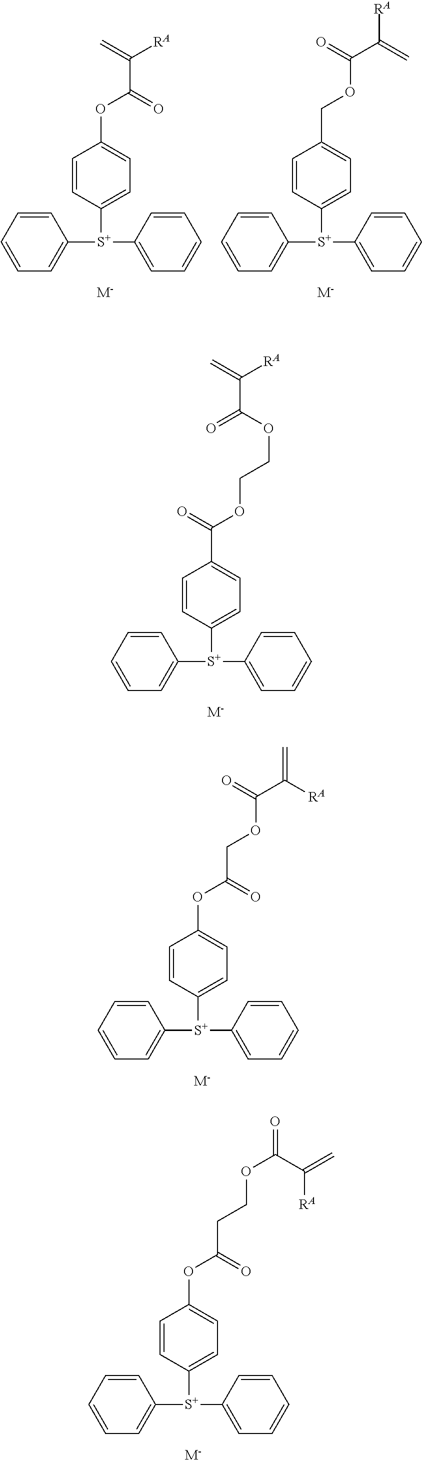

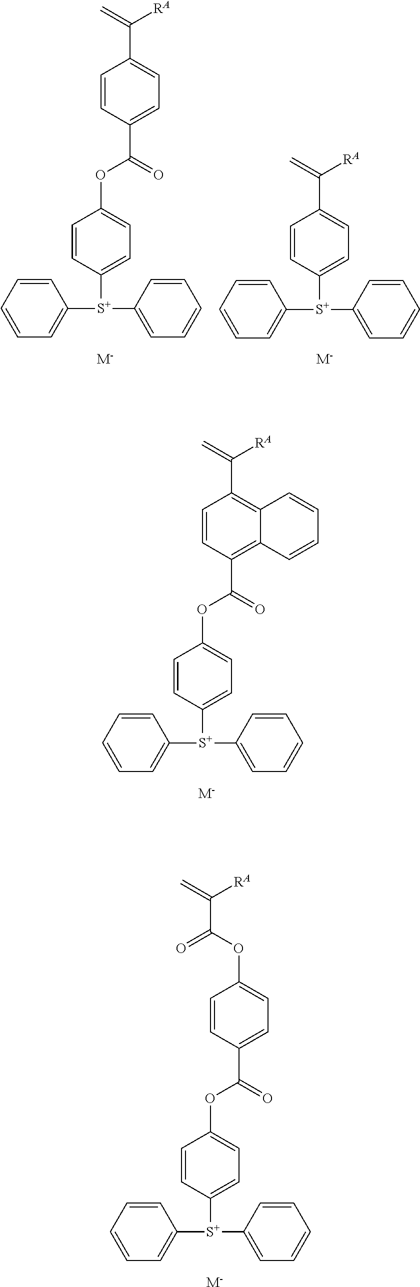

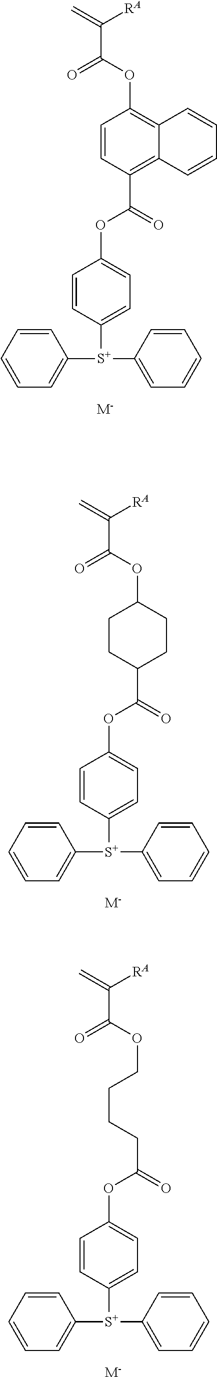

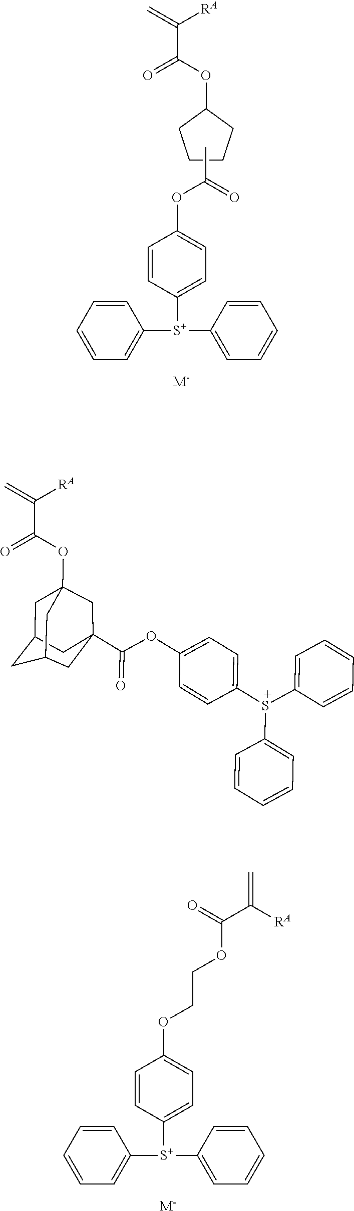

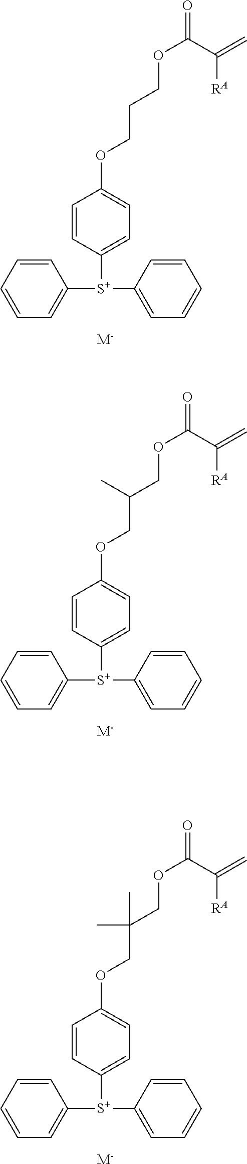

Examples of monomers to give the repeating unit-d2 include the following, but are not limited thereto. Incidentally, in the following formulae, R^{A} is as defined above.

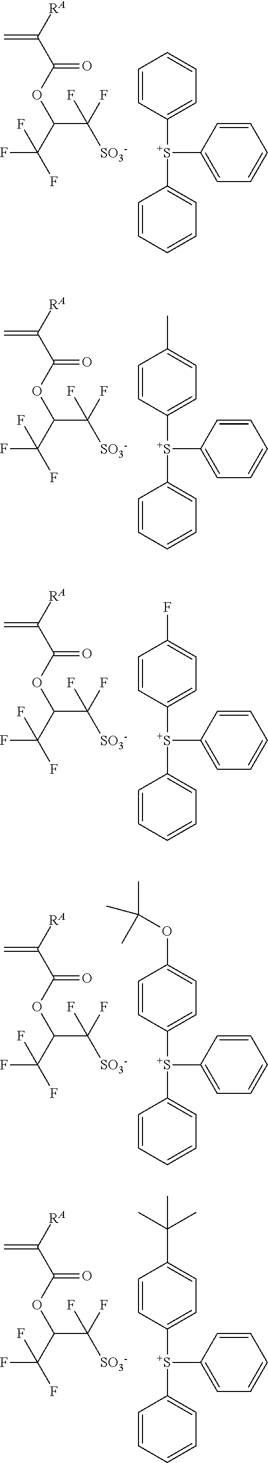

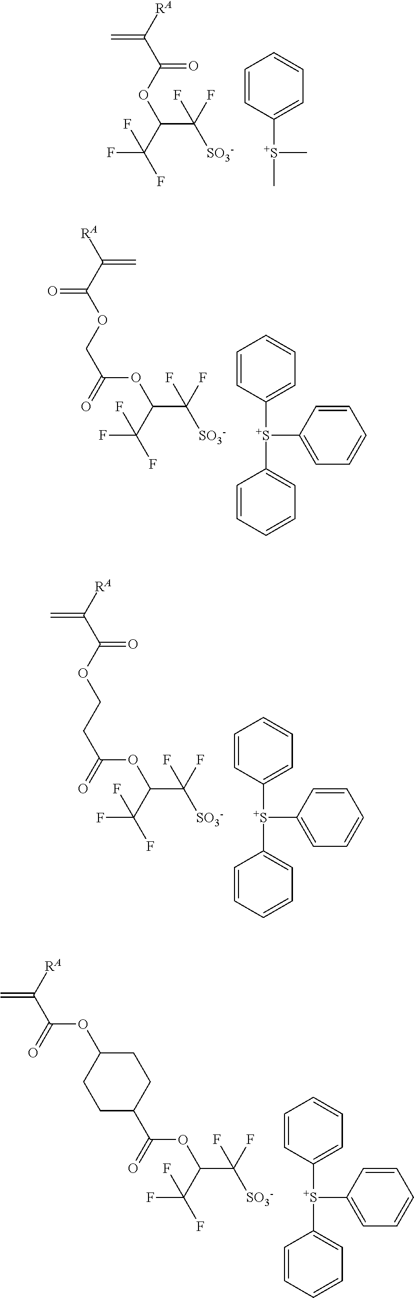

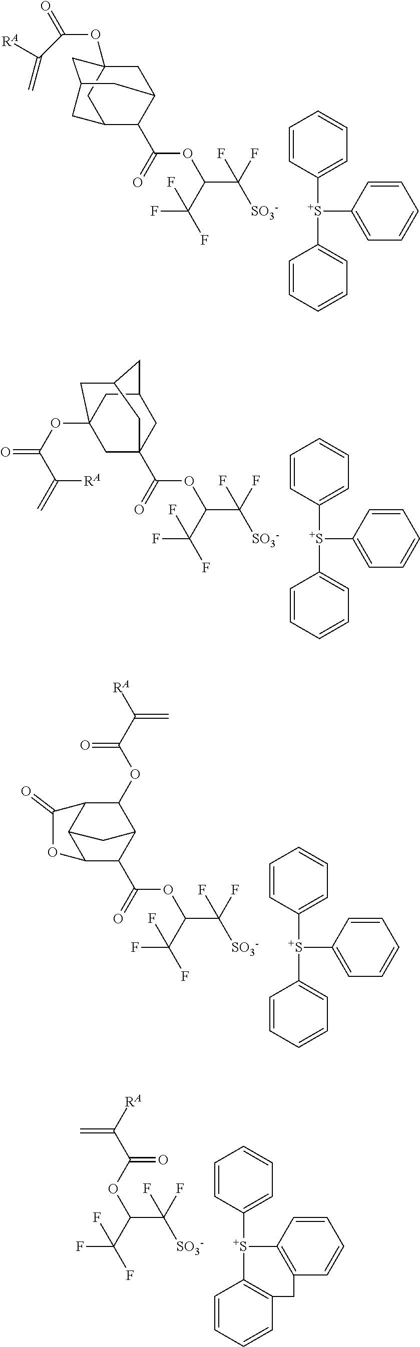

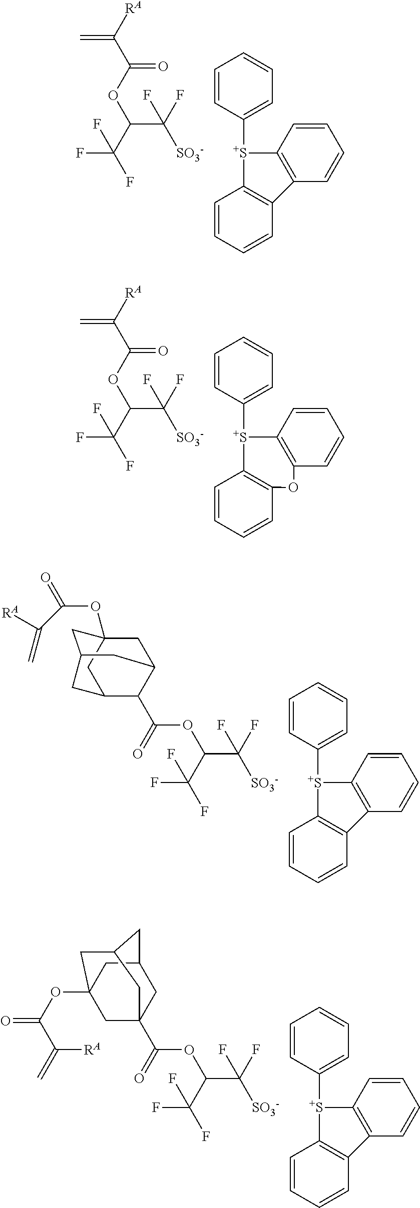

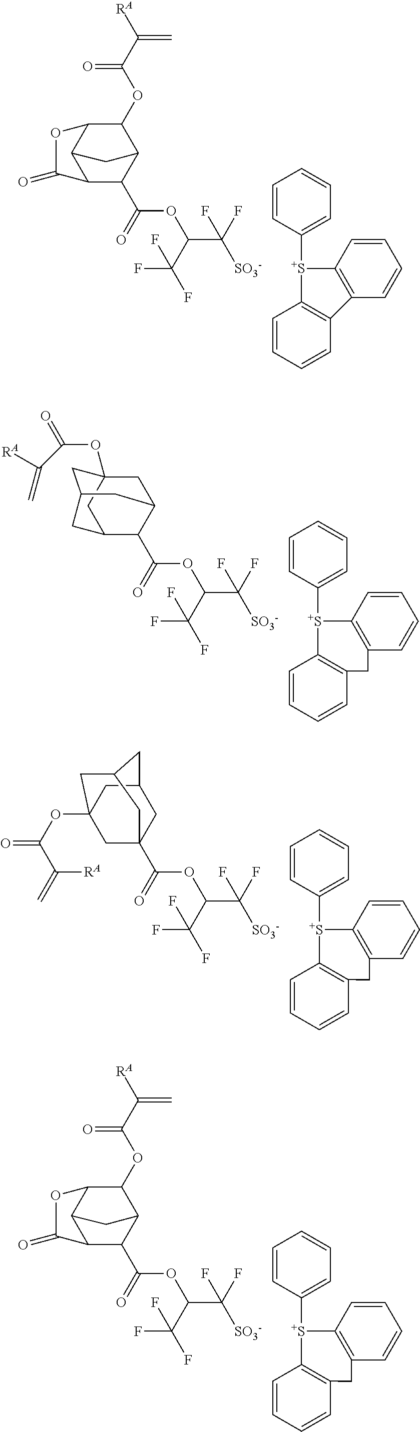

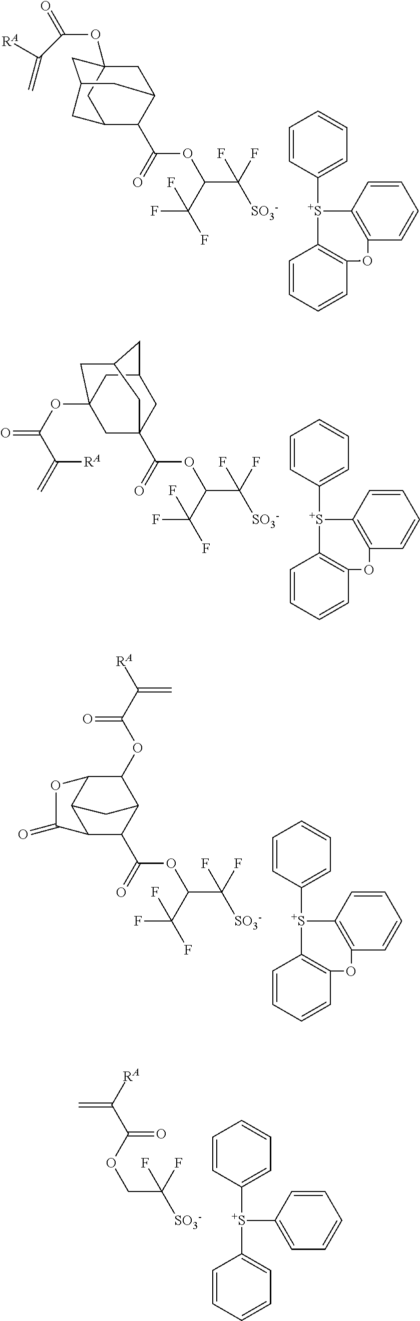

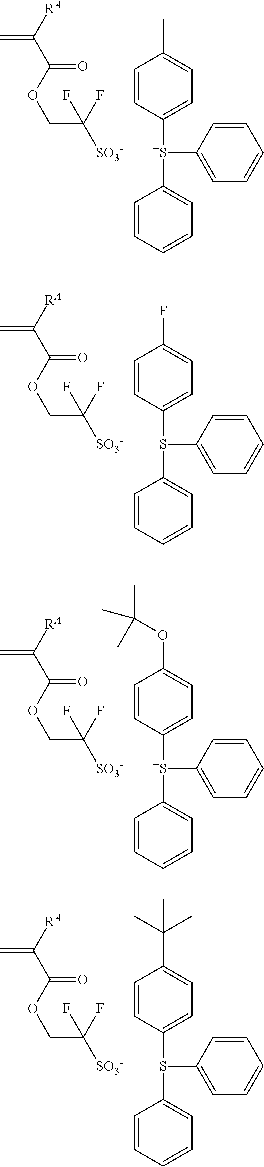

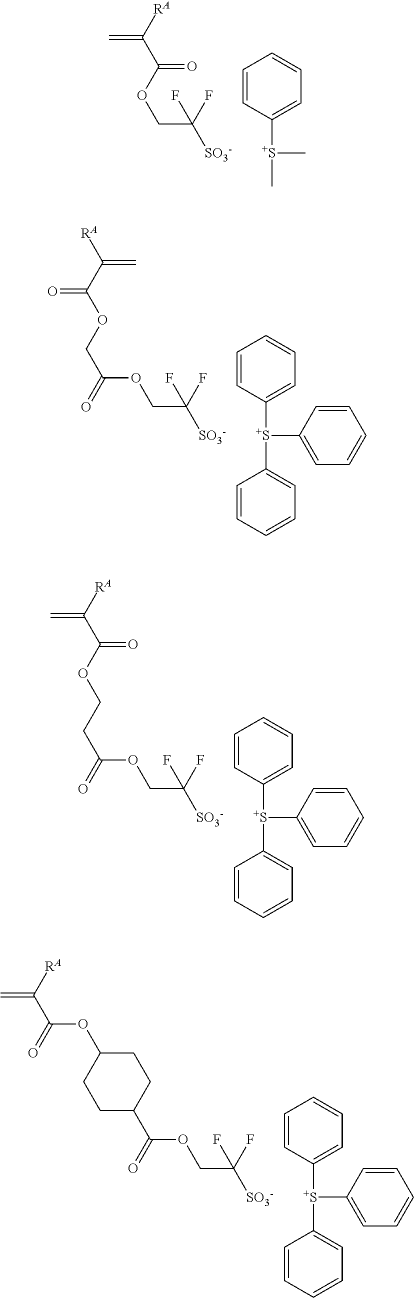

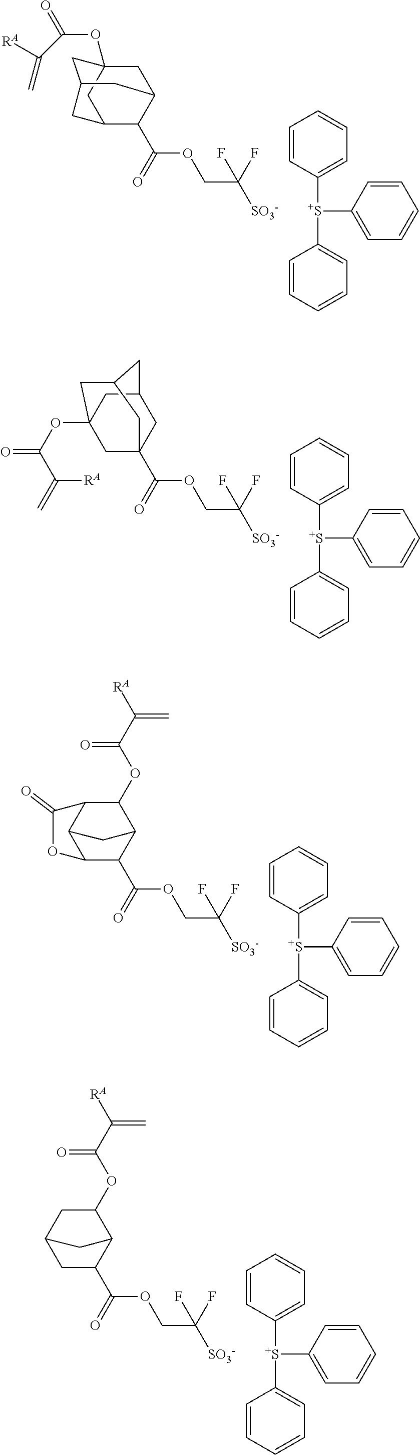

Favorable examples of monomers to give the repeating unit-d2 also include those having an anion shown below. Incidentally, in the following formulae, R^{A} is as defined above.

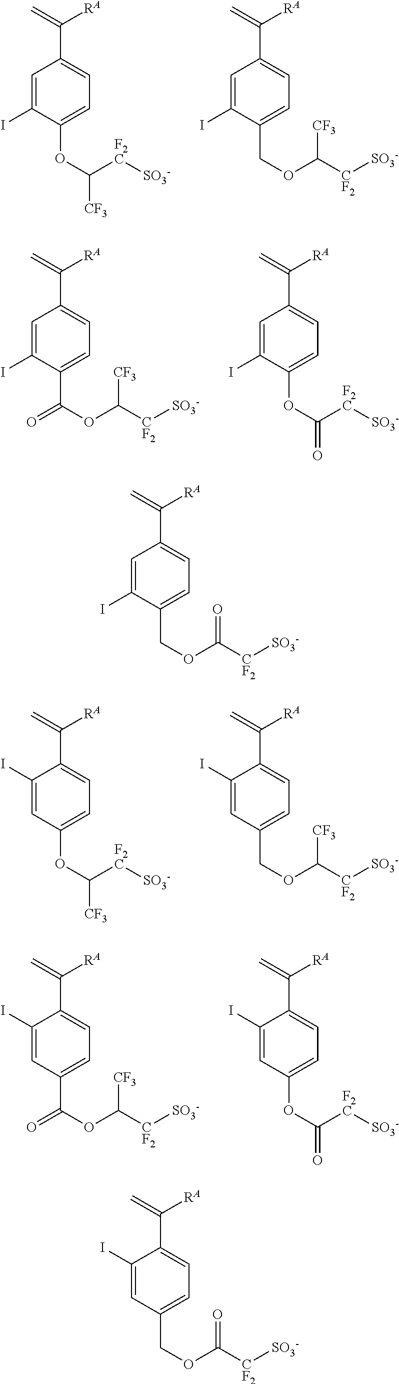

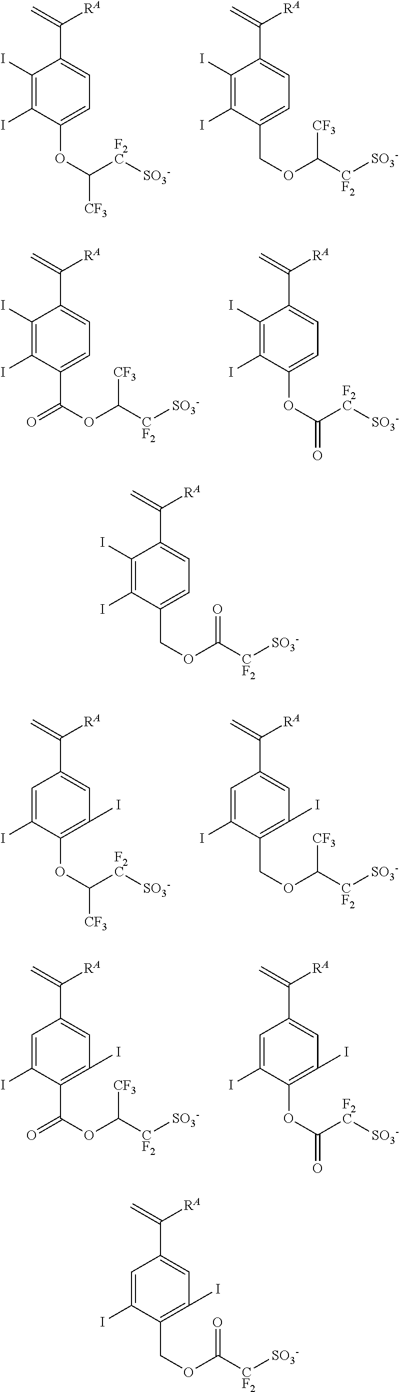

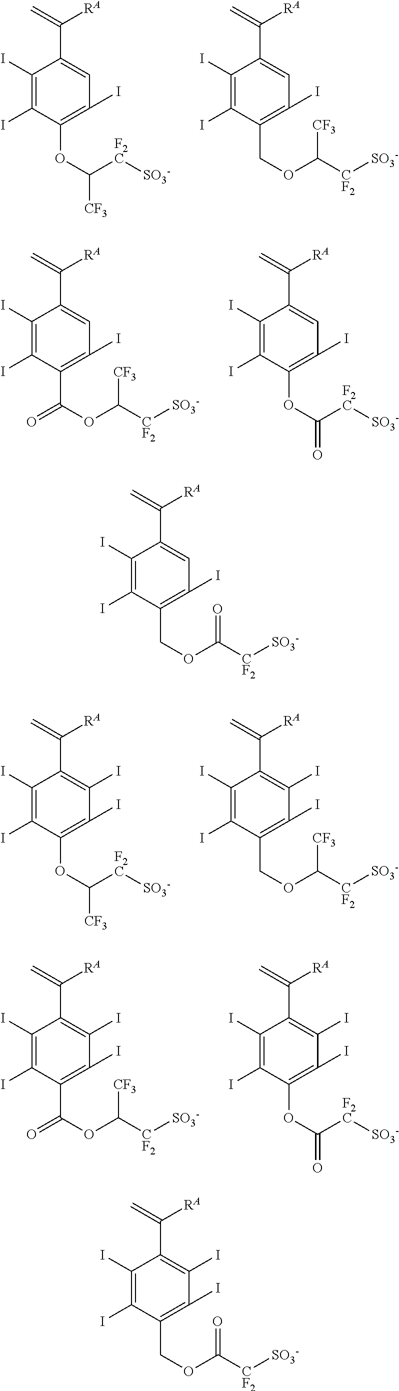

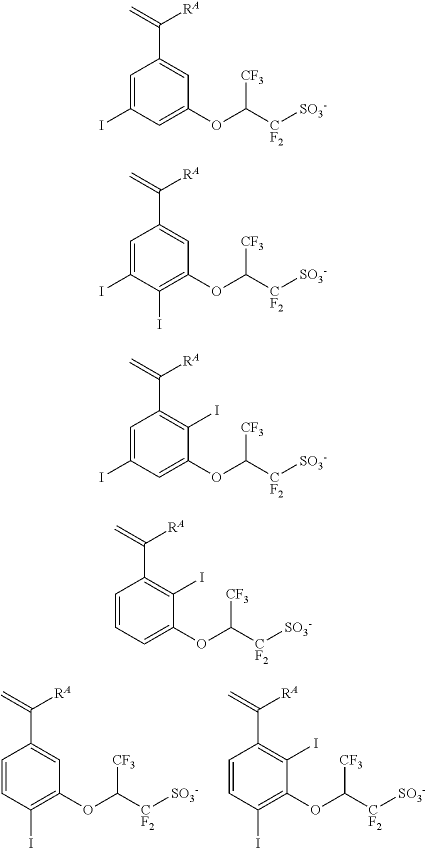

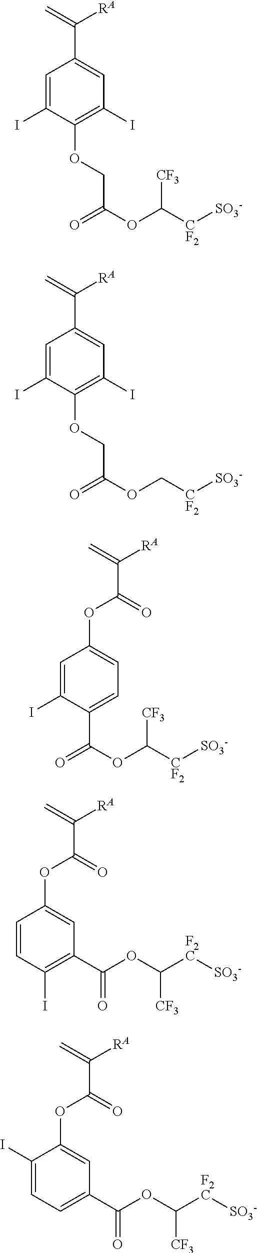

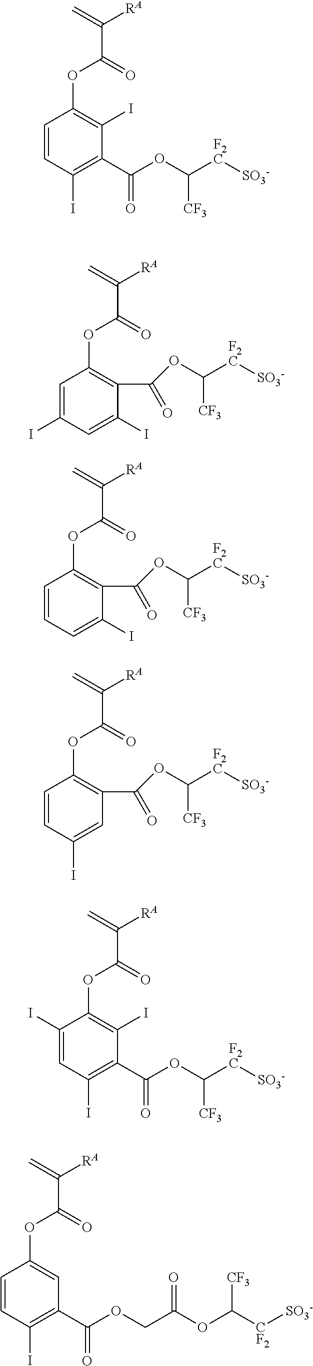

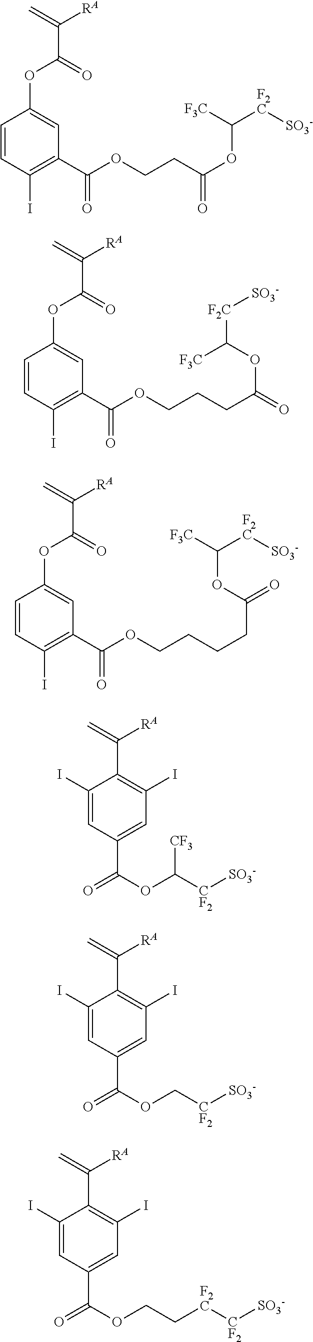

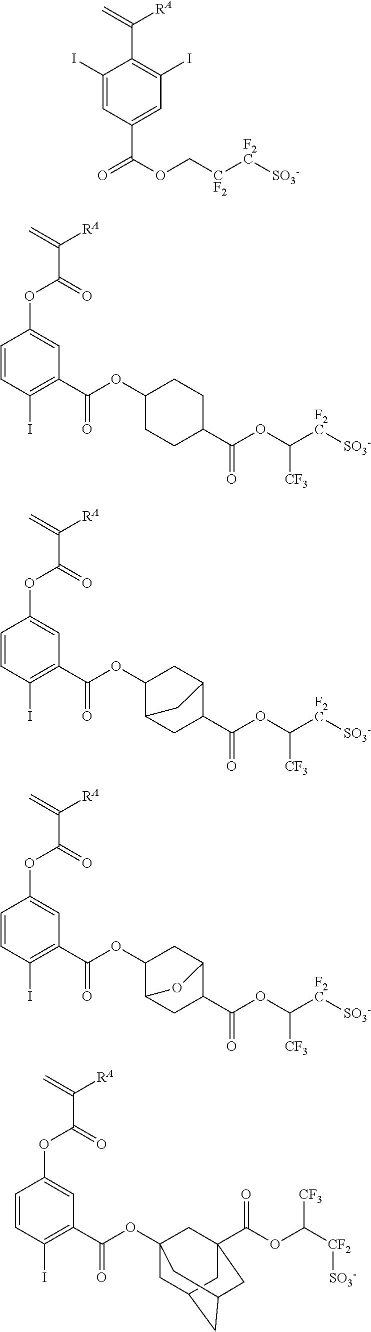

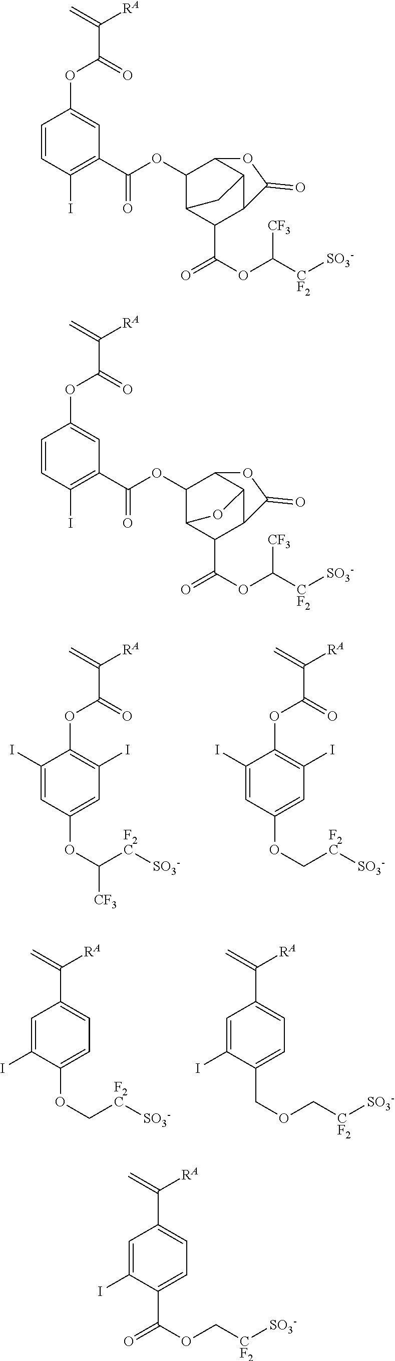

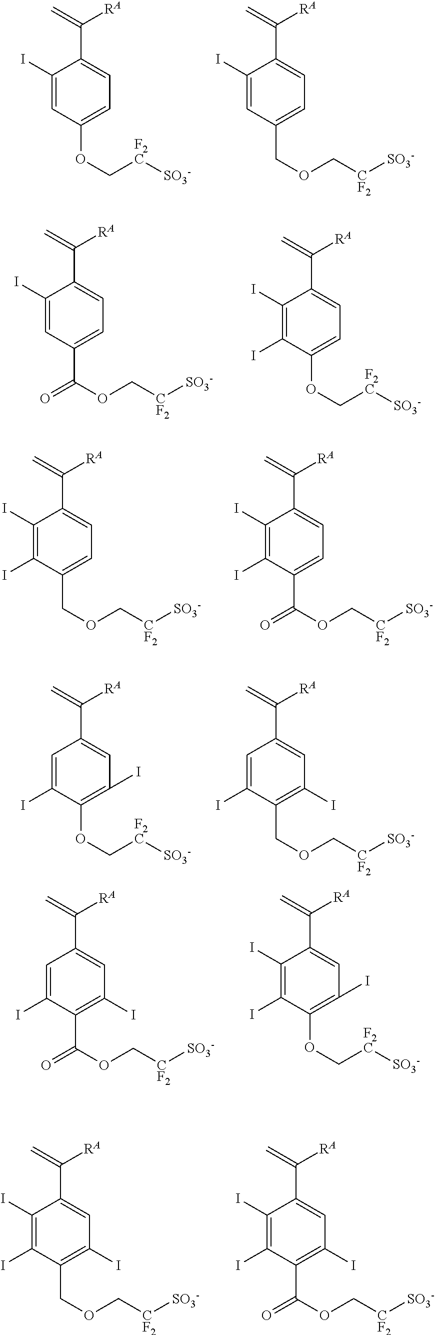

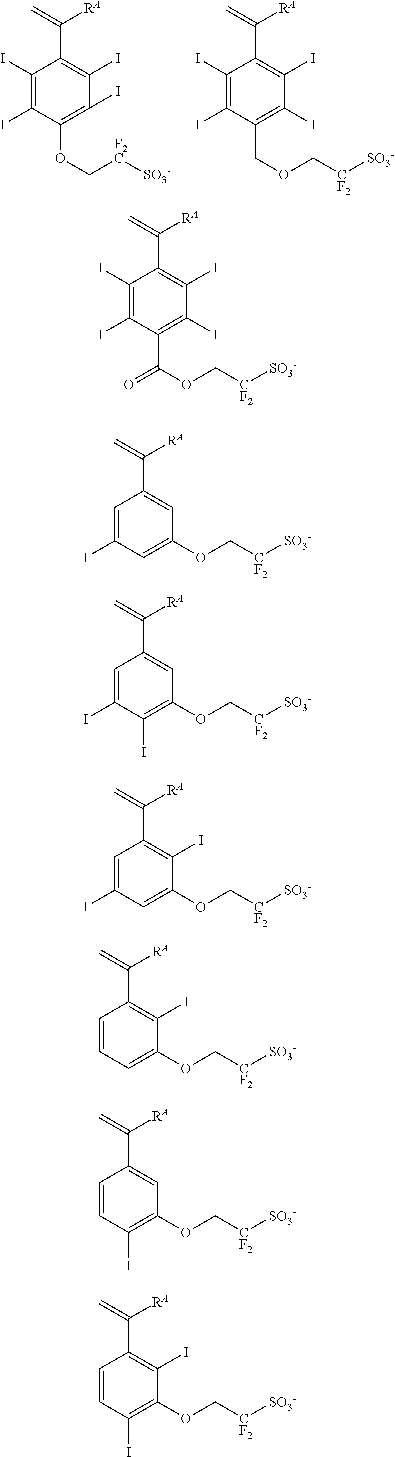

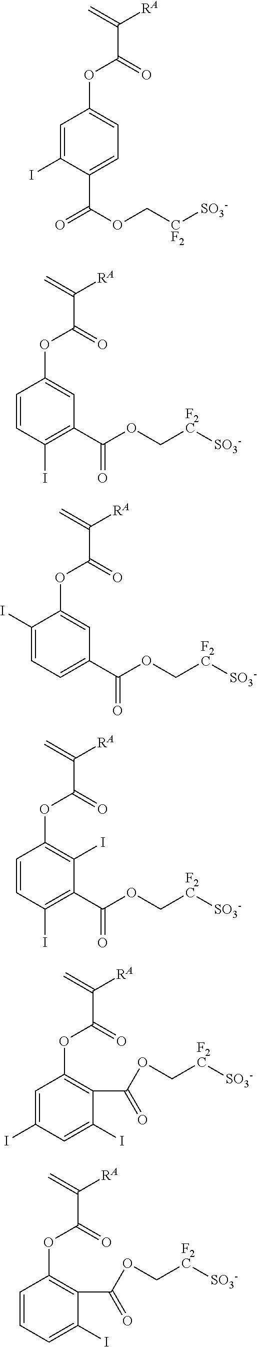

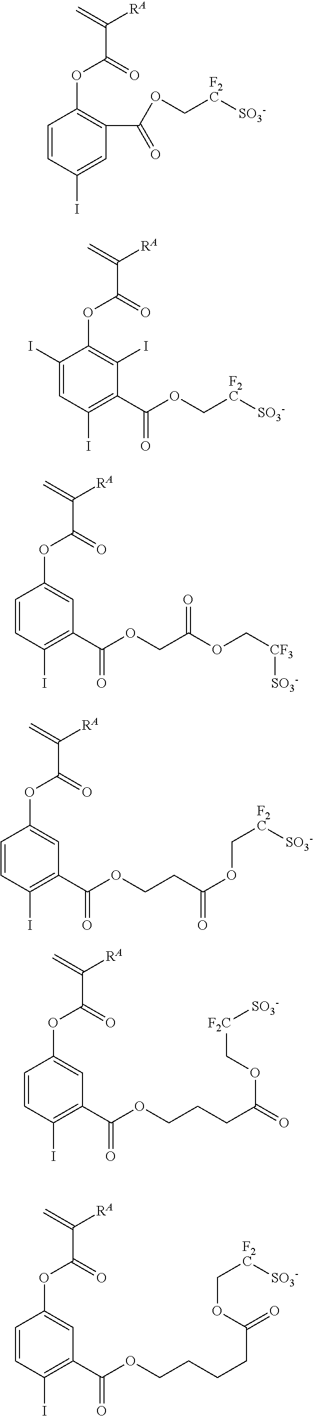

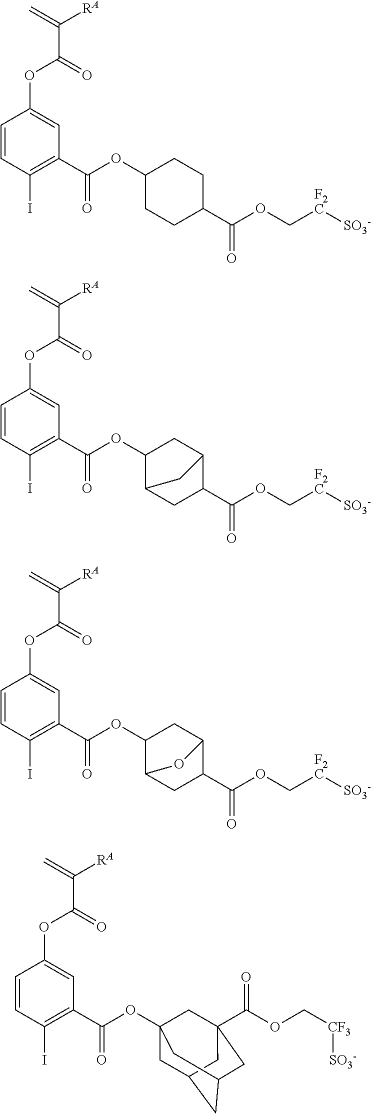

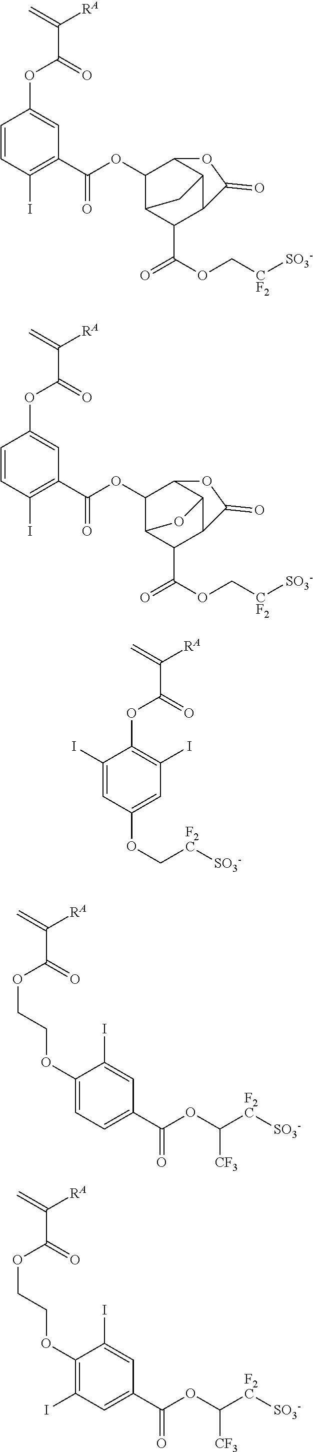

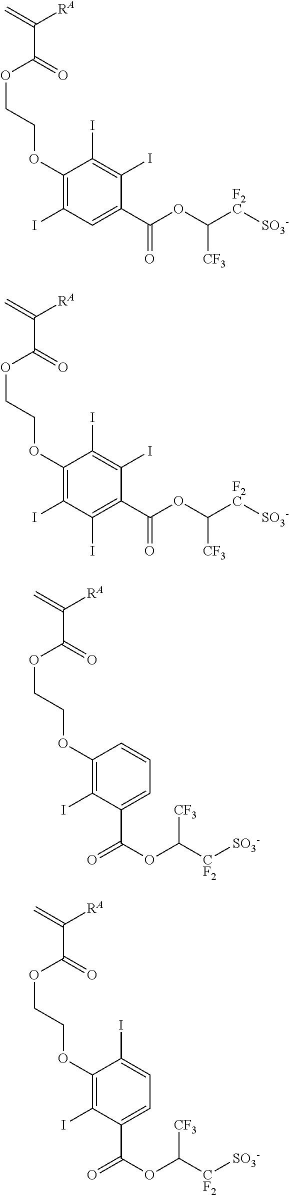

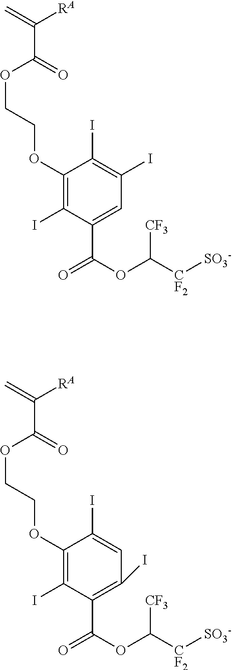

Examples of monomers to give the repeating unit-d3 include the following, but are not limited thereto. Incidentally, in the following formulae, R^{A} is as defined above.

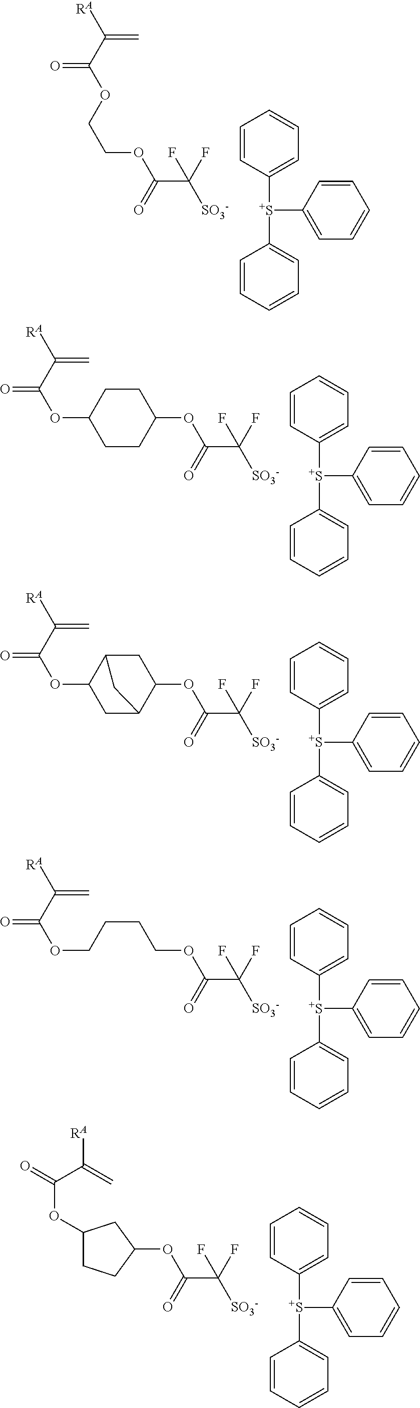

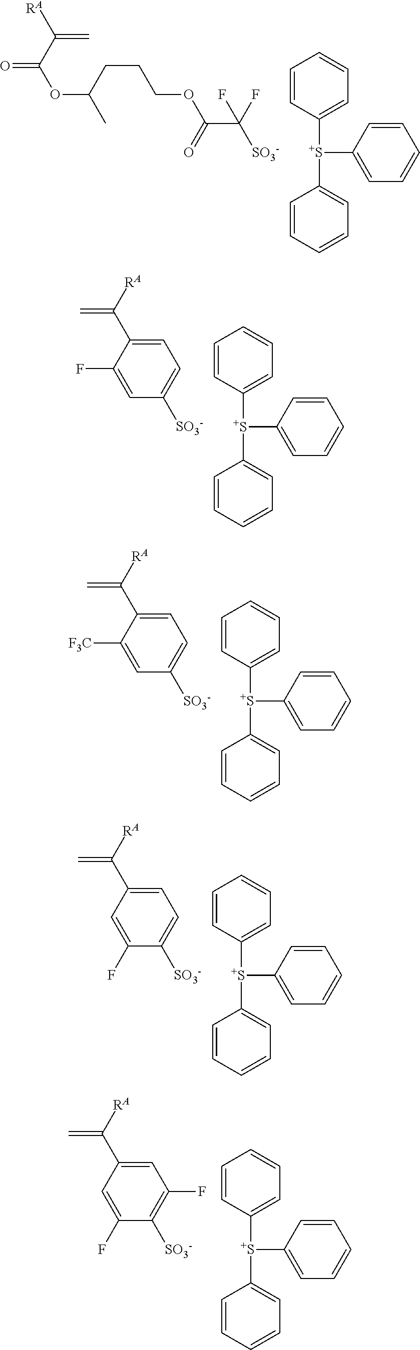

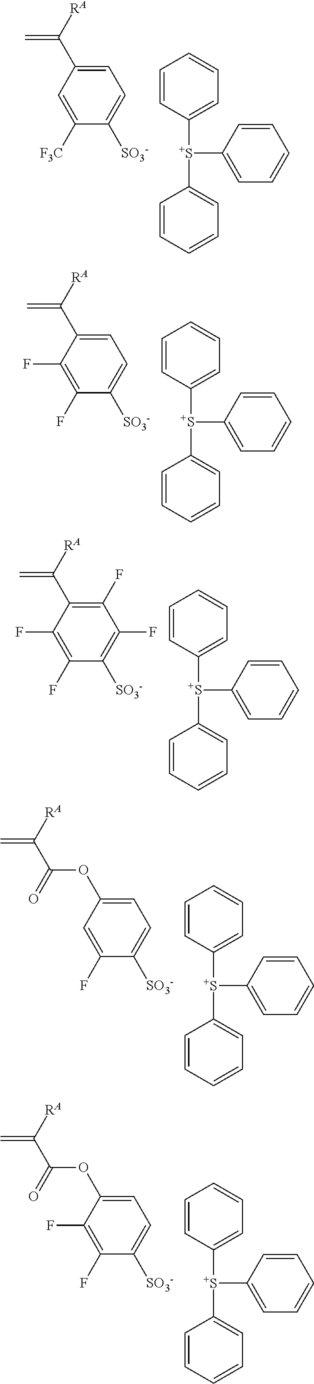

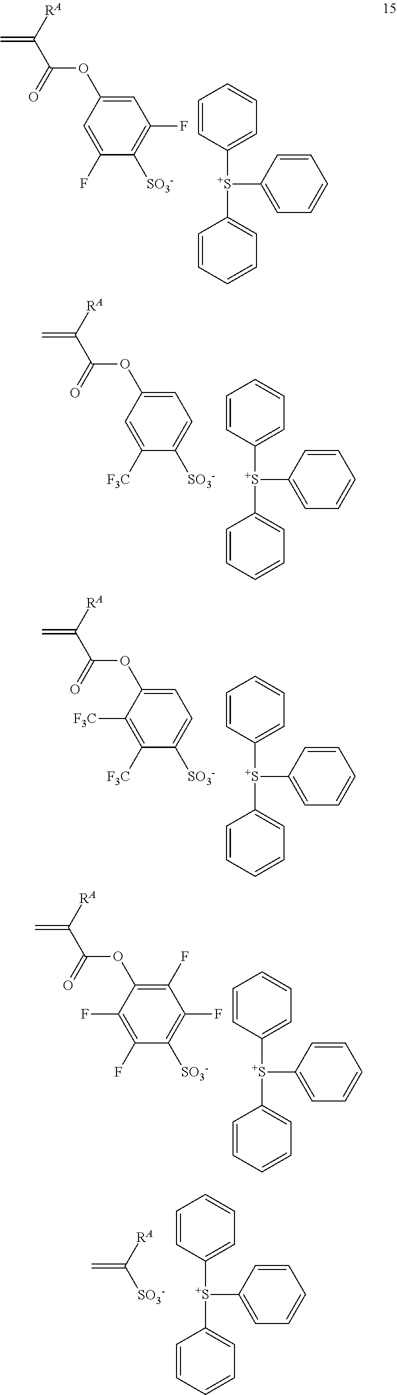

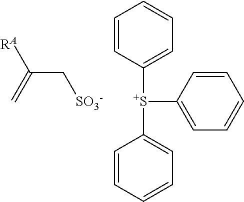

The repeating units-d1 to -d3 have the function of acid generators. When an acid generator is bonded to a polymer main chain, acid diffusion can be reduced, and thus, it is possible to prevent the degradation of resolution due to blurring caused by acid diffusion. In addition, by the acid generator being dispersed uniformly, LWR can be improved. Note that when a resist material containing the repeating unit-d is used, the additive-type acid generator described later may be omitted.

It is possible for the inventive resist material to have all functions within a single polymer by copolymerization between: the repeating unit-a, having the function of a quencher and having a sulfonium salt or iodonium salt of a substituted or unsubstituted iodinated monovalent aromatic carboxylic acid bonded to the polymer main chain via an ester bond; and the repeating unit-d of d1 to d3, each having the function of an acid generator. In this case, materials contained other than the polymer may be only an organic solvent and a surfactant in some cases, and this simple constitution of ingredients has an advantage of allowing high productivity.

The polymerization rate of a quencher of a sulfonium salt or iodonium salt of an iodinated monovalent aromatic carboxylic acid having a double bond is of the same level as the polymerization rate of an acid generator of a sulfonium salt or iodonium salt, and therefore, edge roughness after development can be improved by the uniform presence of the quencher and the acid generator in the polymer. When an iodine atom is contained in the anion moiety of the acid generator, the edge roughness can be improved further by the enhancement of contrast in acid generation caused by an increase in the number of photons absorbed. The repeating unit-a, having the sulfonium salt or iodonium salt of the iodinated monovalent aromatic carboxylic acid, in the inventive resist material exhibits an effect of improving edge roughness by the enhancement of contrast in quencher decomposition caused by an increase in the number of photons absorbed by iodine absorption.

The resist material may further include a repeating unit-e containing no amino groups and containing an iodine atom. Examples of monomers to give the repeating unit-e include the following, but are not limited thereto. Incidentally, in the following formulae, R^{A} is as defined above.

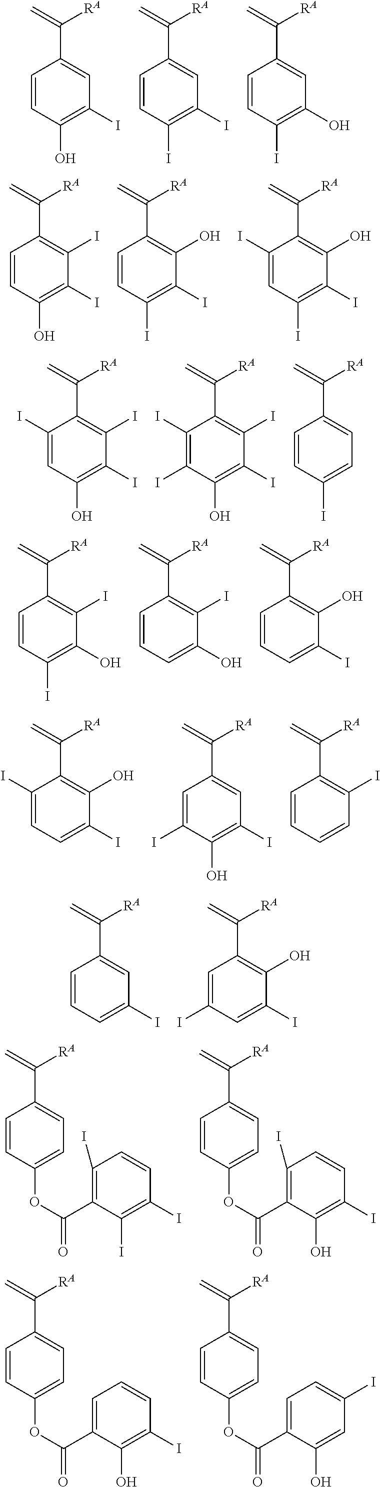

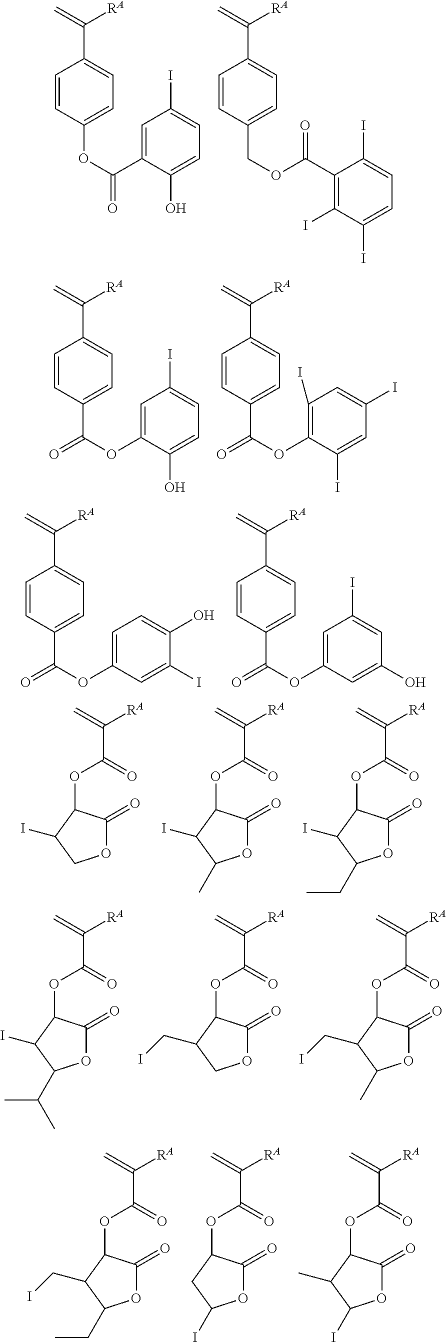

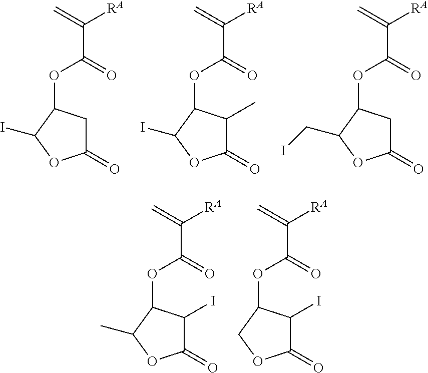

The resist material may also contain a repeating unit-f other than the repeating units described above. Examples of the repeating unit-f include those derived from styrene, vinylnaphthalene, indene, acenaphthylene, coumarin, coumarone, etc.

In the resist material, the content ratios of the repeating units-a1, -a2, -b1, -b2, -c, -d1, -d2, -d3, -e, and -f are preferably 0≤a1≤1.0, 0≤a2≤1.0, 0<a1+a2≤1.0, 0≤b1≤0.5, 0≤b2≤0.5, 0≤b1+b2≤0.9, 0≤c≤0.9, 0≤d1≤0.5, 0≤d2≤0.5, 0≤d3≤0.5, 0≤d1+d2+d3≤0.5, 0≤e≤0.5, and 0≤f≤0.5; more preferably 0.001≤a1≤0.8, 0.001≤a2≤0.8, 0.001≤a1+a2≤0.8, 0≤b1≤0.8, 0≤b2≤0.8, 0.1≤b1+b2≤0.8, 0≤c≤0.8, 0≤d1≤0.4, 0≤d2≤0.4, 0≤d3≤0.4, 0≤d1+d2+d3≤0.4, 0≤e≤0.4, and 0≤f≤0.4; and further preferably 0.005≤a1≤0.7, 0.005≤a2≤0.7, 0.005≤a1+a2≤0.7, 0≤b1≤0.7, 0≤b2≤0.7, 0≤b1+b2≤0.7, 0≤c≤0.7, 0≤d1≤0.3, 0≤d2≤0.3, 0≤d3≤0.3, 0≤d1+d2+d3≤0.3, 0≤e≤0.3, and 0≤f≤0.3, provided that a1+a2+b1+b2+c+d1+d2+d3+e+f=1.0.

The resist material may be synthesized, for example, by subjecting the monomers to give the repeating units described above to heat polymerization in an organic solvent to which a radical polymerization initiator has been added.

Examples of the organic solvent used in the polymerization include toluene, benzene, tetrahydrofuran (THF), diethyl ether, dioxane, propylene glycol monomethyl ether, γ-butyrolactone, mixed solvents thereof, etc. Examples of the polymerization initiator include 2,2′-azobisisobutyronitrile (AIBN), 2,2′-azobis(2,4-dimethylvaleronitrile), dimethyl-2,2-azobis(2-methylpropionate), benzoyl peroxide, lauroyl peroxide, etc. The temperature during the polymerization is preferably 50 to 80° C. The reaction time is preferably 2 to 100 hours, more preferably 5 to 20 hours.

In the case where the monomer containing a hydroxy group is copolymerized, the process may include: substituting the hydroxy group with an acetal group susceptible to deprotection with acid, such as an ethoxyethoxy group, prior to the polymerization; and performing the deprotection with weak acid and water after the polymerization. Alternatively, the process may include: substituting the hydroxy group with an acetyl group, a formyl group, a pivaloyl group, or the like prior to the polymerization; and performing alkaline hydrolysis after the polymerization.

In a case where hydroxystyrene or hydroxyvinylnaphthalene is copolymerized, at first, acetoxystyrene or acetoxyvinylnaphthalene may be used in place of hydroxystyrene or hydroxyvinylnaphthalene; after the polymerization, the acetoxy group may be deprotected by the alkaline hydrolysis to convert the acetoxystyrene or acetoxyvinylnaphthalene to hydroxystyrene or hydroxyvinylnaphthalene.

In the alkaline hydrolysis, a base such as ammonia water or triethylamine is usable. The reaction temperature is preferably −20 to 100° C., more preferably 0 to 60° C. The reaction time is preferably 0.2 to 100 hours, more preferably 0.5 to 20 hours.

The resist material has a polystyrene-based weight-average molecular weight (Mw) of preferably 1,000 to 500,000, more preferably 1,000 to 100,000, and further preferably 2,000 to 30,000, determined by gel permeation chromatography (GPC) using THE as an eluent. When the Mw is 1,000 or more, the resist material is provided with excellent heat resistance, and when 500,000 or less, alkali solubility is sustained, and a trailing phenomenon does not occur after pattern formation.

Further, if the resist material has a wide molecular weight distribution (Mw/Mn), low-molecular-weight and high-molecular-weight polymers are present, and therefore, there are risks of foreign matters being found on the pattern after the exposure and the pattern profile being degraded. The finer the pattern rule, the stronger the influences of Mw and Mw/Mn. Hence, in order to obtain a resist material suitably used for finer pattern dimensions, the resist material preferably has a narrow dispersity Mw/Mn of 1.0 to 2.0, particularly preferably 1.0 to 1.5.

To obtain a polymer having a narrow dispersity, it is possible to use, not only ordinary radical polymerization, but also living radical polymerization. Examples of living radical polymerization include living radical polymerization using nitroxide radicals (Nitroxide-Mediated radical Polymerization: NMP), Atom Transfer Radical Polymerization (ATRP), and Reversible Addition-Fragmentation chain Transfer (RAFT) polymerization.

The resist material may contain two or more kinds of polymers that differ in composition ratio, Mw, and Mw/Mn. In addition, a polymer containing the repeating unit-a and a polymer not containing the repeating unit-a may be blended together.

### Resist Composition

The present invention provides a resist composition containing the above-described resist material. The inventive resist composition preferably further contains one or more selected from a photo-acid generator, an organic solvent, a quencher, and a surfactant. In the following, each component to be contained in the resist composition will be described.

### Acid Generator

The inventive resist material may further contain an acid generator (hereinafter, also referred to as additive-type acid generator) that generates a strong acid. Here, the term strong acid means a compound that has sufficient acidity to cause a deprotection reaction of the acid-labile group of the resist material. Examples of the acid generator include compounds that generate acids in response to actinic light or radiation (photo-acid generators). The photo-acid generator can be any photo-acid generator as long as the compound generates an acid upon high-energy beam irradiation. Preferably, the photo-acid generator generates a sulfonic acid, imide acid, or methide acid. Suitable photo-acid generators include sulfonium salt, iodonium salt, sulfonyldiazomethane, N-sulfonyloxyimide, oxime-O-sulfonate type acid generators, etc. Specific examples of the photo-acid generator include ones disclosed in paragraphs [0122] to [0142] of JP 2008-111103 A.

Moreover, a sulfonium salt shown by the following general formula (1-1) and an iodonium salt shown by the following general formula (1-2) can also be used suitably as photo-acid generators.

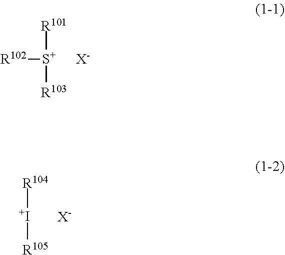

In the general formulae (1-1) and (1-2), R¹⁰¹ to R¹⁰⁵ each independently represent a monovalent hydrocarbon group having 1 to 20 carbon atoms and optionally containing a heteroatom. In addition, any two of R¹⁰¹, R¹⁰², and R¹⁰³ are optionally bonded to each other to form a ring together with a sulfur atom bonded thereto. The monovalent hydrocarbon group may be linear, branched, or cyclic, and specific examples thereof include the monovalent hydrocarbon groups given above.

In the general formulae (1-1) and (1-2), X represents an anion selected from the following general formulae (1A) to (1D).

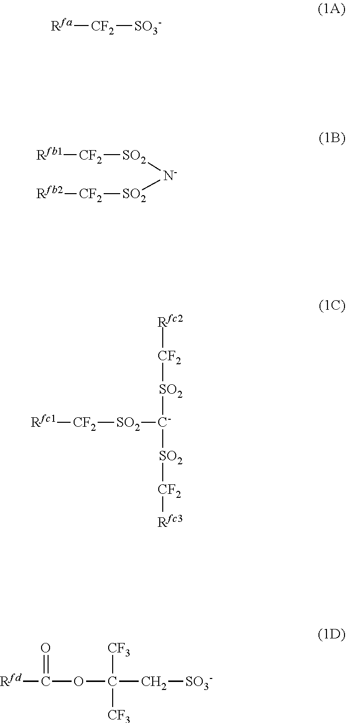

In the general formula (1A), R^{fa} represents a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include groups given later as hydrocarbyl groups represented by R¹⁰⁷ in the general formula (1A′) described below.

As the anion represented by the general formula (1A), an anion represented by the following general formula (1A′) is preferable.

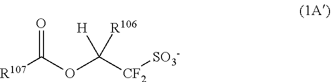

In the general formula (1A′), R¹⁰⁶ represents a hydrogen atom or a trifluoromethyl group, preferably a trifluoromethyl group. R¹⁰⁷ represents a hydrocarbyl group having 1 to 38 carbon atoms and optionally containing a heteroatom. The heteroatom is preferably an oxygen atom, a nitrogen atom, a sulfur atom, a halogen atom, or the like, more preferably an oxygen atom. The hydrocarbyl group particularly preferably has 6 to 30 carbon atoms from the viewpoint of achieving high resolution in fine pattern formation.

The hydrocarbyl group represented by R¹⁰⁷ may be saturated or unsaturated, and may be linear, branched, or cyclic. Specific examples thereof include alkyl groups, such as a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosanyl group; cyclic saturated hydrocarbyl groups, such as a cyclopentyl group, a cyclohexyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group; unsaturated hydrocarbyl groups, such as an allyl group and a 3-cyclohexenyl group; aryl groups, such as a phenyl group, a 1-naphthyl group, and a 2-naphthyl group; and aralkyl groups, such as a benzyl group and a diphenylmethyl group.

Furthermore, part or all of the hydrogen atoms of these groups may be substituted with a group containing a heteroatom, such as an oxygen atom, a sulfur atom, a nitrogen atom, or a halogen atom, and part of the carbon atoms of these groups may be substituted with a group containing a heteroatom such as an oxygen atom, a sulfur atom, or a nitrogen atom. The resulting hydrocarbyl group may contain a hydroxy group, a cyano group, a carbonyl group, an ether bond, an ester bond, a sulfonic acid ester bond, a carbonate group, a lactone ring, a sultone ring, a carboxylic anhydride, a haloalkyl group, etc. Examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, a 3-oxocyclohexyl group, etc.

The synthesis of the sulfonium salt containing the anion shown by the general formula (1A′) is described in detail in JP 2007-145797 A, JP 2008-106045 A, JP 2009-007327 A, JP 2009-258695 A, etc. In addition, sulfonium salts disclosed in JP 2010-215608 A, JP 2012-041320 A, JP 2012-106986 A, JP 2012-153644 A, etc. are also suitably used.

Examples of the anion represented by the general formula (1A) include those given as examples of the anion represented by a formula (1A) in JP 2018-197853 A.

In the general formula (1B), R^{fb1} and R^{fb2} each independently represent a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given as examples in the description of the R¹⁰⁷ in the general formula (1A′). R^{fb1} and R^{fb2} are preferably a fluorine atom or a linear fluorinated alkyl group having 1 to 4 carbon atoms. Alternatively, R^{fb1} and R^{fb2} may bond with each other to form a ring together with a group (—CF₂—SO₂—N—SO₂—CF₂—) bonded therewith. In this event, the group obtained by R^{fb1} and R^{fb2} being bonded to each other is preferably a fluorinated ethylene group or a fluorinated propylene group.

In the general formula (1C), R^{fc1}, R^{fc2}, and R^{fc3} each independently represent a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given as examples in the description of the R¹⁰⁷ in the general formula (1A′). R^{fc1}, R^{fc2}, and R^{fc3} are preferably a fluorine atom or a linear fluorinated alkyl group having 1 to 4 carbon atoms. Alternatively, R^{fc1} and R^{fc2} may bond with each other to form a ring together with a group (—CF₂—SO₂—C—SO₂—CF₂—) bonded therewith. In this event, the group obtained by R^{fc1} and R^{fc2} being bonded with each other is preferably a fluorinated ethylene group or a fluorinated propylene group.

In the general formula (1D), R^{fd} represents a hydrocarbyl group having 1 to 40 carbon atoms and optionally containing a heteroatom. The hydrocarbyl group may be saturated or unsaturated, and may be linear, branched, or cyclic. Specific examples thereof include those given as examples in the description of the R¹⁰⁷ in the general formula (1A′). The synthesis of the sulfonium salt containing the anion shown by the general formula (1D) is described in detail in JP 2010-215608 A and JP 2014-133723 A.

Examples of the anion represented by the general formula (1D) include those given as examples of the anion represented by a formula (1D) in JP 2018-197853 A.

Note that the photo-acid generator containing the anion shown by the general formula (1D) does not have a fluorine atom at α position of the sulfo group, but has two trifluoromethyl groups at β position, thereby providing sufficient acidity to cut the acid-labile group in the resist material. Thus, this photo-acid generator is utilizable.

Furthermore, one shown by the following general formula (2) can also be used suitably as a photo-acid generator.

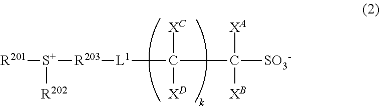

In the general formula (2), R²⁰¹ and R²⁰² each independently represent a hydrocarbyl group having 1 to 30 carbon atoms and optionally containing a heteroatom. R²⁰³ represents a hydrocarbylene group having 1 to 30 carbon atoms and optionally containing a heteroatom. In addition, R²⁰¹ and R²⁰² or R²⁰¹ and R²⁰³ may be bonded with each other to form a ring together with a sulfur atom bonded therewith. In this event, examples of the ring include those given as examples of rings that can be formed by R¹⁰¹ and R¹⁰² being bonded to each other together with the sulfur atom bonded thereto in the description of the general formula (1-1).

The hydrocarbyl groups represented by R²⁰¹ and R²⁰² may be saturated or unsaturated, and may be linear, branched, or cyclic. Specific examples thereof include: alkyl groups, such as a methyl group, an ethyl group, a propyl group, an isopropyl group, an n-butyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, and an adamantyl group; aryl groups, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; etc. Furthermore, part or all of the hydrogen atoms of these groups may be substituted with a group containing a heteroatom, such as an oxygen atom, a sulfur atom, a nitrogen atom, or a halogen atom, and part of the carbon atoms of these groups may be substituted with a group containing a heteroatom such as an oxygen atom, a sulfur atom, or a nitrogen atom. The resulting hydrocarbyl group may contain a hydroxy group, a cyano group, a carbonyl group, an ether bond, an ester bond, a sulfonic acid ester bond, a carbonate group, a lactone ring, a sultone ring, a carboxylic anhydride, a haloalkyl group, etc.

The hydrocarbylene group represented by R²⁰³ may be saturated or unsaturated, and may be linear, branched, or cyclic. Specific examples thereof include alkanediyl groups, such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; cyclic saturated hydrocarbylene groups, such as a cyclopentanediyl group, a cyclohexanediyl group, a norbornanediyl group, and an adamantanediyl group; arylene groups, such as a phenylene group, a methylphenylene group, an ethylphenylene group, an n-propylphenylene group, an isopropylphenylene group, an n-butylphenylene group, an isobutylphenylene group, a sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, a sec-butylnaphthylene group, and a tert-butylnaphthylene group; etc. Furthermore, part or all of the hydrogen atoms of these groups may be substituted with a group containing a heteroatom, such as an oxygen atom, a sulfur atom, a nitrogen atom, or a halogen atom, and part of the carbon atoms of these groups may be substituted with a group containing a heteroatom such as an oxygen atom, a sulfur atom, or a nitrogen atom. The resulting hydrocarbylene group may contain a hydroxy group, a cyano group, a carbonyl group, an ether bond, an ester bond, a sulfonic acid ester bond, a carbonate group, a lactone ring, a sultone ring, a carboxylic anhydride, a haloalkyl group, etc. As the heteroatom, an oxygen atom is preferable.

In the general formula (2), L¹ represents a single bond, an ether bond, or a hydrocarbylene group having 1 to 20 carbon atoms and optionally containing a heteroatom. The hydrocarbylene group may be saturated or unsaturated, and may be linear, branched, or cyclic. Specific examples thereof include those given as examples of the hydrocarbylene group represented by R²⁰³.

In the general formula (2), X^{A}, X^{B}, X^{C}, and X^{D} each independently represent a hydrogen atom, a fluorine atom, or a trifluoromethyl group, provided that at least one of X^{A}, X^{B}, X^{C}, and X^{D} is a fluorine atom or a trifluoromethyl group.

In the general formula (2), “k” represents an integer of 0 to 3.

As the photo-acid generator shown by the general formula (2), those shown by the following general formula (2′) are preferable.

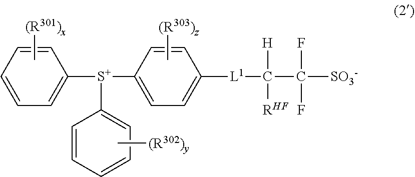

In the general formula (2′), L¹ is as defined above. R^{HF} represents a hydrogen atom or a trifluoromethyl group, preferably a trifluoromethyl group. R³⁰¹, R³⁰², and R³⁰³ each independently represent a hydrogen atom or a hydrocarbyl group having 1 to 20 carbon atoms and optionally containing a heteroatom. The hydrocarbyl group may be saturated or unsaturated, and may be linear, branched, or cyclic. Specific examples thereof include those given as examples in the description of the R¹⁰⁷ in the general formula (1A′). “x” and “y” each independently represent an integer of 0 to 5, and “z” represents an integer of 0 to 4.

Examples of the photo-acid generator represented by the general formula (2) include those given as examples of a photo-acid generator represented by a formula (2) in JP 2017-026980 A.

The photo-acid generators containing the anion represented by the general formula (1A′) or (1D) are particularly preferable because of small acid diffusion and excellent solubility to a resist solvent. A photo-acid generator represented by the general formula (2′) is also particularly preferable because the acid diffusion is quite small.

Furthermore, as the photo-acid generator, it is also possible to use a sulfonium salt or an iodonium salt having an anion including an aromatic ring substituted with an iodine atom or a bromine atom. Examples of such salts include those represented by the following general formula (3-1) or (3-2).

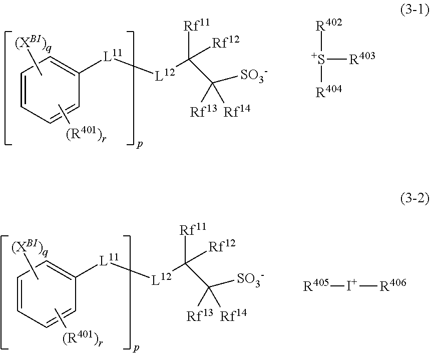

In the general formulae (3-1) and (3-2), “p” represents an integer that satisfies 1≤p≤3. “q” and “r” represent integers that satisfy 1≤q≤5, 0≤r≤3, and 1≤q+r≤5. “q” is preferably an integer that satisfies 1≤q≤3, and is more preferably 2 or 3. “r” is preferably an integer that satisfies 0≤r≤2.

In the general formulae (3-1) and (3-2), X^{BI} represents an iodine atom or a bromine atom, and when “q” is 2 or more, the X^{BI}s may be identical to or different from one another.

In the general formulae (3-1) and (3-2), L¹¹ represents a single bond, an ether bond, an ester bond, or a saturated hydrocarbylene group having 1 to 6 carbon atoms and optionally including an ether bond or an ester bond. The saturated hydrocarbylene group may be linear, branched, or cyclic.

In the general formulae (3-1) and (3-2), L¹² represents a single bond or a divalent linking group having 1 to 20 carbon atoms when “r” is 1, and represents a trivalent or tetravalent linking group having 1 to 20 carbon atoms when “r” is 2 or 3, the linking group optionally containing an oxygen atom, a sulfur atom, or a nitrogen atom.

In the general formulae (3-1) and (3-2), R⁴⁰¹ represents a hydroxy group, a carboxy group, a fluorine atom, a chlorine atom, a bromine atom, an amino group, a saturated hydrocarbyl group having 1 to 20 carbon atoms, a saturated hydrocarbyloxy group having 1 to 20 carbon atoms, a saturated hydrocarbyloxycarbonyl group having 2 to 10 carbon atoms, a saturated hydrocarbylcarbonyloxy group having 2 to 20 carbon atoms, a saturated hydrocarbylsulfonyloxy group having 1 to 20 carbon atoms, —NR^{401A}—C(═O)—R^{401B}, or —NR^{401A}—C(═O)—O—R^{401B}. The hydrocarbyl group, the hydrocarbyloxy group, the hydrocarbyloxycarbonyl group, the hydrocarbylcarbonyloxy group, and the hydrocarbylsulfonyloxy group optionally have a fluorine atom, a chlorine atom, a bromine atom, a hydroxy group, an amino group, or an ether bond. R^{401A} represents a hydrogen atom or a saturated hydrocarbyl group having 1 to 6 carbon atoms, and optionally contains a halogen atom, a hydroxy group, an alkoxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{401B} represents an aliphatic hydrocarbyl group having 1 to 16 carbon atoms or an aryl group having 6 to 12 carbon atoms, and optionally contains a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be saturated or unsaturated, and may be linear, branched, or cyclic. The saturated hydrocarbyl group, the saturated hydrocarbyloxy group, the saturated hydrocarbyloxycarbonyl group, the saturated hydrocarbylcarbonyl group, and the saturated hydrocarbylcarbonyloxy group may be linear, branched, or cyclic. When “p” and/or “r” is 2 or more, the multiple R⁴⁰¹ may be identical to or different from one another.

In particular, a hydroxy group, —NR^{401A}—C(—O)—R^{401B}, —NR^{401A}—C(═O)—O—R^{401B}, a fluorine atom, a chlorine atom, a bromine atom, a methyl group, a methoxy group, etc. are preferable as R⁴⁰¹.

In the general formulae (3-1) and (3-2), Rf¹¹ to Rf¹⁴ each independently represent a hydrogen atom, a fluorine atom, or a trifluoromethyl group, provided that at least one of Rf¹¹ to Rf¹⁴ is a fluorine atom or a trifluoromethyl group. Rf¹¹ and Rf¹² may also be combined to form a carbonyl group. In particular, Rf¹³ and Rf¹⁴ are preferably both a fluorine atom.

In the general formulae (3-1) and (3-2), R⁴⁰², R⁴⁰³, R⁴⁰⁴, R⁴⁰⁵, and R⁴⁰⁶ each independently represent a fluorine atom, a chlorine atom, a bromine atom, an iodine atom, or a hydrocarbyl group having 1 to 20 carbon atoms and optionally containing a heteroatom. The hydrocarbyl group may be saturated or unsaturated, and may be linear, branched, or cyclic. Specific examples thereof include those given as examples of the hydrocarbyl groups represented by R¹⁰¹ to R¹⁰⁵ in the description of the general formulae (1-1) and (1-2). Furthermore, part or all of the hydrogen atoms of these groups may be substituted with a hydroxy group, a carboxy group, a halogen atom, a cyano group, a nitro group, a mercapto group, a sultone group, a sulfone group, or a sulfonium salt-containing group; and part of the carbon atoms of the groups may be substituted with an ether bond, an ester bond, a carbonyl group, an amide bond, a carbonate group, or a sulfonic acid ester bond. Furthermore, R⁴⁰² and R⁴⁰³ may be bonded to each other to form a ring together with the sulfur atom bonded thereto. In this event, examples of the ring include those given as examples of the rings that can be formed by R¹⁰¹ and R¹⁰² being bonded to each other together with the sulfur atom bonded thereto in the description of the general formula (1-1).

Examples of the cation of the sulfonium salt represented by the general formula (3-1) include those given as examples of the cation of a sulfonium salt represented by the general formula (1-1). Meanwhile, examples of the cation of the iodonium salt represented by the general formula (3-2) include those given as examples of the cation of the iodonium salt represented by the general formula (1-2).

Examples of the anion of the onium salt represented by the general formula (3-1) or (3-2) include those shown below, but are not limited thereto. Incidentally, in the following formulae, X^{BI} is as defined above.

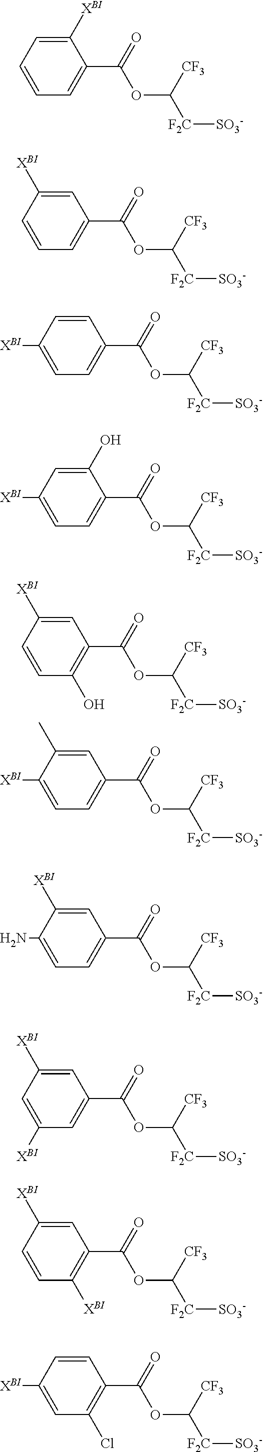

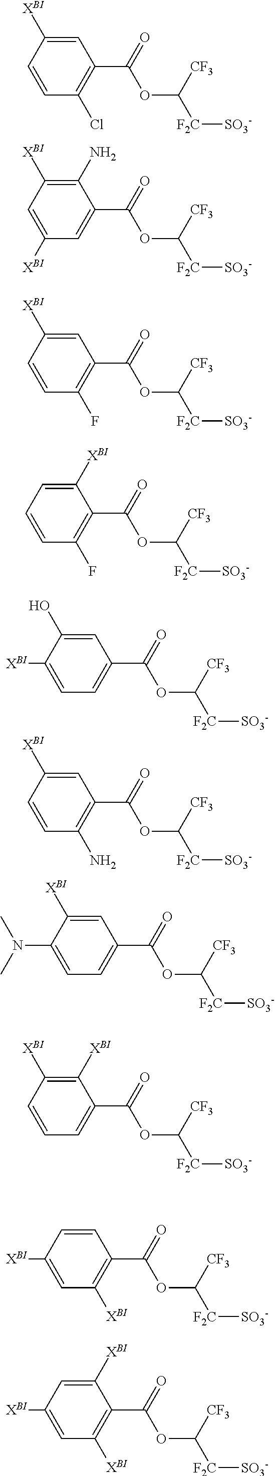

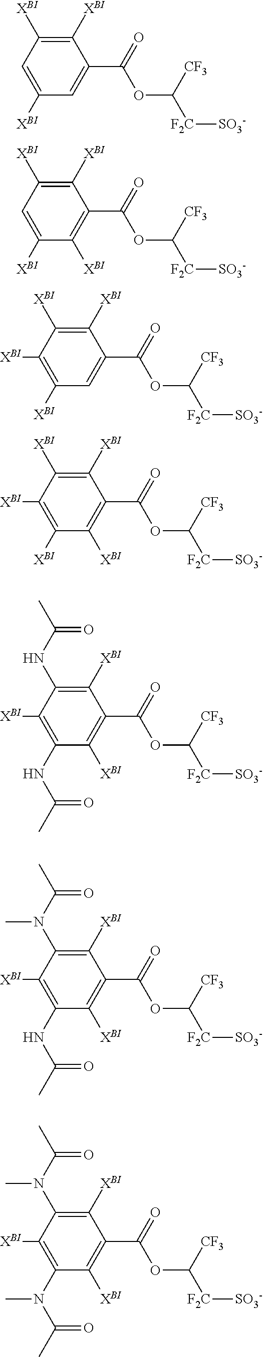

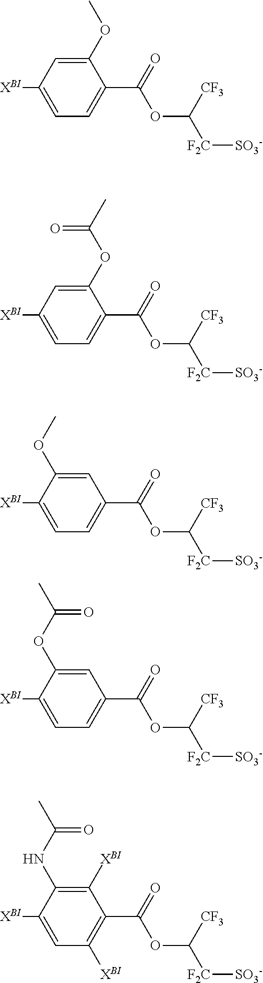

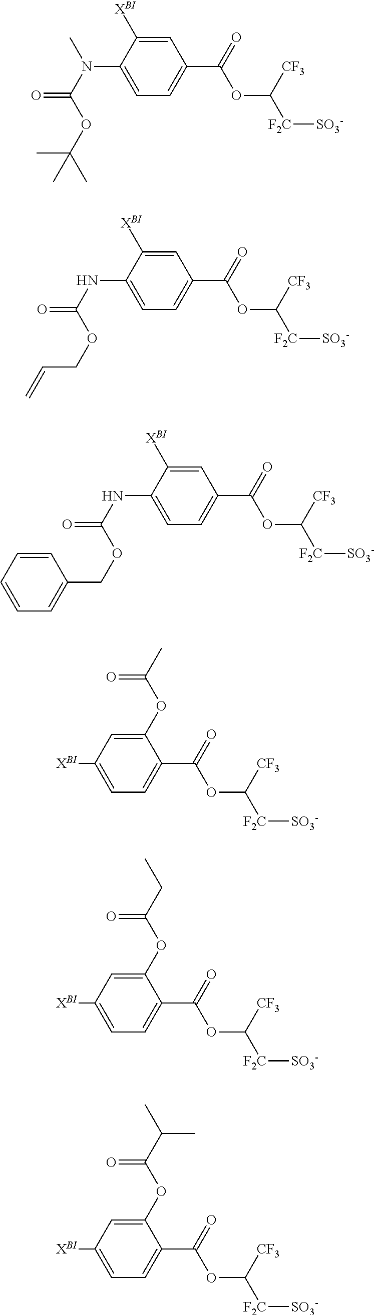

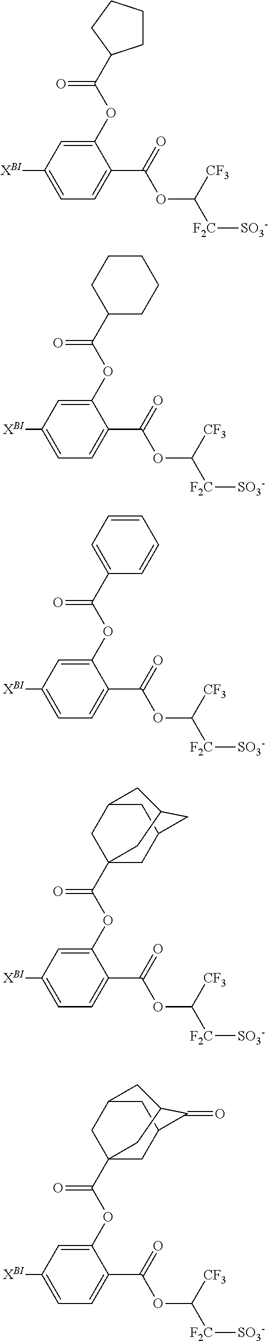

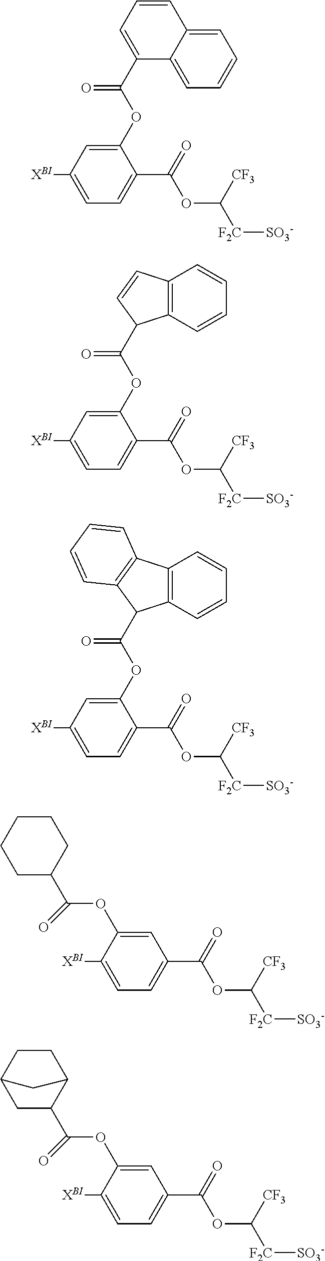

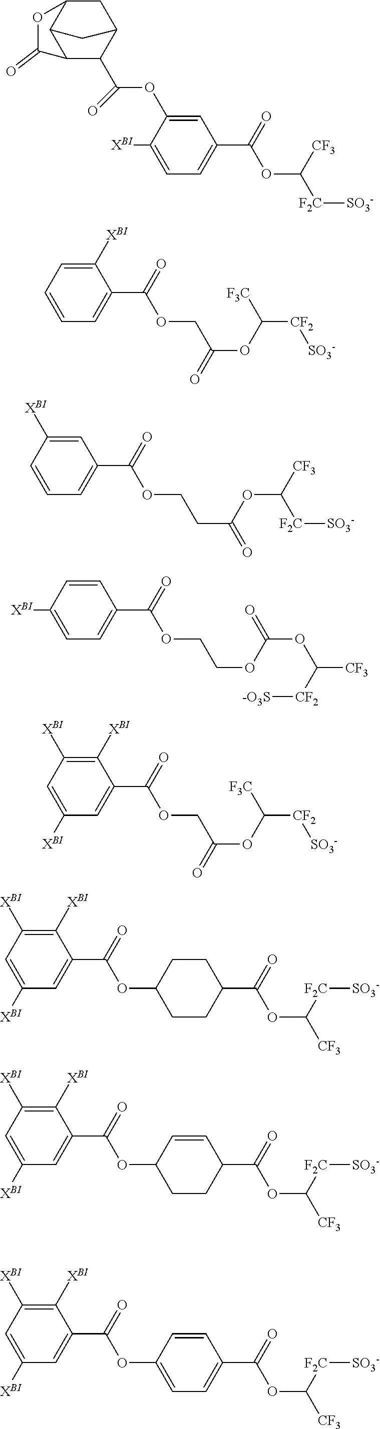

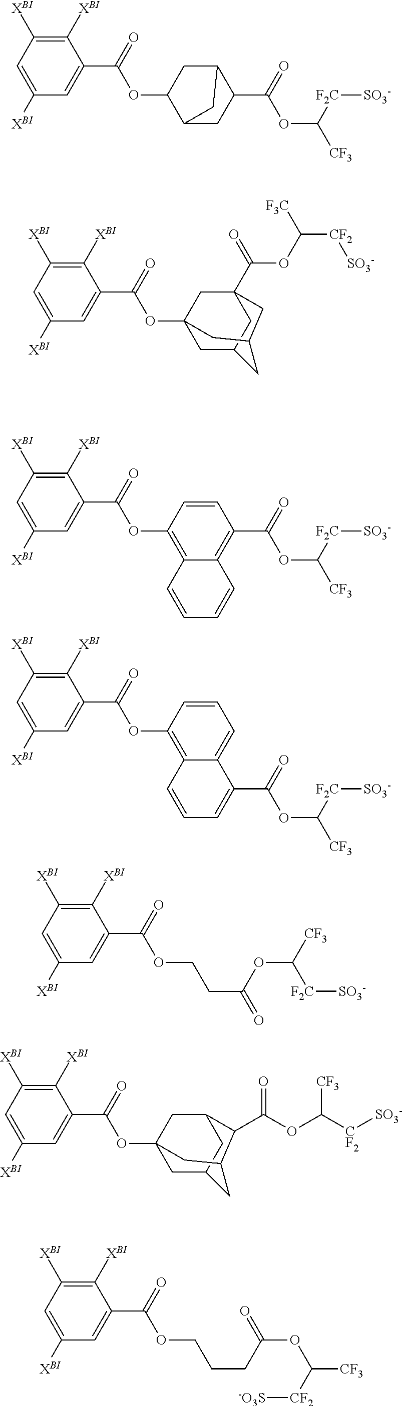

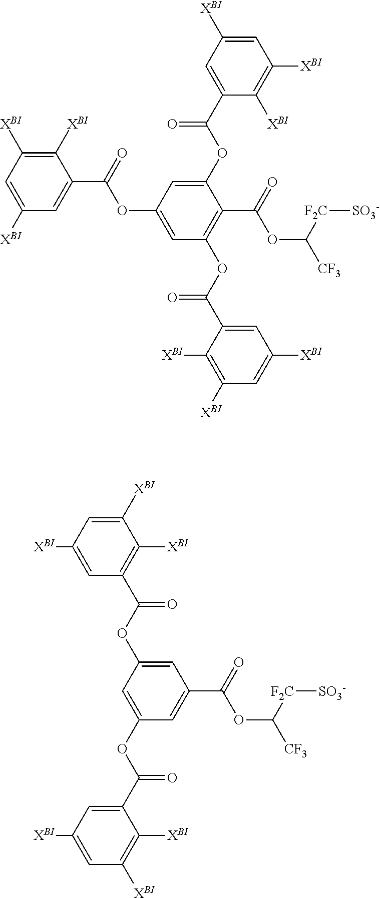

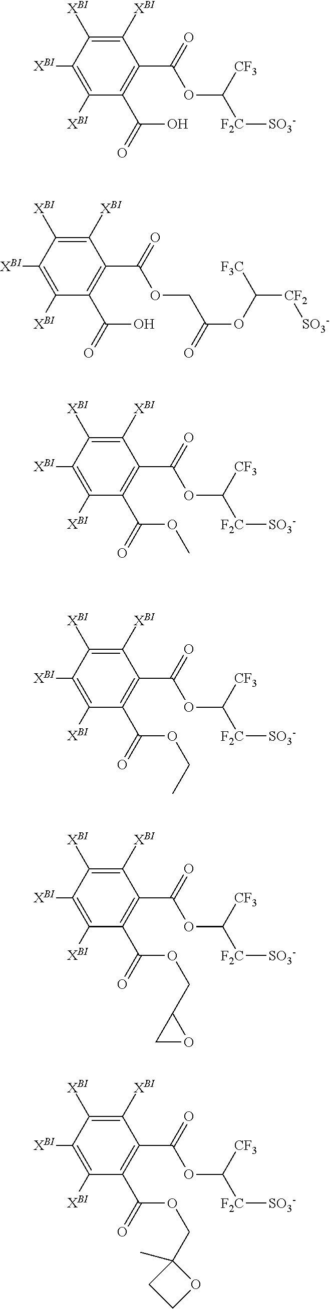

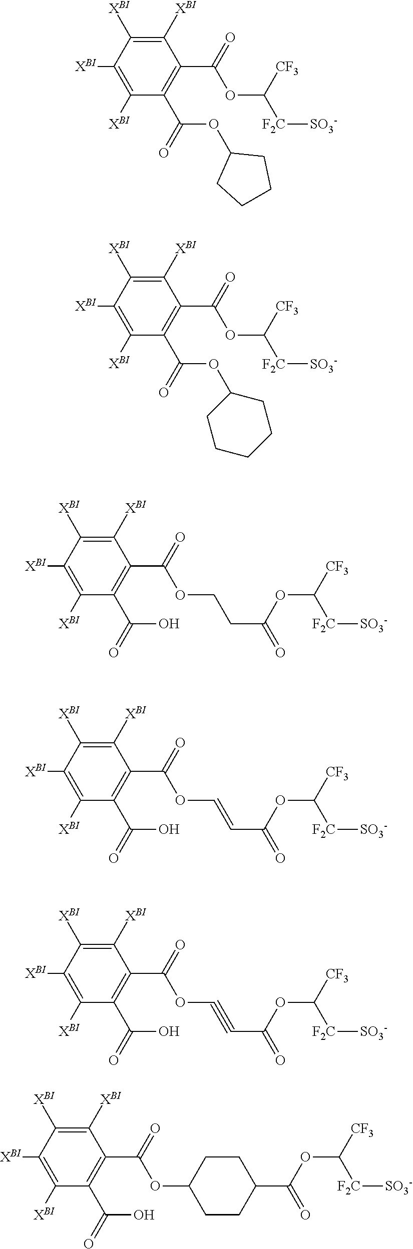

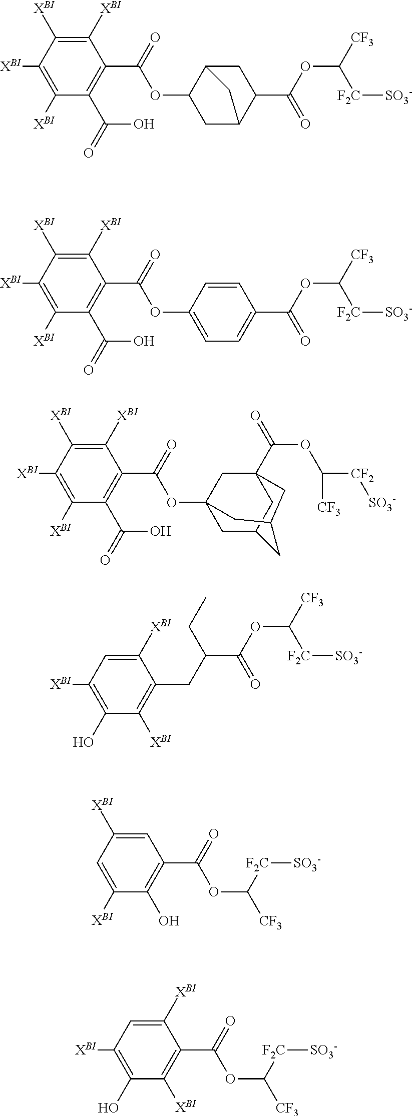

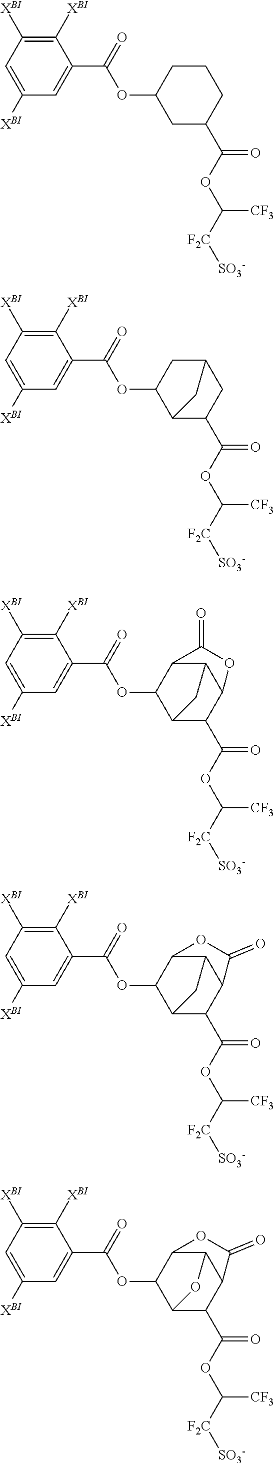

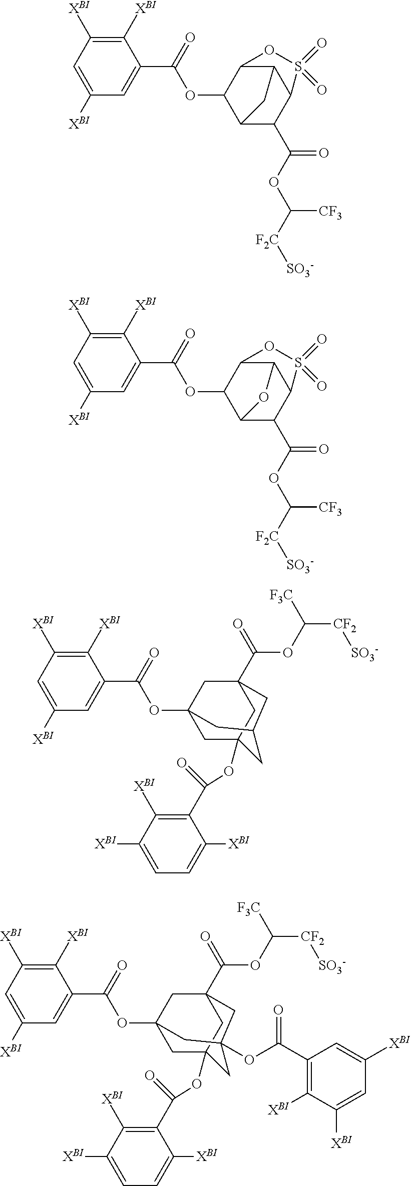

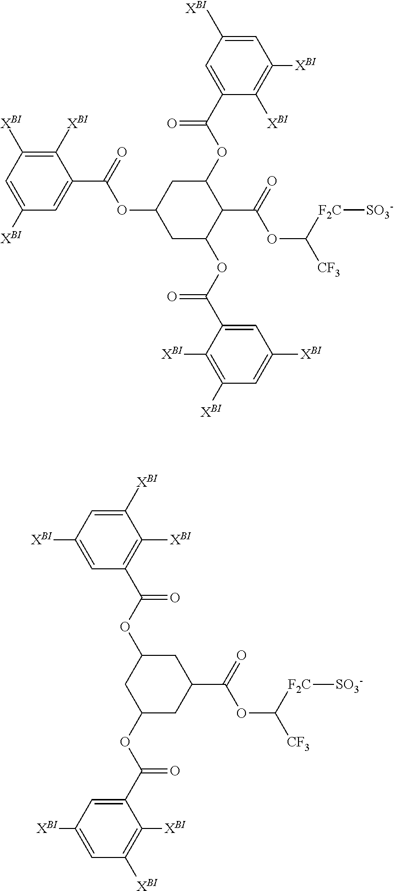

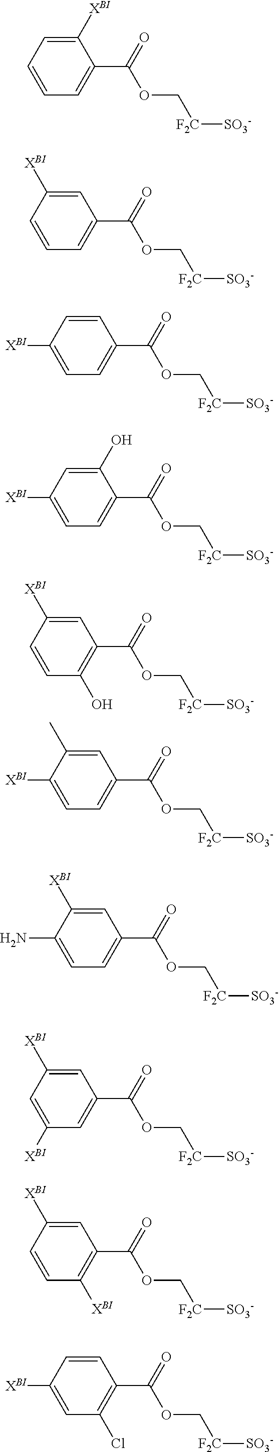

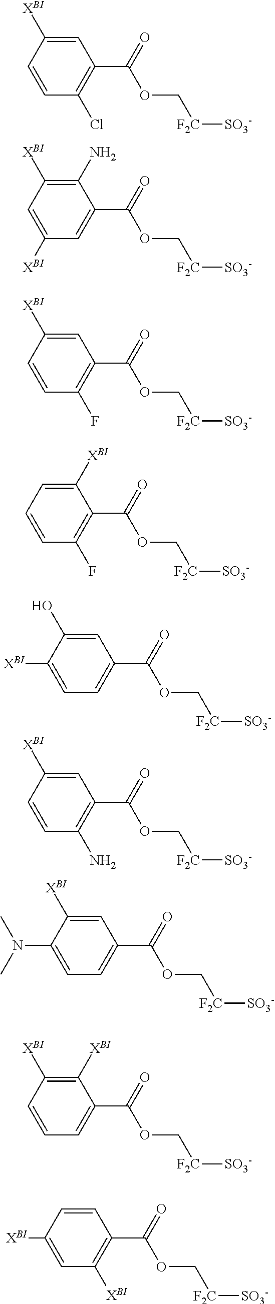

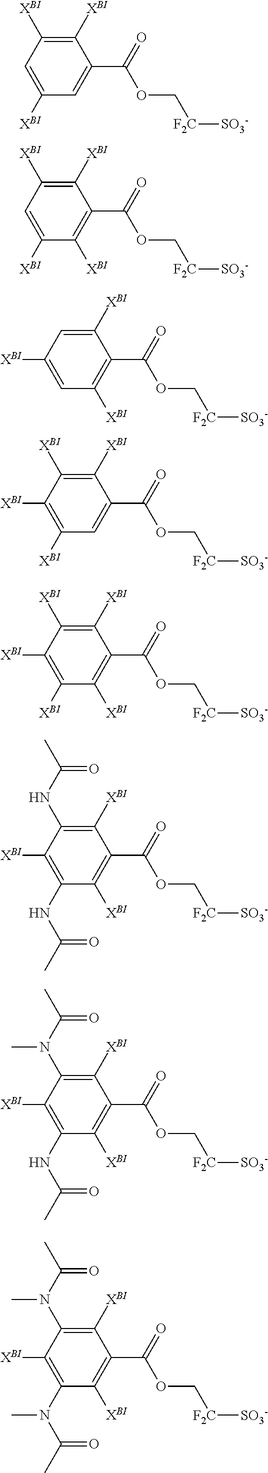

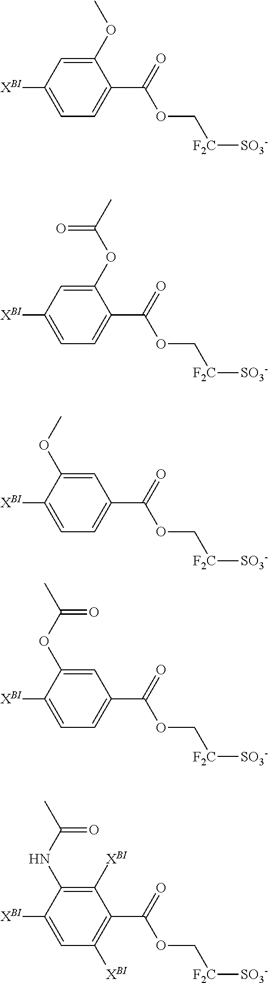

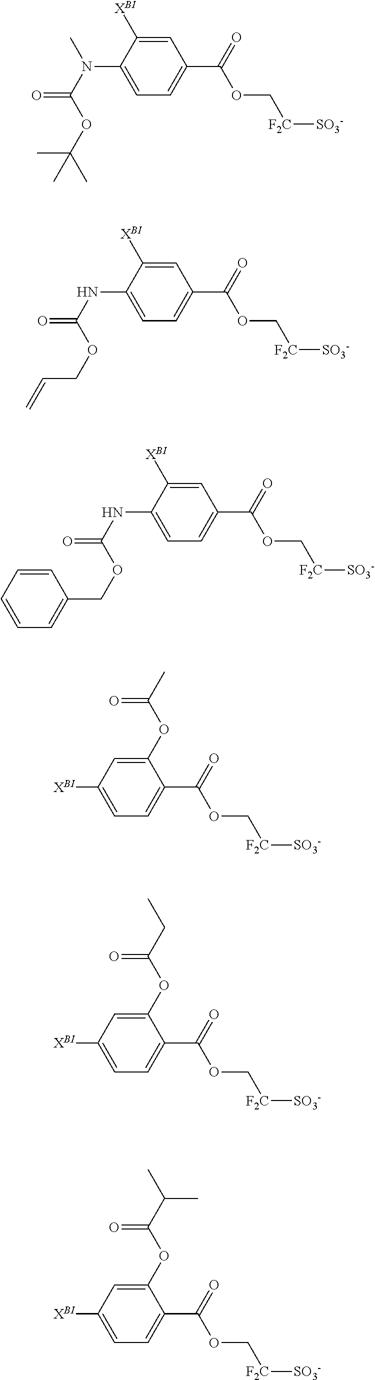

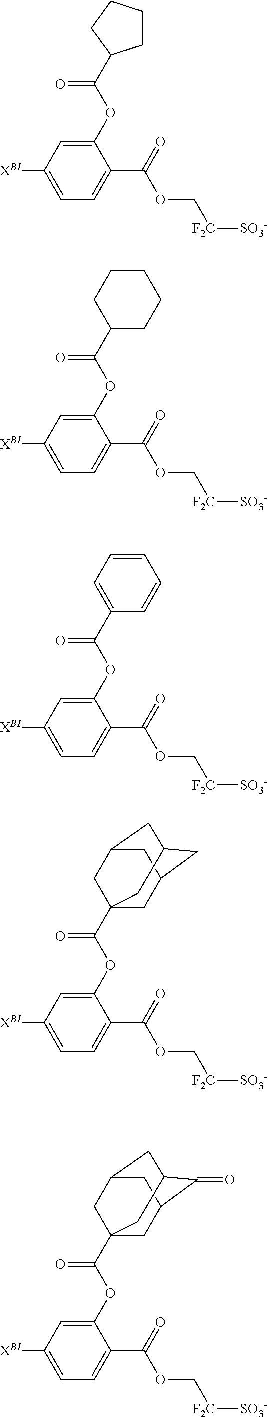

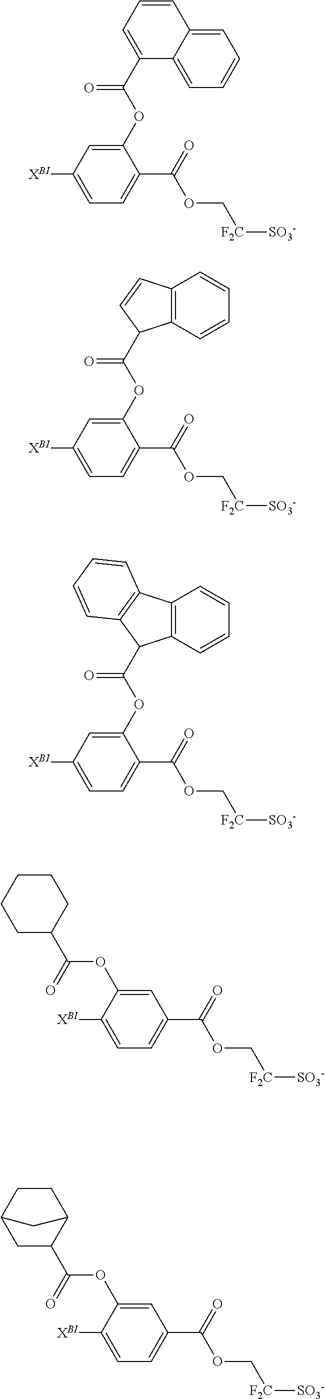

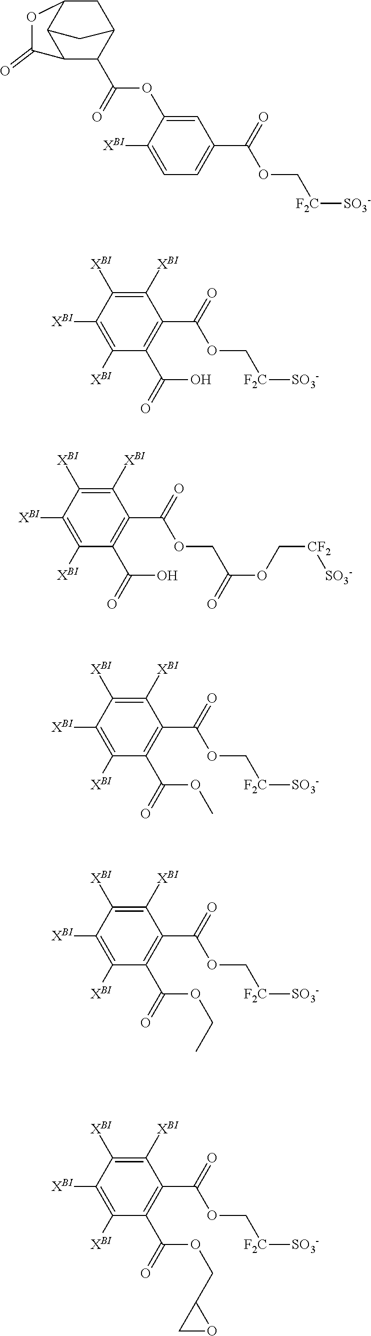

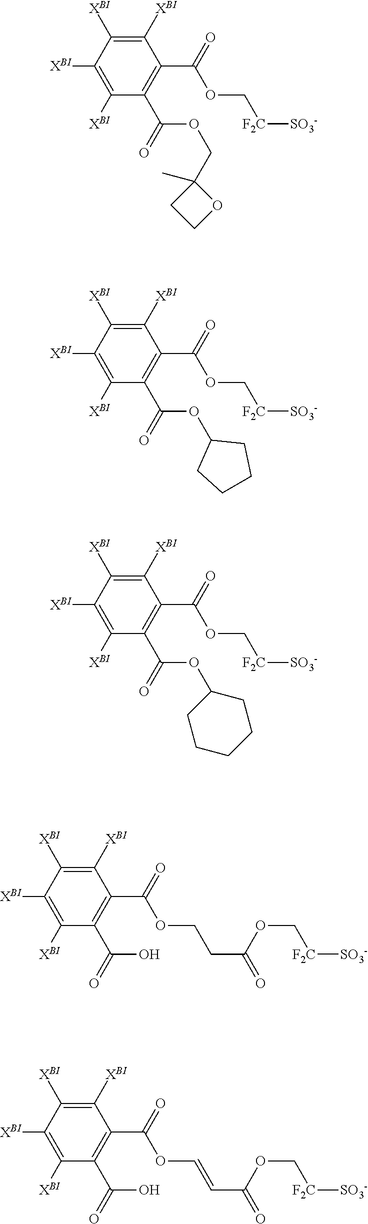

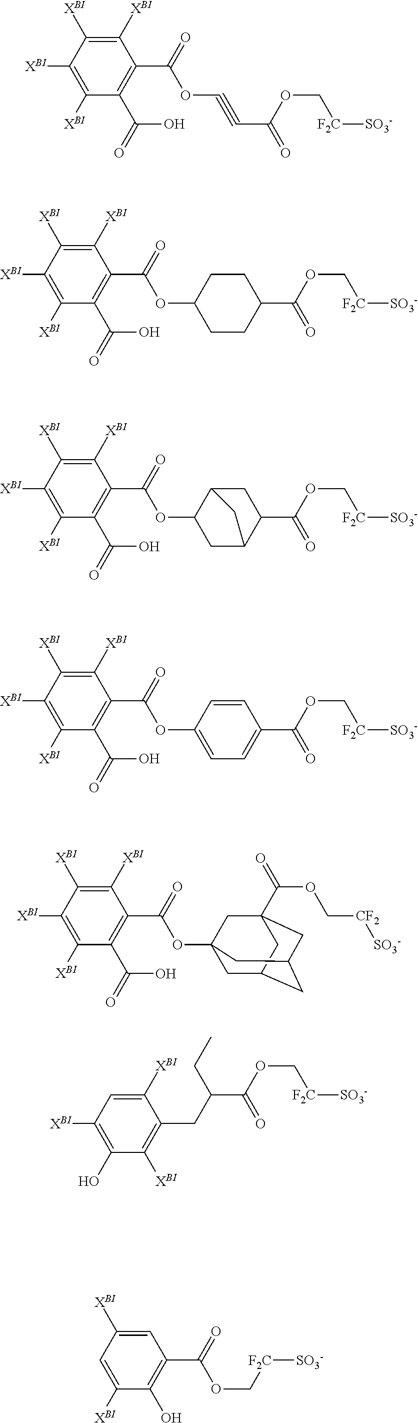

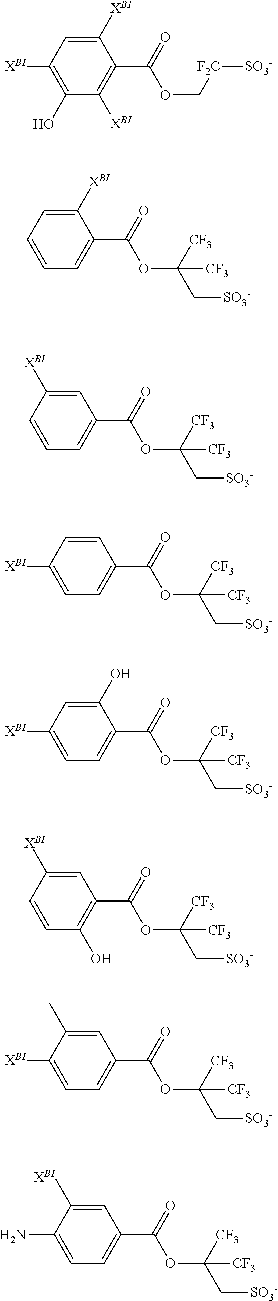

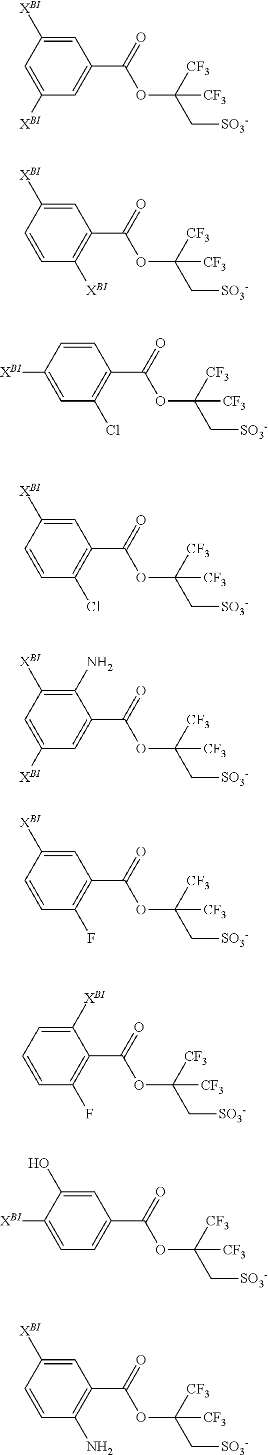

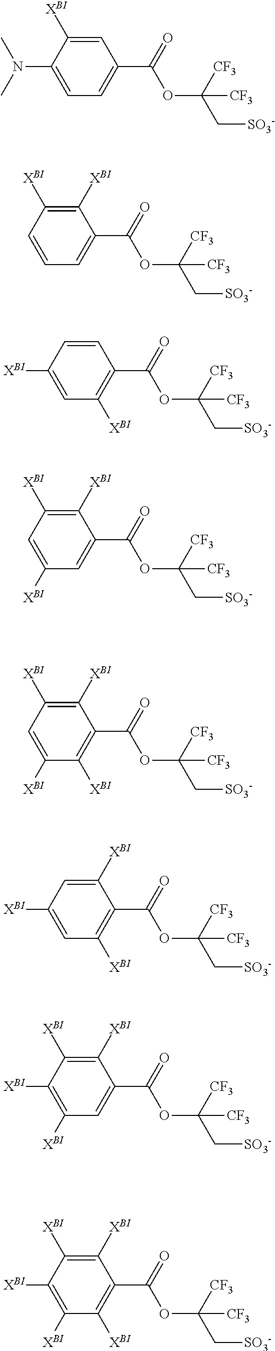

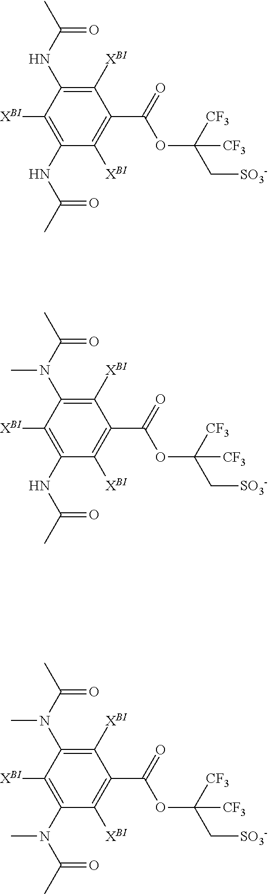

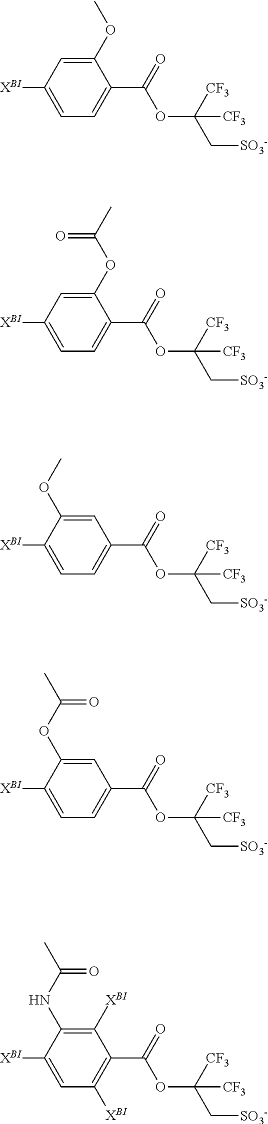

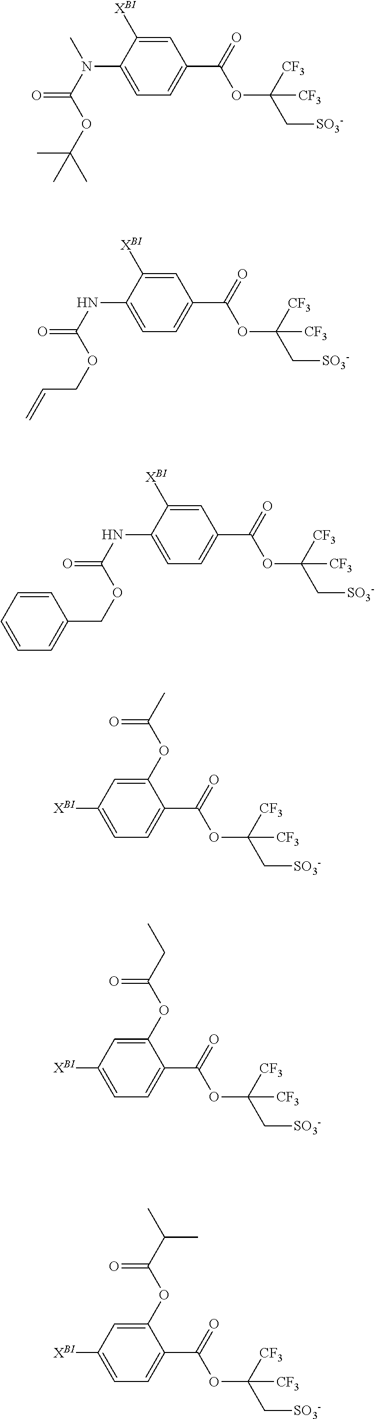

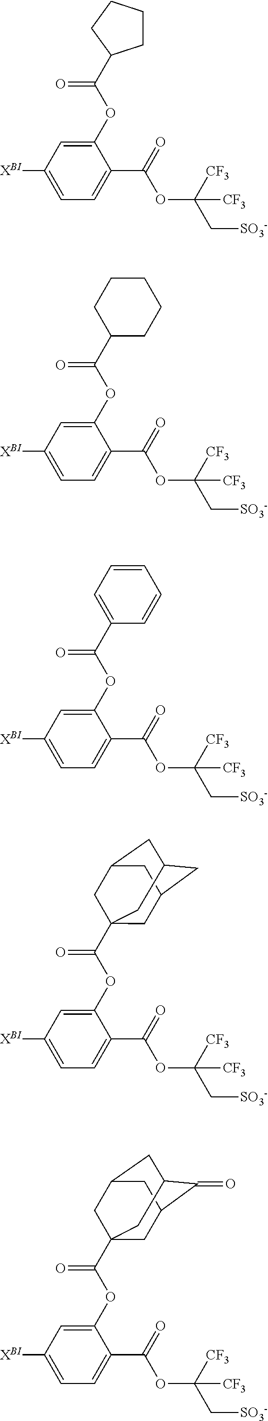

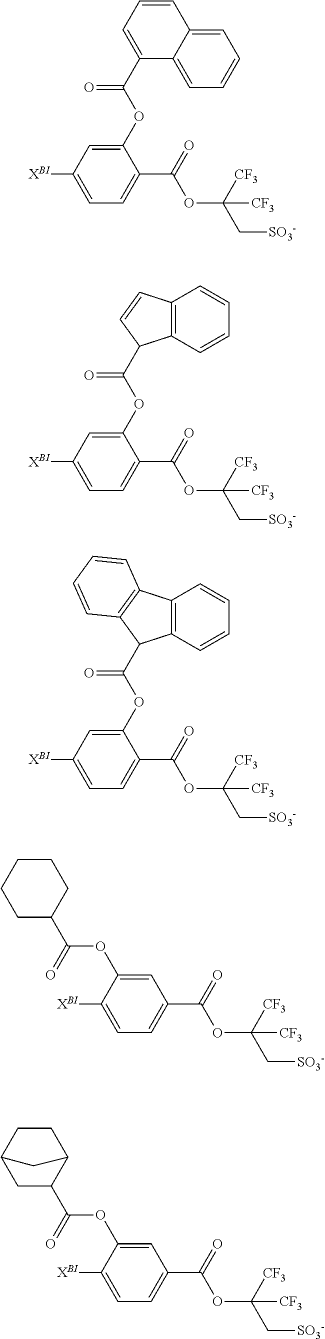

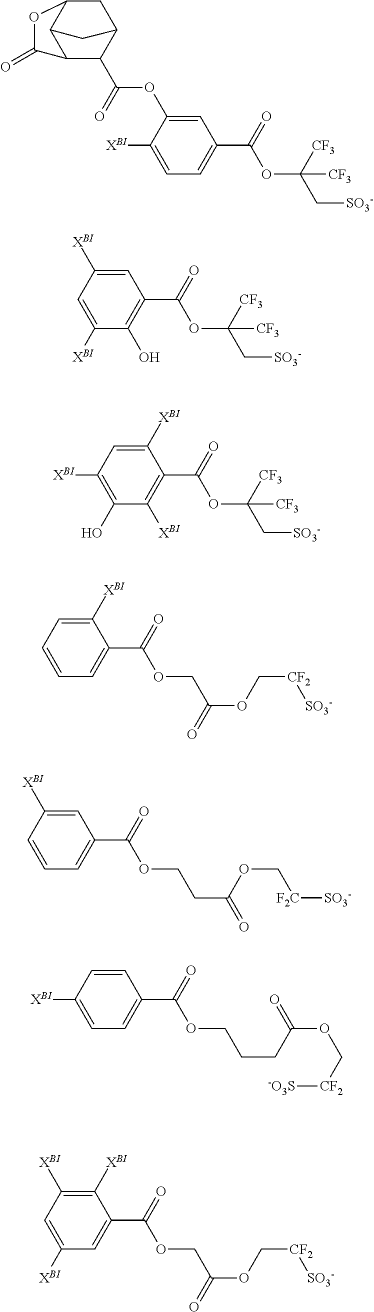

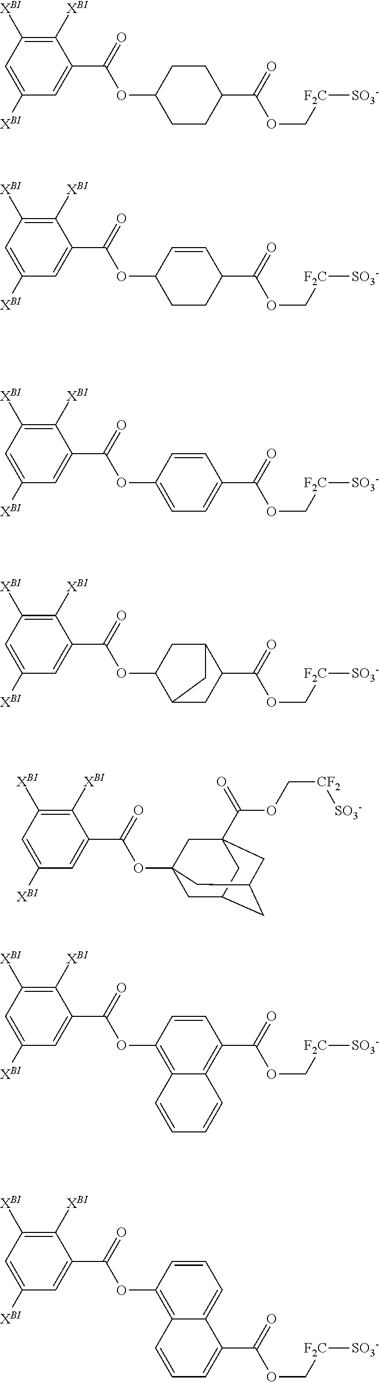

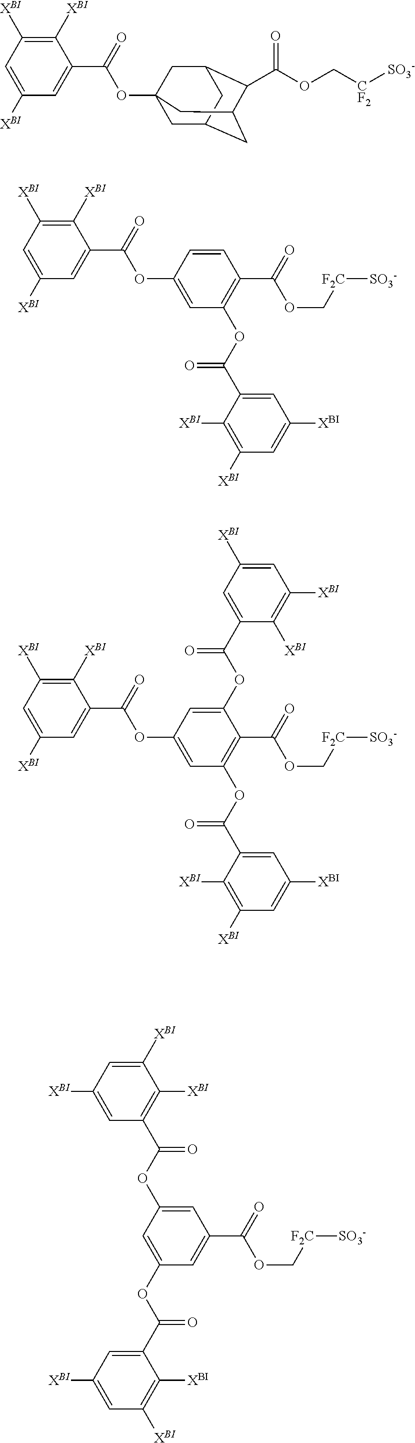

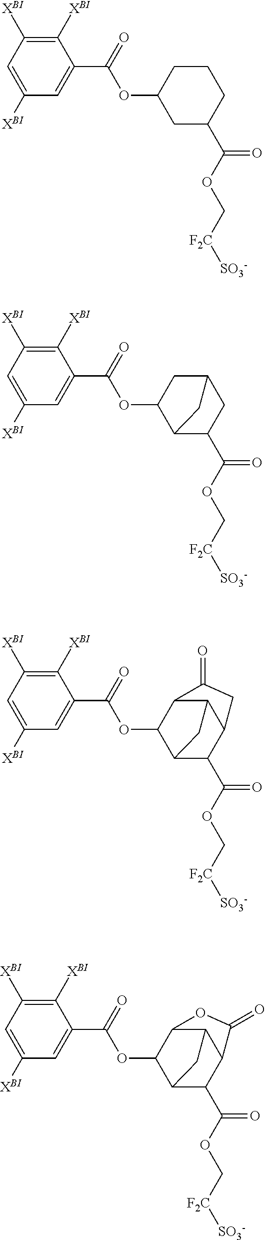

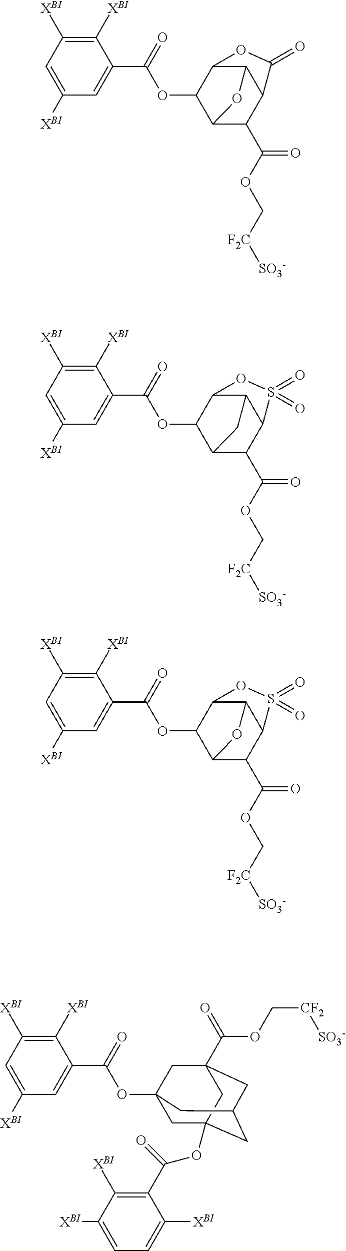

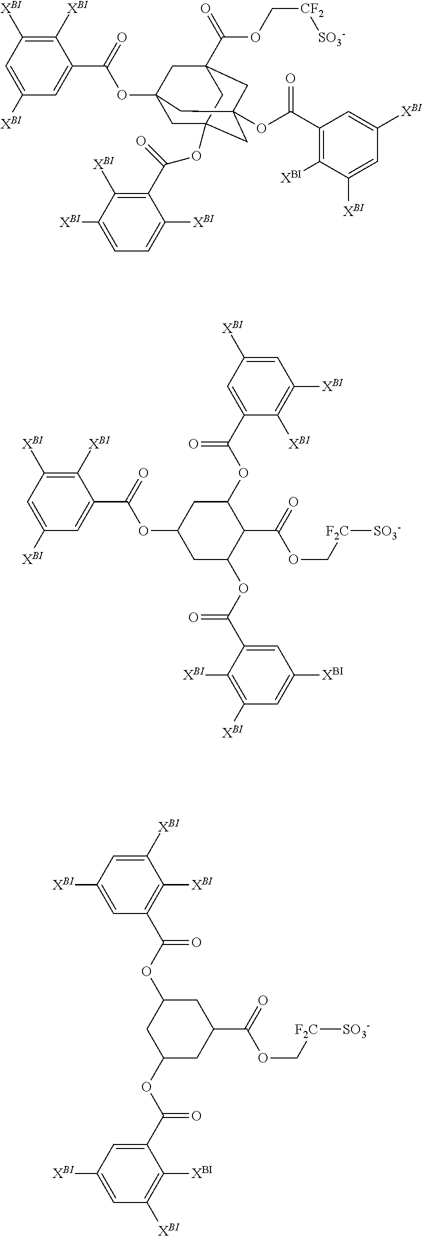

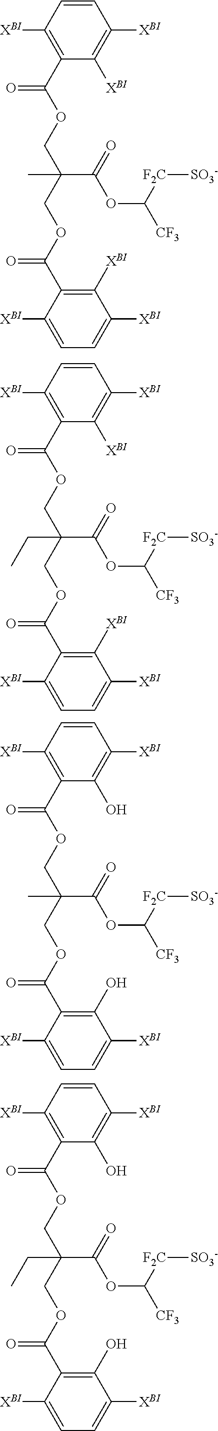

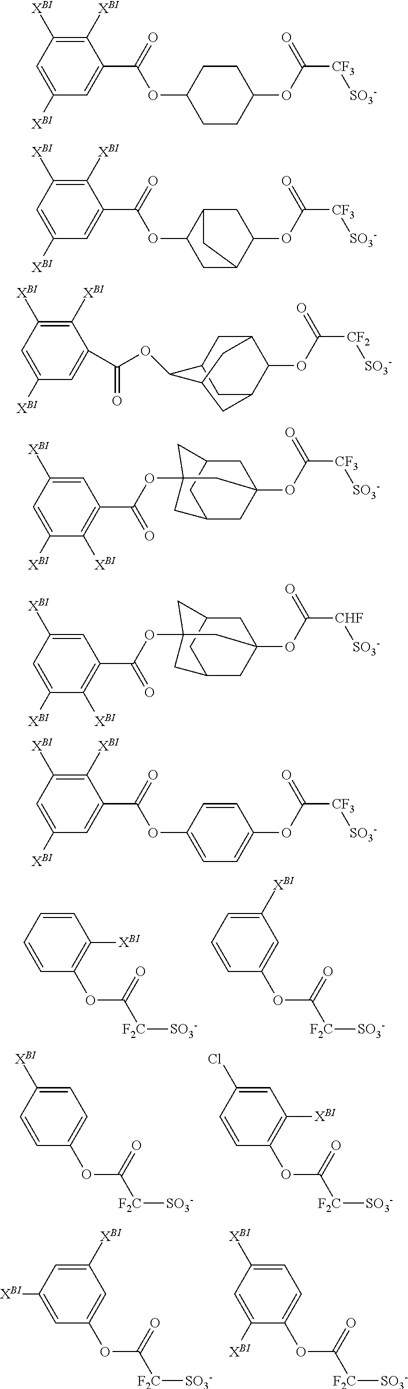

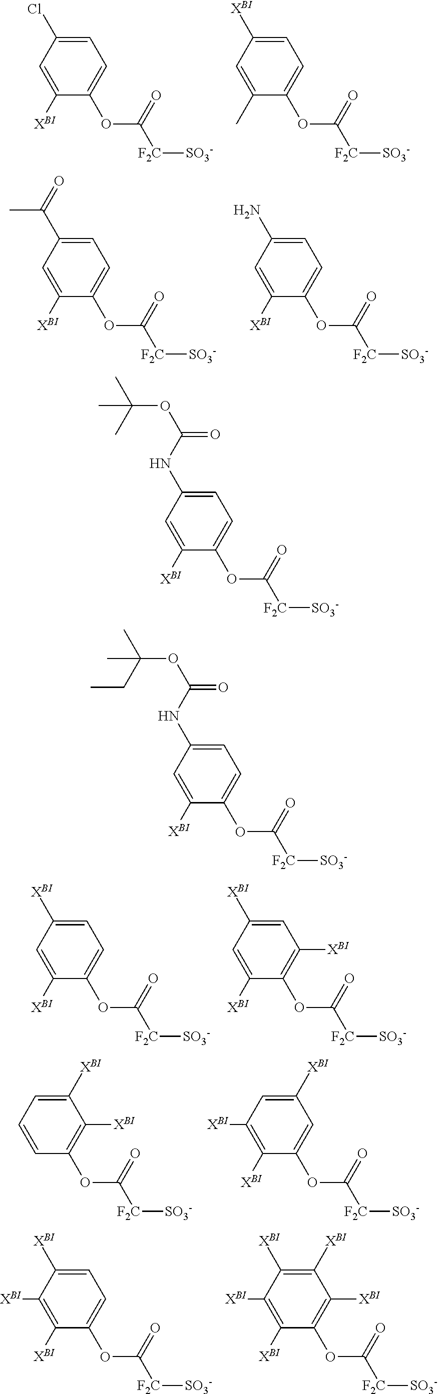

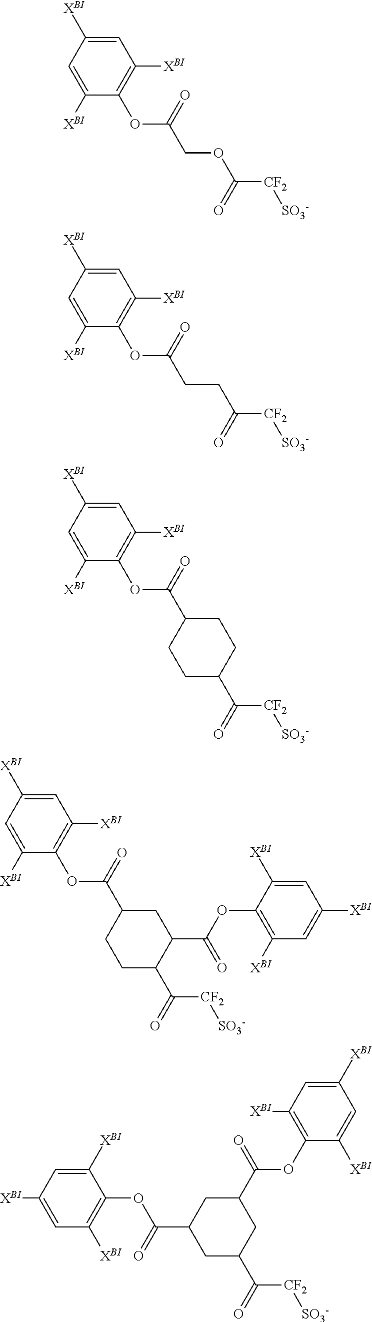

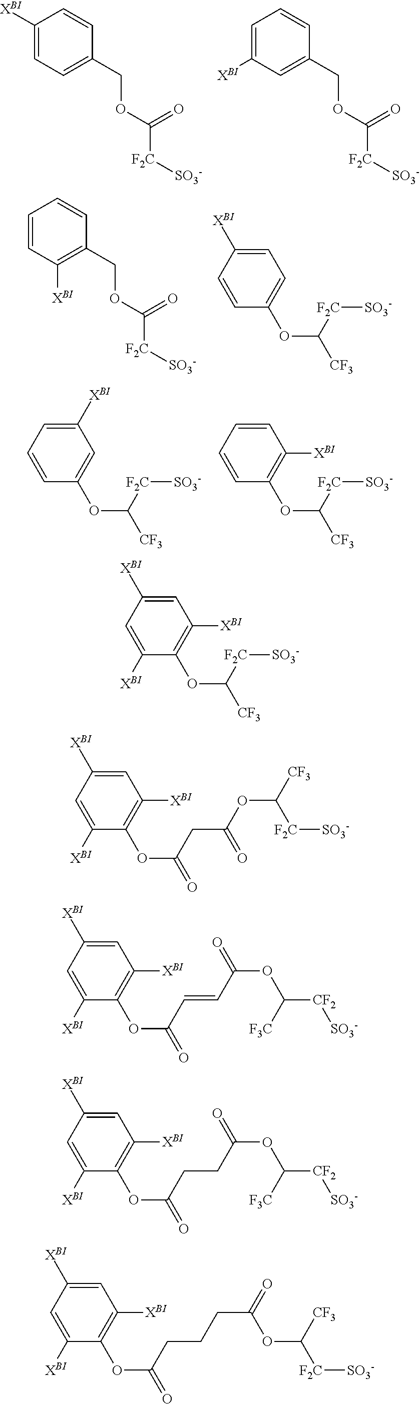

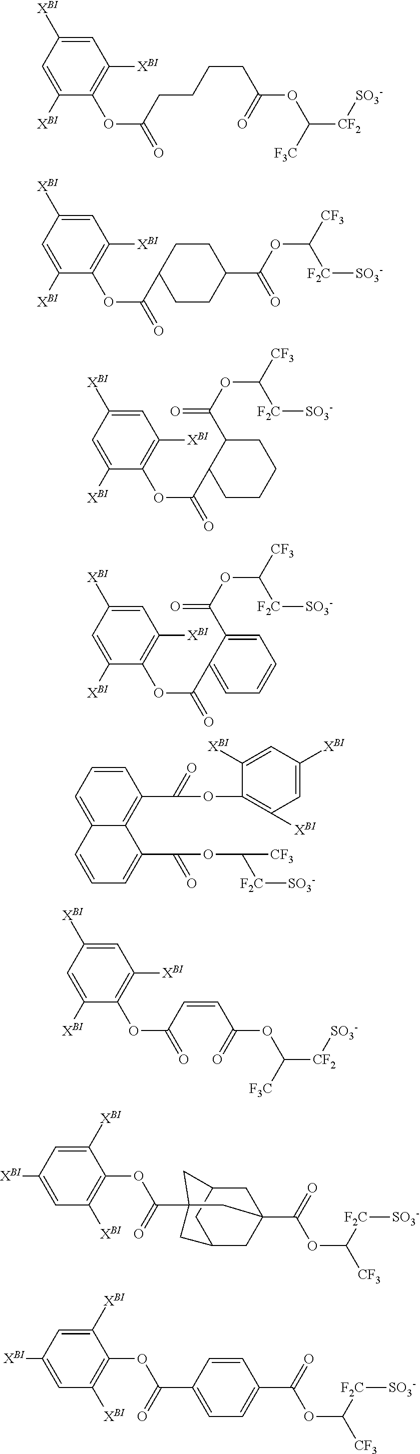

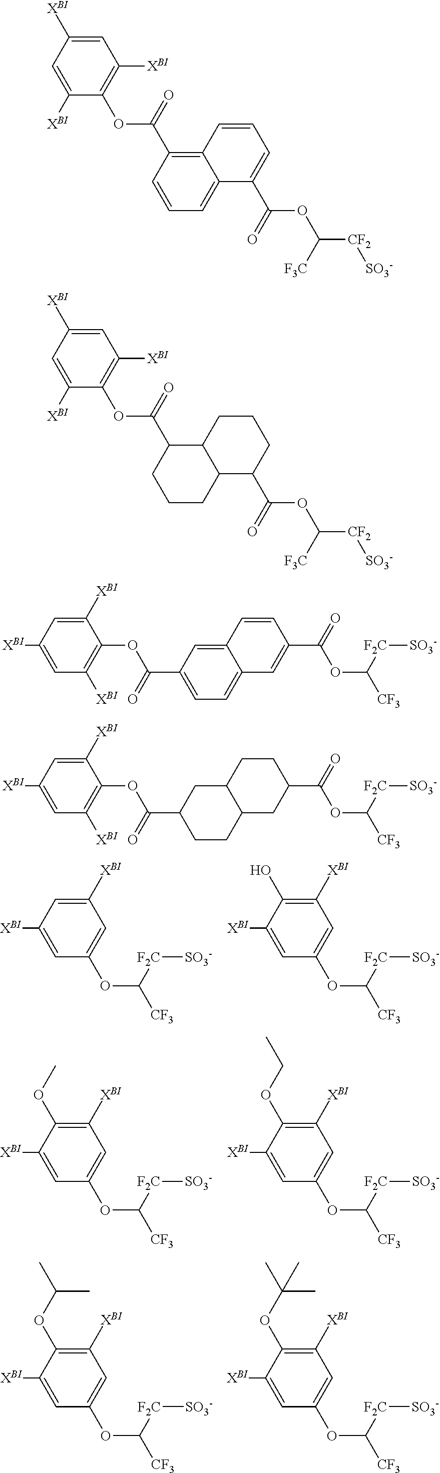

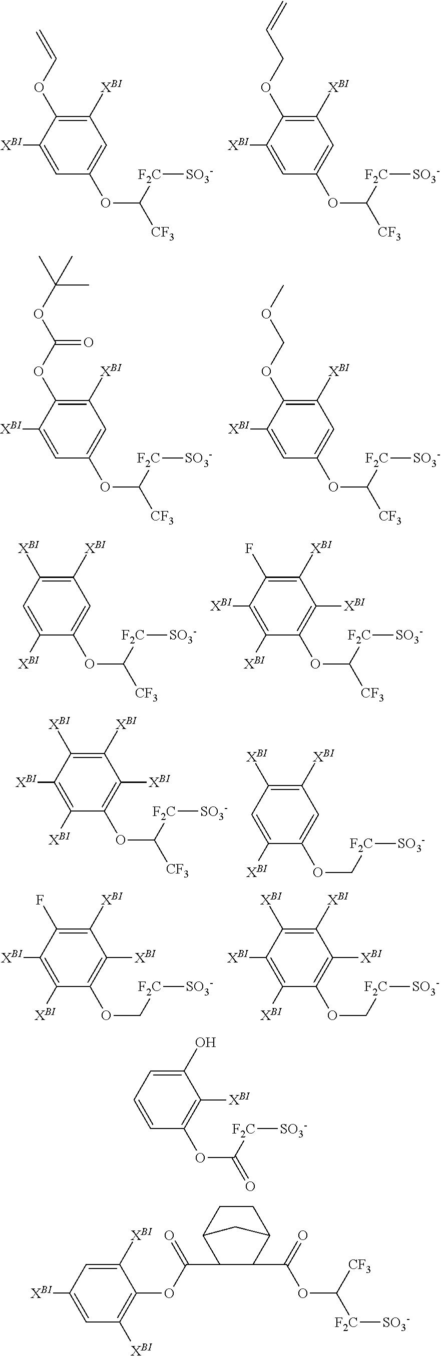

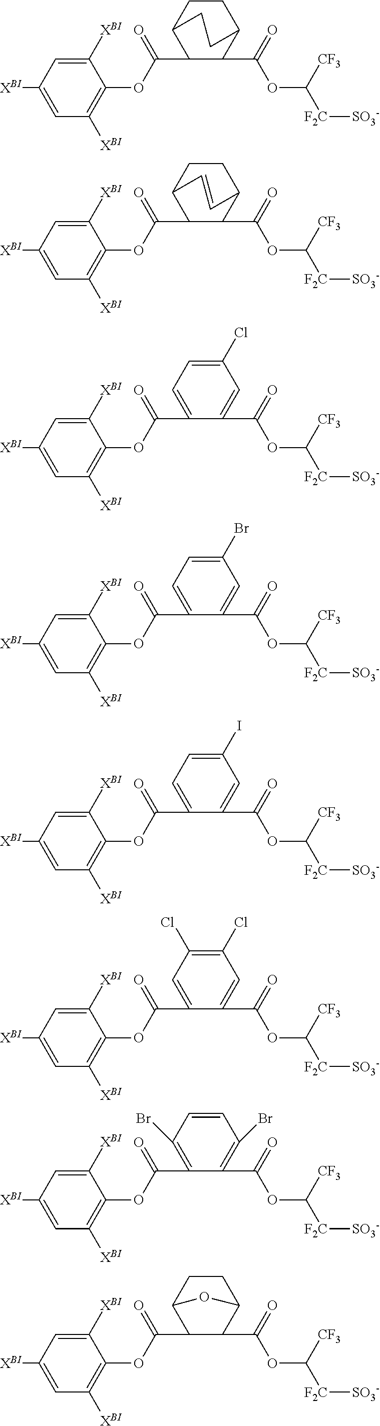

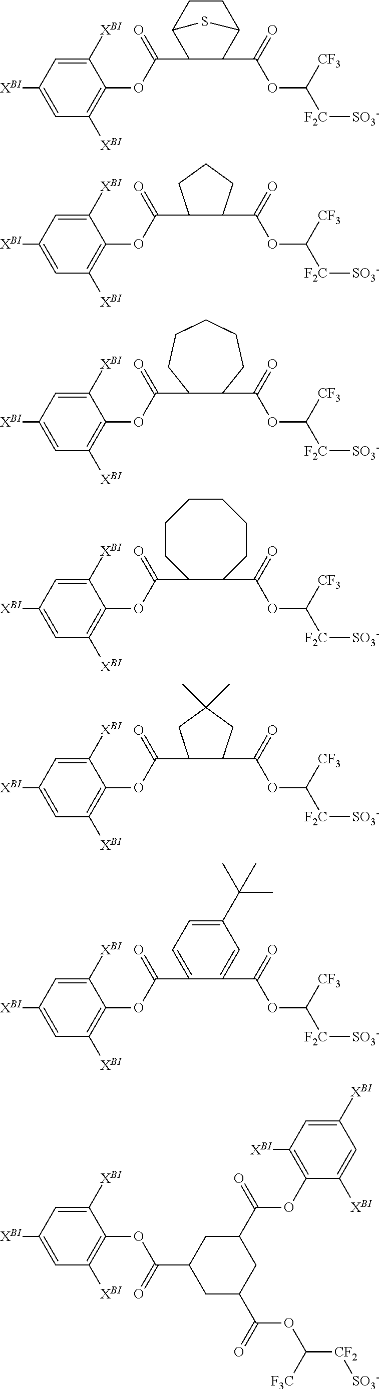

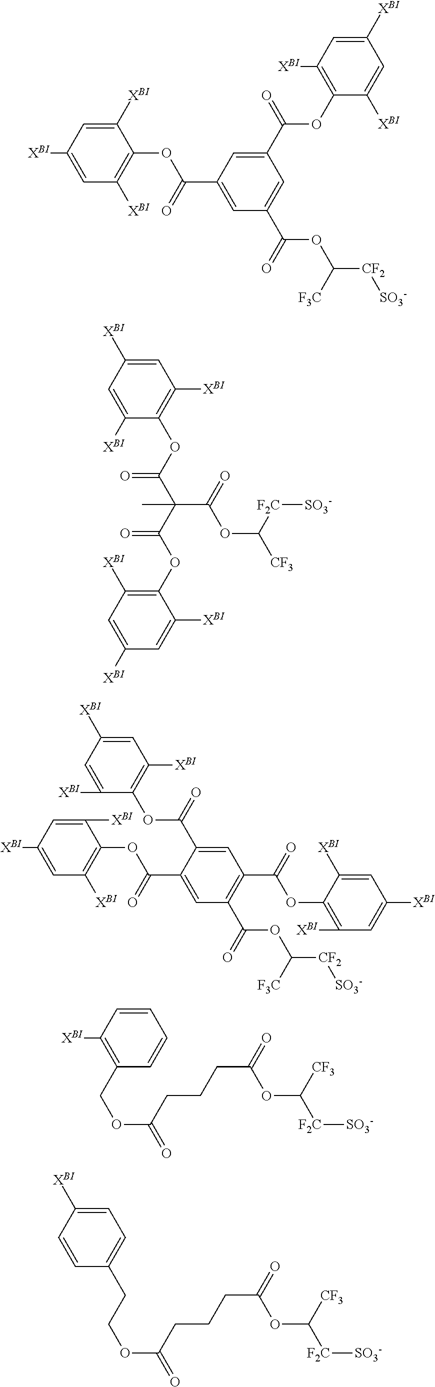

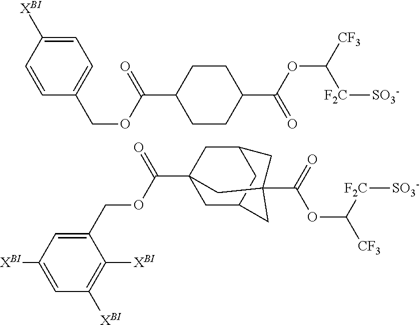

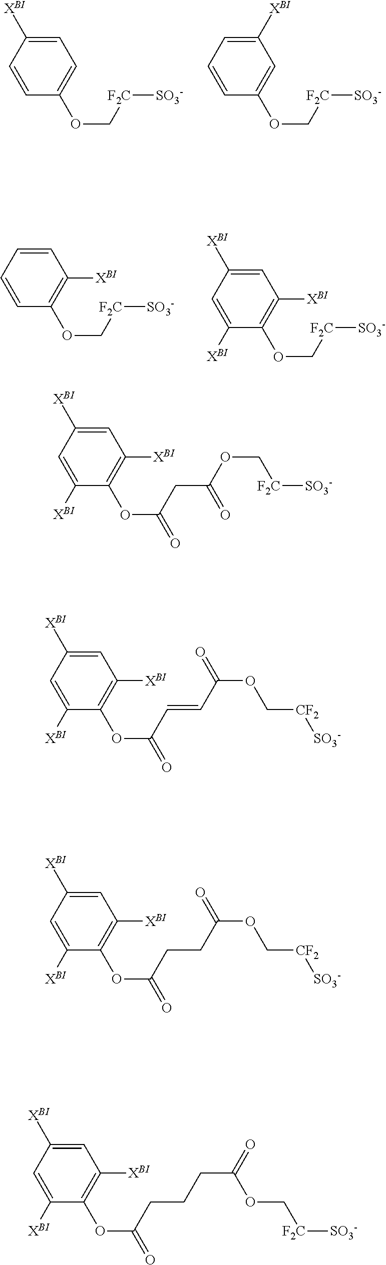

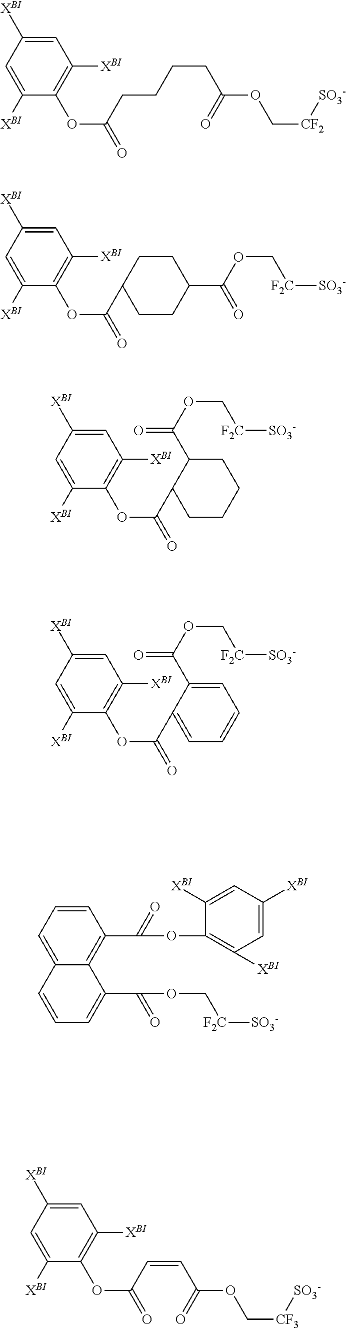

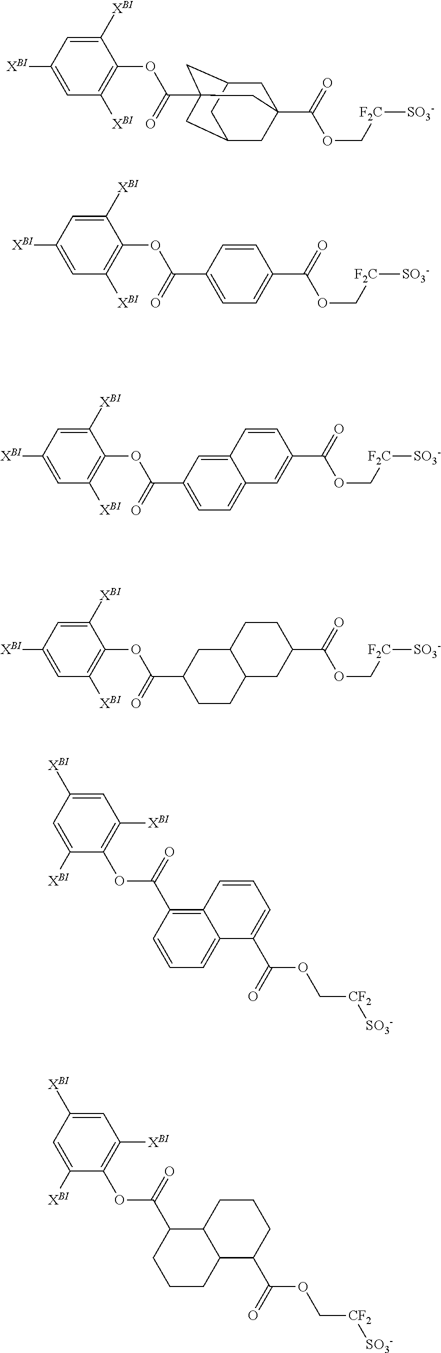

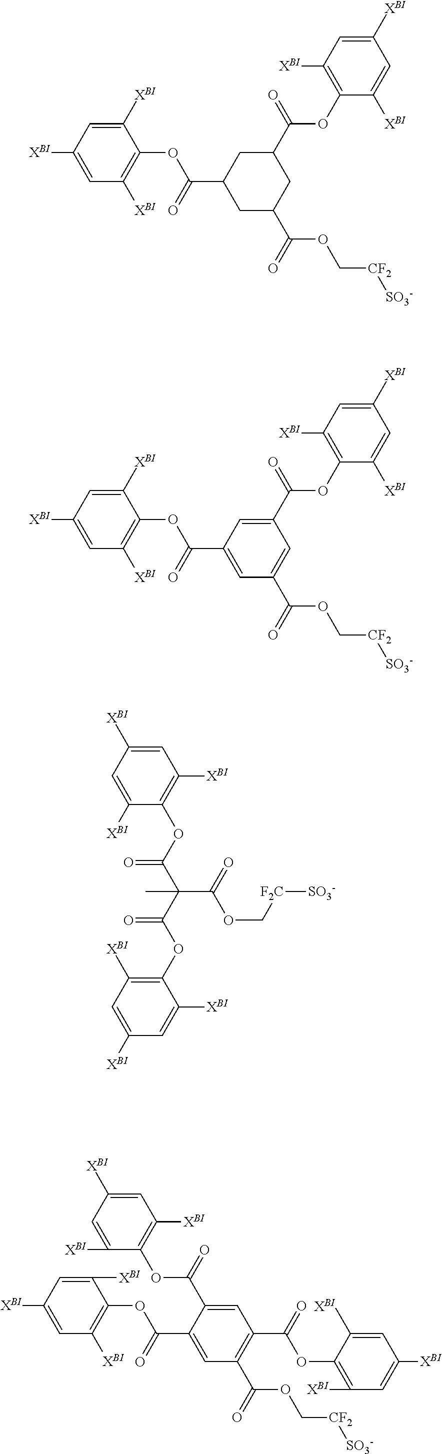

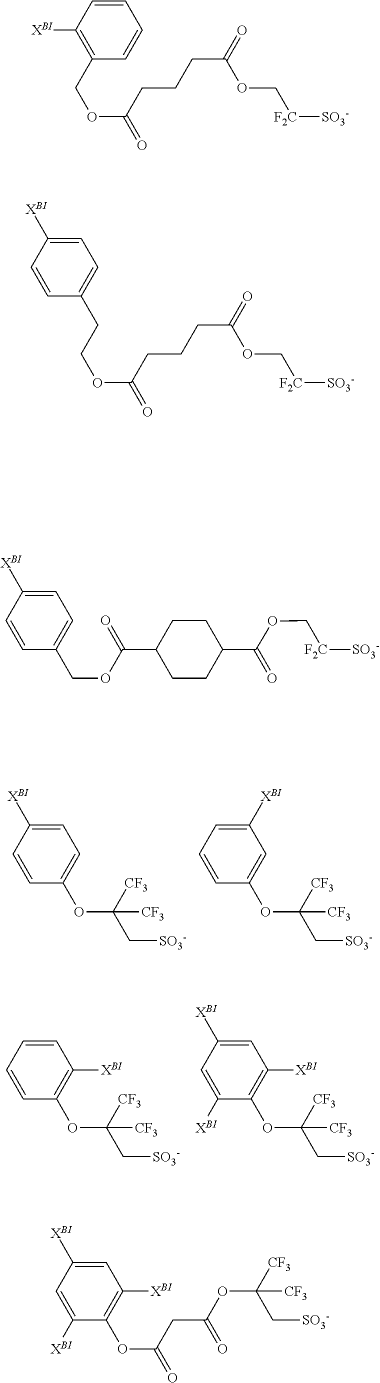

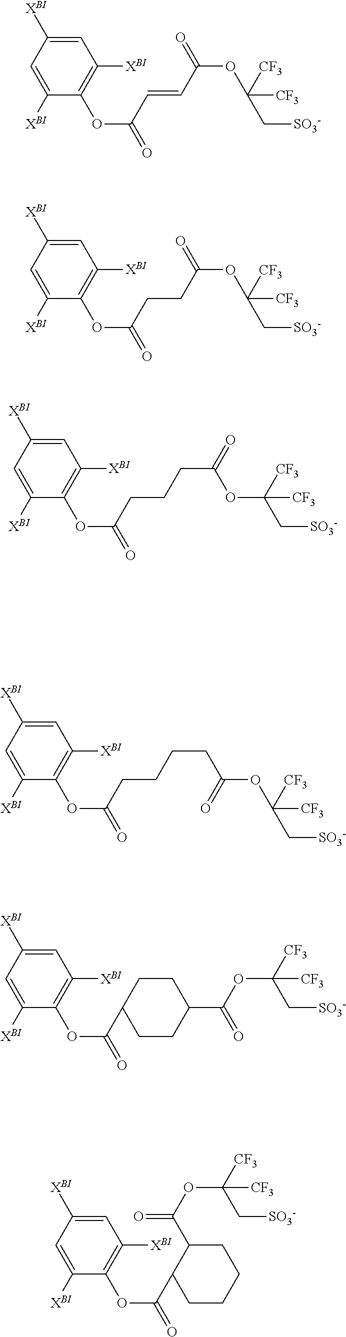

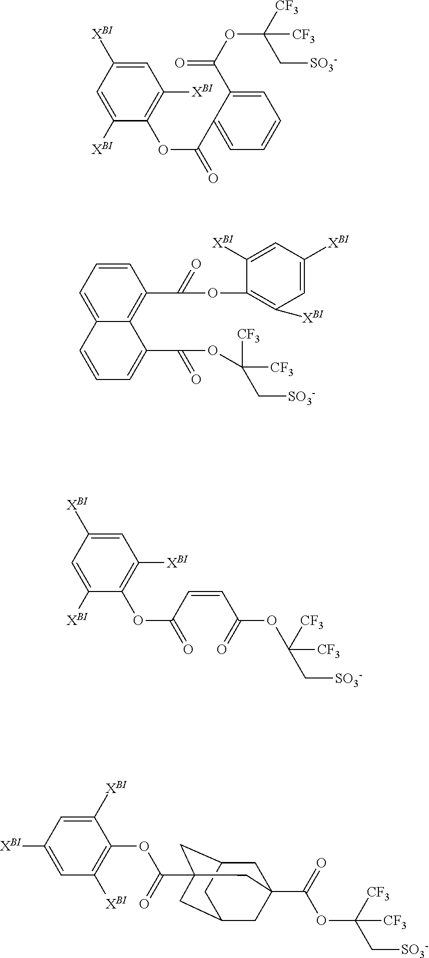

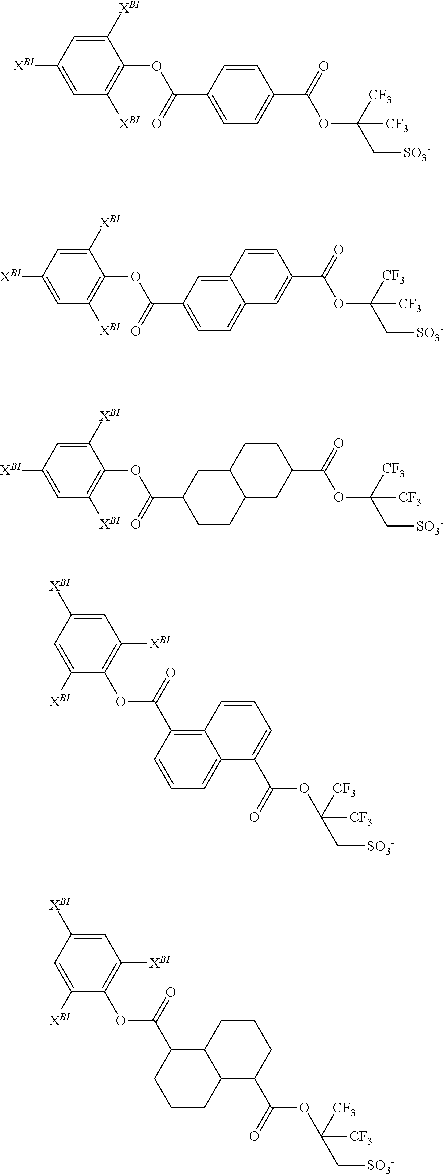

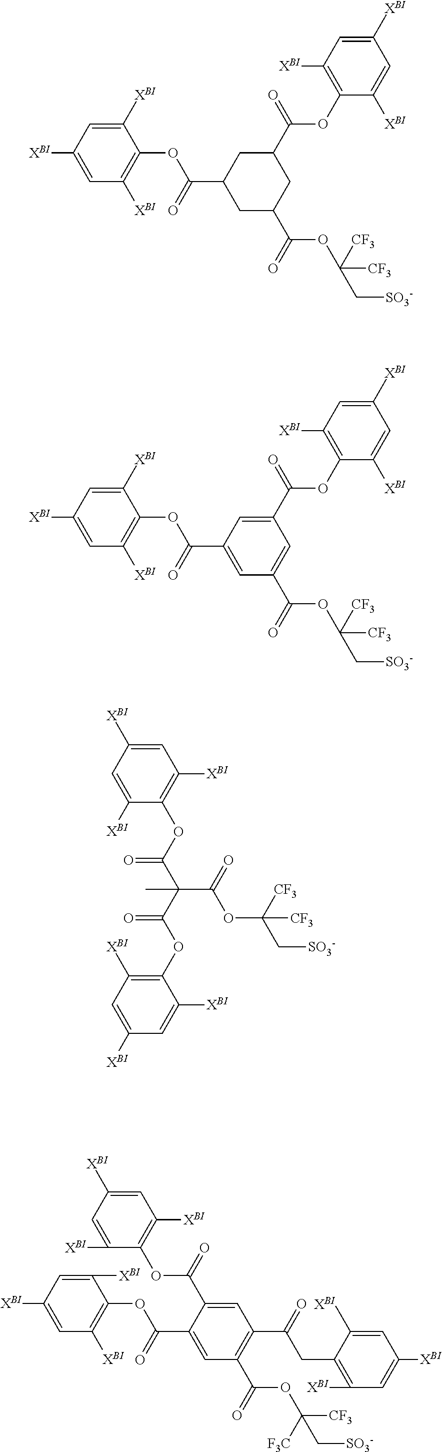

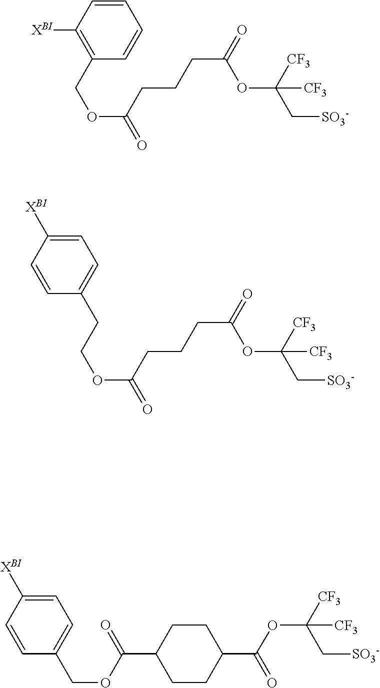

In the inventive resist composition, the additive-type acid generator is preferably contained in an amount of 0.1 to 50 parts by mass, more preferably 1 to 40 parts by mass based on 100 parts by mass of the resist material. By the resist material containing the repeating units-d1 to -d3 and/or containing the additive-type acid generator, the inventive resist composition can function as a chemically-amplified resist composition.

### Organic Solvent

The inventive resist composition may contain an organic solvent. The organic solvent is not particularly limited as long as it is capable of dissolving the above-described components and the components described below. Examples of such an organic solvent include ones disclosed in paragraphs [0144] and [0145] of JP 2008-111103 A: ketones, such as cyclohexanone, cyclopentanone, methyl-2-n-pentyl ketone, and 2-heptanone; alcohols, such as 3-methoxybutanol, 3-methyl-3-methoxybutanol, 1-methoxy-2-propanol, 1-ethoxy-2-propanol, and diacetone alcohol; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones, such as γ-butyrolactone; and mixed solvents thereof.

In the inventive resist composition, the organic solvent is preferably contained in an amount of 100 to 10,000 parts by mass, more preferably 200 to 8,000 parts by mass based on 100 parts by mass of the resist material.

### Other Components

In addition to the above-described components, a surfactant, a dissolution inhibitor, and so forth can be blended in appropriate combination depending on the purpose to formulate a resist composition. Thereby, in an exposed area of the resist material, the dissolution rate to a developer is accelerated by the catalytic reaction, so that the resist composition successfully has very high sensitivity. In this case, the resist film has high dissolution contrast and high resolution, exposure latitude, excellent process adaptability, and favorable pattern profile after exposure. Particularly, the resist composition is capable of suppressing acid diffusion, resulting in a small difference in profile between isolated and nested. Because of these advantages, the inventive resist composition is highly practical and very effective as a resist material for VLSI.

Examples of the surfactant include ones disclosed in paragraphs [0165] and [0166] of JP 2008-111103 A. Adding a surfactant can further enhance or control the coatability of the resist composition. One kind of the surfactant may be used, or two or more kinds thereof may be used in combination. In the inventive resist composition, the surfactant is preferably contained in an amount of 0.0001 to 10 parts by mass based on 100 parts by mass of the resist material.

Blending a dissolution inhibitor can further increase the difference in dissolution rate between exposed and unexposed areas, and further enhance the resolution.

Examples of the dissolution inhibitor include a compound which contains two or more phenolic hydroxy groups per molecule, and in which 0 to 100 mol % of all the hydrogen atoms of the phenolic hydroxy groups are substituted with acid-labile groups; and a compound which contains a carboxy group in a molecule, and in which 50 to 100 mol % of all the hydrogen atoms of such carboxy groups are substituted with acid-labile groups on average. The compounds each have a molecular weight of preferably 100 to 1,000, more preferably 150 to 800. Specific examples include compounds obtained by substituting acid-labile groups for hydrogen atoms of hydroxy groups or carboxy groups of bisphenol A, trisphenol, phenolphthalein, cresol novolak, naphthalenecarboxylic acid, adamantanecarboxylic acid, or cholic acid; etc. Examples of such compounds are disclosed in paragraphs [0155] to [0178] of JP 2008-122932 A.

The dissolution inhibitor is preferably contained in an amount of 0 to 50 parts by mass, more preferably 5 to 40 parts by mass based on 100 parts by mass of the resist material. One kind of the dissolution inhibitor may be used, or two or more kinds thereof may be used in combination.

The inventive resist composition may also contain a quencher (hereinafter, referred to as other quenchers). Examples of the other quenchers include conventional basic compounds. Examples of the conventional basic compounds include primary, secondary, and tertiary aliphatic amines, mixed amines, aromatic amines, heterocyclic amines, nitrogen-containing compounds having a carboxy group, nitrogen-containing compounds having a sulfonyl group, nitrogen-containing compounds having a hydroxy group, nitrogen-containing compounds having a hydroxyphenyl group, alcoholic nitrogen-containing compounds, amides, imides, carbamates, etc. Particularly preferable are primary, secondary, and tertiary amine compounds disclosed in paragraphs [0146] to [0164] of JP 2008-111103 A; in particular, amine compounds having a hydroxy group, an ether bond, an ester bond, a lactone ring, a cyano group, or a sulfonic acid ester bond; compounds having a carbamate group disclosed in JP 3790649 B2; etc. Adding such a basic compound can, for example, further suppress the acid diffusion rate in the resist film and correct the shape.

Other examples of the other quenchers include onium salts, such as sulfonium salts, iodonium salts, and ammonium salts, of carboxylic acids and sulfonic acids which are not fluorinated at a position, as disclosed in JP 2008-158339 A. While α-fluorinated sulfonic acid, imide acid, or methide acid is necessary to deprotect the acid-labile group of carboxylic acid ester, a carboxylic acid or sulfonic acid not fluorinated at a position is released by salt exchange with the onium salt not fluorinated at a position. Such carboxylic acid and sulfonic acid not fluorinated at a position hardly induce deprotection reaction, and thus function as quenchers.

Other examples of the other quenchers further include a polymeric quencher disclosed in JP 2008-239918 A. This quencher is oriented on the surface of an applied resist, and enhances the rectangularity of a resist after patterning. The polymeric quencher also has effects of preventing rounding of a pattern top and film thickness loss of a pattern when a top coat for immersion exposure is applied.

In the inventive resist composition, the other quenchers are preferably contained in an amount of 0 to 5 parts by mass, more preferably 0 to 4 parts by mass based on 100 parts by mass of the resist material. One kind of the other quenchers may be used, or two or more kinds thereof may be used in combination.

The inventive resist composition may be blended with a water-repellency enhancer for enhancing the water repellency on the resist surface after spin-coating. The water-repellency enhancer can be employed in immersion lithography with no top coat. The water-repellency enhancer is preferably a polymer compound containing a fluorinated alkyl group, a polymer compound containing a 1,1,1,3,3,3-hexafluoro-2-propanol residue with a particular structure, etc., more preferably ones exemplified in JP 2007-297590 A, JP 2008-111103 A, etc. The water-repellency enhancer needs to be dissolved in an organic solvent developer. The water-repellency enhancer having a particular 1,1,1,3,3,3-hexafluoro-2-propanol residue mentioned above has favorable solubility to developers. A polymer compound containing a repeating unit with an amino group or amine salt as a water-repellency enhancer exhibits high effects of preventing acid evaporation during post-exposure baking (PEB) and opening failure of a hole pattern after development. One kind of the water-repellency enhancer may be used, or two or more kinds thereof may be used in combination. In the inventive resist composition, the water-repellency enhancer is preferably contained in an amount of 0 to 20 parts by mass, more preferably 0.5 to 10 parts by mass based on 100 parts by mass of the resist material.

The inventive resist composition may also contain an acetylene alcohol. Examples of the acetylene alcohol include ones disclosed in paragraphs [0179] to [0182] of JP 2008-122932 A. In the inventive resist composition, the acetylene alcohol is preferably contained in an amount of 0 to 5 parts by mass based on 100 parts by mass of the resist material.

### Patterning Process

When the inventive resist composition is used for manufacturing various integrated circuits, known lithography techniques are applicable. That is, the present invention provides a patterning process including the steps of:
forming a resist film on a substrate by using the above-described resist composition;
exposing the resist film to a high-energy beam; and
developing the exposed resist film by using a developer.

For example, the inventive resist composition is applied onto a substrate (such as Si, SiO₂, SiN, SION, TIN, WSi, BPSG, SOG, and organic antireflective film) for manufacturing an integrated circuit or a substrate (such as Cr, CrO, CrON, MoSi₂, and SiO₂) for manufacturing a mask circuit by an appropriate coating process such as spin coating, roll coating, flow coating, dip coating, spray coating, or doctor coating so that the coating film can have a thickness of 0.01 to 2 μm. The resultant is prebaked on a hot plate preferably at 60 to 150° C. for 10 seconds to 30 minutes, more preferably at 80 to 120° C. for 30 seconds to 20 minutes. In this manner, a resist film is formed.

Then, the resist film is exposed using a high-energy beam. Examples of the high-energy beam include ultraviolet ray, deep ultraviolet ray, EB (electron beam), EUV (extreme ultraviolet ray), X-ray, soft X-ray, excimer laser, i-line, γ-ray, synchrotron radiation, etc. In particular, it is preferable to use i-line, KrF excimer laser beam, ArF excimer laser beam, electron beam, or extreme ultraviolet ray at a wavelength of 3 to 15 nm. When ultraviolet ray, deep ultraviolet ray, EUV, X-ray, soft X-ray, excimer laser, γ-ray, synchrotron radiation, or the like is employed as the high-energy beam, the irradiation is performed while using a mask for forming a target pattern at an exposure dose of preferably about 1 to 200 mJ/cm², more preferably about 10 to 100 mJ/cm². When EB is employed as the high-energy beam, the exposure dose is preferably about 0.1 to 100 μC/cm², more preferably about 0.5 to 50 μC/cm², and the writing is performed directly or while using a mask for forming a target pattern. Note that the inventive resist composition is particularly suitable for fine patterning with a KrF excimer laser, an ArF excimer laser, an EB, an EUV, X-ray, soft X-ray, γ-ray, or synchrotron radiation among the high-energy beams, and is particularly suitable for fine patterning with an EB or an EUV.

The exposure may be followed by PEB on a hot plate, preferably at 50 to 150° C. for 10 seconds to 30 minutes, more preferably 60 to 120° C. for 30 seconds to 20 minutes.

After the exposure or PEB, development is performed using a developer of 0.1 to 10 mass %, preferably 2 to 5 mass %, aqueous alkaline solution, such as tetramethylammonium hydroxide (TMAH), tetraethylammonium hydroxide (TEAH), tetrapropylammonium hydroxide (TPAH), and tetrabutylammonium hydroxide (TBAH), for 3 seconds to 3 minutes, preferably 5 seconds to 2 minutes, by a conventional technique, such as a dip, puddle, or spray method. Thereby, the portion irradiated with the light is dissolved by the developer, while the unexposed portion remains undissolved. In this way, the target positive pattern is formed on the substrate.

The resist composition containing a resist material that contains an acid-labile group can also be used to perform negative development to obtain a negative pattern by organic solvent development. Examples of the developer used in this event include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, benzyl formate, phenylethyl formate, methyl 3-phenylpropionate, benzyl propionate, phenylethyl acetate, 2-phenylethyl acetate, etc. One of these organic solvents can be used, or two or more thereof can be used in mixture.

When the development is completed, rinsing can be performed. The rinsing liquid is preferably a solvent that is miscible with the developer but does not dissolve the resist film. As such a solvent, it is preferable to use an alcohol having 3 to 10 carbon atoms, an ether compound having 8 to 12 carbon atoms, and an alkane, alkene, alkyne, and aromatic solvent, each having 6 to 12 carbon atoms.

Specific examples of the alcohol having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, 1-octanol, etc.

Examples of the ether compound having 8 to 12 carbon atoms include di-n-butyl ether, diisobutyl ether, di-sec-butyl ether, di-n-pentyl ether, diisopentyl ether, di-sec-pentyl ether, di-tert-pentyl ether, di-n-hexyl ether, etc.

Examples of the alkane having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, cyclononane, etc. Examples of the alkene having 6 to 12 carbon atoms include hexene, heptene, octene, cyclohexene, methylcyclohexene, dimethylcyclohexene, cycloheptene, cyclooctene, etc. Examples of the alkyne having 6 to 12 carbon atoms include hexyne, heptyne, octyne, etc.

Examples of the aromatic solvent include toluene, xylene, ethylbenzene, isopropylbenzene, tert-butylbenzene, mesitylene, etc.

The rinsing can reduce resist pattern collapse and defect formation. Meanwhile, the rinsing is not necessarily essential, and the amount of the solvent used can be reduced by not performing the rinsing.

After the development, a hole pattern or trench pattern can be shrunk by thermal flow, RELACS process, or DSA process. A shrink agent is applied onto the hole pattern, and the shrink agent undergoes crosslinking on the resist surface by diffusion of the acid catalyst from the resist layer during baking, so that the shrink agent is attached to sidewalls of the hole pattern. The baking temperature is preferably 70 to 180° C., more preferably 80 to 170° C. The baking time is preferably 10 to 300 seconds. The extra shrink agent is removed, and thus the hole pattern is shrunk.

### EXAMPLES

Hereinafter, the present invention will be described specifically with reference to Synthesis Examples, Examples, and Comparative Examples. However, the present invention is not limited to the following Examples.

### Synthesis of Monomers

### Synthesis Examples

### [Synthesis Example 1-1] Synthesis of Monomer 1

Monomer 1 was synthesized according to the following scheme.

### (1) Synthesis of Compound 2

Compound 1 (580 g), 2-chloroethanol (520 g), potassium carbonate (726 g), sodium bromide (31.5 g), and DMF (3,970 g) were mixed together and stirred at 90° C. for 20 hours. After the reaction was completed, the mixture was ice-cooled, pure water (3,600 g) was added thereto, and the mixture was stirred for 30 minutes. After that, methyl isobutyl ketone (4,500 g) was added thereto, and the mixture was stirred for 1 hour. The organic layer was collected, then a common aqueous work-up was performed, the solvent was evaporated, hexane (3,500 g) was added, and the mixture was stirred for 2 hours to precipitate a crystal. Subsequently, the resultant was filtered to obtain Compound 2 (604 g) as a solid.

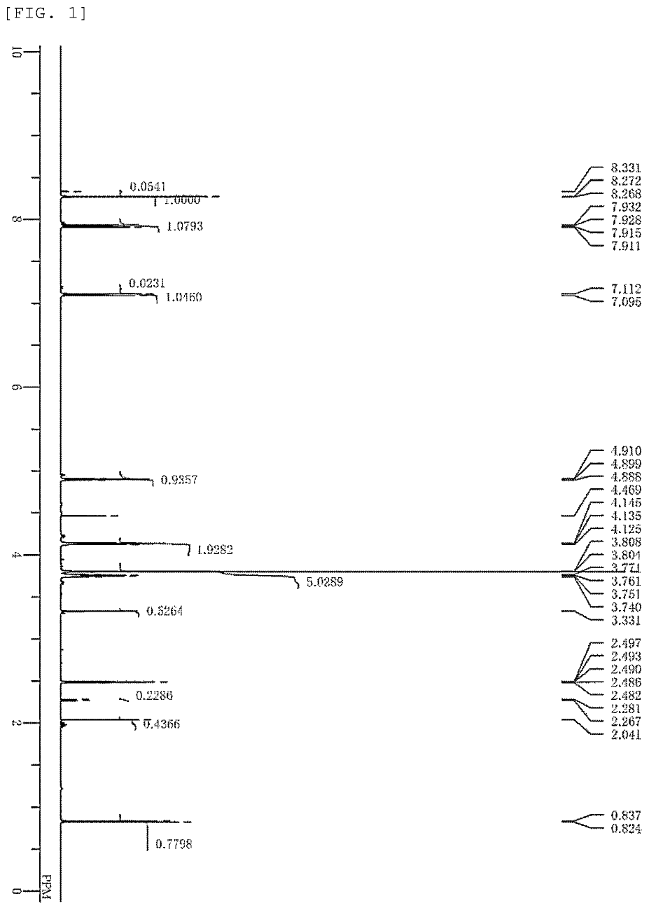

A ¹H-NMR (500 MHZ, DMSO-d₆) spectrum of Compound 2 is shown in FIG. 1.

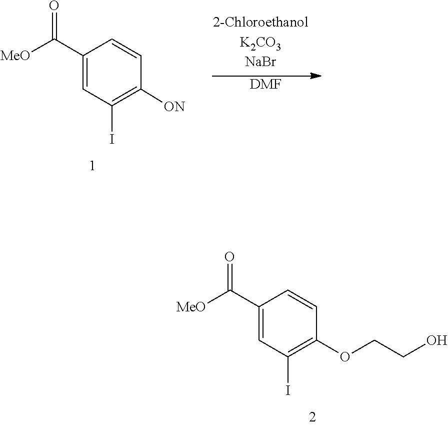

### (2) Synthesis of Compound 3

Compound 2 (600 g) and tetrahydrofuran (2,800 g) were stirred together under ice-cooling, and a 25% aqueous solution of sodium hydroxide (330 g) and pure water (900 g) were added dropwise. The mixture was stirred at 40° C. for 16 hours. After the reaction was completed, the organic solvent was evaporated, and the aqueous solution was washed with TBME. A 20% hydrochloric acid (430 g) and hexane (1 L) were added thereto, and the mixture was stirred to precipitate a crystal. Subsequently, the resultant was filtered to obtain Compound 3 (560 g) as a solid.

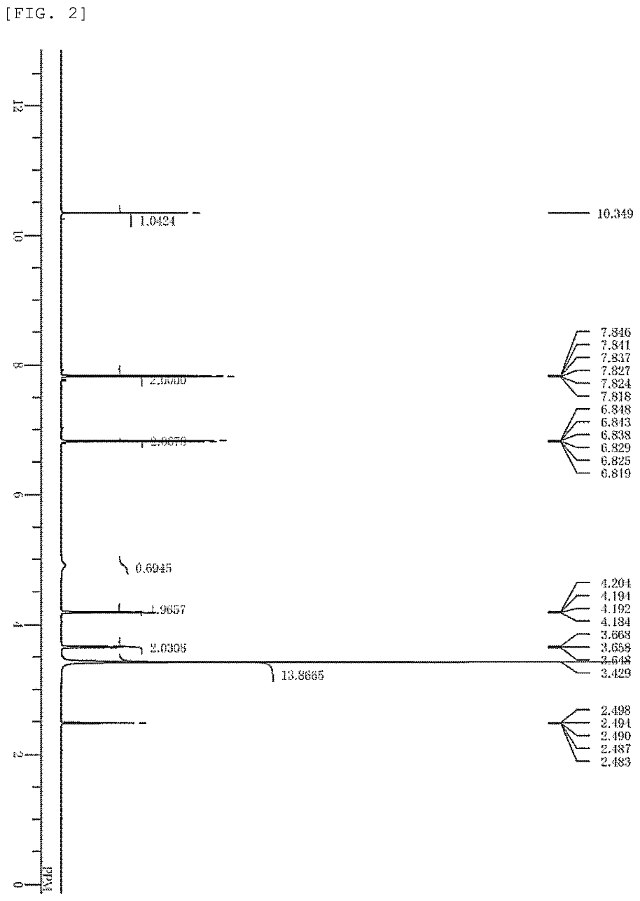

A ¹H-NMR (500 MHz, DMSO-d₆) spectrum of Compound 3 is shown in FIG. 2.

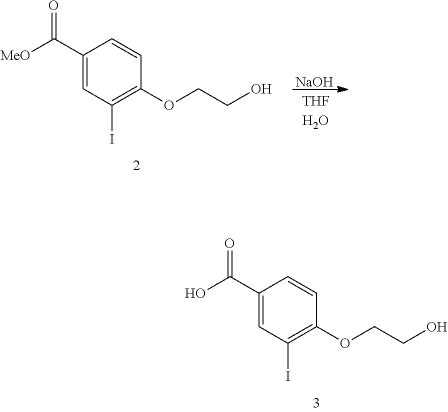

### (3) Synthesis of Compound 4

Compound 3 (560 g), methacrylic anhydride (340 g), acetonitrile (3,000 g), and a polymerization inhibitor (1,000 ppm/theoretical yield) were dissolved, and under ice-cooling, a mixture of triethylamine (442 g), DMAP (22 g), and acetonitrile (600 g) was added thereto dropwise. Subsequently, the mixture was stirred under ice-cooling for 3 hours. After the reaction was completed, 5% aqueous sodium bicarbonate (1,900 g) was added under ice-cooling, and the mixture was stirred for 20 minutes. Then, a 20% aqueous solution of hydrochloric acid (1,070 g) and pure water (5,300 g) were added thereto, and the mixture was stirred to precipitate a crystal. A solid was obtained by filtration, and the solid was dissolved in ethyl acetate (4,500 g). The organic layer was collected, then washed with saturated saline and pure water, and the organic layer was subjected to activated carbon treatment. The solvent was evaporated, hexane (5.7 L) was added to the resultant, and the mixture was stirred to precipitate a crystal. Subsequently the resultant was filtered to obtain Compound 4 (460 g) as a solid.

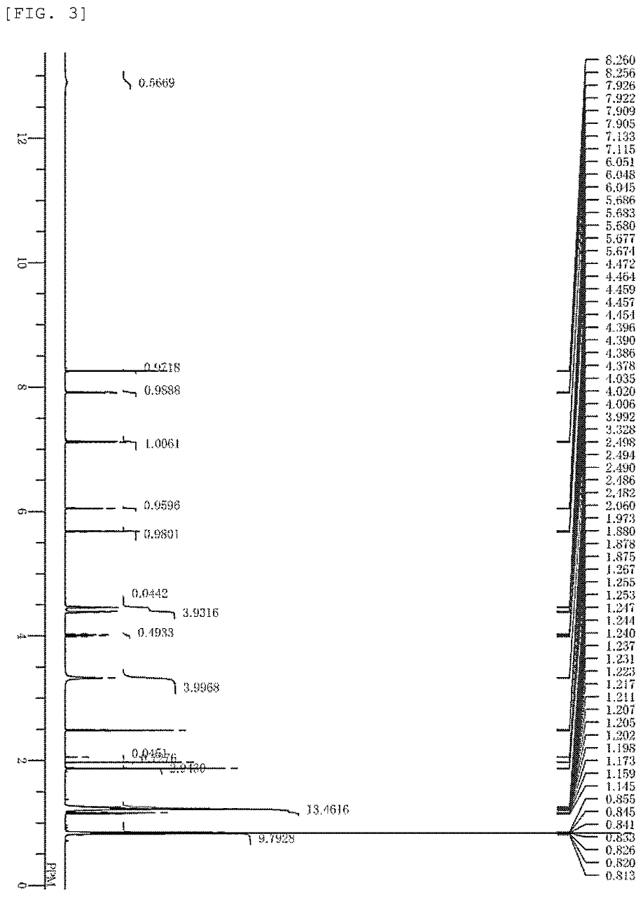

A ¹H-NMR (500 MHZ, DMSO-d₆) spectrum of Compound 4 is shown in FIG. 3.

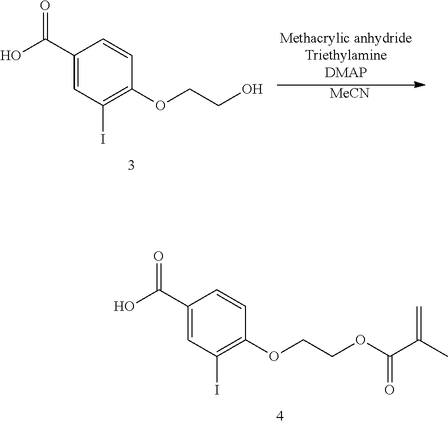 [SMILES: "MeCN" ; O=C(O)c1ccc(OCCO)c(I)c1 ; C=C(C)C(=O)OCCOc1ccc(C(=O)O)cc1I]

### (4) Synthesis of Monomer 1

To Compound 4 (38 g) were added potassium carbonate (17 g), 1-pentanol (40 g), and pure water (90 g), and the mixture was stirred at room temperature for 1 hour. After that, Compound 5 (44 g) and methylene chloride (300 g) were added thereto, and the mixture was stirred for 2 hours. The organic layer was collected, then a common aqueous work-up was performed, a polymerization inhibitor (100 ppm/theoretical yield) was added to the resultant, and the solvent was evaporated to obtain Monomer 1 as an oil (60 g). The obtained Monomer 1 was dissolved in γ-butyrolactone (56 g) to obtain a γ-butyrolactone solution.

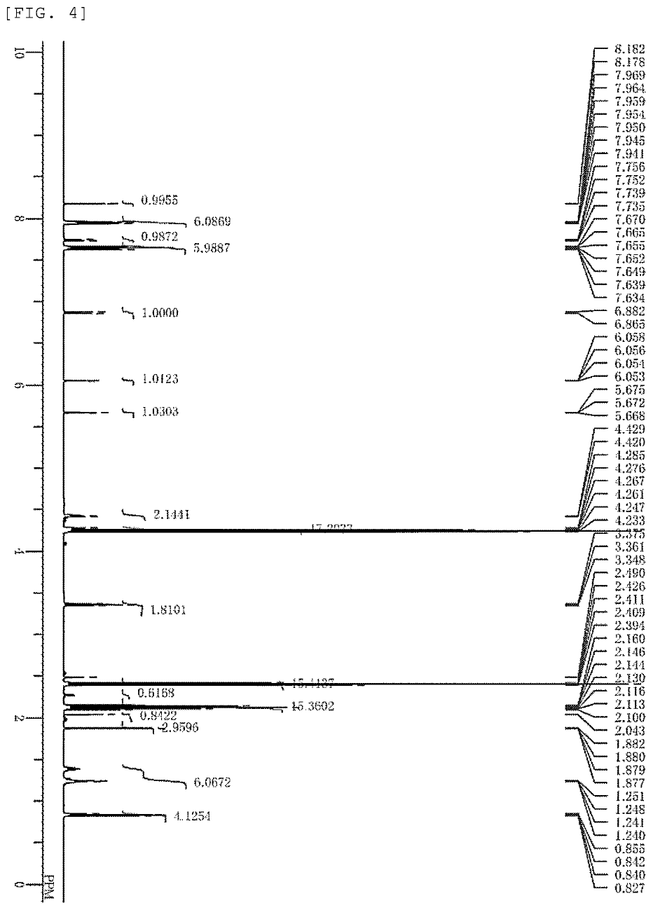

A ¹H-NMR (500 MHz, DMSO-d₆) spectrum of Monomer 1 is shown in FIG. 4. Besides the peak of the target product, the peaks of the used solvents (1-pentanol, methyl isobutyl ketone, and γ-butyrolactone) have been detected.

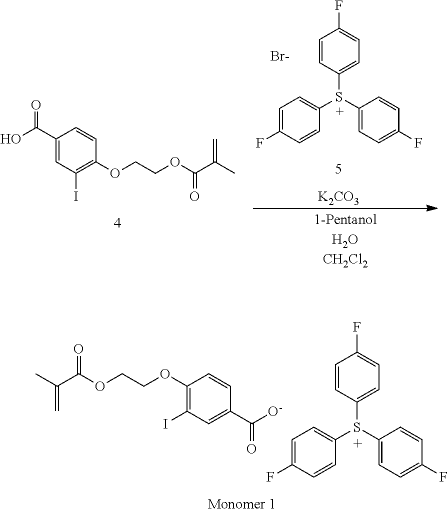

The following Monomers 2 to 15 and Comparative Monomers 1 to 3 were obtained by ion exchange between a sulfonium bromide and an iodinated benzoic acid compound having a polymerizable double bond or a benzoic acid compound. Monomer 1 is also shown.

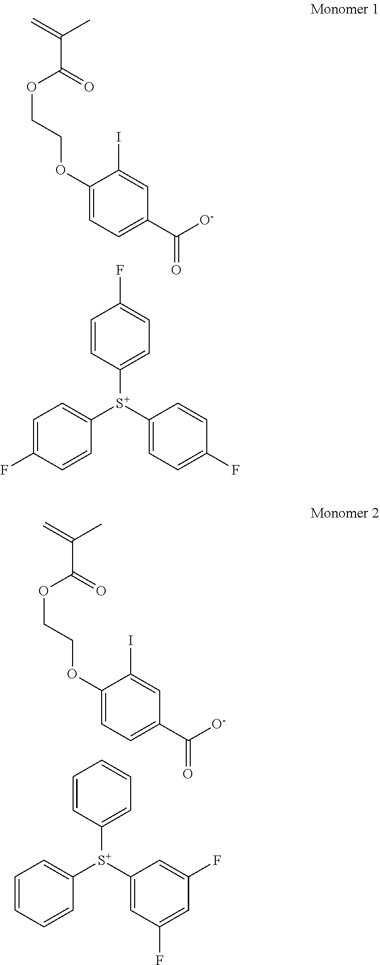 [SMILES: Fc1ccc([S+](c2ccc(F)cc2)c2ccc(F)cc2)cc1 ; C=C(C)C(=O)OCCOc1ccc(C(=O)[O-])cc1I ; Fc1cc(F)cc([S+](c2ccccc2)c2ccccc2)c1 ; C=C(C)C(=O)OCCOc1ccc(C(=O)[O-])cc1I]

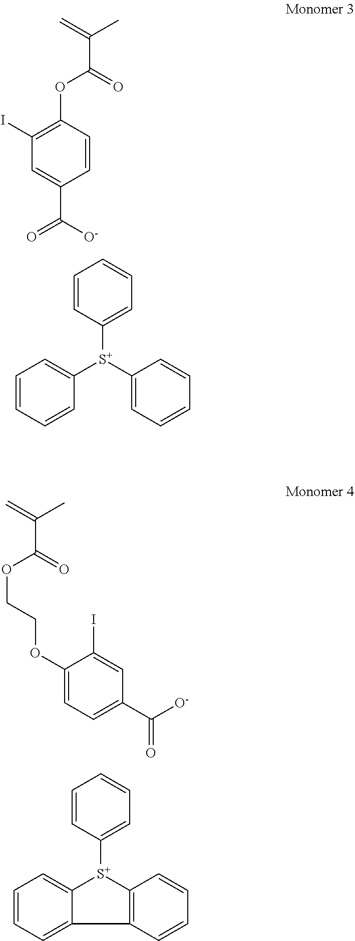 [SMILES: C=C(C)C(=O)Oc1ccc(C(=O)[O-])cc1I ; c1ccc([S+](c2ccccc2)c2ccccc2)cc1 ; C=C(C)C(=O)OCCOc1ccc(C(=O)[O-])cc1I ; c1ccc(-[s+]2c3ccccc3c3ccccc32)cc1]

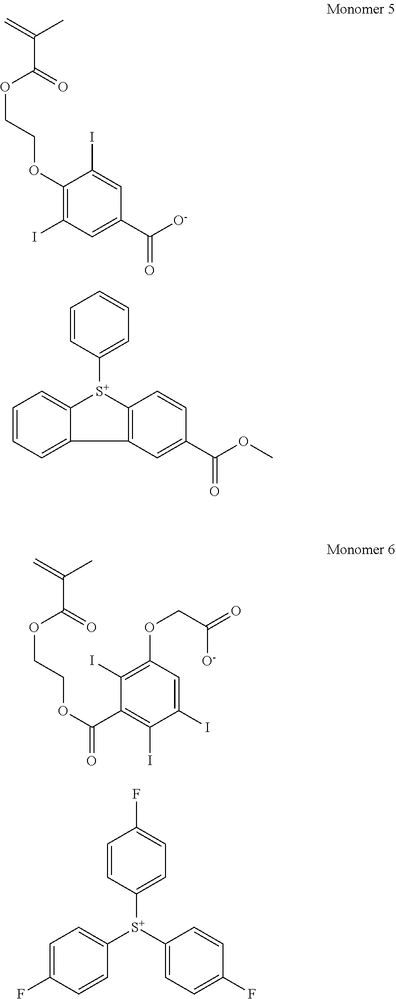 [SMILES: C=C(C)C(=O)OCCOc1c(I)cc(C(=O)[O-])cc1I ; COC(=O)c1ccc2c(c1)c1ccccc1[s+]2-c1ccccc1 ; C=C(C)C(=O)OCCOC(=O)c1c(I)c(I)cc(OCC(=O)[O-])c1I ; Fc1ccc([S+](c2ccc(F)cc2)c2ccc(F)cc2)cc1]

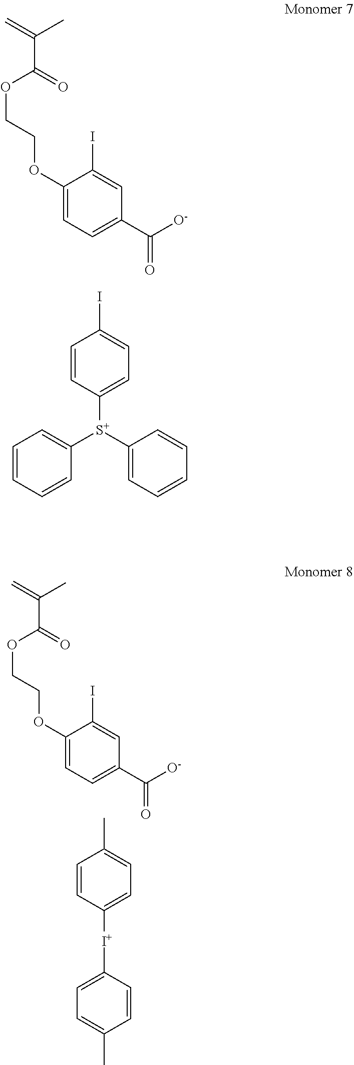 [SMILES: C=C(C)C(=O)OCCOc1ccc(C(=O)[O-])cc1I ; Ic1ccc([S+](c2ccccc2)c2ccccc2)cc1 ; C=C(C)C(=O)OCCOc1ccc(C(=O)[O-])cc1I ; Cc1ccc([I+]c2ccc(C)cc2)cc1]

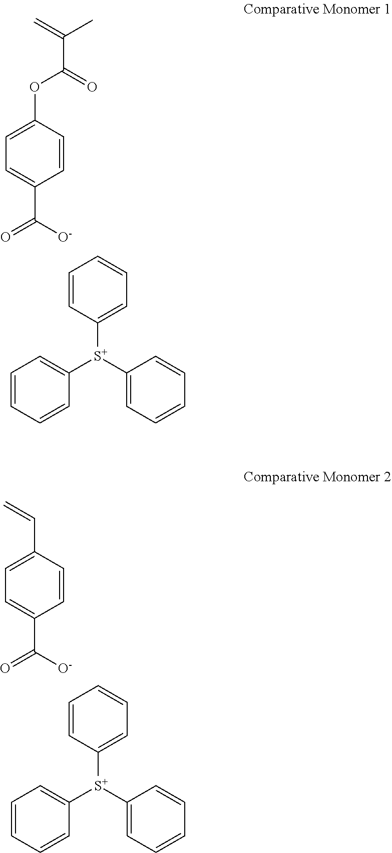 [SMILES: C=C(C)C(=O)Oc1ccc(C(=O)[O-])cc1 ; c1ccc([S+](c2ccccc2)c2ccccc2)cc1 ; C=Cc1ccc(C(=O)[O-])cc1 ; c1ccc([S+](c2ccccc2)c2ccccc2)cc1]

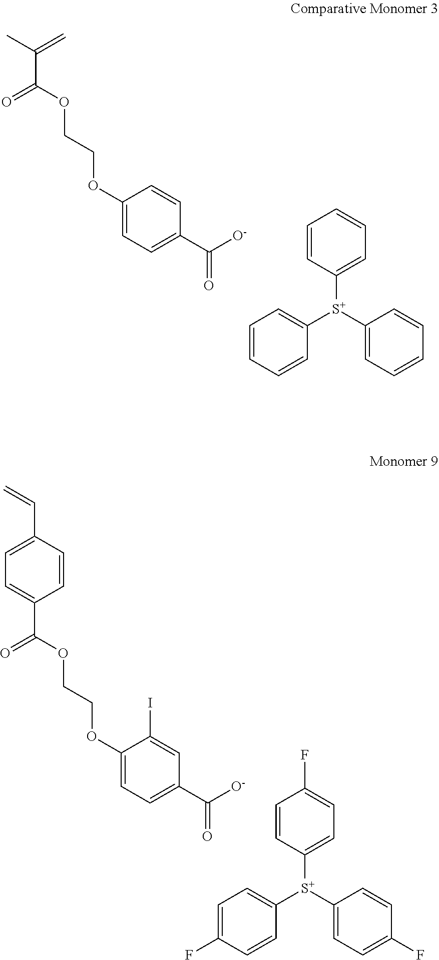 [SMILES: C=C(C)C(=O)OCCOc1ccc(C(=O)[O-])cc1 ; c1ccc([S+](c2ccccc2)c2ccccc2)cc1 ; C=Cc1ccc(C(=O)OCCOc2ccc(C(=O)[O-])cc2I)cc1 ; Fc1ccc([S+](c2ccc(F)cc2)c2ccc(F)cc2)cc1]

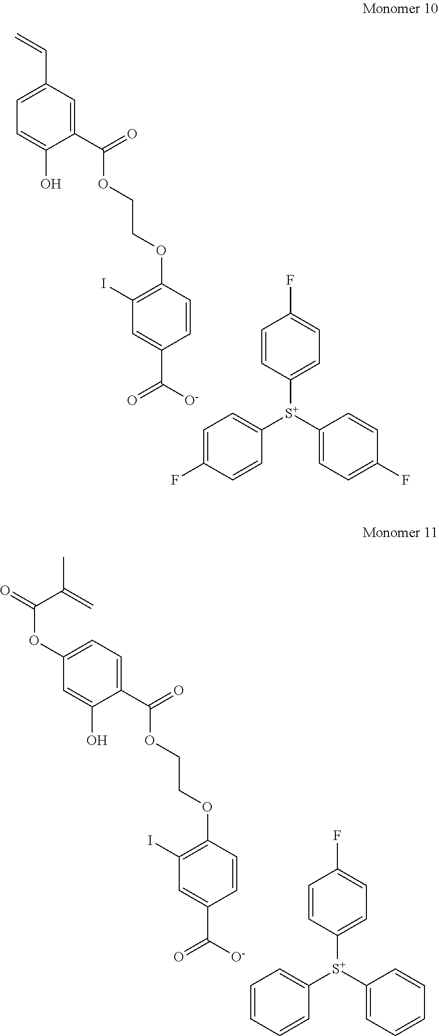 [SMILES: C=Cc1ccc(O)c(C(=O)OCCOc2ccc(C(=O)[O-])cc2I)c1 ; Fc1ccc([S+](c2ccc(F)cc2)c2ccc(F)cc2)cc1 ; C=C(C)C(=O)Oc1ccc(C(=O)OCCOc2ccc(C(=O)[O-])cc2I)c(O)c1 ; Fc1ccc([S+](c2ccccc2)c2ccccc2)cc1]

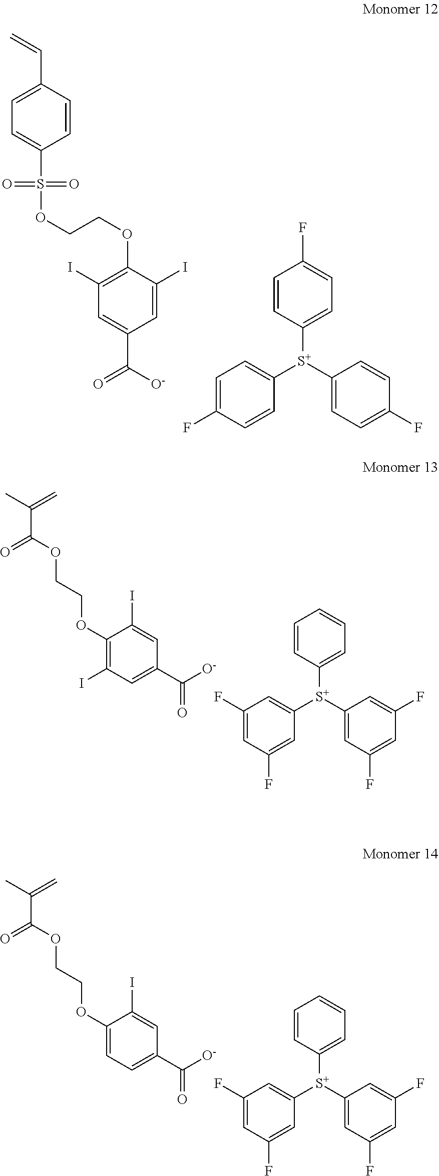 [SMILES: C=Cc1ccc(S(=O)(=O)OCCOc2c(I)cc(C(=O)[O-])cc2I)cc1 ; Fc1ccc([S+](c2ccc(F)cc2)c2ccc(F)cc2)cc1 ; C=C(C)C(=O)OCCOc1c(I)cc(C(=O)[O-])cc1I ; Fc1cc(F)cc([S+](c2ccccc2)c2cc(F)cc(F)c2)c1 ; C=C(C)C(=O)OCCOc1ccc(C(=O)[O-])cc1I ; Fc1cc(F)cc([S+](c2ccccc2)c2cc(F)cc(F)c2)c1]

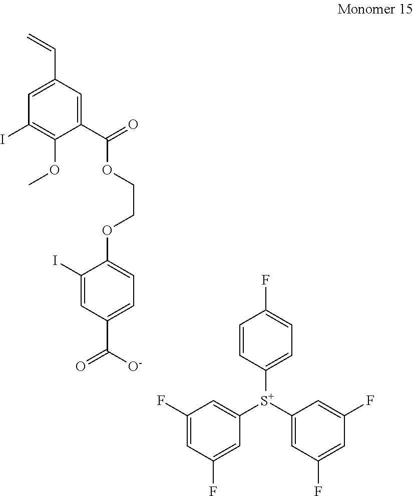 [SMILES: C=Cc1cc(I)c(OC)c(C(=O)OCCOc2ccc(C(=O)[O-])cc2I)c1 ; Fc1ccc([S+](c2cc(F)cc(F)c2)c2cc(F)cc(F)c2)cc1]

### [2] Synthesis of Polymers

The acid-labile group monomer (ALG Monomer 1) and PAG Monomers 1 to 8 used for the synthesis of polymers are as follows. The Mw of the polymers is a value measured in terms of polystyrene by GPC using THF as an eluent.

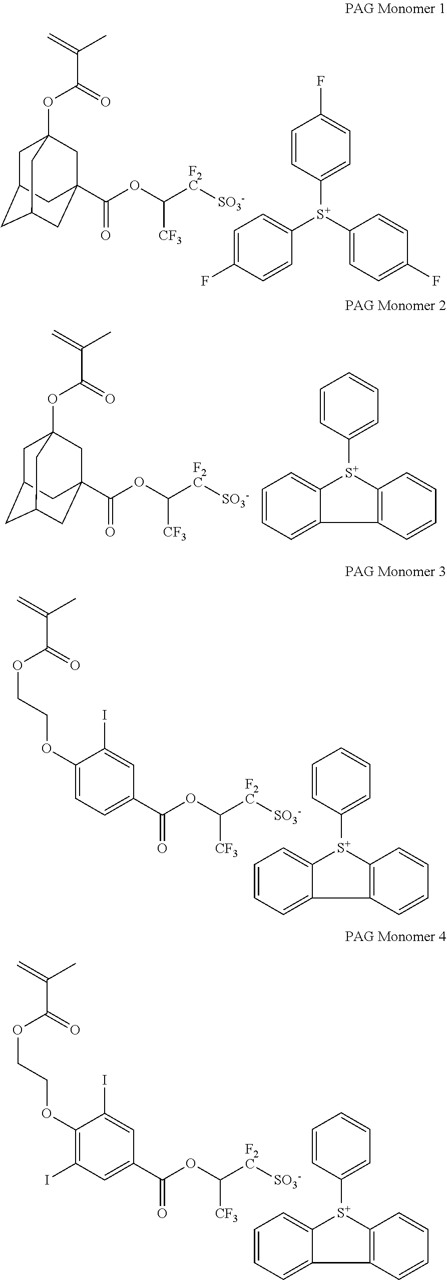

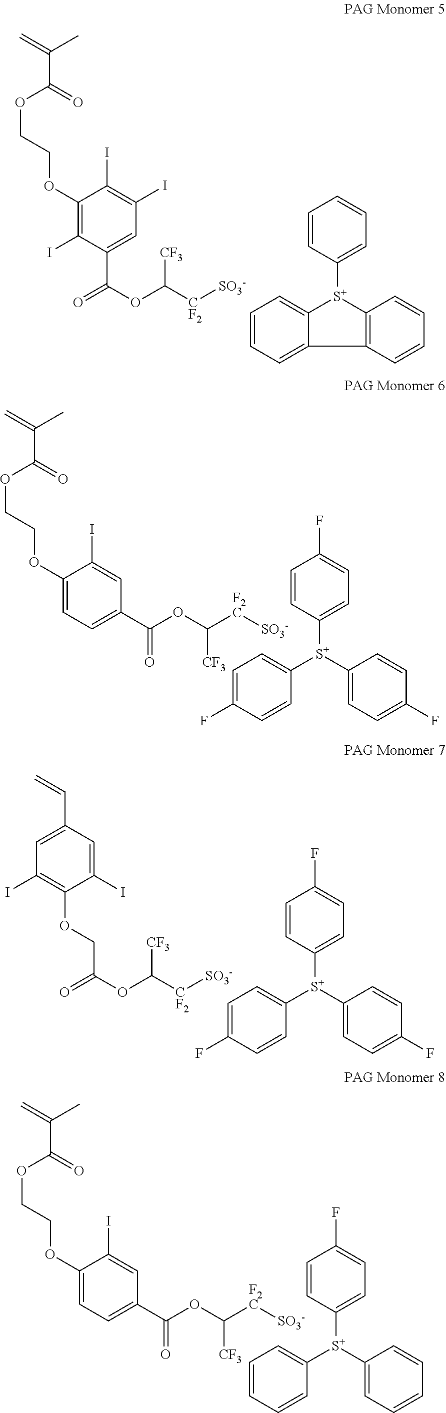

### [Synthesis Example 2-1] Synthesis of Polymer 1

A 2-L flask was charged with 3.5 g of Monomer 1, 8.4 g of 1-methyl-1-cyclopentyl methacrylate, 5.4 g of 4-hydroxystyrene, and 40 g of THF as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 1 was obtained. The composition of Polymer 1 was identified by ¹³C-NMR and ¹H-NMR, and the Mw and Mw/Mn were identified by GPC.

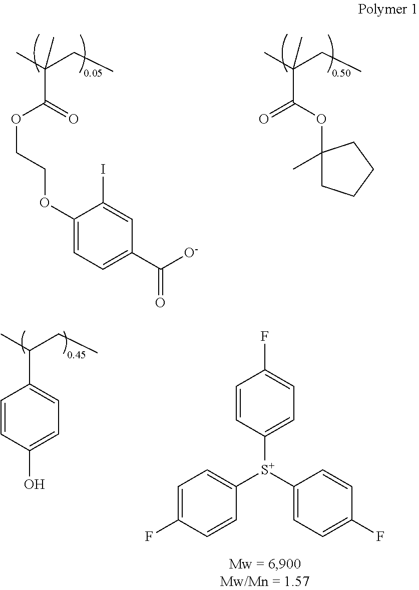

### [Synthesis Example 2-2] Synthesis of Polymer 2

A 2-L flask was charged with 3.4 g of Monomer 2, 7.3 g of 1-methyl-1-cyclohexyl methacrylate, 4.8 g of 4-hydroxystyrene, 11.0 g of PAG Monomer 2, and 40 g of THE as a solvent. This reaction container was cooled to
−70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 2 was obtained. The composition of Polymer 2 was identified by ¹³C-NMR and ¹H-NMR, and the Mw and Mw/Mn were identified by GPC.

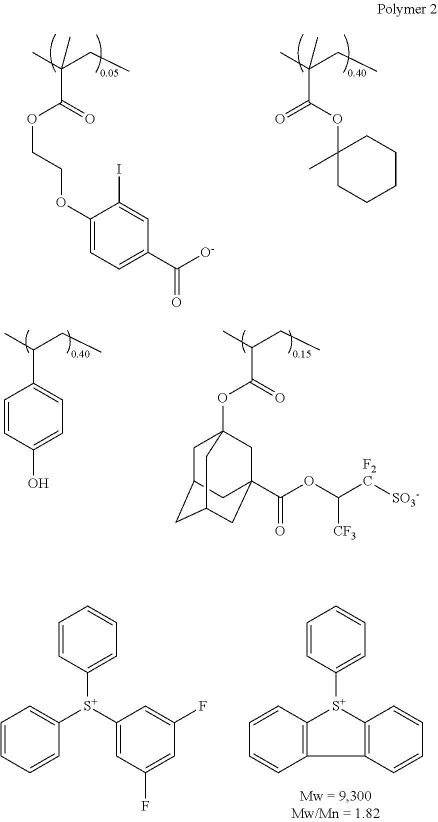

### [Synthesis Example 2-3] Synthesis of Polymer 3

A 2-L flask was charged with 3.0 g of Monomer 3, 8.4 g of 1-methyl-1-cyclopentyl methacrylate, 3.6 g of 4-hydroxystyrene, 11.9 g of PAG Monomer 1, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 3 was obtained. The composition of Polymer 3 was identified by ¹³C-NMR and ¹H-NMR, and the Mw and Mw/Mn were identified by GPC.

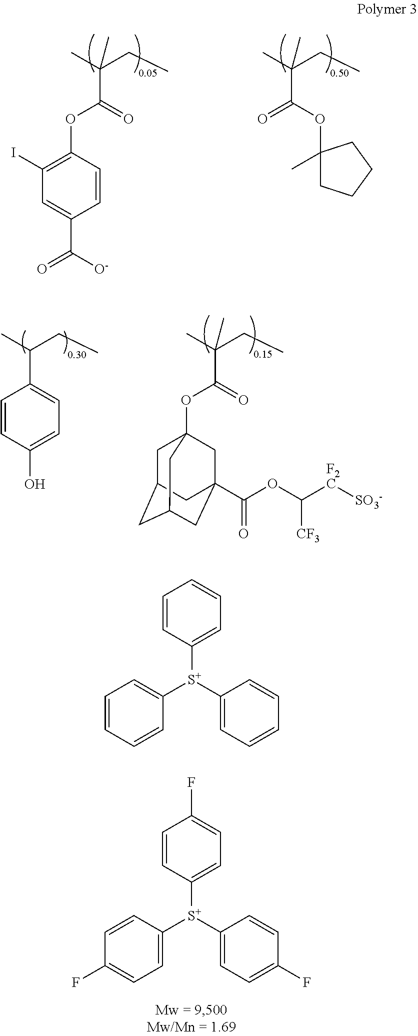

### [Synthesis Example 2-4] Synthesis of Polymer 4

A 2-L flask was charged with 3.2 g of Monomer 4, 8.4 g of 1-methyl-1-cyclopentyl methacrylate, 3.6 g of 3-hydroxystyrene, 11.0 g of PAG Monomer 2, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 4 was obtained. The composition of Polymer 4 was identified by ¹³C-NMR and ¹H-NMR, and the Mw and Mw/Mn were identified by GPC.

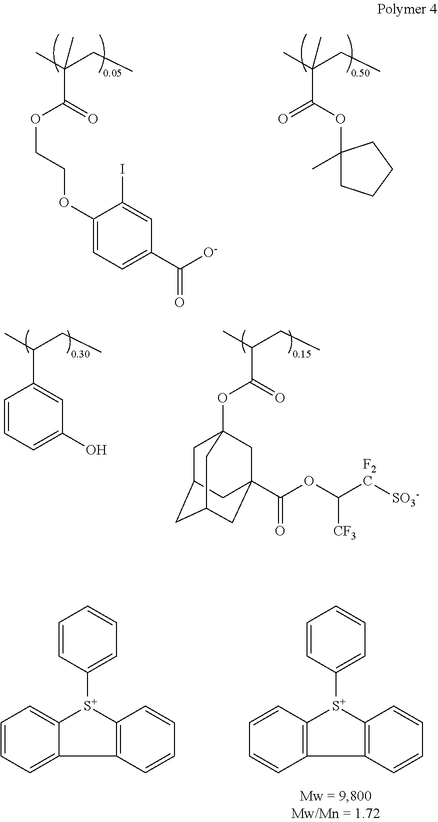

### [Synthesis Example 2-5] Synthesis of Polymer 5

A 2-L flask was charged with 4.1 g of Monomer 5, 8.4 g of 1-methyl-1-cyclopentyl methacrylate, 4.2 g of 4-hydroxystyrene, 8.5 g of PAG Monomer 3, and 40 g of THF as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 5 was obtained. The composition of Polymer 5 was identified by ¹³C-NMR and ¹H-NMR, and the Mw and Mw/Mn were identified by GPC.

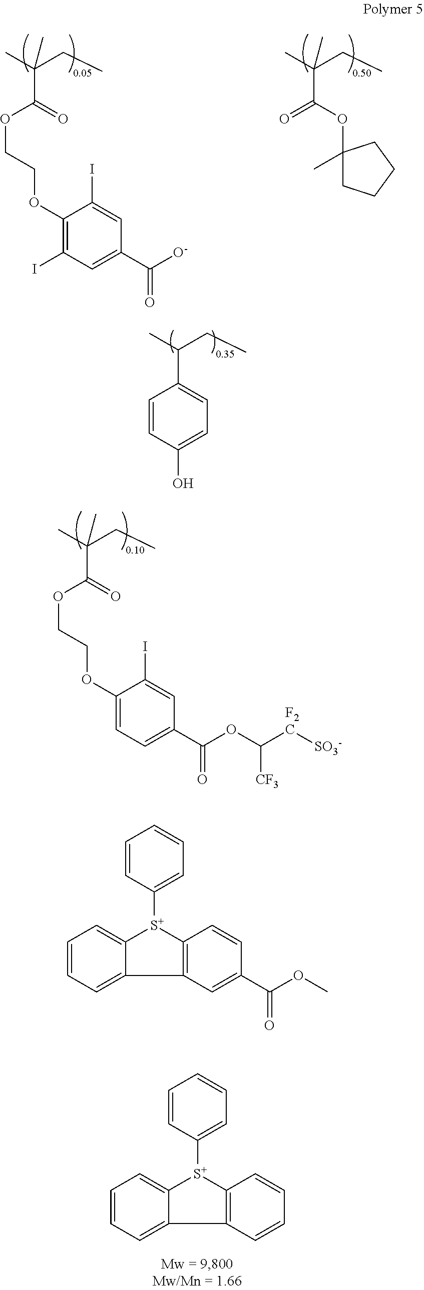

### [Synthesis Example 2-6] Synthesis of Polymer 6

A 2-L flask was charged with 4.9 g of Monomer 6, 8.9 g of ALG Monomer 1, 5.4 g of 4-hydroxystyrene, 10.2 g of PAG Monomer 4, and 40 g of THF as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 6 was obtained. The composition of Polymer 6 was identified by ¹³C-NMR and ¹H-NMR, and the Mw and Mw/Mn were identified by GPC.

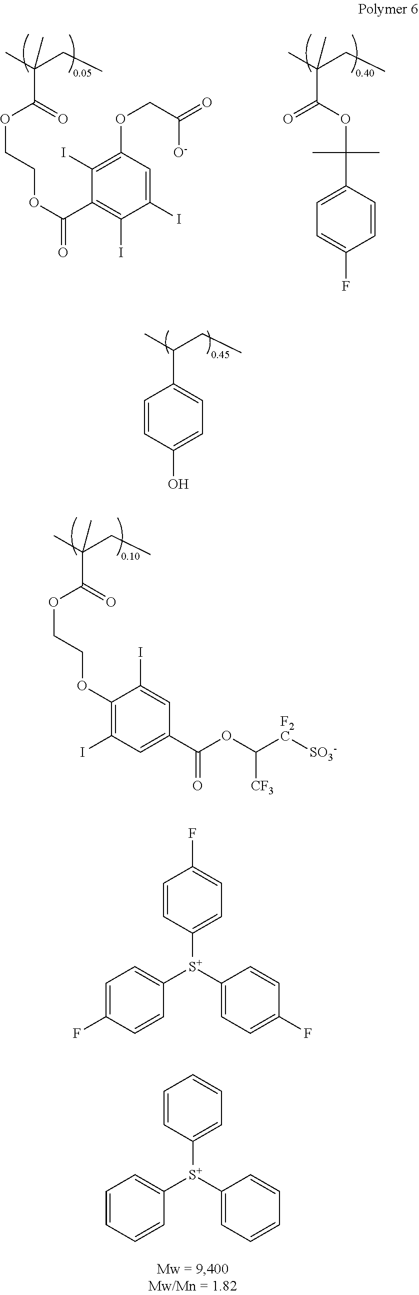

### [Synthesis Example 2-7] Synthesis of Polymer 7

A 2-L flask was charged with 3.8 g of Monomer 7, 8.4 g of 1-methyl-1-cyclopentyl methacrylate, 4.8 g of 4-hydroxystyrene, 5.5 g of PAG Monomer 5, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 7 was obtained. The composition of Polymer 7 was identified by ¹³C-NMR and ¹H-NMR, and the Mw and Mw/Mn were identified by GPC.

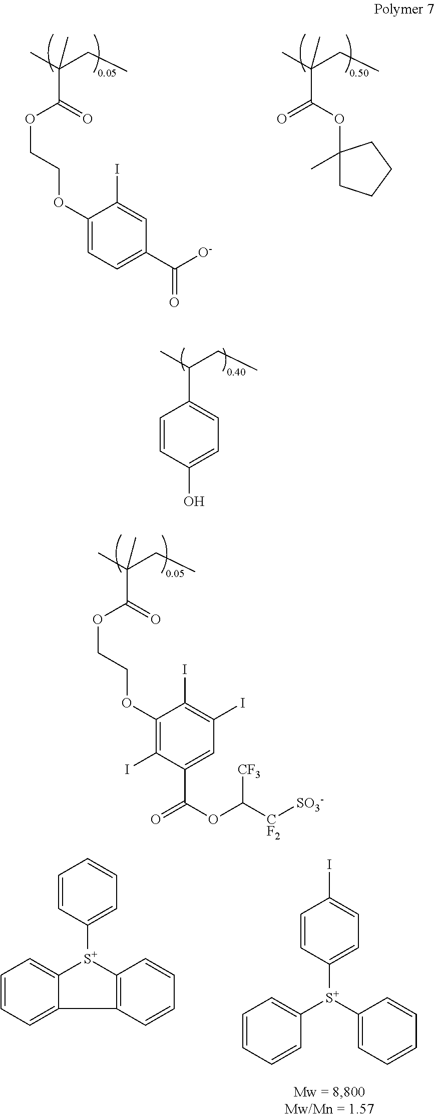

### [Synthesis Example 2-8] Synthesis of Polymer 8

A 2-L flask was charged with 3.4 g of Monomer 8, 8.9 g of ALG Monomer 1, 5.4 g of 4-hydroxystyrene, 9.7 g of PAG Monomer 4, and 40 g of THF as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 8 was obtained. The composition of Polymer 8 was identified by ¹³C-NMR and ¹H-NMR, and the Mw and Mw/Mn were identified by GPC.

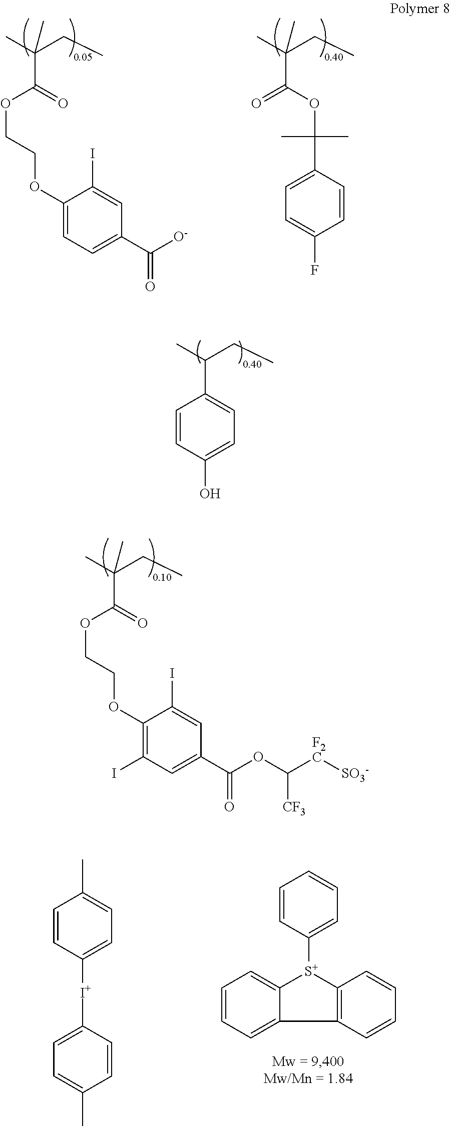

### [Synthesis Example 2-9] Synthesis of Polymer 9

A 2-L flask was charged with 3.5 g of Monomer 1, 8.4 g of 1-methyl-1-cyclopentyl methacrylate, 4.2 g of 4-hydroxystyrene, 9.4 g of PAG Monomer 6, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 9 was obtained. The composition of Polymer 9 was identified by ¹³C-NMR and ¹H-NMR, and the Mw and Mw/Mn were identified by GPC.

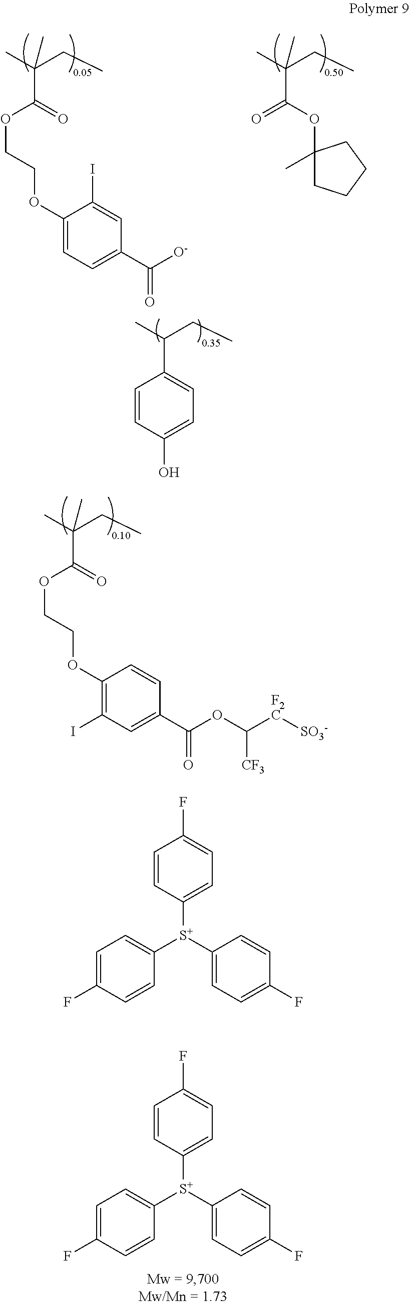

### [Synthesis Example 2-10] Synthesis of Polymer 10

A 2-L flask was charged with 3.8 g of Monomer 9, 8.4 g of 1-methyl-1-cyclopentyl methacrylate, 4.2 g of 4-hydroxystyrene, 9.4 g of PAG Monomer 6, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 10 was obtained. The composition of Polymer 10 was identified by ¹³C-NMR and ¹H-NMR, and the Mw and Mw/Mn were identified by GPC.

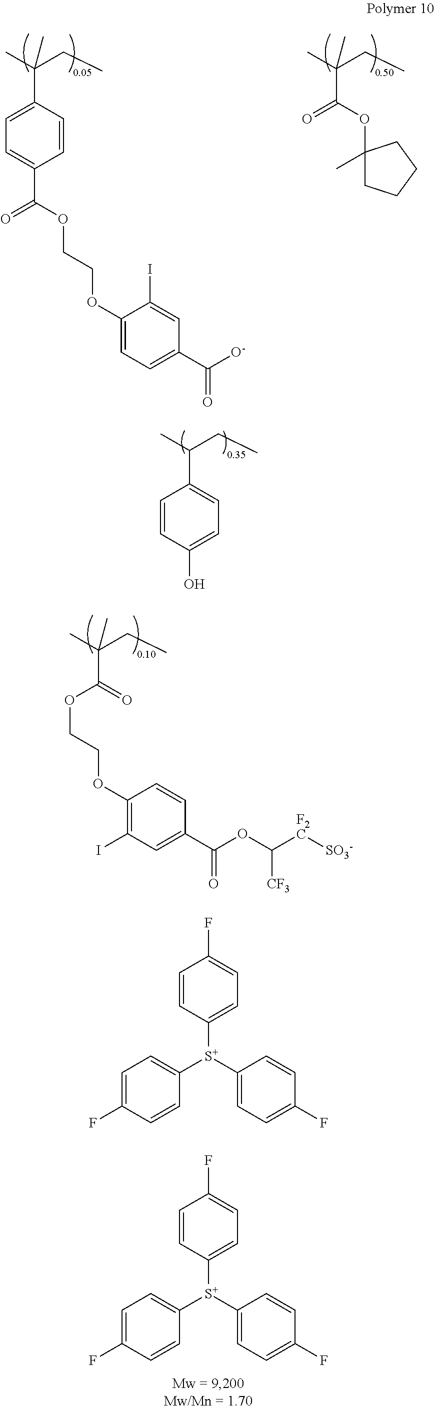

### [Synthesis Example 2-11] Synthesis of Polymer 11

A 2-L flask was charged with 3.5 g of Monomer 1, 8.4 g of 1-methyl-1-cyclopentyl methacrylate, 4.2 g of 4-hydroxystyrene, 9.6 g of PAG Monomer 7, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 11 was obtained. The composition of Polymer 11 was identified by ¹³C-NMR and ¹H-NMR, and the Mw and Mw/Mn were identified by GPC.

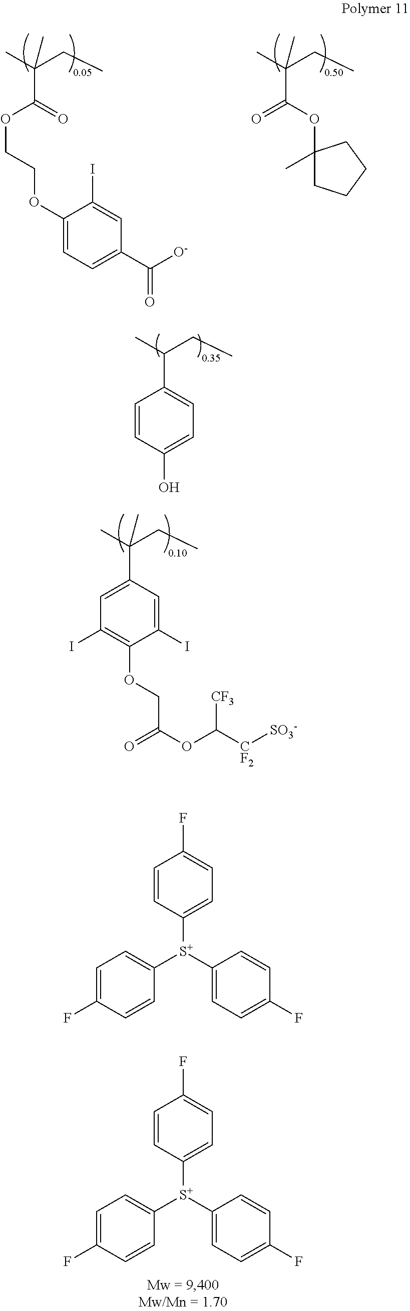

### [Synthesis Example 2-12] Synthesis of Polymer 12

A 2-L flask was charged with 3.8 g of Monomer 9, 8.4 g of 1-methyl-1-cyclopentyl methacrylate, 4.2 g of 4-hydroxystyrene, 9.6 g of PAG Monomer 7, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 12 was obtained. The composition of Polymer 12 was identified by ¹³C-NMR and ¹H-NMR, and the Mw and Mw/Mn were identified by GPC.

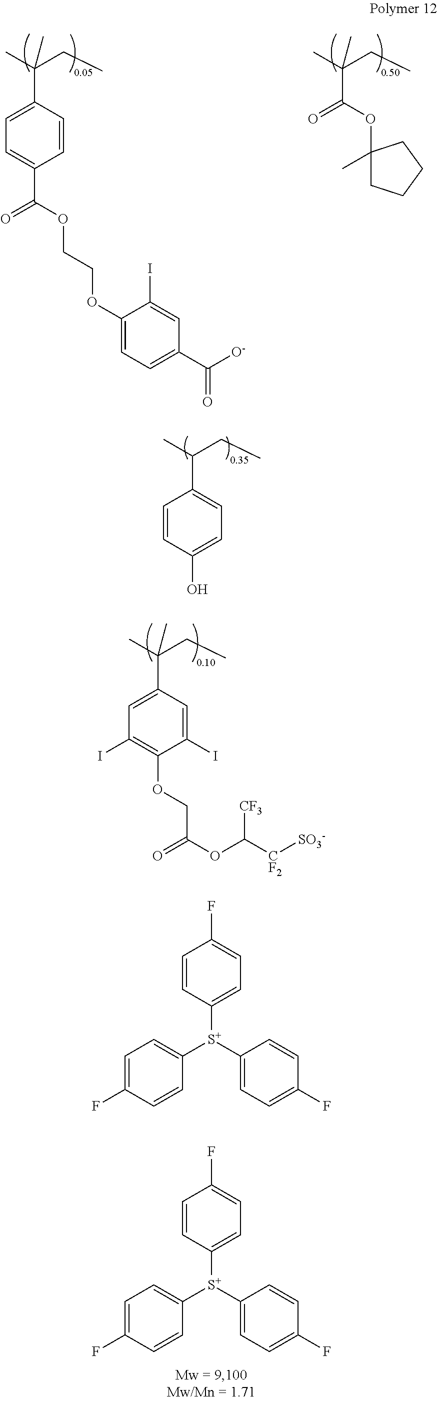

### [Synthesis Example 2-13] Synthesis of Polymer 13

A 2-L flask was charged with 3.8 g of Monomer 10, 9.0 g of 1-vinyl-1-cyclopentyl methacrylate, 4.2 g of 3-hydroxystyrene, 9.4 g of PAG Monomer 6, and 40 g of THE as a solvent. This reaction container was cooled to
−70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 13 was obtained. The composition of Polymer 13 was identified by ¹³C-NMR and ¹H-NMR, and the Mw and Mw/Mn were identified by GPC.

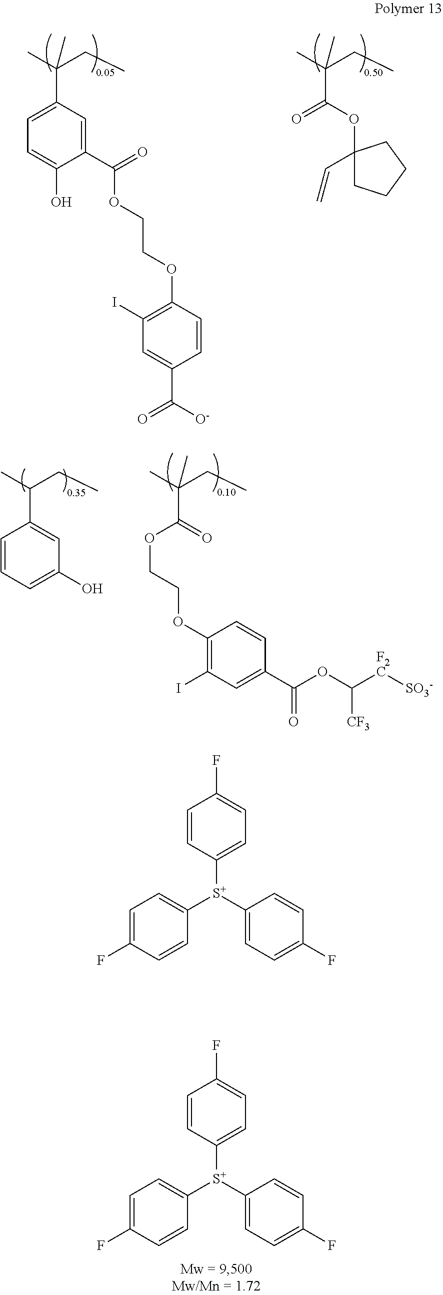

### [Synthesis Example 2-14] Synthesis of Polymer 14

A 2-L flask was charged with 4.5 g of Monomer 11, 8.9 g of 1-ethynyl-1-cyclopentyl methacrylate, 4.2 g of 3-hydroxystyrene, 9.8 g of PAG Monomer 8, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 14 was obtained. The composition of Polymer 14 was identified by ¹³C-NMR and ¹H-NMR, and the Mw and Mw/Mn were identified by GPC.

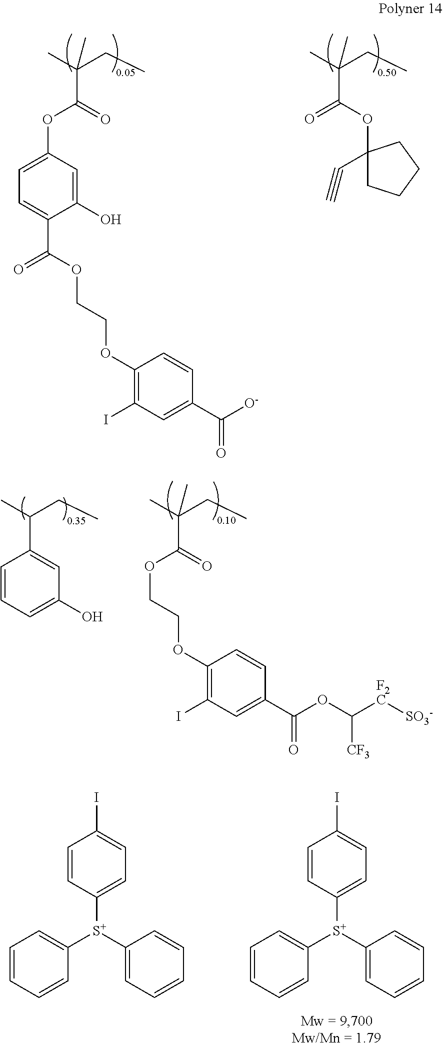

### [Synthesis Example 2-15] Synthesis of Polymer 15

A 2-L flask was charged with 4.6 g of Monomer 12, 8.9 g of 1-ethynyl-1-cyclopentyl methacrylate, 4.2 g of 3-hydroxystyrene, 9.8 g of PAG Monomer 8, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 15 was obtained. The composition of Polymer 15 was identified by ¹³C-NMR and ¹H-NMR, and the Mw and Mw/Mn were identified by GPC.

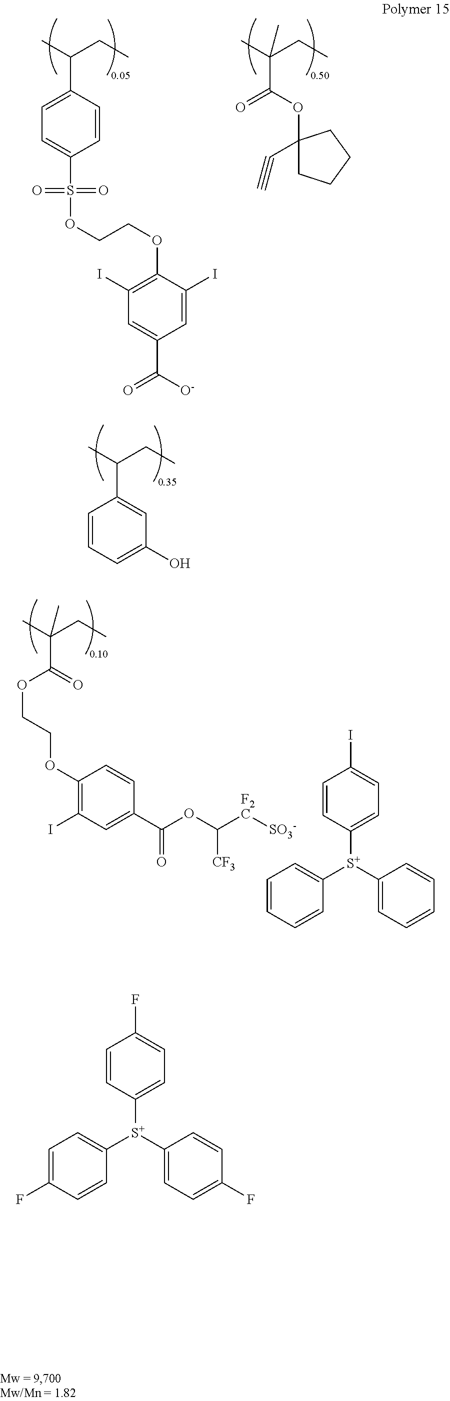

### [Synthesis Example 2-16] Synthesis of Polymer 16

A 2-L flask was charged with 4.2 g of Monomer 13, 8.9 g of ALG Monomer 1, 4.2 g of 3-hydroxystyrene, 9.4 g of PAG Monomer 6, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 16 was obtained. The composition of Polymer 16 was identified by ¹³C-NMR and ¹H-NMR, and the Mw and Mw/Mn were identified by GPC.

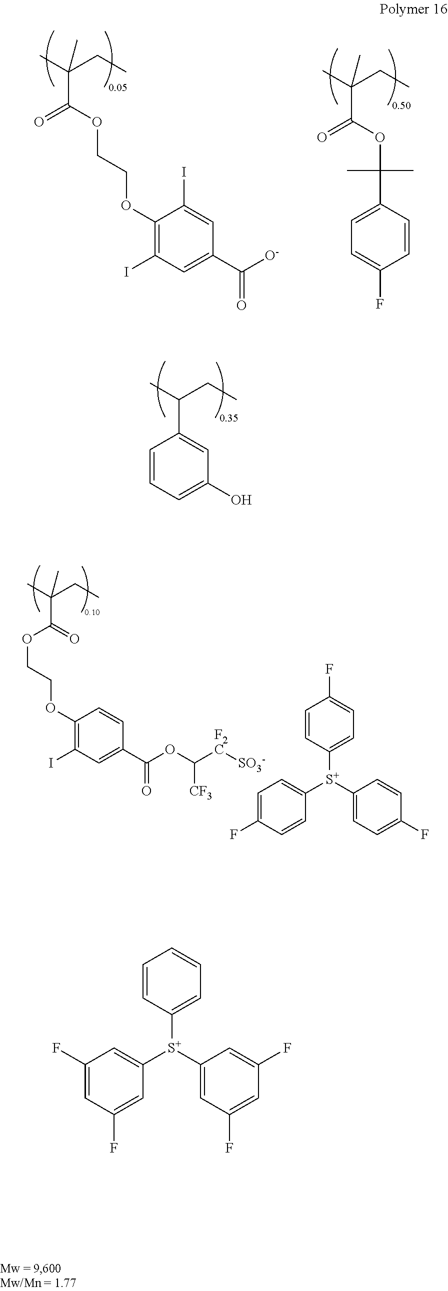

### [Synthesis Example 2-17] Synthesis of Polymer 17

A 2-L flask was charged with 4.2 g of Monomer 14, 8.9 g of ALG Monomer 1, 4.2 g of 3-hydroxystyrene, 9.4 g of PAG Monomer 6, and 40 g of THE as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 17 was obtained. The composition of Polymer 17 was identified by ¹³C-NMR and ¹H-NMR, and the Mw and Mw/Mn were identified by GPC.

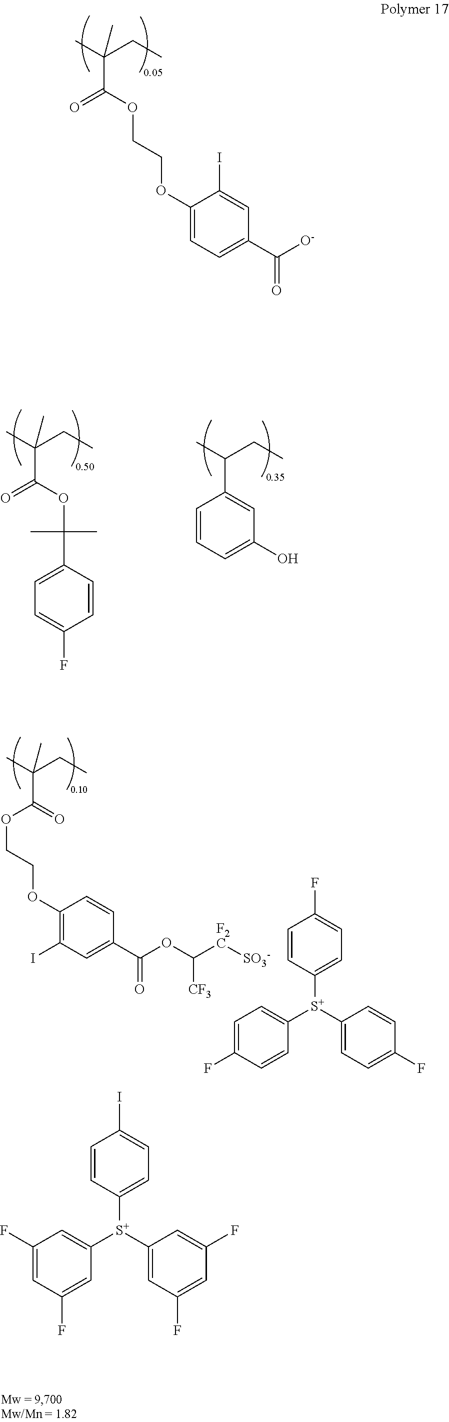

### [Synthesis Example 2-18] Synthesis of Polymer 18

A 2-L flask was charged with 4.7 g of Monomer 15, 8.9 g of ALG Monomer 1, 4.2 g of 3-hydroxystyrene, 9.4 g of PAG Monomer 6, and 40 g of THF as a solvent. This reaction container was cooled to −70° C. under nitrogen atmosphere. Vacuum degassing and nitrogen blowing were repeated three times. After the temperature was raised to room temperature, 1.2 g of AIBN was added as a polymerization initiator, the temperature was raised to 60° C., and the reaction was allowed to take place for 15 hours. The reaction solution was poured into 1 L of isopropyl alcohol. A deposited white solid was separated by filtration. The obtained white solid was dried at 60° C. under reduced pressure. Thus, Polymer 18 was obtained. The composition of Polymer 18 was identified by ¹³C-NMR and ¹H-NMR, and the Mw and Mw/Mn were identified by GPC.

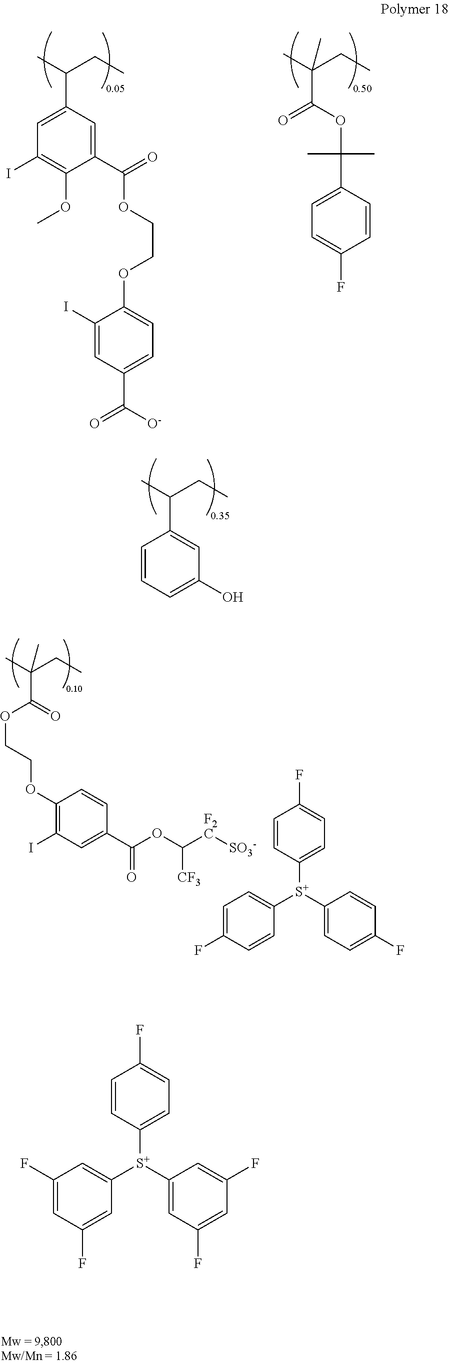

### [Comparative Synthesis Example 1] Synthesis of Comparative Polymer 1

Comparative Polymer 1 was obtained by the same procedure as in Synthesis Example 2-1, except that Comparative Monomer 1 was used instead of Monomer 1. The composition of Comparative Polymer 1 was identified by ¹³C-NMR and ¹H-NMR, and the Mw and Mw/Mn were identified by GPC.

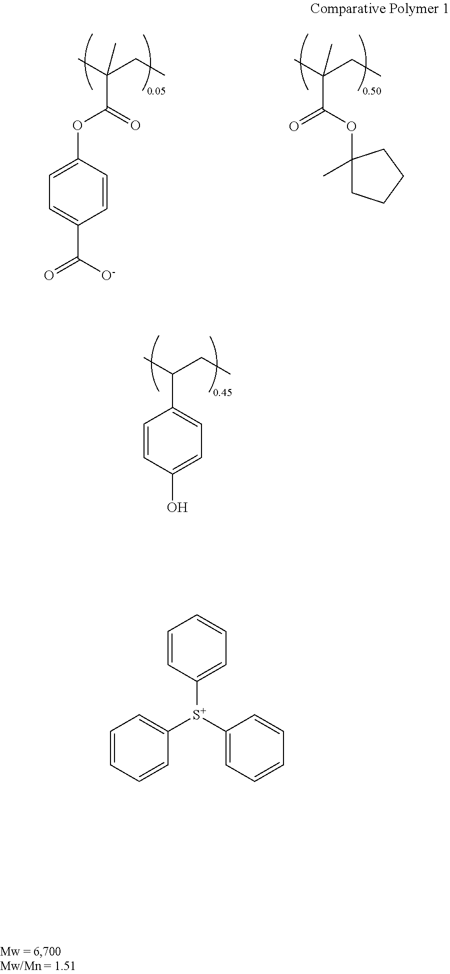

### [Comparative Synthesis Example 2] Synthesis of Comparative Polymer 2

Comparative Polymer 2 was obtained by the same procedure as in Synthesis Example 2-1, except that Comparative Monomer 2 was used instead of Monomer 1. The composition of Comparative Polymer 2 was identified by ¹³C-NMR and ¹H-NMR, and the Mw and Mw/Mn were identified by GPC.

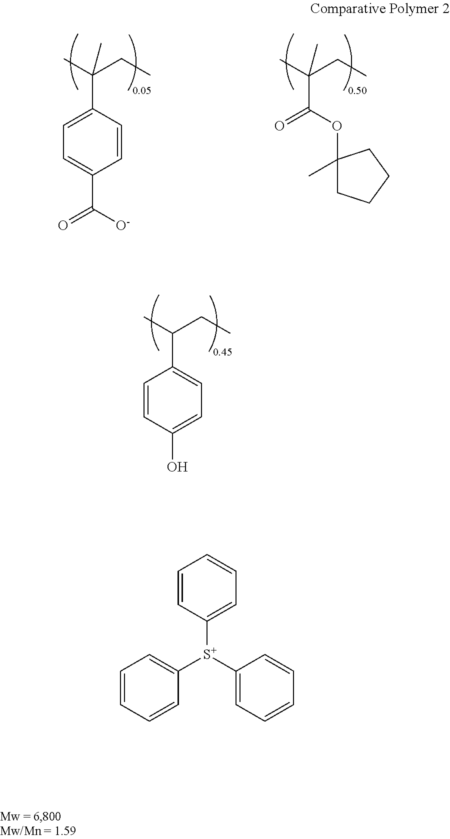

### [Comparative Synthesis Example 3] Synthesis of Comparative Polymer 3

Comparative Polymer 3 was obtained by the same procedure as in Synthesis Example 2-1, except that Monomer 1 was not used. The composition of Comparative Polymer 3 was identified by ¹³C-NMR and 1H-NMR, and the Mw and Mw/Mn were identified by GPC.

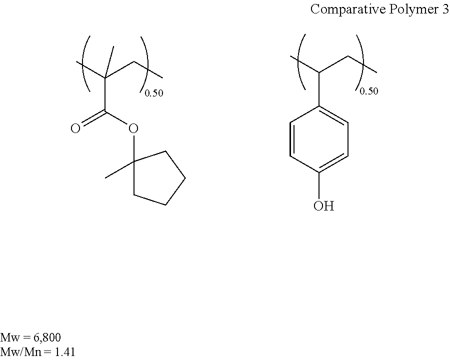

### [Comparative Synthesis Example 4] Synthesis of Comparative Polymer 4

Comparative Polymer 4 was obtained by the same procedure as in Synthesis Example 2-4, except that Monomer 4 was not used. The composition of Comparative Polymer 4 was identified by ¹³C-NMR and 1H-NMR, and the Mw and Mw/Mn were identified by GPC.

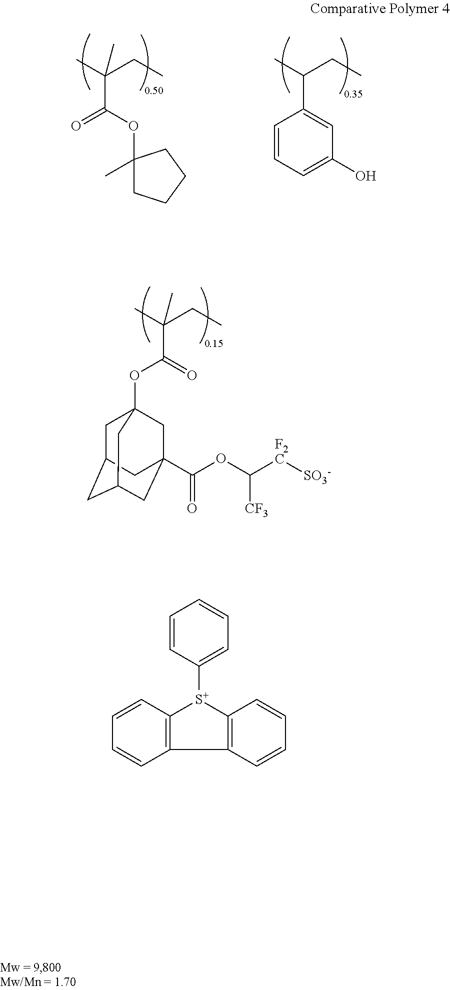

### [Comparative Synthesis Example 5] Synthesis of Comparative Polymer 5

Comparative Polymer 5 was obtained by the same procedure as in Synthesis Example 2-1, except that Comparative Monomer 3 was used instead of Monomer 1. The composition of Comparative Polymer 5 was identified by ¹³C-NMR and ¹H-NMR, and the Mw and Mw/Mn were identified by GPC.

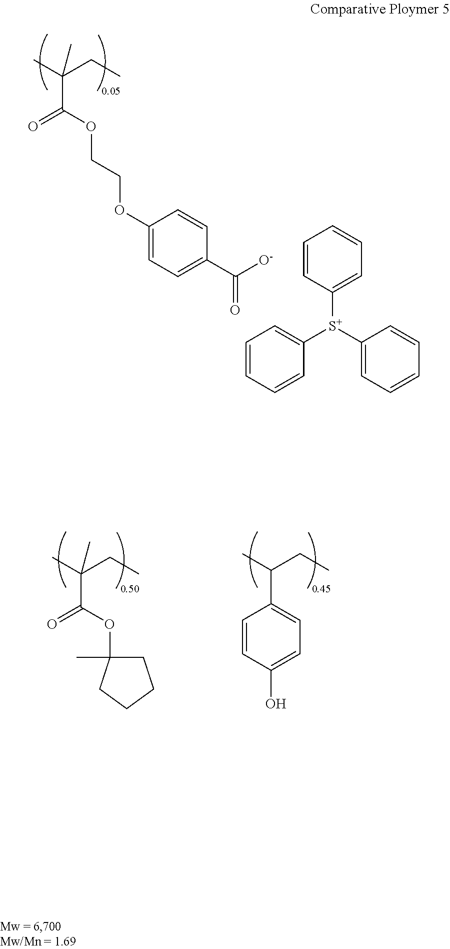

### Examples 1 to 22, Comparative Examples 1 to 5

According to the composition shown in Tables 1 and 2, components were dissolved in an organic solvent in which 50 ppm of a surfactant Polyfox 636 (manufactured by OMNOVA Solutions Inc.) had been dissolved. The resulting solution was filtered through a filter having a pore size of 0.2 μm. In this manner, resist compositions were prepared.

The components in Tables 1 and 2 are as follows.
Organic solvents:
   PGMEA (propylene glycol monomethyl ether acetate)
   DAA (diacetone alcohol)
   EL (ethyl lactate)
Acid generator: PAG-1 (see structural formula below)
Quenchers: Q-1, Q-2, and Comparative Quencher RQ-1 (see structural formulae below)

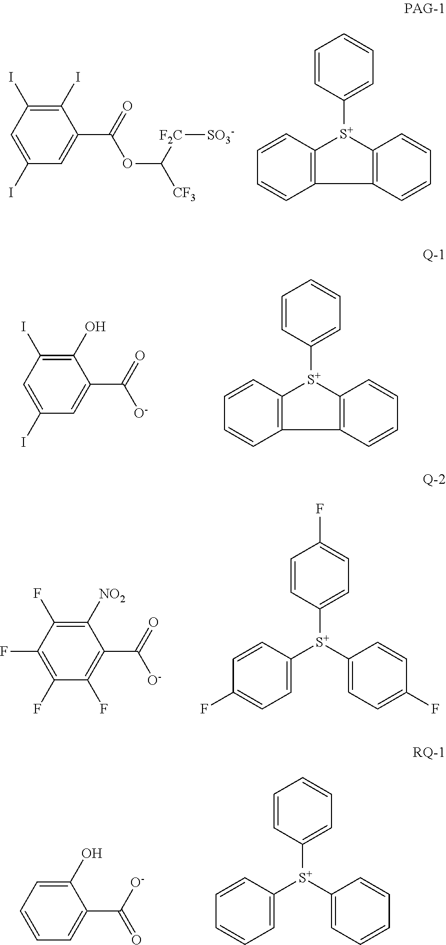

### EUV Exposure Evaluation

A Si substrate with a silicon-containing spin-on hard mask SHB-A940 (silicon content: 43 mass %) formed to have a film thickness of 20 nm was spin-coated with one of the resist compositions shown in Tables 1 and 2. The resultant was prebaked using a hot plate at 105° C. for 60 seconds to prepare a resist film having a film thickness of 50 nm. The resist film was exposed using an EUV scanner NXE3400 (manufactured by ASML, NA: 0.33, σ: 0.9/0.6, quadrupole illumination, with a mask having a hole pattern with a pitch of 46 nm and +20% bias (on-wafer size)), followed by PEB on the hot plate at a temperature shown in Tables 1 and 2 for 60 seconds, and development with a 2.38 mass % aqueous TMAH solution for 30 seconds to obtain a hole pattern with a dimension of 23 nm.

An exposure dose at which the hole dimension of 23 nm was formed was measured and determined as sensitivity. Moreover, the dimensions of 50 holes were measured using a CD-SEM (CG6300) manufactured by Hitachi, Ltd., and CDU (dimensional variation 30) was calculated.

The results are shown in Tables 1 and 2.

TABLE 1

| | | Acid | | | | | |
|---|---|---|---|---|---|---|---|
| | Polymer | generator | Quencher | | PEB | | |
| | (parts by | (parts by | (parts by | Organic solvent | temperature | Sensitivity | CDU |
| | mass) | mass) | mass) | (parts by mass) | (° C.) | (mJ/cm²) | (nm) |
| Example 1 | Polymer 1 | PAG-1 | — | PGMEA (2,000) | 80 | 33 | 2.7 |
| | (100) | (25.0) | | DAA (500) | | | |
| Example 2 | Polymer 2 | — | — | PGMEA (2,000) | 80 | 32 | 2.4 |
| | (100) | | | DAA (500) | | | |
| Example 3 | Polymer 3 | — | — | PGMEA (2,000) | 80 | 32 | 2.9 |
| | (100) | | | DAA (500) | | | |
| Example 4 | Polymer 4 | — | — | PGMEA (2,000) | 80 | 31 | 2.4 |
| | (100) | | | DAA (500) | | | |
| Example 5 | Polymer 5 | — | — | PGMEA (2,000) | 80 | 29 | 2.5 |
| | (100) | | | DAA (500) | | | |
| Example 6 | Polymer 6 | — | — | PGMEA (2,000) | 80 | 32 | 2.4 |
| | (100) | | | DAA (500) | | | |
| Example 7 | Polymer 7 | PAG-1 | — | EL (2,000) | 80 | 27 | 2.6 |
| | (100) | (10.0) | | PGMEA (500) | | | |
| Example 8 | Polymer 7 | PAG-1 | Q-1 | EL (2,000) | 80 | 29 | 2.5 |
| | (100) | (15.0) | (3.25) | PGMEA (500) | | | |
| Example 9 | Polymer 7 | PAG-1 | Q-2 | EL (2,000) | 80 | 29 | 2.6 |
| | (100) | (15.0) | (2.78) | PGMEA (500) | | | |
| Example 10 | Polymer 8 | — | — | PGMEA (2,000) | 80 | 32 | 2.4 |
| | (100) | | | DAA (500) | | | |
| Example 11 | Polymer 9 | — | — | PGMEA (2,000) | 80 | 30 | 2.5 |
| | (100) | | | DAA (500) | | | |
| Example 12 | Polymer 4 | — | Q-1 | PGMEA (2,000) | 80 | 34 | 2.1 |
| | (100) | | (0.25) | DAA (500) | | | |
| Example 13 | Polymer 5 | — | Q-1 | PGMEA (2,000) | 80 | 33 | 2.2 |
| | (100) | | (0.25) | DAA (500) | | | |
| Example 14 | Polymer 10 | — | — | PGMEA (2,000) | 80 | 32 | 2.4 |
| | (100) | | | DAA (500) | | | |
| Example 15 | Polymer 11 | — | — | PGMEA (2,000) | 80 | 33 | 2.6 |
| | (100) | | | DAA (500) | | | |
| Example 16 | Polymer 12 | — | — | PGMEA (2,000) | 80 | 30 | 2.5 |
| | (100) | | | DAA (500) | | | |
| Example 17 | Polymer 13 | — | — | PGMEA (2,000) | 80 | 33 | 2.5 |
| | (100) | | | DAA (500) | | | |
| Example 18 | Polymer 14 | — | — | PGMEA (1,000) | 80 | 34 | 2.4 |
| | (100) | | | DAA (500) | | | |
| | | | | EL (1,000) | | | |
| Example 19 | Polymer 15 | — | — | PGMEA (2,000) | 80 | 33 | 2.6 |
| | (100) | | | DAA (500) | | | |
| Example 20 | Polymer 16 | — | — | PGMEA (2,000) | 80 | 30 | 2.5 |
| | (100) | | | DAA (500) | | | |
| Example 21 | Polymer 17 | — | — | PGMEA (2,000) | 80 | 31 | 2.4 |
| | (100) | | | DAA (500) | | | |
| Example 22 | Polymer 18 | — | — | PGMEA (2,000) | 80 | 30 | 2.4 |
| | (100) | | | DAA (500) | | | |

TABLE 2

| | | Acid | | | | | |
|---|---|---|---|---|---|---|---|
| | | generator | Quencher | | PEB | | |
| | Polymer | (parts by | (parts by | Organic solvent | temperature | Sensitivity | CDU |
| | (parts by mass) | mass) | mass) | (parts by mass) | (° C.) | (mJ/cm²) | (nm) |
| Comparative | Comparative | PAG-1 | — | PGMEA (2,000) | 80 | 45 | 4.1 |
| Example 1 | Polymer 1 | (25.0) | | DAA (500) | | | |
| | (100) | | | | | | |
| Comparative | Comparative | PAG-1 | — | PGMEA (2,000) | 80 | 48 | 4.3 |
| Example 2 | Polymer 2 | (25.0) | | DAA (500) | | | |
| | (100) | | | | | | |
| Comparative | Comparative | PAG-1 | RQ-1 | PGMEA (2,000) | 80 | 41 | 4.0 |
| Example 3 | Polymer 3 | (25.0) | (4.00) | DAA (500) | | | |
| | (100) | | | | | | |
| Comparative | Comparative | — | RQ-1 | PGMEA (2,000) | 80 | 34 | 3.3 |
| Example 4 | Polymer 4 | | (4.00) | DAA (500) | | | |
| | (100) | | | | | | |
| Comparative | Comparative | PAG-1 | — | PGMEA (2,000) | 80 | 46 | 3.8 |
| Example 5 | Polymer 5 | (25.0) | | DAA (500) | | | |
| | (100) | | | | | | |

From the results of Tables 1 and 2, it was revealed that the resist compositions containing the inventive resist material containing a polymer including a repeating unit-a, having a sulfonium salt or iodonium salt structure of a monovalent aromatic carboxylic acid having a substituent iodine atom satisfied sufficient sensitivity and critical dimension uniformity (Examples 1 to 22). On the other hand, in Comparative Examples 1 to 5, where resist compositions not containing the inventive resist material were used, poor results were obtained regarding sensitivity and critical dimension uniformity.

The present description includes the following embodiments.

[1]: A resist material comprising a repeating unit-a containing a sulfonium salt or iodonium salt of a monovalent aromatic carboxylic acid that is iodinated, is substituted or unsubstituted, and is bonded to a polymer main chain via an ester bond.

[2]: The resist material of the above [1], wherein the repeating unit-a contains a repeating unit represented by the following general formula (a)-1 or (a)-2,

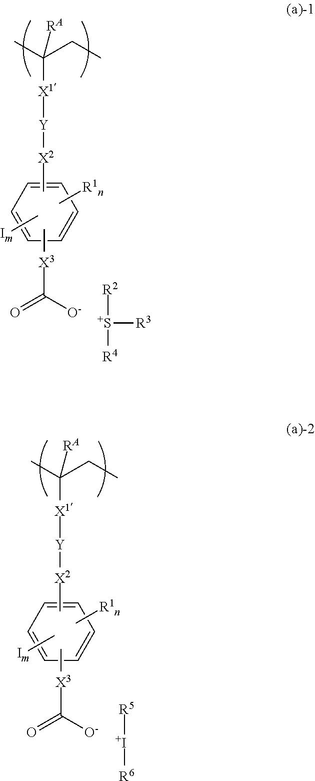

wherein R^{A} represents a hydrogen atom or a methyl group; X^{1′} represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group; X² represents a single bond or a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; Y represents a group selected from an ester group and a sulfonic acid ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

[3]: The resist material of the above [2], in the general formulae (a)-1 and (a)-2, the X² represents a linear, branched, or cyclic alkylene group having 1 to 12 carbon atoms and optionally containing one or more selected from an ester group, an ether group, an amide group, a lactone ring, a sultone ring, and a halogen atom.

[4]: The resist material of any one of the above [1] to [3], further comprising a repeating unit-b, in which a hydrogen atom of a carboxy group, a phenolic hydroxy group, or both is substituted with an acid-labile group.

[5]: The resist material of the above [4], wherein the repeating unit-b is at least one kind of repeating unit selected from repeating units represented by the following general formulae (b1) and (b2),

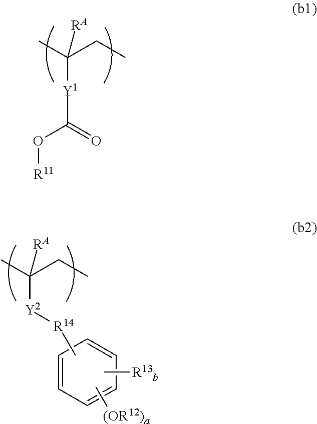

wherein each R^{A} independently represents a hydrogen atom or a methyl group; Y¹ represents a single bond, a phenylene group, a naphthylene group, or a linking group having 1 to 12 carbon atoms and having an ester bond, an ether bond, or a lactone ring; Y² represents a single bond, an ester bond, or an amide bond; R¹¹ and R¹² each represent an acid-labile group; R¹³ represents a fluorine atom, a trifluoromethyl group, a cyano group, or an alkyl group having 1 to 6 carbon atoms; R¹⁴ represents a single bond or a linear or branched alkanediyl group having 1 to 6 carbon atoms, part of carbon atoms of the alkanediyl group optionally being substituted with an ether bond or an ester bond; “a” represents 1 or 2; and “b” represents an integer of 0 to 4.

[6]: The resist material of any one of the above [1] to [5], further comprising a repeating unit-c having an adhesive group selected from a hydroxy group, a carboxy group, a lactone ring, a carbonate group, a thiocarbonate group, a carbonyl group, a cyclic acetal group, an ether bond, an ester bond, a sulfonic acid ester bond, a cyano group, an amide group, —O—C(═O)—S—, and —O—C(═O)—NH—.

[7]: The resist material of any one of the above [1] to [6], further comprising at least one kind of repeating unit-d selected from repeating units represented by the following general formulae (d1) to (d3),

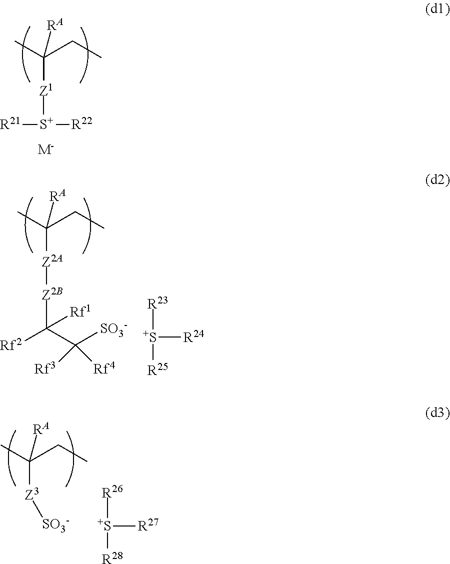

wherein each R^{A} independently represents a hydrogen atom or a methyl group; Z¹ represents a single bond, a phenylene group, —O—Z¹¹—, —C(═O)—O—Z¹¹—, or —C(═O)—NH—Z¹¹—; Z¹¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, and a hydroxy group; Z^{2A} represents a single bond or an ester bond; Z^{2B} represents a single bond or a divalent group having 1 to 12 carbon atoms and optionally containing one or more selected from an ester bond, an ether bond, a lactone ring, a bromine atom, and an iodine atom; Z³ represents a single bond, a methylene group, an ethylene group, a phenylene group, a fluorinated phenylene group, —O—Z³¹—, —C(═O)—O—Z³¹—, or —C(═O)—NH—Z³¹—; Z³¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, a halogen atom, and a hydroxy group; Rf¹ to Rf⁴ each independently represent a hydrogen atom, a fluorine atom, or a trifluoromethyl group, provided that at least one of Rf¹ to Rf⁴ is a fluorine atom; R²¹ to R²⁸ each independently represent a monovalent hydrocarbon group having 1 to 20 carbon atoms and optionally containing a heteroatom; any two of R²³, R²⁴, and R²⁵ or any two of R²⁶, R²⁷, and R²⁸ are optionally bonded to each other to form a ring together with a sulfur atom bonded thereto; and M⁻ represents a non-nucleophilic counter ion.

[8]: The resist material of the above [7], wherein at least one iodine atom is contained in the Z^{2B}.

[9]: The resist material of any one of the above [1] to [8], wherein the resist material has a molecular weight of 1,000 to 100,000.

[10]: A resist composition comprising the resist material of any one of the above [1] to [9].

[11]: The resist composition of the above [10], further comprising one or more selected from an acid generator, an organic solvent, a quencher, and a surfactant.

[12]: A patterning process comprising the steps of:
forming a resist film on a substrate by using the resist composition of the above or [11];
exposing the resist film to a high-energy beam; and
developing the exposed resist film by using a developer.

[13]: The patterning process of the above [12], wherein the high-energy beam is an i-line, a KrF excimer laser beam, an ArF excimer laser beam, an electron beam, or an extreme ultraviolet ray having a wavelength of 3 to 15 nm.

[14]: A monomer represented by the following general formula (a)-1M or (a)-2M,

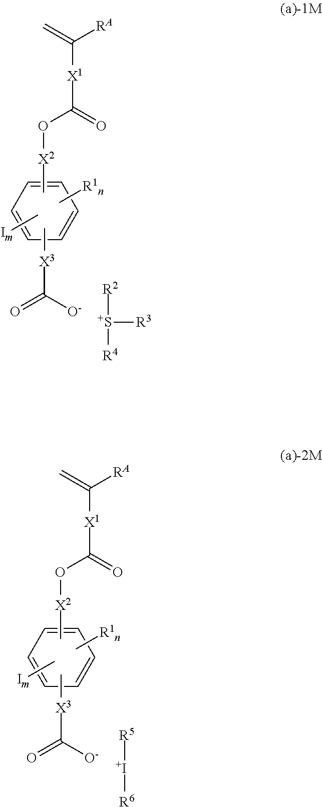

wherein R^{A} represents a hydrogen atom or a methyl group; X¹ represents a single bond, a phenylene group, a naphthylene group, or a linking group having 1 to 12 carbon atoms, including an ester bond or an ether bond, and optionally having a halogen atom; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

[15]: A monomer represented by the following general formula (a)-1′M or (a)-2′M,

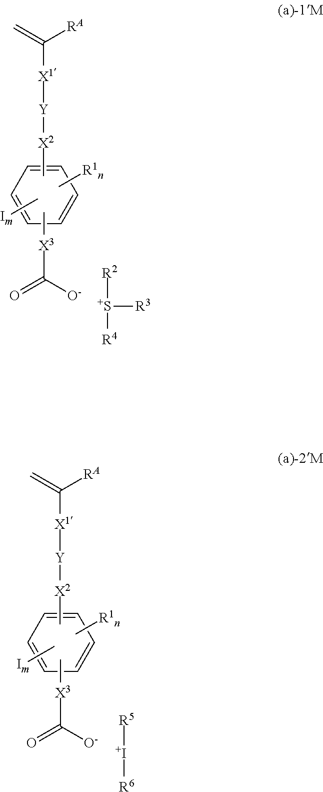

wherein R^{A} represents a hydrogen atom or a methyl group; X¹ represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; Y represents a group selected from an ester group and a sulfonic acid ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. A resist material comprising a repeating unit-a containing a sulfonium salt or iodonium salt of a monovalent aromatic carboxylic acid that is iodinated, is substituted or unsubstituted, and is bonded to a polymer main chain via an ester bond.

2. The resist material according to claim 1, wherein the repeating unit-a contains a repeating unit represented by the following general formula (a)-1 or (a)-2, 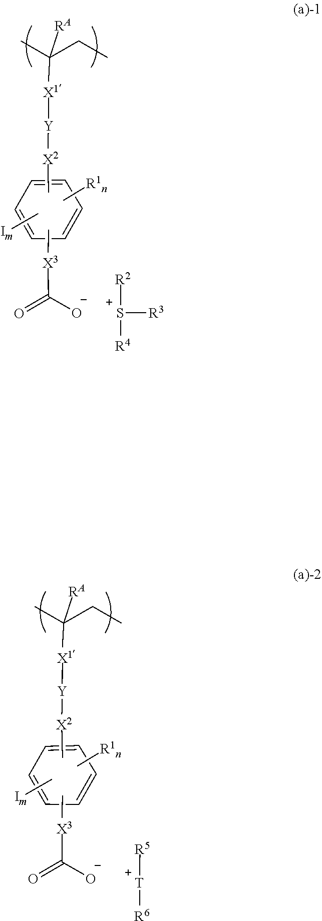
wherein R^{A} represents a hydrogen atom or a methyl group; X^{1′} represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group; X² represents a single bond or a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; Y represents a group selected from an ester group and a sulfonic acid ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

3. The resist material according to claim 2, in the general formulae (a)-1 and (a)-2, the X² represents a linear, branched, or cyclic alkylene group having 1 to 12 carbon atoms and optionally containing one or more selected from an ester group, an ether group, an amide group, a lactone ring, a sultone ring, and a halogen atom.

4. The resist material according to claim 1, further comprising a repeating unit-b, in which a hydrogen atom of a carboxy group, a phenolic hydroxy group, or both is substituted with an acid-labile group.

5. The resist material according to claim 4, wherein the repeating unit-b is at least one kind of repeating unit selected from repeating units represented by the following general formulae (b1) and (b2), 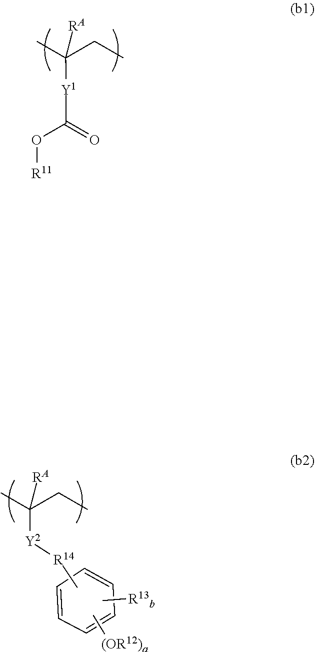
wherein each R^{A} independently represents a hydrogen atom or a methyl group; Y¹ represents a single bond, a phenylene group, a naphthylene group, or a linking group having 1 to 12 carbon atoms and having an ester bond, an ether bond, or a lactone ring; Y² represents a single bond, an ester bond, or an amide bond; R¹¹ and R¹² each represent an acid-labile group; R¹³ represents a fluorine atom, a trifluoromethyl group, a cyano group, or an alkyl group having 1 to 6 carbon atoms; R¹⁴ represents a single bond or a linear or branched alkanediyl group having 1 to 6 carbon atoms, part of carbon atoms of the alkanediyl group optionally being substituted with an ether bond or an ester bond; “a” represents 1 or 2; and “b” represents an integer of 0 to 4.

6. The resist material according to claim 1, further comprising a repeating unit-c having an adhesive group selected from a hydroxy group, a carboxy group, a lactone ring, a carbonate group, a thiocarbonate group, a carbonyl group, a cyclic acetal group, an ether bond, an ester bond, a sulfonic acid ester bond, a cyano group, an amide group, —O—C(═O)—S—, and —O—C(═O)—NH—.

7. The resist material according to claim 1, further comprising at least one kind of repeating unit-d selected from repeating units represented by the following general formulae (d1) to (d3), 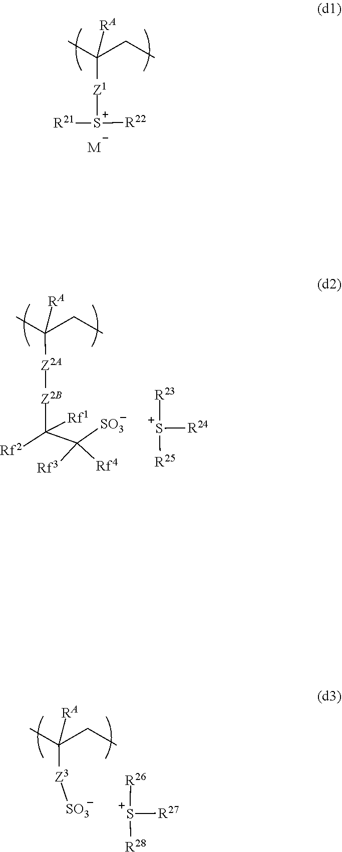
wherein each R^{A} independently represents a hydrogen atom or a methyl group; Z¹ represents a single bond, a phenylene group, —O—Z¹¹—, —C(═O)—O—Z¹¹—, or —C(═O)—NH—Z¹¹—; Z¹¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, and a hydroxy group; Z²A represents a single bond or an ester bond; Z²B represents a single bond or a divalent group having 1 to 12 carbon atoms and optionally containing one or more selected from an ester bond, an ether bond, a lactone ring, a bromine atom, and an iodine atom; Z³ represents a single bond, a methylene group, an ethylene group, a phenylene group, a fluorinated phenylene group, —O—Z³¹—, —C(═O)—O—Z³¹—, or
—C(═O)—NH—Z³¹—; Z³¹ represents an alkanediyl group having 1 to 6 carbon atoms, an alkenediyl group having 2 to 6 carbon atoms, or a phenylene group, and optionally contains one or more selected from a carbonyl group, an ester bond, an ether bond, a halogen atom, and a hydroxy group; Rf¹ to Rf⁴ each independently represent a hydrogen atom, a fluorine atom, or a trifluoromethyl group, provided that at least one of Rf¹ to Rf⁴ is a fluorine atom; R²¹ to R²⁸ each independently represent a monovalent hydrocarbon group having 1 to 20 carbon atoms and optionally containing a heteroatom; any two of R²³, R²⁴, and R²⁵ or any two of R²⁶, R²⁷, and R²⁸ are optionally bonded to each other to form a ring together with a sulfur atom bonded thereto; and M represents a non-nucleophilic counter ion.

8. The resist material according to claim 7, wherein at least one iodine atom is contained in the Z^{2B}.

9. The resist material according to claim 1, wherein the resist material has a molecular weight of 1,000 to 100,000.

10. A resist composition comprising the resist material according to claim 1.

11. The resist composition according to claim 10, further comprising one or more selected from an acid generator, an organic solvent, a quencher, and a surfactant.

12. A patterning process comprising the steps of:
forming a resist film on a substrate by using the resist composition according to claim 10;
exposing the resist film to a high-energy beam; and
developing the exposed resist film by using a developer.

13. The patterning process according to claim 12, wherein the high-energy beam is an i-line, a KrF excimer laser beam, an ArF excimer laser beam, an electron beam, or an extreme ultraviolet ray having a wavelength of 3 to 15 nm.

14. A monomer represented by the following general formula (a)-1M or (a)-2M, 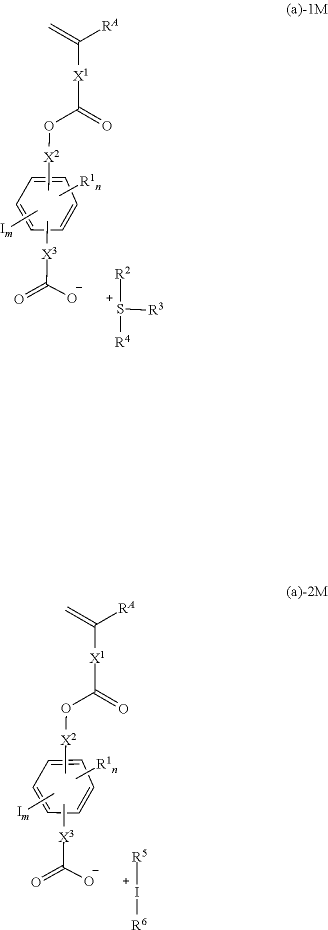
wherein R^{A} represents a hydrogen atom or a methyl group; X¹ represents a single bond, a phenylene group, a naphthylene group, or a linking group having 1 to 12 carbon atoms, including an ester bond or an ether bond, and optionally having a halogen atom; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.

15. A monomer represented by the following general formula (a)-1′M or (a)-2′M, 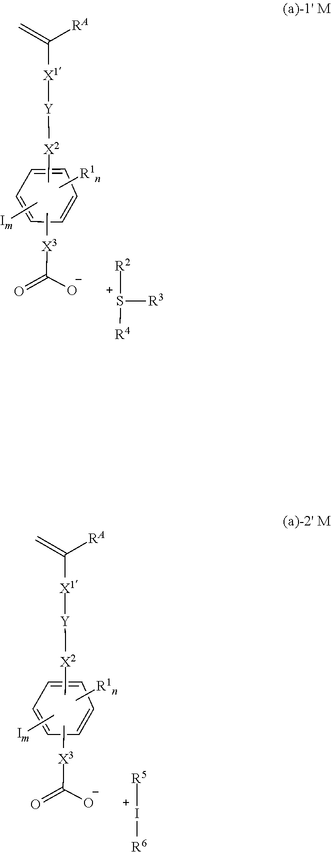
wherein R^{A} represents a hydrogen atom or a methyl group; X^{1′} represents a single bond, a phenylene group, a naphthylene group, or a linking group having 1 to 12 carbon atoms, including an ester bond or an ether bond, and optionally having one or more selected from a halogen atom, a hydroxy group, an alkoxy group, an acyloxy group, a nitro group, and a cyano group; X² represents a linear, branched, or cyclic alkylene group having 1 to 12 carbon atoms and optionally containing one or more selected from an ester group, an ether group, an amide group, a lactone ring, a sultone ring, and a halogen atom; X³ represents a single bond or a linear or branched alkylene group having 1 to 6 carbon atoms, containing no fluorine atom, and optionally having one or more selected from an ether group and an ester group; Y represents a group selected from an ester group and a sulfonic acid ester group; each R¹ independently represents a linear or branched alkyl group, alkoxy group, or acyloxy group having 1 to 4 carbon atoms, or a halogen atom other than an iodine atom; “m” represents an integer of 1 to 4; “n” represents an integer of 0 to 3; R² to R⁶ each independently represent a monovalent hydrocarbon group having 1 to 25 carbon atoms and optionally containing a heteroatom; and any two of R², R³, and R⁴ are optionally bonded to each other to form a ring together with a sulfur atom bonded thereto.
